(19) United States
(12) Patent Application Publication
(10) Pub. No.: US 2025/0340565 A1
(43) Pub. Date: Nov. 6, 2025

(54) CD73 INHIBITOR COMPOUNDS

(71) Applicant: AdoRx Therapeutics Limited, Edinburgh (GB)

(72) Inventors: Clive McCarthy, Edinburgh (GB); Robert Heap, Cheshire (GB); Edward Richard Walker, Oxfordshire (GB); Pearl McMahon, Oxfordshire (GB)

(21) Appl. No.: 18/869,980

(22) PCT Filed: May 26, 2023
(86) PCT No.: PCT/GB23/51393

§ 371 Date: Nov. 27, 2024

(30) Foreign Application Priority Data
May 30, 2022 (GB) 2207972.7
Oct. 14, 2022 (GB) 2215245.8

(51) Int. Cl. C07D 495/04; A61K 31/513; A61K 31/52; A61K 31/53; A61K 31/5377; A61K 31/5386; A61K 45/06; C07D 471/04; C07D 473/34; C07D 487/04; C07D 491/048; C07D 513/04; C07D 519/00

(52) U.S. Cl. CPC C07D 495/04; A61K 31/513; A61K 31/52; A61K 31/53; A61K 31/5377; A61K 31/5386; A61K 45/06; C07D 471/04; C07D 473/34; C07D 487/04; C07D 491/048; C07D 513/04; C07D 519/00

(57) ABSTRACT

The present invention relates to compounds of formula I shown below:

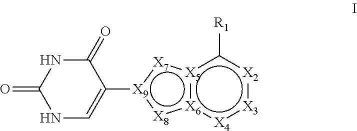

wherein R₁, X₂, X₃, X₄, X₅ X₆, X₇, X₈ and X₉ are each as defined in the application. The present invention also relates to processes for the preparation of these compounds, to pharmaceutical compositions comprising them, and to their use in the treatment of diseases or conditions in which adenosine CD73 activity is implicated, such as, for example, cancer.

### INTRODUCTION

The present invention relates to certain compounds that function as inhibitors of the CD73, also known as 5′-nucleotidase or ecto-5′nucleotidase. The present invention also relates to processes for the preparation of these compounds, to pharmaceutical compositions comprising them, and to their use in the treatment of diseases or conditions in which CD73 activity is implicated, such as, for example, cancer.

### BACKGROUND OF THE INVENTION

Purinergic signalling plays a critical role in physiological processes and imbalance can lead to various pathophysiological conditions. Extracellular adenosine trisphosphate and adenosine levels are regulated by a complex network of enzymes and transporters. Downstream signalling is mediated by ligand gated ion channels and via G-protein coupled receptors. The cell surface protein CD39 acts at an early point in the pathway by catalyzing the hydrolysis of adenosine trisphosphate (ATP) to adenosine diphosphate (ADP) and subsequently the conversion of ADP to AMP. CD73 is a cell surface ecto-5′-nucleotidase encoded by the NT5E gene. CD73 is widely expressed and is responsible for the conversion of adenosine monophosphate (AMP) to adenosine. Aberrant purinergic signalling is observed in a variety of disease including cancer, autoimmune disorders, inflammation, fibrosis, bleeding disorders and atherosclerosis. The disruption of CD73 enzyme activity is seen as a promising approach to balancing ATP mediated immune cell activation and adenosine induced immunosuppression.

In cancer, increased levels of adenosine within the tumour create an immunosuppressive microenvironment. CD73 expression levels are increased in various human tumour types including ovarian cancer, melanoma, prostate cancer, breast cancer, colon cancer, head and neck cancer, leukemia, hepatocellular carcinoma and glioblastoma [Arab and Hadjati, 2019]. High CD73 expression has been associated with poor cancer outcome [Leone and Emens, 2018; Hammami et al, 2019]. CD73 expression can change in response to treatment with chemotherapy and targeted therapies and is also controlled by hypoxia through activation by the transcription factor HIF1a. In addition to expression on tumour cells, CD73 expression on leukocyte and myeloid cells has been summarised by Roh et al (Roh et al, 2020). CD73 is expressed on regulatory T cells and on exhausted or anergic T effector cells. Increased expression of CD73 is observed on myeloid derived suppressor cells in cancer patients.

The growth of tumours is altered in mice harbouring a deletion in the gene encoding CD73 [Yegutkin et al, 2011]. CD73 ablation significantly suppresses the growth of MC38 colon cancer cells, EG7 lymphoma and AT-3 mammary tumour cells and B16F10 melanoma in syngeneic models [Stagg et al, 2011]. Using adoptive transfer studies demonstrated that the protumourigenic effects of CD73 were partly due to expression on hematopoetic cells (e.g. regulatory T cells) and partly due to expression on non-hematopoetic cells such as endothelial cells. Furthermore, this study showed that tumour growth and metastasis could be reduced using a monoclonal antibody targeting CD73. Blockade using the CD73 inhibitor a,b-methyleneadenosine 5′-diphosphate was also shown to reduce tumour growth and decreased metastasis in mice models [Stagg t et al. 2011]. A number of therapeutic antibodies targeting CD73 with distinct mechanisms of action are in clinical development [Allard et al 2018]. MEDI9447 (AstraZeneca) is a human monoclonal antibody that non-competitively inhibits CD73 function by blocking dimerization and by preventing CD73 from transitioning to a catalytically active conformation [Geoghegan J. C. et al]. MEDI9447 was shown to inhibit tumour growth as a single agent and in combination with anti-PD-1 antibodies in animal studies using syngeneic tumours [Hay C. M. et al 2016]. Another antibody, BMS-986179 enhances the internalisation of CD73.

Small molecular weight chemical inhibitors of CD73 have also entered clinical trials including AB680 (Arcus Biosiences) and LY3475070 (Eli Lilly). AB680 is a potent, reversible inhibitor of CD73 catalytic activity [Lawson K. V. et al 2020]. OP-5244 (Oric Pharmaceuticals) is a sub-nanomolar CD73 inhibitor that has been shown to inhibit adenosine production from both cancer cells and T cells in vitro and reversed immunosuppression in mouse models [Du X. et al 2020].

However, there remains a need for further compounds that are potent CD73 inhibitors. In particular, there is a need for compounds that are orally administered, potent and selective CD73 inhibitors.

The present invention was devised with the foregoing in mind.

### REFERENCES

Allard D. et al. (2019) Immunol Lett 205; 31-39.
Arab S. and Hadjait J. (2019) Immune Netw 19 (4) e23
Du, X. et al. (2020) J Med Chem 63; 10433-10459
Geoghegan J. C. et al (2016) MAbs 8; 454-67
Hammami A. et al. (2019) Seminars in Immunology 42
Hay C. M. et al (2016) Oncoimmunology 5; e1208875
Lawson K. V. et al (2020) J Med Chem 63, 20, 11448
Leone R. D. and Emens L. A. (2018) J Immunother Cancer 6(57)
Roh M. et al. (2020) Current Opinion in Pharmacology 53, 1-11
Stagg J. et al. (2011) Cancer Research 71, 2892-900
Yegutkin G. G. et al. (2011) Eur J Immunol 41 (5); 1231-41

### SUMMARY OF THE INVENTION

According to a first aspect of the present invention, there is provided a compound, or a pharmaceutically acceptable salt, hydrate or solvate thereof, as defined herein.

According to a further aspect of the present invention, there is provided a pharmaceutical composition comprising a compound as defined herein, or a pharmaceutically acceptable salt, hydrate or solvate thereof, in admixture with a pharmaceutically acceptable diluent or carrier.

According to a further aspect of the present invention, there is provided a method of inhibiting CD73 in vitro or in vivo, said method comprising contacting a cell with an effective amount of a compound or a pharmaceutically acceptable salt, hydrate or solvate thereof as defined herein.

According to a further aspect of the present invention, there is provided a method of selectively inhibiting CD73 in vitro or in vivo, said method comprising contacting a cell with an effective amount of a compound, or a pharmaceutically acceptable salt, hydrate or solvate thereof, as defined herein.

According to a further aspect of the present invention, there is provided a method of inhibiting cell proliferation, in vitro or in vivo, said method comprising contacting a cell with an effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating a disease or disorder associated CD73 activity in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, as defined herein for use as an inhibitor of CD73 activity. Certain compounds of the invention are selective CD73 inhibitors.

According to a further aspect of the present invention, there is provided a compound, or a pharmaceutically acceptable salt, hydrate or solvate thereof, as defined herein for use in the treatment of a disease or disorder in which CD73 activity is implicated.

According to a further aspect of the present invention, there is provided the use of a compound, or a pharmaceutically acceptable salt, hydrate or solvate thereof, as defined herein in the manufacture of a medicament for the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provide the use of a compound, or a pharmaceutically acceptable salt, hydrate or solvate thereof, as defined herein in the manufacture of a medicament for the treatment of cancer. Suitably, the cancer is a human cancer. Suitably, the compound or pharmaceutical composition is administered in combination with one or more additional anticancer agents (e.g. checkpoint inhibitors and/or cytotoxic agents).

According to a further aspect of the present invention, there is provided a use of a compound, or a pharmaceutically acceptable salt, hydrate or solvate thereof, as defined herein in the manufacture of a medicament for use as an inhibitor of CD73 activity.

According to a further aspect of the present invention, there is provided a use of a compound, or a pharmaceutically acceptable salt, hydrate or solvate thereof, as defined herein in the manufacture of a medicament for the treatment of a disease or disorder in which CD73 activity is implicated.

According to a further aspect of the present invention, there is provided a process for preparing a compound, or a pharmaceutically acceptable salt, hydrate or solvate thereof, as defined herein.

According to a further aspect of the present invention, there is provided a compound, or a pharmaceutically acceptable salt, hydrate or solvate thereof, obtainable by, or obtained by, or directly obtained by a process of preparing a compound as defined herein.

According to a further aspect of the present invention, there are provided novel intermediates as defined herein which are suitable for use in any one of the synthetic methods set out herein.

Features, including optional, suitable, and preferred features in relation to one aspect of the invention may also be features, including optional, suitable and preferred features in relation to any other aspect of the invention.

### DETAILED DESCRIPTION OF THE INVENTION

### Definitions

Unless otherwise stated, the following terms used in the specification and claims have the following meanings set out below.

It is to be appreciated that references to “treating” or “treatment” include prophylaxis as well as the alleviation of established symptoms of a condition. “Treating” or “treatment” of a state, disorder or condition therefore includes: (1) preventing or delaying the appearance of clinical symptoms of the state, disorder or condition developing in a human that may be afflicted with or predisposed to the state, disorder or condition but does not yet experience or display clinical or subclinical symptoms of the state, disorder or condition, (2) inhibiting the state, disorder or condition, i.e., arresting, reducing or delaying the development of the disease or a relapse thereof (in case of maintenance treatment) or at least one clinical or subclinical symptom thereof, or (3) relieving or attenuating the disease, i.e., causing regression of the state, disorder or condition or at least one of its clinical or subclinical symptoms.

A “therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

In this specification the term “alkyl” includes both straight and branched chain alkyl groups. References to individual alkyl groups such as “propyl” are specific for the straight chain version only and references to individual branched chain alkyl groups such as “isopropyl” are specific for the branched chain version only. For Example, “(1-6C)alkyl” includes (1-4C)alkyl, (1-3C)alkyl, methyl, ethyl, n-propyl, isopropyl, n-butyl, sec-butyl, and t-butyl, as well as pentyl and hexyl isomers. A similar convention applies to other radicals, for example “phenyl(1-6C)alkyl” includes phenyl(1-4C)alkyl, benzyl, 1-phenylethyl and 2-phenylethyl etc.

The term “(m-nC)” or “(m-nC) group” used alone or as a prefix, refers to any group having m to n carbon atoms.

An “alkylene,” “alkenylene,” or “alkynylene” group is an alkyl, alkenyl, or alkynyl group that is positioned between and serves to connect two other chemical groups. Thus, “(1-6C)alkylene” means a linear saturated divalent hydrocarbon radical of one to six carbon atoms or a branched saturated divalent hydrocarbon radical of three to six carbon atoms, for example, methylene, ethylene, propylene, 2-methylpropylene, pentylene, and the like.

“(2-6C)alkenylene” means a linear divalent hydrocarbon radical of two to six carbon atoms or a branched divalent hydrocarbon radical of three to six carbon atoms, containing at least one double bond, for example, as in ethenylene, 2,4-pentadienylene, and the like.

“(2-6C)alkynylene” means a linear divalent hydrocarbon radical of two to six carbon atoms or a branched divalent hydrocarbon radical of three to six carbon atoms, containing at least one triple bond, for example, as in ethynylene, propynylene, and butynylene and the like.

“(3-8C)cycloalkyl” means a hydrocarbon ring containing from 3 to 8 carbon atoms, for example, cyclopropyl, cyclobutyl, cyclopentyl, cyclohexyl, cycloheptyl or bicyclo[2.2.1]heptyl.

“(3-8C)cycloalkenyl” means a hydrocarbon ring containing at least one double bond, for example, cyclobutenyl, cyclopentenyl, cyclohexenyl or cycloheptenyl, such as 3-cyclohexen-1-yl, or cyclooctenyl.

“(3-8C)cycloalkyl-(1-6C)alkyl” means a (3-8C)cycloalkyl group covalently attached to a (1-6C)alkylene group, both of which are defined herein.

The term “halo” or “halogeno” refers to fluoro, chloro, bromo and iodo.

The term “heterocyclyl”, “heterocyclic” or “heterocycle” means a non-aromatic saturated or partially saturated monocyclic, fused, bridged, or spiro bicyclic heterocyclic ring system(s). Monocyclic heterocyclic rings contain from about 3 to 12 (suitably from 3 to 7) ring atoms, with from 1 to 5 (suitably 1, 2 or 3) heteroatoms selected from nitrogen, oxygen or sulfur in the ring. Bicyclic heterocycles contain from 7 to 17 member atoms, suitably 7 to 12 member atoms, in the ring. Bicyclic heterocyclic(s) rings may be fused, spiro, or bridged ring systems. Examples of heterocyclic groups include cyclic ethers such as oxiranyl, oxetanyl, tetrahydrofuranyl, dioxanyl, and substituted cyclic ethers. Heterocycles containing nitrogen include, for example, azetidinyl, pyrrolidinyl, piperidinyl, piperazinyl, tetrahydrotriazinyl, tetrahydropyrazolyl, and the like. Typical sulfur containing heterocycles include tetrahydrothienyl, dihydro-1,3-dithiol, tetrahydro-2H-thiopyran, and hexahydrothiepine. Other heterocycles include dihydrooxathiolyl, tetrahydrooxazolyl, tetrahydro-oxadiazolyl, tetrahydrodioxazolyl, tetrahydrooxathiazolyl, hexahydrotriazinyl, tetrahydrooxazinyl, morpholinyl, thiomorpholinyl, tetrahydropyrimidinyl, dioxolinyl, octahydrobenzofuranyl, octahydrobenzimidazolyl, and octahydrobenzothiazolyl. For heterocycles containing sulfur, the oxidized sulfur heterocycles containing SO or SO₂ groups are also included. Examples include the sulfoxide and sulfone forms of tetrahydrothienyl and thiomorpholinyl such as tetrahydrothiene 1,1-dioxide and thiomorpholinyl 1,1-dioxide. A suitable value for a heterocyclyl group which bears 1 or 2 oxo (═O) or thioxo (═S) substituents is, for example, 2-oxopyrrolidinyl, 2-thioxopyrrolidinyl, 2-oxoimidazolidinyl, 2-thioxoimidazolidinyl, 2-oxopiperidinyl, 2,5-dioxopyrrolidinyl, 2,5-dioxoimidazolidinyl or 2,6-dioxopiperidinyl. Particular heterocyclyl groups are saturated monocyclic 3 to 7 membered heterocyclyls containing 1, 2 or 3 heteroatoms selected from nitrogen, oxygen or sulfur, for example azetidinyl, tetrahydrofuranyl, tetrahydropyranyl, pyrrolidinyl, morpholinyl, tetrahydrothienyl, tetrahydrothienyl 1,1-dioxide, thiomorpholinyl, thiomorpholinyl 1,1-dioxide, piperidinyl, homopiperidinyl, piperazinyl or homopiperazinyl. As the skilled person would appreciate, any heterocycle may be linked to another group via any suitable atom, such as via a carbon or nitrogen atom. However, reference herein to piperidino or morpholino refers to a piperidin-1-yl or morpholin-4-yl ring that is linked via the ring nitrogen.

By “bridged ring systems” is meant ring systems in which two rings share more than two atoms, see for example Advanced Organic Chemistry, by Jerry March, 4^{th} Edition, Wiley Interscience, pages 131-133, 1992. Examples of bridged heterocyclyl ring systems include, aza-bicyclo[2.2.1]heptane, 2-oxa-5-azabicyclo[2.2.1]heptane, aza-bicyclo[2.2.2]octane, aza-bicyclo[3.2.1]octane and quinuclidine.

By “spiro bi-cyclic ring systems” we mean that the two ring systems share one common spiro carbon atom, i.e. the heterocyclic ring is linked to a further carbocyclic or heterocyclic ring through a single common spiro carbon atom. Examples of spiro ring systems include 6-azaspiro[3.4]octane, 2-oxa-6-azaspiro[3.4]octane, 2-azaspiro[3.3]heptanes, 2-oxa-6-azaspiro[3.3]heptanes, 7-oxa-2-azaspiro[3.5]nonane, 6-oxa-2-azaspiro[3.4]octane, 2-oxa-7-azaspiro[3.5]nonane and 2-oxa-6-azaspiro[3.5]nonane.

“Heterocyclyl(1-6C)alkyl” means a heterocyclyl group covalently attached to a (1-6C)alkylene group, both of which are defined herein.

The term “heteroaryl” or “heteroaromatic” means an aromatic mono-, bi-, or polycyclic ring incorporating one or more (for example 1-4, particularly 1, 2 or 3) heteroatoms selected from nitrogen, oxygen or sulfur. The term heteroaryl includes both monovalent species and divalent species. Examples of heteroaryl groups are monocyclic and bicyclic groups containing from five to twelve ring members, and more usually from five to ten ring members. The heteroaryl group can be, for example, a 5- or 6-membered monocyclic ring or a 9- or 10-membered bicyclic ring, for example a bicyclic structure formed from fused five and six membered rings or two fused six membered rings. Each ring may contain up to about four heteroatoms typically selected from nitrogen, sulfur and oxygen. Typically, the heteroaryl ring will contain up to 3 heteroatoms, more usually up to 2, for example a single heteroatom. In one embodiment, the heteroaryl ring contains at least one ring nitrogen atom. The nitrogen atoms in the heteroaryl rings can be basic, as in the case of an imidazole or pyridine, or essentially non-basic as in the case of an indole or pyrrole nitrogen. In general, the number of basic nitrogen atoms present in the heteroaryl group, including any amino group substituents of the ring, will be less than five.

Examples of heteroaryl include furyl, pyrrolyl, thienyl, oxazolyl, isoxazolyl, imidazolyl, pyrazolyl, thiazolyl, isothiazolyl, oxadiazolyl, thiadiazolyl, triazolyl, tetrazolyl, pyridyl, pyridazinyl, pyrimidinyl, pyrazinyl, 1,3,5-triazenyl, benzofuranyl, indolyl, isoindolyl, benzothienyl, benzoxazolyl, benzimidazolyl, benzothiazolyl, benzothiazolyl, indazolyl, purinyl, benzofurazanyl, quinolyl, isoquinolyl, quinazolinyl, quinoxalinyl, cinnolinyl, pteridinyl, naphthyridinyl, carbazolyl, phenazinyl, benzisoquinolinyl, pyridopyrazinyl, thieno[2,3b]-furanyl-, 2H-furo[3,2b]-pyranyl-, 5H-pyrido[2,3-d]-ooxazinyl-, 1H-pyrazolo[4,3-d]-oxazolyl, 4H-imidazo[4,5d]thiazolyl, pyrazino[2,3d]pyridazinyl, -imidazo[2,1b]thiazolyl, -imidazo[1,2b][1,2,4]-triazinyl. “Heteroaryl” also covers partially aromatic bi- or polycyclic ring systems wherein at least one ring is an aromatic ring and one or more of the other ring(s) is a nonaromatic, saturated or partially saturated ring, provided at least one ring contains one or more heteroatoms selected from nitrogen, oxygen or -sulfur-. Examples of partially aromatic heteroaryl groups include for example, tetrahydroisoquinolinyl, tetrahydroquinolinyl, 2-oxo-1,2,3,4-tetrahydroquinolinyl, dihydrobenzthienyl, dihydrobenzfuranyl, 2,3-dihydro-benzo[1,4]dioxinyl, benzo[1,3]dioxolyl, 2,2-dioxo-1,3-dihydro-2-benzothienyl, 4,5,6,7-tetrahydrobenzofuranyl, indolinyl, 1,2,3,4-tetrahydro-1,8-naphthyridinyl, 1,2,3,4-tetrahydropyrido[2,3-b]pyrazinyl, 3,4-dihydro-2H-pyrido[3,2-b][1,4]oxazinyl and 6,8-dihydro-5H-[1,2,4]triazolo[4,3-a]pyrazinyl.

Examples of five membered heteroaryl groups include but are not limited to pyrrolyl, furanyl, thienyl, imidazolyl, furazanyl, oxazolyl, oxadiazolyl, oxatriazolyl, isoxazolyl, thiazolyl, isothiazolyl, pyrazolyl, triazolyl and tetrazolyl groups.

Examples of six membered heteroaryl groups include but are not limited to pyridyl, pyrazinyl, pyridazinyl, pyrimidinyl and triazinyl.

A bicyclic heteroaryl group may be, for example, a group selected from:
a benzene ring fused to a 5- or 6-membered ring containing 1, 2 or 3 ring heteroatoms;
a pyridine ring fused to a 5- or 6-membered ring containing 1, 2 or 3 ring heteroatoms;
a pyrimidine ring fused to a 5- or 6-membered ring containing 1 or 2 ring heteroatoms;
a pyrrole ring fused to a 5- or 6-membered ring containing 1, 2 or 3 ring heteroatoms;
a pyrazole ring fused to a 5- or 6-membered ring containing 1 or 2 ring heteroatoms;
a pyrazine ring fused to a 5- or 6-membered ring containing 1 or 2 ring heteroatoms;
an imidazole ring fused to a 5- or 6-membered ring containing 1 or 2 ring heteroatoms;
an oxazole ring fused to a 5- or 6-membered ring containing 1 or 2 ring heteroatoms;
an isoxazole ring fused to a 5- or 6-membered ring containing 1 or 2 ring heteroatoms;
a thiazole ring fused to a 5- or 6-membered ring containing 1 or 2 ring heteroatoms;
an isothiazole ring fused to a 5- or 6-membered ring containing 1 or 2 ring heteroatoms;
a thiophene ring fused to a 5- or 6-membered ring containing 1, 2 or 3 ring heteroatoms;
a furan ring fused to a 5- or 6-membered ring containing 1, 2 or 3 ring heteroatoms;
a cyclohexyl ring fused to a 5- or 6-membered heteroaromatic ring containing 1, 2 or 3 ring heteroatoms; and
a cyclopentyl ring fused to a 5- or 6-membered heteroaromatic ring containing 1, 2 or 3 ring heteroatoms.

Particular Examples of bicyclic heteroaryl groups containing a six membered ring fused to a five membered ring include but are not limited to benzfuranyl, benzthiophenyl, benzimidazolyl, benzoxazolyl, benzisoxazolyl, benzthiazolyl, benzisothiazolyl, isobenzofuranyl, indolyl, isoindolyl, indolizinyl, indolinyl, isoindolinyl, purinyl (e.g., adeninyl, guaninyl), indazolyl, benzodioxolyl and pyrazolopyridinyl groups.

Particular Examples of bicyclic heteroaryl groups containing two fused six membered rings include but are not limited to quinolinyl, isoquinolinyl, chromanyl, thiochromanyl, chromenyl, isochromenyl, chromanyl, isochromanyl, benzodioxanyl, quinolizinyl, benzoxazinyl, benzodiazinyl, pyridopyridinyl, quinoxalinyl, quinazolinyl, cinnolinyl, phthalazinyl, naphthyridinyl and pteridinyl groups.

“Heteroaryl(1-6C)alkyl” means a heteroaryl group covalently attached to a (1-6C)alkylene group, both of which are defined herein. Examples of heteroaralkyl groups include pyridin-3-ylmethyl, 3-(benzofuran-2-yl)propyl, and the like.

The term “aryl” means a cyclic or polycyclic aromatic ring having from 5 to 12 carbon atoms. The term aryl includes both monovalent species and divalent species. Examples of aryl groups include, but are not limited to, phenyl, biphenyl, naphthyl and the like. In particular embodiment, an aryl is phenyl.

The term “aryl(1-6C)alkyl” means an aryl group covalently attached to a (1-6C)alkylene group, both of which are defined herein. Examples of aryl-(1-6C)alkyl groups include benzyl, phenylethyl, and the like.

This specification also makes use of several composite terms to describe groups comprising more than one functionality. Such terms will be understood by a person skilled in the art. For Example heterocyclyl(m-nC)alkyl comprises (m-nC)alkyl substituted by heterocyclyl.

The term “optionally substituted” refers to either groups, structures, or molecules that are substituted and those that are not substituted. The term “wherein a/any CH, CH₂, CH₃ group or heteroatom (i.e. NH) within a R¹ group is optionally substituted” suitably means that (any) one of the hydrogen radicals of the R¹ group is substituted by a relevant stipulated group.

Where optional substituents are chosen from “one or more” groups it is to be understood that this definition includes all substituents being chosen from one of the specified groups or the substituents being chosen from two or more of the specified groups.

The phrase “compound of the invention” means those compounds which are disclosed herein, both generically and specifically.

### Compounds of the Invention

In a first aspect, the present invention relates to compounds, or pharmaceutically acceptable salts, hydrates or solvates thereof, having the structural formula I shown below:

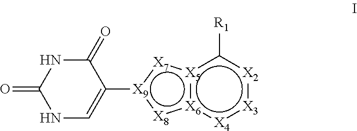

wherein:
R₁ is selected from:
(i) —NR_{1A}R_{1B};
   wherein R_{1A} and R_{1B} are each independently selected from hydrogen, (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl;
   or
   or R_{1A} and R_{1B} are linked such that, together with the nitrogen atom to which they are attached, they form a heterocyclic ring;
      and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
(ii) a carbon-linked heterocyclyl optionally substituted by one or more Rₓ;
(iii) —O—R_{1C};
   wherein R_{1C} is selected from (1-6C)alkyl, (3-8C)cycloalkyl, (3-8C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
(iv) (1-6C)alkyl optionally substituted by one or more Rₓ;
(v) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, wherein any alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl or heterocyclyl(1-2C)alkyl group is optionally substituted by one or more Rₓ;
(vi) a carbon or nitrogen-linked heteroaryl ring optionally substituted by one or two substituents independently selected from fluoro, (1-4C)alkyl, (1-4C)haloalkyl, (1-4C)alkoxy, (1-4C)haloalkoxy, hydroxy or (1-4C)hydroxyalkyl;
wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, (CH₂)_{q1}C(O)OR_{1D}, O(CH₂)_{q1}C(O)R_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
      wherein q1 is 0, 1, 2 or 3; and
      wherein R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl;
      and R_{1E} is selected from hydrogen, (1-4C)alkyl, (3-6C)cycloalkyl, (3-6C)cycloalkyl(1-4C)alkyl, aryl or aryl(1-4C)alkyl;
      or R_{1D} and R_{1E} are linked such that, together with the atom or atoms to which they are attached, they form a 4 to 10 membered heterocyclic ring;
   and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
      wherein q2 is 0, 1, 2 or 3; and
      wherein R_{1F} and R_{1G} are each independently selected from hydrogen or (1-4C)alkyl optionally substituted by halo; and
X₂ is selected from N or CR₂; wherein R₂ is selected from hydrogen, halo, (1-3C)alkyl, (1-3C)alkoxy, (1-3C)haloalkyl, (1-3C)haloalkoxy or cyano;
X₃ is selected from N or CR₃; wherein R₃ is selected from hydrogen, halo (1-3C)alkyl, (1-3C)alkoxy, (1-3C)haloalkyl, (1-3C)haloalkoxy or cyano,
X₄ is selected from N or CR₄; wherein R₄ is selected from hydrogen, halo (e.g. fluoro or chloro), (1-3C)alkyl, (1-3C)alkoxy, (1-3C)haloalkyl, (1-3C)haloalkoxy or cyano;
X₅ is selected from N or C;
X₆ is selected from N or C;
X₇ is selected from N, NR_{7N}, CR₇, O or S; wherein:
   R₇ is selected from hydrogen, halo, methyl or methoxy;
   R_{7N} is selected from hydrogen or methyl;
X₈ is selected from N, NR_{8N}, CR₈, O or S; wherein:
   R₈ is selected from hydrogen, halo, methyl or methoxy;
   R_{8N} is selected from hydrogen or methyl;
X₉ is selected from N or C;
with the proviso that:
   (i) X₅ and X₆ cannot both be N;
   (ii) only one of X₇ and X₈ can be O or S;
   (iii) no more than four of the atoms in groups X₂, X₃, X₄, X₅, X₆, X₇, X₈ and X₉ which, together with the carbon atom attached to R₁, make up the bicyclic ring, may be a heteroatom.

In an aspect, the present invention relates to compounds, or pharmaceutically acceptable salts, hydrates or solvates thereof, having the structural formula I-I shown below (a sub formula of formula I):

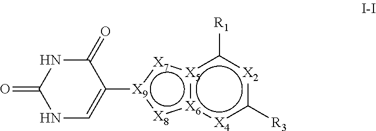

wherein:
R₁ is selected from:
(i) —NR_{1A}R_{1B};
   wherein R_{1A} and R_{1B} are each independently selected from hydrogen, (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl;
   or R_{1A} and R_{1B} are linked such that, together with the nitrogen atom to which they are attached, they form a heterocyclic ring;
      and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
(ii) a carbon-linked heterocyclyl optionally substituted by one or more Rₓ;
(iii) —O—R_{1C};
   wherein R_{1C} is selected from (1-6C)alkyl, (3-8C)cycloalkyl, (3-8C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
(iv) (1-6C)alkyl optionally substituted by one or more Rₓ;
(v) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, wherein any alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl or heterocyclyl(1-2C)alkyl group is optionally substituted by one or more Rₓ;
(vi) a carbon or nitrogen-linked heteroaryl ring optionally substituted by one or two substituents independently selected from fluoro, (1-4C)alkyl, (1-4C)haloalkyl, (1-4C)alkoxy, (1-4C)haloalkoxy, hydroxy or (1-4C)hydroxyalkyl;
wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, (CH₂)_{q1}C(O)OR_{1D}, O(CH₂)_{q1}C(O)R_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
      wherein q1 is 0, 1, 2 or 3; and
      wherein R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl;
      and R_{1E} is selected from hydrogen, (1-4C)alkyl, (3-6C)cycloalkyl, (3-6C)cycloalkyl(1-4C)alkyl, aryl or aryl(1-4C)alkyl;
      or R_{1D} and R_{1E} are linked such that, together with the atom or atoms to which they are attached, they form a 4 to 10 membered heterocyclic ring;
   and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl group formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
      wherein q2 is 0, 1, 2 or 3; and
      wherein R_{1F} and R_{1G} are each independently selected from hydrogen or (1-4C)alkyl optionally substituted by halo; and
X₂ is selected from N or CR₂; wherein R₂ is selected from hydrogen, halo, (1-3C)alkyl, (1-3C)alkoxy, (1-3C)haloalkyl, (1-3C)haloalkoxy or cyano;
R₃ is selected from hydrogen, halo, (1-3C)alkyl, (1-3C)alkoxy, (1-3C)haloalkyl, (1-3C)haloalkoxy or cyano,
X₄ is selected from N or CR₄; wherein R₄ is selected from hydrogen, halo, (1-3C)alkyl, (1-3C)alkoxy, (1-3C)haloalkyl, (1-3C)haloalkoxy or cyano;
X₅ is selected from N or C;
X₆ is selected from N or C;
X₇ is selected from N, NR_{7N}, CR₇, O or S; wherein:
   R₇ is selected from hydrogen, halo, methyl or methoxy;
   R_{7N} is selected from hydrogen or methyl;
X₈ is selected from N, NH, CH, O or S;
X₉ is selected from N or C;
with the proviso that:
   (i) X₅ and X₆ cannot both be N;
   (ii) only one of X₇ and X₈ can be O or S;
   (iii) no more than four of the atoms in groups X₂, X₄, X₅, X₆, X₇, X₈ and X₉ which, together with the carbon atoms attached to R₁ and R₃, make up the bicyclic ring, may be a heteroatom.

Particular compounds of the invention include, for example, compounds of the formula I or I-I, or pharmaceutically acceptable salts, hydrates and/or solvates thereof, wherein, unless otherwise stated, each of R₁, X₂, X₃, X₄, X₅, X₆, X₇, X₈ and X₉, and any associated sub groupshave any of the meanings defined hereinbefore or in any of paragraphs (1) to (24) hereinafter:
(1) R₁ is selected from:
   (i) —NR_{1A}R_{1B};
      wherein R_{1A} and R_{1B} are each independently selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, or R_{1A} and R_{1B} are linked such that, together with the nitrogen atom to which they are attached, they form a heterocyclic ring;
      and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
   (ii) a carbon-linked heterocyclyl optionally substituted by one or more Rₓ;
   (iii) —O—R_{1C};
      wherein R_{1C} is selected from (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
   (iv) (1-6C)alkyl optionally substituted by one or more Rₓ;
   (v) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, (3-6C)cycloalkyl(1-2C)alkyl, aryl, heteroaryl, or heterocyclyl;
   (vi) a carbon or nitrogen-linked monocyclic heteroaryl ring optionally substituted by one or two substituents independently selected from fluoro, (1-3C)alkyl, (1-3C)haloalkyl, (1-3C)alkoxy, (1-3C)haloalkoxy, hydroxy or (1-3C)hydroxyalkyl;
   wherein each Rₓ is independently selected from:
      halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
      wherein
         q1 is 0, 1, or 2;
         R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl; and
         R_{1E} is selected from hydrogen, (1-4C)alkyl, (3-6C)cycloalkyl, (3-6C)cycloalkyl(1-4C)alkyl, aryl or aryl(1-4C)alkyl;
         or R_{1D} and R_{1E} are linked such that, together with the atom or atoms to which they are attached, they form a 4 to 10 membered heterocyclic ring;
      and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
      wherein q2 is 0, 1, or 2; and
wherein R_{1F} and R_{1G} are each independently selected from hydrogen or (1-4C)alkyl optionally substituted by halo.
(2) R₁ is selected from:
   (i) —NR_{1A}R_{1B};
      wherein R_{1A} and R_{1B} are each independently selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl,
      or R_{1A} and R_{1B} are linked such that, together with the nitrogen atom to which they are attached, they form a heterocyclic ring;
      and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl or heterocyclyl group formed by R_{1A} and R_{1B} is optionally substituted by one or more Rₓ;
   (ii) a carbon-linked heterocyclyl optionally substituted by one or more Rₓ;
   (iii) —O—R_{1C};
      wherein R_{1C} is selected from (1-6C)alkyl, (3-6C)cycloalkyl, (3-6C)cycloalkyl(1-2C)alkyl, phenyl, or phenyl(1-2C)alkyl, each of which is optionally substituted by one or more Rₓ;
   (iv) (3C)cycloalkyl optionally substituted by one or two substituents independently selected from fluoro, (1-4C)alkyl, (1-2C)fluoroalkyl, methoxy, trifluoromethoxy, hydroxy, hydroxymethyl or phenyl;
   wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 10-membered heterocyclyl, (4- to 10-membered)heterocyclyl(1-4C)alkyl, or a spiro-fused (3-6C)cycloalkyl or 4- to 6-membered heterocyclyl;
      wherein q1 is 0, 1, or 2; and
      wherein R_{1D} is selected from hydrogen, (1-4C)alkyl, (3-6C)cycloalkyl, (3-6C)cycloalkyl(1-4C)alkyl, phenyl, phenyl(1-4C)alkyl, heteroaryl, heteroaryl(1-4C)alkyl, heterocyclyl, or heterocyclyl(1-4C)alkyl;
      and R_{1E} is selected from hydrogen or (1-4C)alkyl;
      or R_{1D} and R_{1E} are linked such that, together with the atom or atoms to which they are attached, they form a 4 to 10 membered heterocyclic ring;
   and wherein any (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 10-membered heterocyclyl, (4- to 10-membered)heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-2C)alkyl, (1-2C)haloalkyl, (1-2C)hydroxyalkyl, (CH₂)_{q2}NR_{1F} R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F};
   wherein q2 is 0 or 1; and
      wherein R_{1F} and R_{1G} are each independently selected from hydrogen or (1-2C)alkyl.
(3) R₁ is selected from:
   (i) —NR_{1A}R_{1B};
      wherein one of R_{1A} and R_{1B} is hydrogen, and the other is selected from (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl;
      or R_{1A} and R_{1B} are linked such that, together with the nitrogen atom to which they are attached, they form a 4 to 10-membered heterocyclic ring system (including 4-, 5- or 6-membered monocyclic heterocyclic rings, 5 to 9 membered bridged heterocyclic rings, 5 to 10 membered bridged fused heterocyclic rings and 5 to 10 membered bridged spirocyclic heterocyclic rings) and wherein any (1-4C)alkyl, (3-6C)cycloalkyl, aryl, aryl(1-2C)alkyl, heteroaryl, heteroaryl(1-2C)alkyl, or 4 to 10 membered heterocyclyl group (including those formed by R_{1A} and R_{1B}) is optionally substituted by one or more Rₓ;
      which is optionally substituted by one or more Rₓ;
   (ii) a carbon-linked 4 to 10-membered heterocyclyl (including 4-, 5- or 6-membered monocyclic heterocyclic rings, fused heterocyclic rings and spirocyclic heterocyclic rings) optionally substituted by one or more Rₓ;
   (iii) —O—R_{1C}; wherein R_{1C} is selected from (1-6C)alkyl, (3-6C)cycloalkyl, (3-6C)cycloalkyl(1-2C)alkyl, phenyl, or phenyl(1-2C)alkyl, each of which is optionally substituted by one or more Rₓ;
   (iv) (3C)cycloalkyl optionally substituted by one or two substituents independently selected from fluoro, (1-4C)alkyl, (1-2C)fluoroalkyl, methoxy, trifluoromethoxy, hydroxy, hydroxymethyl or phenyl;
   wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl(1-4C)alkyl, phenyl, phenyl(1-4C)alkyl, (5- or 6-membered)heteroaryl(1-4C)alkyl, 4- to 9-membered heterocyclyl, (4- to 9-membered)heterocyclyl(1-3C)alkyl, or a spiro-fused (3-6C)cycloalkyl or 4- to 6-membered heterocyclyl;
      wherein q1 is 0 or 1; and
      wherein R_{1D} is selected from hydrogen, (1-4C)alkyl, (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 9-membered heterocyclyl, or (4- to 9-membered)heterocyclyl(1-4C)alkyl;
      and R_{1E} is selected from hydrogen or (1-4C)alkyl;
      or R_{1D} and R_{1E} are linked such that, together with the atom or atoms to which they are attached, they form a 4 to 10 membered heterocyclic ring;
   and wherein any (1-4C)alkyl, (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 9-membered heterocyclyl, or (4- to 9-membered)heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-2C)alkyl, (1-2C)haloalkyl, (1-2C)hydroxyalkyl, NR_{1F}R_{1G}, OR_{1F}, C(O)R_{1F}, C(O)OR_{1F}, OC(O)R_{1F}, C(O)N(R_{1G})R_{1F}, N(R_{1G})C(O)R_{1F}, or S(O)ₚR_{1F} (where p is 0, 1 or 2);
      wherein R_{1F} and R_{1G} are each independently selected from hydrogen or (1-2C)alkyl.
(4) R₁ is selected from:
   (i) —NR_{1A}R_{1B};
      wherein one of R_{1A} and R_{1B} hydrogen, and the other is selected from (1-4C)alkyl, aryl(1-2C)alkyl or heteroaryl(1-2C)alkyl, each of which being optionally substituted by one or more Rₓ; or
      R_{1A} and R_{1B} are linked such that, together with the nitrogen atom to which they are attached, they form a 4-, 5- or 6-membered monocyclic heterocyclic ring, a 5 to 10-membered fused heterocyclic ring, a 5 to 10-membered bridged heterocyclic ring or a 5 to 10-membered spirocyclic heterocyclic ring, each of which being optionally substituted by one or more Rₓ;
   (ii) a carbon-linked 4 to 10-membered heterocyclyl (including 4-, 5- or 6-membered monocyclic heterocyclic rings, fused heterocyclic rings, bridged heterocyclic rings and spirocyclic heterocyclic rings) optionally substituted by one or more Rₓ;
   (iii) (3C)cycloalkyl optionally substituted by one or two substituents independently selected from fluoro, (1-4C)alkyl, (1-2C)fluoroalkyl, methoxy, trifluoromethoxy, hydroxy, hydroxymethyl or phenyl;
   wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl(1-3C)alkyl, phenyl, phenyl(1-3C)alkyl, (5- or 6-membered)heteroaryl(1-3C)alkyl, (4- to 6-membered)heterocyclyl(1-3C)alkyl, or a spiro-fused (3-6C)cycloalkyl or 4- to 6-membered heterocyclyl;
   wherein q1 is 0 or 1;
      wherein R_{1D} is selected from hydrogen, (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
      and R_{1E} is selected from hydrogen or (1-2C)alkyl;
      or R_{1D} and R_{1E} are linked such that, together with the atom or atoms to which they are attached, they form a 4 to 9 membered heterocyclic ring;
   and wherein any (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, NR_{1F}R_{1G}, OR_{1F}, C(O)R_{1F} and C(O)OR_{1F} (e.g. halo, cyano, hydroxy or (1-4C)alkyl);
      wherein R_{1F} and R_{1G} are each independently selected from hydrogen or (1-2C)alkyl.
(5) R₁ is selected from:
   (i) —NR_{1A}R_{1B};
      wherein one of R_{1A} and R_{1B} hydrogen, and the other is selected from (1-4C)alkyl or phenyl(1-2C)alkyl, each of which being optionally substituted by one or more Rₓ; or
      R_{1A} and R_{1B} are linked such that, together with the nitrogen atom to which they are attached, they form a 4-, 5- or 6-membered monocyclic heterocyclic ring, a 6 to 10 membered fused heterocyclic ring, a 6 to 10 membered bridged heterocyclic ring or a 6 to 10 membered spirocyclic heterocyclic ring, each of which being optionally substituted by one or more Rₓ; or
   (ii) a carbon-linked a 4-, 5- or 6-membered monocyclic heterocyclic ring, a 6 to 10 membered fused heterocyclic ring, a 6 to 10 membered bridged heterocyclic ring or a 6 to 10 membered spirocyclic heterocyclic ring, each of which being optionally substituted by one or more Rₓ;
   (iii) (3C)cycloalkyl optionally substituted by one or two substituents independently selected from fluoro, (1-4C)alkyl, (1-2C)fluoroalkyl, methoxy, trifluoromethoxy, hydroxy, hydroxymethyl or phenyl;
   wherein each Rₓ is independently selected from halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D} or (CH₂)_{q1}N(R_{1E})C(O)R_{1D};
   wherein q1 is 0 or 1;
      wherein R_{1D} is selected from (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
      and R_{1E} is selected from hydrogen or (1-2C)alkyl;
      or R_{1D} and R_{1E} are linked such that, together with the atom or atoms to which they are attached, they form a 4 to 8 membered heterocyclic ring;
      and wherein any (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-2C)alkyl, (1-2C)haloalkyl, (1-2C)hydroxyalkyl, NR_{1F}R_{1G}, OR_{1F}, C(O)R_{1F} and C(O)OR_{1F} (e.g. halo, cyano, hydroxy or (1-4C)alkyl);
      wherein R_{1F} and R_{1G} are each independently selected from hydrogen or (1-2C)alkyl.
(6) R₁ is selected from:
   (i) —NR_{1A}R_{1B};
      wherein one of R_{1A} and R_{1B} hydrogen, and the other is selected from (1-4C)alkyl or phenyl(1-2C)alkyl, each of which being optionally substituted by one or more Rₓ; or
      R_{1A} and R_{1B} are linked such that, together with the nitrogen atom to which they are attached, they form a 4-, 5- or 6-membered monocyclic heterocyclic ring, a 7 to 9 membered fused heterocyclic ring or a 7 to 9 membered spirocyclic heterocyclic ring, each of which being optionally substituted by one or more Rₓ; or
   (ii) a carbon-linked 4-, 5- or 6-membered monocyclic heterocyclic ring, a 7 to 9 membered fused heterocyclic ring or a 7 to 9 membered spirocyclic heterocyclic ring, each of which being optionally substituted by one or more Rₓ;
   (iii) (3C)cycloalkyl optionally substituted by one or two substituents independently selected from fluoro, (1-4C)alkyl, (1-2C)fluoroalkyl, methoxy, trifluoromethoxy, hydroxy, hydroxymethyl or phenyl;
   wherein each Rₓ is independently selected from halo, cyano, (1-4C)alkyl, (CH₂)_{q1}OR_{1D}, O(CH₂)_{q1}C(O)R_{1D} or O(CH₂)_{q1}C(O)N(R_{1E})R_{1D}, wherein q1 is 0 or 1;
      wherein R_{1D} is selected from (1-4C)alkyl, phenyl, phenyl(1-2C)alkyl, 5- or 6-membered heteroaryl, (5- or 6-membered)heteroaryl(1-2C)alkyl, 4- to 9-membered heterocyclyl, or (4- to 9-membered)heterocyclyl(1-2C)alkyl;
      and R_{1E} is selected from hydrogen or methyl;
      or R_{1D} and R_{1E} are linked such that, together with the atom or atoms to which they are attached, they form a 4 to 8 membered heterocyclic ring;
   and wherein any (1-4C)alkyl, phenyl, phenyl(1-2C)alkyl, (5- or 6-membered)heteroaryl, (5- or 6-membered)heteroaryl(1-2C)alkyl, (4- to 9-membered)heterocyclyl or (4- to 9-membered)heterocyclyl(1-2C)alkyl group present in a Rₓ, R_{1D} and R_{1E} substituent, or a heterocyclyl groups formed when R_{1D} and R_{1E} are linked, is optionally further substituted by halo, cyano or hydroxy (1-2C)alkyl or (1-2C)haloalkyl.
(7) R₁ is is —NR_{1A}R_{1B};
   wherein one of R_{1A} and R_{1B} hydrogen, and the other is other is selected from (1-4C)alkyl or phenyl(1-2C)alkyl, each of which being optionally substituted by one or more Rₓ; or
   wherein R_{1A} and R_{1B} are linked such that, together with the nitrogen atom to which they are attached, they form a 4- or 5-membered heterocyclic ring system, a 7 to 9 membered fused heterocyclic ring or a 7 to 9 membered spirocyclic heterocyclic ring each of which being optionally substituted by one or more Rₓ;
   wherein each Rₓ is independently selected from halo, cyano, (1-2C)alkyl, OR_{1D}, OC(O)R_{1D} or OC(O)N(R_{1E})R_{1D};
      wherein R_{1D} is selected from (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
      and R_{1E} is selected from hydrogen or methyl;
      or R_{1D} and R_{1E} are linked such that, together with the atom or atoms to which they are attached, they form a 4 to 8 membered heterocyclic ring;
   and wherein any (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ or R_{1D} substituent, or a heterocyclyl groups formed when R_{1D} and R_{1E} are linked, is optionally further substituted by halo, cyano or hydroxy (1-2C)alkyl or (1-2C)haloalkyl.
(8) R₁ is is —NR_{1A}R_{1B};
   wherein R_{1A} and R_{1B} are linked such that, together with the nitrogen atom to which they are attached, they form a 4- or 5-membered nitrogen-linked heterocyclic ring, a 7 to 9 membered fused heterocyclic ring or a 7 to 9 membered spirocyclic heterocyclic ring, each of which being optionally substituted by one or more Rₓ;
   wherein each Rₓ is independently selected from halo, (1-2C)alkyl, OR_{1D}, OC(O)R_{1D} or OC(O)N(R_{1E})R_{1D};
      wherein R_{1D} is selected from (1-4C)alkyl, phenyl(1-2C)alkyl, 6-membered heteroaryl, (5- or 6-membered)heteroaryl(1-2C)alkyl, 6- to 9-membered heterocyclyl, or (6- to 9-membered)heterocyclyl(1-2C)alkyl;
      and R_{1E} is hydrogen,
      or R_{1D} and R_{1E} are linked such that, together with the atom or atoms to which they are attached, they form a 4 to 8 membered heterocyclic ring;
   and wherein any (1-4C)alkyl, phenyl(1-2C)alkyl, 5- or 6-membered heteroaryl, (5- or 6-membered)heteroaryl(1-2C)alkyl, 4- to 9-membered heterocyclyl, or (4- to 9-membered)heterocyclyl(1-2C)alkyl group present in a Rₓ or R_{1D} substituent, or a heterocyclyl group formed when R_{1D} and R_{1E} are linked, is optionally further substituted by halo, cyano or hydroxy (1-2C)alkyl or (1-2C)haloalkyl.
(9) R₁ is a group of the formula:

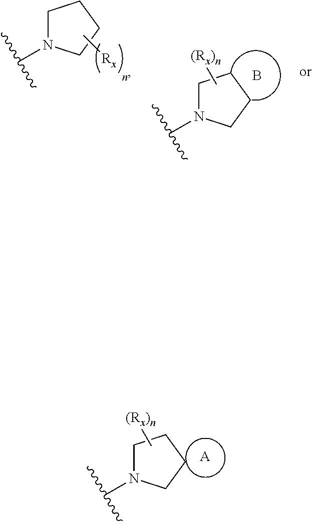

wherein n is 0, 1, 2, 3 or 4; and
each occurrence of Rₓ is independently as defined in any one of the preceding paragraphs;
Ring A, together with the carbon atom to which it is attached, is a spiro-fused 3 to 6 membered cycloalkyl or heterocyclyl ring, optionally substituted by one or more Rₓ; and
Ring B, together with the carbon atoms to which it is attached, is a fused 3 to 6 membered cycloalkyl or heterocyclyl ring, optionally substituted by one or more Rₓ.
(10) R₁ is a group of the formula:

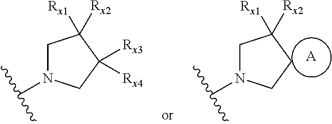

wherein:
Rₓ₁ and Rₓ₂ are independently selected from hydrogen or halo;
Rₓ₃ and Rₓ₄ are independently selected from hydrogen, halo, methyl or methoxy;
or one of Rₓ₃ and Rₓ₄ is hydrogen, and the other is OR_{1D}, wherein R_{1D} is selected from phenyl(1-2C)alkyl, (5- or 6-membered)heteroaryl(1-2C)alkyl, or (4- to 6-membered)heterocyclyl(1-2C)alkyl; wherein the phenyl(1-2C)alkyl, (5- or 6-membered)heteroaryl(1-2C)alkyl, or (4- to 6-membered)heterocyclyl(1-2C)alkyl are each optionally substituted by one or more of halo, cyano, hydroxy or methyl; and
Ring A is a spiro-fused 3 to 6 membered cycloalkyl or heterocyclyl ring, each of which being optionally substituted with one or more substituents selected from halo, methyl or methoxy.
(11) R₁ is a group of the formula:

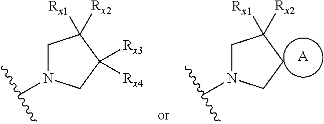

wherein:
Rₓ₁ and Rₓ₂ are independently selected from hydrogen or fluoro;
Rₓ₃ and Rₓ₄ are independently selected from hydrogen, halo or methyl;
or one of Rₓ₃ and Rₓ₄ is hydrogen, and the other is OR_{1D}, wherein R_{1D} is (6-membered)heterocyclyl(1-2C)alkyl; wherein the (6-membered)heterocyclyl(1-2C)alkyl is optionally substituted by one or more of halo, cyano or methyl; and
Ring A is a spiro-fused 3 to 5 membered cycloalkyl or heterocyclyl ring, each of which being optionally substituted with one or more substituents selected from halo or methyl.
(12) R₁ is a group of the formula:

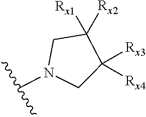

wherein:
Rₓ₁, and Rₓ₂ are either both hydrogen or are both fluoro;
Rₓ₃ and Rₓ₄ are selected from hydrogen, halo or methyl;
or one of Rₓ₃ and Rₓ₄ is hydrogen, and the other is OR_{1D}, wherein R_{1D} is (6-membered)heterocyclyl(1-2C)alkyl; wherein the (6-membered)heterocyclyl(1-2C)alkyl is optionally substituted by one or more of halo, cyano or methyl.
(13) R₁ is selected from:

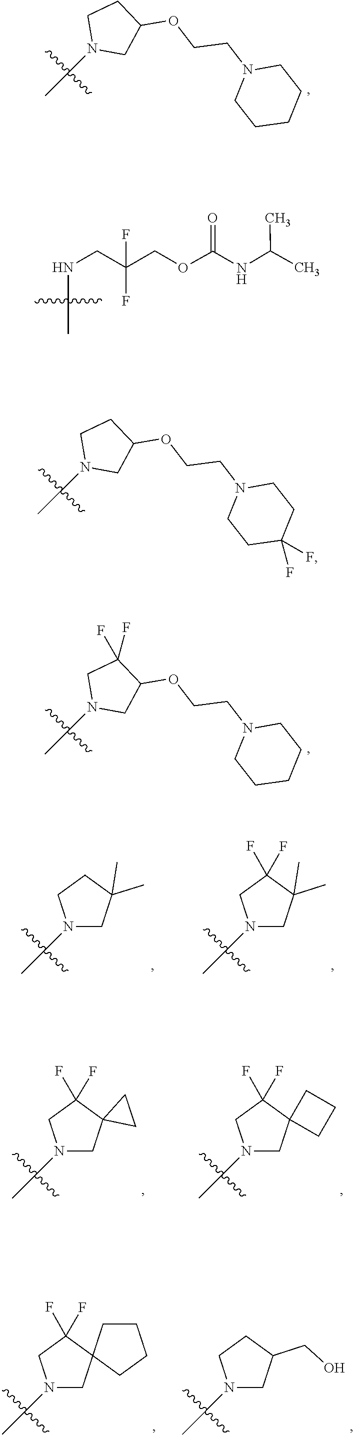

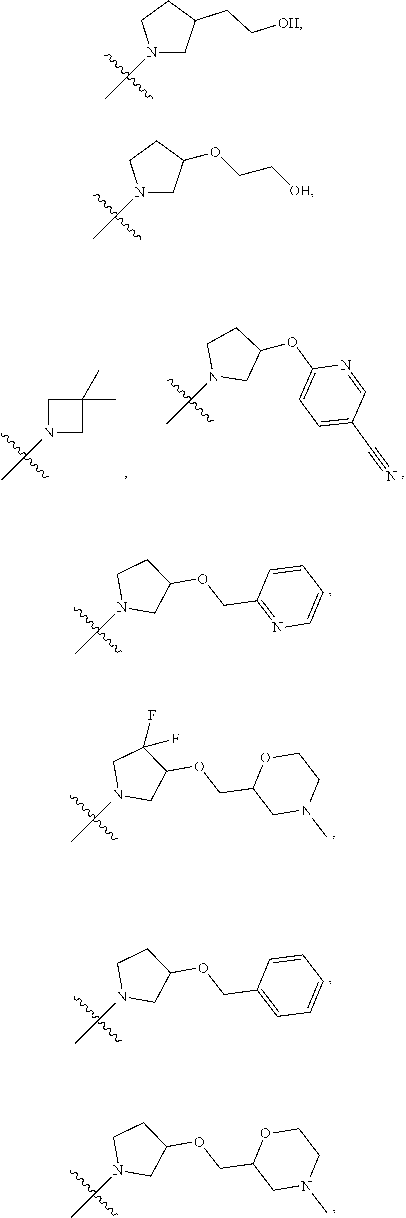

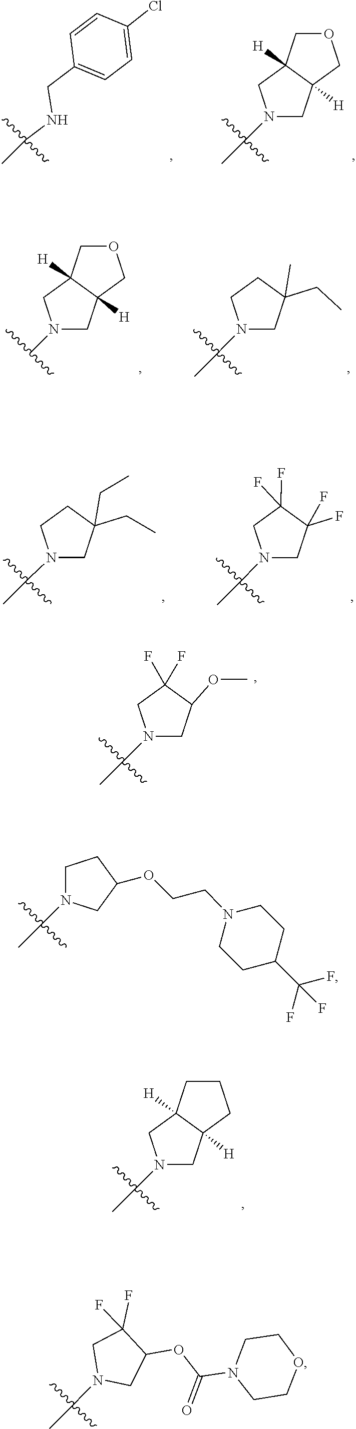

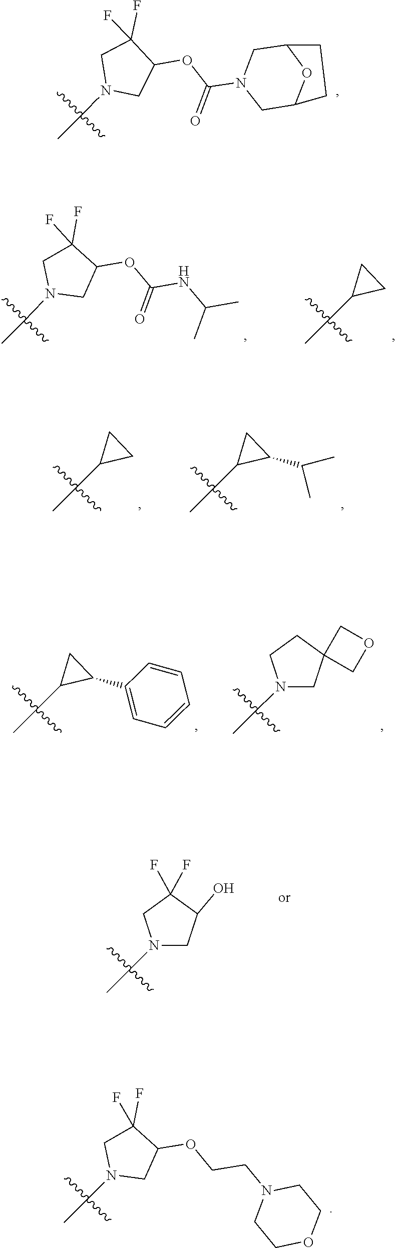

(14)R₁ is selected from:

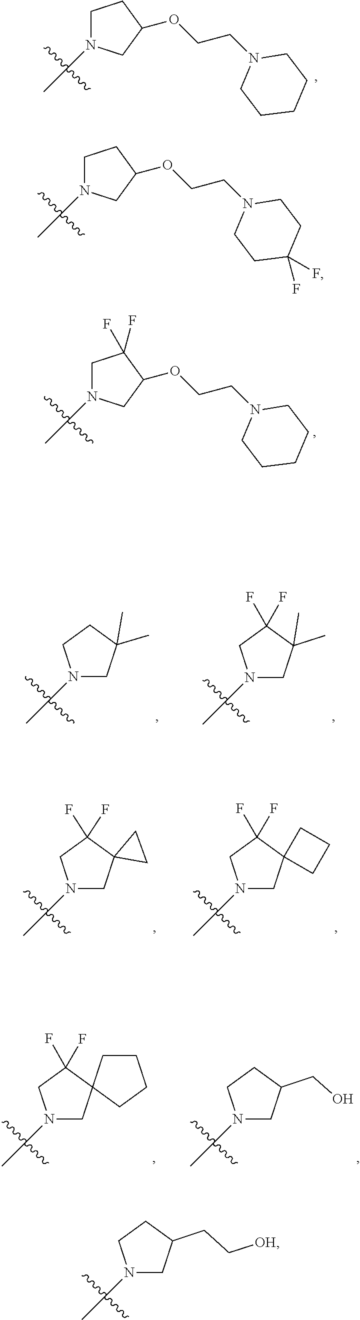

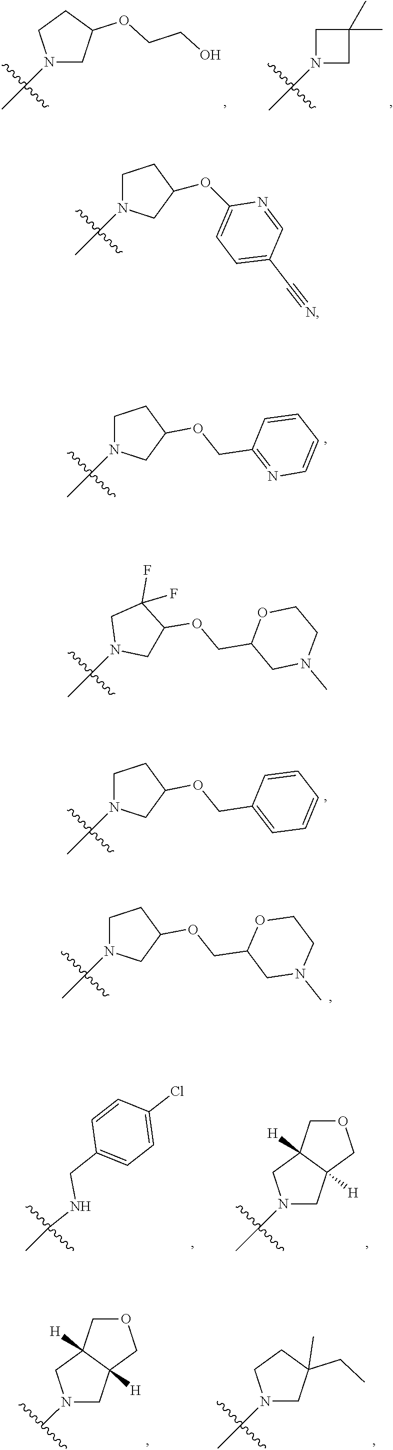

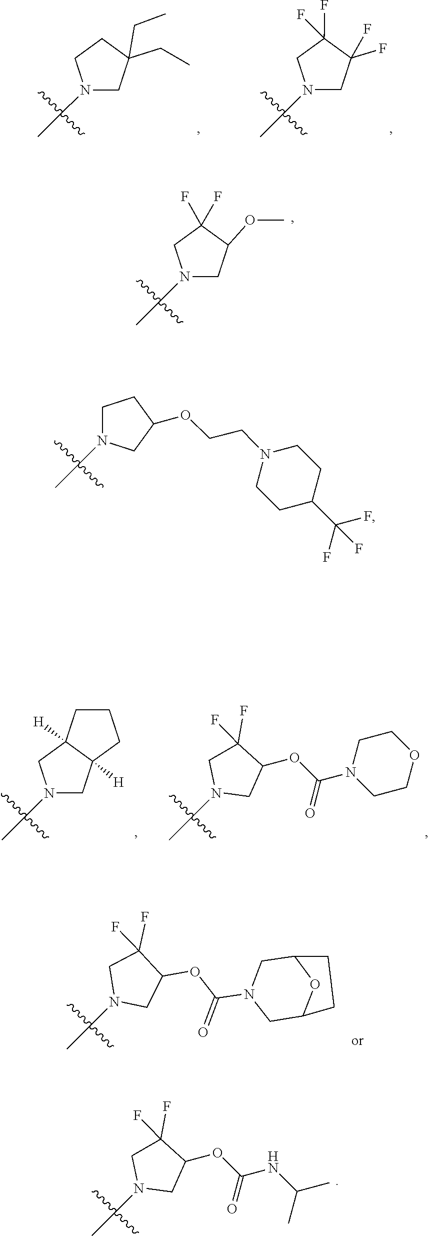

(15) X₂ is selected from N or CR₂; wherein R₂ is selected from hydrogen, halo, methyl, methoxy, halomethyl, halomethoxy or cyano;
X₃ is selected from N or CR₃; wherein R₃ is selected from hydrogen, halo methyl, methoxy, halomethyl, halomethoxy or cyano,
X₄ is selected from N or CR₄; wherein R₄ is selected from hydrogen, halo, (1-2C)alkyl, (1-2C)alkoxy, (1-2C)haloalkyl, (1-2C)haloalkoxy or cyano;
X₅ is selected from N or C;
X₆ is selected from N or C;
X₇ is selected from N, NR_{7N}, CR₇, O or S; wherein:
   R₇ is selected from hydrogen, halo, methyl or methoxy;
   R_{7N} is selected from hydrogen or methyl;
X₈ is selected from N, NR_{8N}, CR₈, O or S; wherein:
   R₈ is selected from hydrogen or halo;
   R_{8N} is selected from hydrogen or methyl;
X₉ is selected from N or C;
with the proviso that:
   (i) X₅ and X₆ cannot both be N;
   (ii) only one of X₇ and X₈ can be O or S;
   (iii) no more than four of the atoms in groups X₂, X₃, X₄, X₅, X₆, X₇, X₈ and X₉ which, together with the carbon atom attached to R₁, make up the bicyclic ring, may be a heteroatom.
(15a) X₂ is selected from N or CR₂; wherein R₂ is selected from hydrogen or cyano;
X₃ is selected from N or CR₃; wherein R₃ is selected from hydrogen, halo or cyano,
X₄ is selected from N or CR₄; wherein R₄ is selected from hydrogen, fluoro, chloro, iodo, methyl, methoxy, fluoromethyl (e.g. CH₂F, CHF₂ or CF₃) or cyano;
X₅ is C;
X₆ is selected from N or C;
X₇ is selected from N, CR₇, or S, wherein R₇ is selected from hydrogen, halo or methyl;
X₈ is selected from N, NR_{8N}, CR₈, O or S; wherein:
   R₈ is selected from hydrogen or halo;
   R_{8N} is hydrogen or methyl;
X₉ is selected from N or C;
with the proviso that:
   (i) X₅ and X₆ cannot both be N;
   (ii) only one of X₇ and X₈ can be O or S;
   (iii) no more than four of the atoms in groups X₂, X₃, X₄, X₆, X₇, X₈ and X₉ which, together with the carbon atom attached to R₁, make up the bicyclic ring, may be a heteroatom.
(16) X₂ is selected from N or CR₂; wherein R₂ is selected from hydrogen or cyano;
X₃ is selected from N or CR₃; wherein R₃ is selected from hydrogen, halo or cyano,
X₄ is selected from N or CR₄; wherein R₄ is selected from hydrogen, fluoro, chloro, methyl, methoxy, fluoromethyl (e.g. CH₂F, CHF₂ or CF₃) or cyano;
X₅ is C;
X₆ is selected from N or C;
X₇ is selected from N, CR₇, or S, wherein R₇ is selected from hydrogen, halo or methyl;
X₈ is selected from N, NR_{8N}, CR₈, O or S; wherein:
   R₈ is selected from hydrogen or halo;
   R_{8N} is hydrogen or methyl;
X₉ is selected from N or C;
with the proviso that:
   (i) X₅ and X₆ cannot both be N;
   (ii) only one of X₇ and X₈ can be O or S;
   (iii) no more than four of the atoms in groups X₂, X₃, X₄, X₆, X₇, X₈ and X₉ which, together with the carbon atom attached to R₁, make up the bicyclic ring, may be a heteroatom.
(16a) X₂ is selected from N or CR₂; wherein R₂ is selected from hydrogen or cyano;
X₃ is selected from N or CR₃; wherein R₃ is selected from hydrogen, halo or cyano,
X₄ is selected from N or CR₄; wherein R₄ is selected from hydrogen, fluoro, chloro, iodo, methyl or fluoromethyl (e.g. CH₂F, CHF₂ or CF₃);
X₅ is C;
X₆ is C or N;
X₇ is N or CR₇, wherein R₇ is hydrogen;
X₈ is selected from N, CR₈ or S, wherein R₈ is hydrogen;
X₉ is selected from N or C;
with the proviso that:
   (i) X₅ and X₆ cannot both be N;
   (ii) only one of X₇ and X₈ can be O or S; and
   (iii) no more than four of the atoms in groups X₂, X₃, X₄, X₆, X₈ and X₉ which, together with the carbon atom attached to R₁, make up the bicyclic ring, may be a heteroatom.
(17) X₂ is selected from N or CR₂; wherein R₂ is selected from hydrogen or cyano;
X₃ is selected from N or CR₃; wherein R₃ is selected from hydrogen, halo or cyano,
X₄ is selected from N or CR₄; wherein R₄ is selected from hydrogen, fluoro, chloro, methyl or fluoromethyl (e.g. CH₂F, CHF₂ or CF₃);
X₅ is C;
X₆ is C or N;
X₇ is N or CR₇, wherein R₇ is hydrogen;
X₈ is selected from N, CR₈ or S, wherein R₈ is hydrogen;
X₉ is selected from N or C;
with the proviso that:
   (i) X₅ and X₆ cannot both be N;
   (ii) only one of X₇ and X₈ can be O or S; and
   (iii) no more than four of the atoms in groups X₂, X₃, X₄, X₆, X₈ and X₉ which, together with the carbon atom attached to R₁, make up the bicyclic ring, may be a heteroatom.
(17a) X₂ is selected from CR₂ or N; wherein R₂ is selected from hydrogen or cyano;
X₃ is selected from N or CR₃; wherein R₃ is selected from hydrogen, halo or cyano,
X₄ is selected from N or CR₄; wherein R₄ is selected from hydrogen, fluoro, chloro, iodo, (e.g. hydrogen or fluoro), methyl or fluoromethyl (e.g. CH₂F, CHF₂ or CF₃);
X₅ is C;
X₆ is N or C;
X₇ is CR₇, wherein R₇ is hydrogen;
X₈ is N;
X₉ is selected from N or C;
with the proviso that:
   i) one of X₂ and X₃ is N, and the other is CR₂ or CR₃ as appropriate; and
   ii) one of X₈ and X₉ is N, and the other is C.
(18) X₂ is selected from CR₂ or N; wherein R₂ is selected from hydrogen or cyano;
X₃ is selected from N or CR₃; wherein R₃ is selected from hydrogen, halo or cyano,
X₄ is selected from N or CR₄; wherein R₄ is selected from hydrogen, fluoro, chloro (e.g. hydrogen or fluoro), methyl or fluoromethyl (e.g. CH₂F, CHF₂ or CF₃);
X₅ is C;
X₆ is N or C;
X₇ is CR₇, wherein R₇ is hydrogen;
X₈ is N;
X₉ is selected from N or C;
with the proviso that:
   i) one of X₂ and X₃ is N, and the other is CR₂ or CR₃ as appropriate; and
   ii) one of X₆ and X₉ is N, and the other is C.
(19) X₂ is N;
X₃ is CR₃, wherein R₃ is selected from hydrogen, halo or cyano,
X₄ is selected from N or CR₄; wherein R₄ is selected from hydrogen, chloro or fluoro (e.g. hydrogen or fluoro);
X₅ is C;
X₆ is C or N;
X₇ is CR₇; wherein R₇ is hydrogen:
X₈ is selected from N, CR₈ or S, wherein R₈ is hydrogen
X₉ is selected from N or C.
(20) X₂ is N;
X₃ is CR₃, wherein R₃ is selected from hydrogen, halo or cyano,
X₄ is N or CR₄; wherein R₄ is selected from hydrogen, chloro or fluoro (e.g. hydrogen or fluoro);
X₅ is C;
X₆ is C or N;
X₇ is CR₇; wherein R₇ is hydrogen:
X₈ is N;
X₉ is selected from N or C.
(21) X₂ is N;
X₃ is CR₃, wherein R₃ is hydrogen,
X₄ is CR₄; wherein R₄ is selected from hydrogen, chloro or fluoro;
X₅ is C;
X₆ is N;
X₇ is CR₇; wherein R₇ is hydrogen:
X₈ is N;
X₉ is C.
(22) X₂ is N;
X₃ is CR₃, wherein R₃ is hydrogen,
X₄ is CR₄; wherein R₄ is hydrogen;
X₅ is C;
X₆ is N;
X₇ is CR₇; wherein R₇ is hydrogen:
X₈ is N;
X₉ is C.
(23) X₂ is N;
X₃ is CR₃, wherein R₃ is hydrogen,
X₄ is CR₄; wherein R₄ is chloro;
X₅ is C;
X₆ is N;
X₇ is CR₇; wherein R₇ is hydrogen:
X₈ is N;
X₉ is C.
(24) X₂ is N;
X₃ is CR₃, wherein R₃ is hydrogen,
X₄ is CR₄; wherein R₄ is fluoro;
X₅ is C;
X₆ is N;
X₇ is CR₇; wherein R₇ is hydrogen:
X₈ is N;
X₉ is C.

Suitably, a heteroaryl or heterocyclyl group as defined herein is a monocyclic heteroaryl or mono, bicyclic or bridged heterocyclyl group comprising one, two or three heteroatoms selected from N, O or S.

Suitably, a heteroaryl is a 5- or 6-membered heteroaryl ring comprising one, two or three heteroatoms selected from N, O or S.

Suitably, a heterocyclyl group is a 4-, 5-, 6-, 7-, 8- or 9-membered heterocyclyl ring comprising one, two or three heteroatoms selected from N, O or S. Most suitably, a heterocyclyl group is a 5-, 6- or 7-membered monocyclic or bicyclic ring comprising one, two or three heteroatoms selected from N, O or S [e.g. morpholinyl (e.g. 4-morpholinyl), pyridinyl, piperazinyl, homopiperazinyl or pyrrolidinonyl].

Suitably, an aryl group is phenyl.

Suitably, R₁ is as defined in any one of paragraphs (1) to (14) above. Most suitably, R₁ is as defined in paragraph (8) or (9) above.

Suitably, X₂, X₃, X₄, X₅, X₆, X₇, X₈ and X₉ are as defined in any one of paragraphs (15) to (18), or (19) to (21) above. More suitably, X₂, X₃, X₄, X₅, X₆, X₇, X₈ and X₉ are as defined in any one of paragraphs (17), (17a), (18) or (19) above. Most suitably, X₂, X₃, X₄, X₅, X₆, X₇, X₈ and X₉ are as defined in paragraph (19) above.

Suitably, X₂, X₃, X₄, X₅, X₆, X₇, X₈ and X₉ are as defined in any one of paragraphs (15) to or (21) above. More suitably, X₂, X₃, X₄, X₅, X₆, X₇, X₈ and X₉ are as defined in any one of paragraphs (18) to (21) above. Even more suitably, X₂, X₃, X₄, X₅, X₆, X₇, X₈ and X₉ are as defined in paragraph (19), (20) or (21) above.

Suitably, R₂ is as defined in any one of paragraphs (15) to (18) above. Most suitably, R₂ is as defined in paragraph (18) above.

Suitably, R₃ is as defined in any one of paragraphs (15) to (21) above. Most suitably, R₃ is as defined in paragraph (21) above.

Suitably, R₄ is as defined in any one of paragraphs (15) to (21) above. Suitably, R₄ is as defined in any one of paragraphs (15a) to (21) above. More suitably, R₄ is as defined in any one of paragraphs (18) to (21). Most suitably, R₄ is as defined in paragraph (21) above.

Suitably, R₄ is as defined in any one of paragraphs (15) to (24) above. Suitably, R₄ is as defined in any one of paragraphs (15a) to (24) above. More suitably, R₄ is as defined in any one of paragraphs (18) to (24). Most suitably, R₄ is as defined in paragraph (21) above.

Suitably, R₄ is as defined in any one of paragraphs (22), (23) or (24) above.

Suitably, R₇ is as defined in any one of paragraphs (15) to (21) above. Most suitably, R₇ is as defined in paragraph (21) above.

Suitably, R₈ is as defined in any one of paragraphs (15) to (19) above. Most suitably, R₈ is as defined in paragraph (19) above.

In a particular group of compounds of Formula I above, R₁ is as defined in any one of paragraphs (1), (2), (3), (4), (5), (6), (7), (8), (9), (10), (11), (12), (13) or (14) above and X₂, X₃, X₄, X₅, X₆, X₇, X₈ and X₉ have any one of the definitions herein.

In a particular group of compounds of Formula I above, R₁ is as defined in paragraph (1) above and X₂, X₃, X₄, X₅, X₆, X₇, X₈ and X₉ have any one of the definitions herein.

In a particular group of compounds of Formula I above, R₁ is as defined in paragraph (2) above and X₂, X₃, X₄, X₅, X₆, X₇, X₈ and X₉ have any one of the definitions herein.

In a particular group of compounds of Formula I above, R₁ is as defined in paragraph (3) above and X₂, X₃, X₄, X₅, X₆, X₇, X₈ and X₉ have any one of the definitions herein.

In a particular group of compounds of Formula I above, R₁ is as defined in paragraph (4) above and X₂, X₃, X₄, X₅, X₆, X₇, X₈ and X₉ have any one of the definitions herein.

In a particular group of compounds of Formula I above, R₁ is as defined in paragraph (5) above and X₂, X₃, X₄, X₅, X₆, X₇, X₈ and X₉ have any one of the definitions herein.

In a particular group of compounds of Formula I above, R₁ is as defined in paragraph (6) above and X₂, X₃, X₄, X₅, X₆, X₇, X₈ and X₉ have any one of the definitions herein.

In a particular group of compounds of Formula I above, R₁ is as defined in paragraph (7) above and X₂, X₃, X₄, X₅, X₆, X₇, X₈ and X₉ have any one of the definitions herein.

In a particular group of compounds of Formula I above, R₁ is as defined in paragraph (8) above and X₂, X₃, X₄, X₅, X₆, X₇, X₈ and X₉ have any one of the definitions herein.

In a particular group of compounds of Formula I above, R₁ is as defined in paragraph (9) above and X₂, X₃, X₄, X₅, X₆, X₇, X₈ and X₉ have any one of the definitions herein.

In a particular group of compounds of Formula I above, R₁ is as defined in paragraph (10) above and X₂, X₃, X₄, X₅, X₆, X₇, X₈ and X₉ have any one of the definitions herein.

In a particular group of compounds of Formula I above, R₁ is as defined in paragraph (11) above and X₂, X₃, X₄, X₅, X₆, X₇, X₈ and X₉ have any one of the definitions herein.

In a particular group of compounds of Formula I above, R₁ is as defined in paragraph (12) above and X₂, X₃, X₄, X₅, X₆, X₇, X₈ and X₉ have any one of the definitions herein.

In a particular group of compounds of Formula I above, R₁ is as defined in paragraph (13) above and X₂, X₃, X₄, X₅, X₆, X₇, X₈ and X₉ have any one of the definitions herein.

In a particular group of compounds of Formula I above, R₁ is as defined in paragraph (14) above and X₂, X₃, X₄, X₅, X₆, X₇, X₈ and X₉ have any one of the definitions herein.

In a particular group of compounds of Formula I above, X₂, X₃, X₄, X₅, X₆, X₇, X₈ and X₉ are as defined in any one of paragraphs (15) to (18), or (19) to (21) above and R₁ has any one of the definitions herein.

In a particular group of compounds of Formula I above, X₂, X₃, X₄, X₅, X₆, X₇, X₈ and X₉ are as defined in paragraph (15) above and R₁ has any one of the definitions herein.

In a particular group of compounds of Formula I above, X₂, X₃, X₄, X₅, X₆, X₇, X₈ and X₉ are as defined in paragraph (16) above and R₁ has any one of the definitions herein.

In a particular group of compounds of Formula I above, X₂, X₃, X₄, X₅, X₆, X₇, X₈ and X₉ are as defined in paragraph (17) above and R₁ has any one of the definitions herein.

In a particular group of compounds of Formula I above, X₂, X₃, X₄, X₅, X₆, X₇, X₈ and X₉ are as defined in paragraph (15a) above and R₁ has any one of the definitions herein.

In a particular group of compounds of Formula I above, X₂, X₃, X₄, X₅, X₆, X₇, X₈ and X₉ are as defined in paragraph (16a) above and R₁ has any one of the definitions herein.

In a particular group of compounds of Formula I above, X₂, X₃, X₄, X₅, X₆, X₇, X₈ and X₉ are as defined in paragraph (17a) above and R₁ has any one of the definitions herein.

In a particular group of compounds of Formula I above, X₂, X₃, X₄, X₅, X₆, X₇, X₈ and X₉ are as defined in paragraph (18) above and R₁ has any one of the definitions herein.

In a particular group of compounds of Formula I above, X₂, X₃, X₄, X₅, X₆, X₇, X₈ and X₉ are as defined in paragraph (19) above and R₁ has any one of the definitions herein.

In a particular group of compounds of Formula I above, X₂, X₃, X₄, X₅, X₆, X₇, X₈ and X₉ are as defined in paragraph (20) above and R₁ has any one of the definitions herein.

In a particular group of compounds of Formula I above, X₂, X₃, X₄, X₅, X₆, X₇, X₈ and X₉ are as defined in paragraph (21) above and R₁ has any one of the definitions herein.

In a particular group of compounds of Formula I above, R₁ is as defined in any one of paragraphs (1), (2), (3), (4), (5), (6), (7), (8), (9), (10), (11), (12), (13), (14) above and X₂, X₃, X₄, X₅, X₆, X₇, X₈ and X₉ have any one of the definitions herein.

In a particular group of compounds of Formula 1-1 above, R₁ is as defined in paragraph (1) above and X₂, R₃, X₄, X₅, X₆, X₇, X₈ and X₉ have any one of the definitions herein.

In a particular group of compounds of Formula 1-1 above, R₁ is as defined in paragraph (2) above and X₂, R₃, X₄, X₅, X₆, X₇, X₈ and X₉ have any one of the definitions herein.

In a particular group of compounds of Formula 1-1 above, R₁ is as defined in paragraph (3) above and X₂, R₃, X₄, X₅, X₆, X₇, X₈ and X₉ have any one of the definitions herein.

In a particular group of compounds of Formula 1-1 above, R₁ is as defined in paragraph (4) above and X₂, R₃, X₄, X₅, X₆, X₇, X₈ and X₉ have any one of the definitions herein.

In a particular group of compounds of Formula 1-1 above, R₁ is as defined in paragraph (5) above and X₂, R₃, X₄, X₅, X₆, X₇, X₈ and X₉ have any one of the definitions herein.

In a particular group of compounds of Formula 1-1 above, R₁ is as defined in paragraph (6) above and X₂, R₃, X₄, X₅, X₆, X₇, X₈ and X₉ have any one of the definitions herein.

In a particular group of compounds of Formula 1-1 above, R₁ is as defined in paragraph (7) above and X₂, R₃, X₄, X₅, X₆, X₇, X₈ and X₉ have any one of the definitions herein.

In a particular group of compounds of Formula 1-1 above, R₁ is as defined in paragraph (8) above and X₂, R₃, X₄, X₅, X₆, X₇, X₈ and X₉ have any one of the definitions herein.

In a particular group of compounds of Formula 1-1 above, R₁ is as defined in paragraph (9) above and X₂, R₃, X₄, X₅, X₆, X₇, X₈ and X₉ have any one of the definitions herein.

In a particular group of compounds of Formula 1-1 above, R₁ is as defined in paragraph (10) above and X₂, R₃, X₄, X₅, X₆, X₇, X₈ and X₉ have any one of the definitions herein.

In a particular group of compounds of Formula 1-1 above, R₁ is as defined in paragraph (11) above and X₂, R₃, X₄, X₅, X₆, X₇, X₈ and X₉ have any one of the definitions herein.

In a particular group of compounds of Formula 1-1 above, R₁ is as defined in paragraph (12) above and X₂, R₃, X₄, X₅, X₆, X₇, X₈ and X₉ have any one of the definitions herein.

In a particular group of compounds of Formula 1-1 above, R₁ is as defined in paragraph (13) above and X₂, R₃, X₄, X₅, X₆, X₇, X₈ and X₉ have any one of the definitions herein.

In a particular group of compounds of Formula 1-1 above, R₁ is as defined in paragraph (14) above and X₂, R₃, X₄, X₅, X₆, X₇, X₈ and X₉ have any one of the definitions herein.

In a particular group of compounds of Formula 1-1 above, X₂, R₃, X₄, X₅, X₆, X₇, X₈ and X₉ are as defined in any one of paragraphs (15) to (18), or (19) to (21) above and R₁ has any one of the definitions herein.

In a particular group of compounds of Formula 1-1 above, X₂, R₃, X₄, X₅, X₆, X₇, X₈ and X₉ are as defined in paragraph (15) above and R₁ has any one of the definitions herein.

In a particular group of compounds of Formula 1-1 above, X₂, R₃, X₄, X₅, X₆, X₇, X₈ and X₉ are as defined in paragraph (16) above and R₁ has any one of the definitions herein.

In a particular group of compounds of Formula 1-1 above, X₂, R₃, X₄, X₅, X₆, X₇, X₈ and X₉ are as defined in paragraph (17) above and R₁ has any one of the definitions herein.

In a particular group of compounds of Formula 1-1 above, X₂, R₃, X₄, X₅, X₆, X₇, X₈ and X₉ are as defined in paragraph (15a) above and R₁ has any one of the definitions herein.

In a particular group of compounds of Formula 1-1 above, X₂, R₃, X₄, X₅, X₆, X₇, X₈ and X₉ are as defined in paragraph (16a) above and R₁ has any one of the definitions herein.

In a particular group of compounds of Formula 1-1 above, X₂, R₃, X₄, X₅, X₆, X₇, X₈ and X₉ are as defined in paragraph (17a) above and R₁ has any one of the definitions herein.

In a particular group of compounds of Formula 1-1 above, X₂, R₃, X₄, X₅, X₆, X₇, X₈ and X₉ are as defined in paragraph (18) above and R₁ has any one of the definitions herein.

In a particular group of compounds of Formula 1-1 above, X₂, R₃, X₄, X₅, X₆, X₇, X₈ and X₉ are as defined in paragraph (19) above and R₁ has any one of the definitions herein.

In a particular group of compounds of Formula 1-1 above, X₂, R₃, X₄, X₅, X₆, X₇, X₈ and X₉ are as defined in paragraph (20) above and R₁ has any one of the definitions herein.

In a particular group of compounds of Formula 1-1 above, X₂, R₃, X₄, X₅, X₆, X₇, X₈ and X₉ are as defined in paragraph (21) above and R₁ has any one of the definitions herein.

In a particular group of compounds of Formula 1-1 above, X₂, R₃, X₄, X₅, X₆, X₇, X₈ and X₉ are as defined in paragraph (22) above and R₁ has any one of the definitions herein.

In a particular group of compounds of Formula 1-1 above, X₂, R₃, X₄, X₅, X₆, X₇, X₈ and X₉ are as defined in paragraph (23) above and R₁ has any one of the definitions herein.

In a particular group of compounds of Formula 1-1 above, X₂, R₃, X₄, X₅, X₆, X₇, X₈ and X₉ are as defined in paragraph (24) above and R₁ has any one of the definitions herein.

In a particular group of compounds of the invention, the compounds have the structural formula Ia, Ib, Ic, Id, Ie, If, Ig, Ih, Ij, Ik, Im, In, Io or Ip [sub-definitions of formula (I)] shown below, or a pharmaceutically acceptable salt, hydrate and/or solvate thereof:

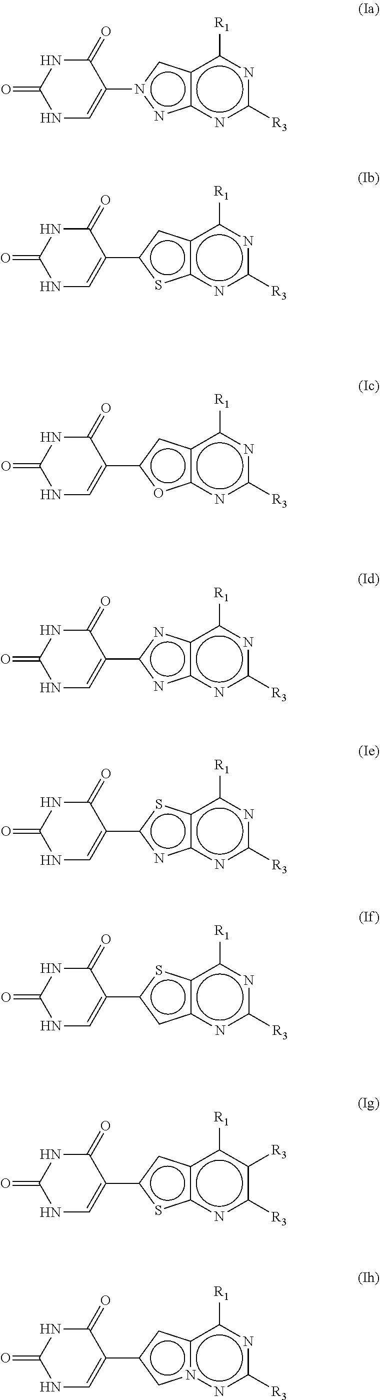

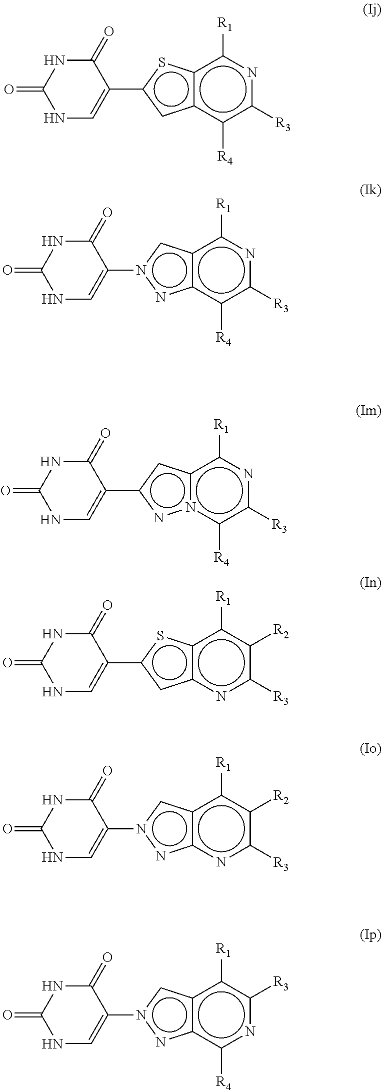

wherein 1, 2, 3 and are as defined herein.

In a particular group of compounds of Formula Ia, Ib, Ic, Id, Ie, If, Ig, Ih, Ij, Ik, Im, In, Io or Ip above, R₁ is as defined in paragraph (1) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (2) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (3) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (4) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (5) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (6) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (7) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (8) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (9) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (10) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (11) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (12) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (13) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₁ is as defined in paragraph (14) above and R₂, R₃ and R₄ have any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in any one of paragraphs (15) to (18), or (19) to (21) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in paragraph (15) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in paragraph (16) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in paragraph (17) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in paragraph (15a) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in paragraph (16a) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in paragraph (17a) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are as defined in paragraph (18) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are each independently as defined in paragraph (19) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are each independently as defined in paragraph (20) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are each independently as defined in paragraph (21) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are each independently as defined in paragraph (22) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are each independently as defined in paragraph (23) above and R₁ has any one of the definitions herein.

In a particular group of compounds of Formula Ia, Ib, Ic, Id, Ie, If, Ig, Ih, Ij, Ik, Im, In, Io or Ip above, R₂, R₃ and R₄ are each independently as defined in paragraph (24) above and R₁ has any one of the definitions herein.

In a particular group of compounds of Formula Ia, Ib, Ic, Id, Ie, If, Ig, Ih, Ij, Ik, Im, In, Io or Ip above:
R₁ is as defined in any one of paragraphs (1) to (14);
R₂, R₃ and R₄ are each independently as defined in any one of paragraphs (15) to (21) above.

In a particular group of compounds of Formula Ia, Ib, Ic, Id, Ie, If, Ig, Ih, Ij, Ik, Im, In, Io or Ip above:
R₁ is as defined in any one of paragraphs (7) to (14);
R₂, R₃ and R₄ are each independently as defined in any one of paragraphs (18) to (21) above.

In a particular group of compounds of Formula Ia, Ib, Ic, Id, Ie, If, Ig, Ih, Ij, Ik, Im, In, Io or Ip above:
R₁ is as defined in any one of paragraphs (9) to (14) above;
R₂, R₃ and R₄ are each independently as defined in any one of paragraphs (18) to (21) above.

In a particular group of compounds of Formula Ia, Ib, Ic, Id, Ie, If, Ig, Ih, Ij, Ik, Im, In, Io or Ip above:
R₁ is as defined in any one of paragraphs (12), (13) or (14);
R₂, R₃ and R₄ are each independently as defined in any one of paragraphs (19) to (21) above.

In a particular group of compounds of Formula Ia, Ib, Ic, Id, Ie, If, Ig, Ih, Ij, Ik, Im, In, Io or Ip above:
R₁ is as defined in any one of paragraphs (12), (13) or (14);
R₂, R₃ and R₄ are each independently as defined in any one of paragraphs (19) to (21) above.

In a particular group of compounds of the invention, the compounds have the structural formula Ia, [a sub-definition of formula (I)]. defined herein.

In a particular group of compounds of the invention, the compounds have the structural formula Ib, [a sub-definition of formula (I)]. defined herein.

In a particular group of compounds of the invention, the compounds have the structural formula Ic, [a sub-definition of formula (I)]. defined herein.

In a particular group of compounds of the invention, the compounds have the structural formula Id, [a sub-definition of formula (I)]. defined herein.

In a particular group of compounds of the invention, the compounds have the structural formula Ie, [a sub-definition of formula (I)]. defined herein.

In a particular group of compounds of the invention, the compounds have the structural formula If, [a sub-definition of formula (I)]. defined herein.

In a particular group of compounds of the invention, the compounds have the structural formula Ig, [a sub-definition of formula (I)]. defined herein.

In a particular group of compounds of the invention, the compounds have the structural formula Ih, [a sub-definition of formula (I)]. defined herein.

In a particular group of compounds of the invention, the compounds have the structural formula Ij, [a sub-definition of formula (I)]. defined herein.

In a particular group of compounds of the invention, the compounds have the structural formula Ik, [a sub-definition of formula (I)]. defined herein.

In a particular group of compounds of the invention, the compounds have the structural formula Im, [a sub-definition of formula (I)]. defined herein.

In a particular group of compounds of the invention, the compounds have the structural formula In, [a sub-definition of formula (I)]. defined herein.

In a particular group of compounds of the invention, the compounds have the structural formula Io, [a sub-definition of formula (I)]. defined herein.

In a particular group of compounds of the invention, the compounds have the structural formula Ip, [a sub-definition of formula (I)]. defined herein.

In a particular group of compounds of the invention, the compounds have the structural formula Ia, [a sub-definition of formula (I)] shown below, or a pharmaceutically acceptable salt, hydrate and/or solvate thereof:

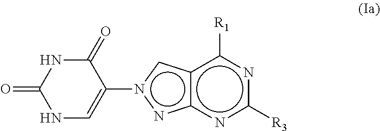

wherein R₁ and R₃ are as defined herein.

In a particular group of compounds of Formula Ia above, R₁ is as defined in paragraph (1) above and R₃ has any one of the definitions herein.

In a particular group of compounds of Formula Ia above, R₁ is as defined in paragraph (2) above and R₃ has any one of the definitions herein.

In a particular group of compounds of Formula Ia above, R₁ is as defined in paragraph (3) above and R₃ has any one of the definitions herein.

In a particular group of compounds of Formula Ia above, R₁ is as defined in paragraph (4) above and R₃ has any one of the definitions herein.

In a particular group of compounds of Formula Ia above, R₁ is as defined in paragraph (5) above and R₃ has any one of the definitions herein.

In a particular group of compounds of Formula Ia above, R₁ is as defined in paragraph (6) above and R₃ has any one of the definitions herein.

In a particular group of compounds of Formula Ia above, R₁ is as defined in paragraph (7) above and R₃ has any one of the definitions herein.

In a particular group of compounds of Formula Ia above, R₁ is as defined in paragraph (8) above and R₃ has any one of the definitions herein.

In a particular group of compounds of Formula Ia above, R₁ is as defined in paragraph (9) above and R₃ has any one of the definitions herein.

In a particular group of compounds of Formula Ia above, R₁ is as defined in paragraph (10) above and R₃ has any one of the definitions herein.

In a particular group of compounds of Formula Ia above, R₁ is as defined in paragraph (11) above and R₃ has any one of the definitions herein.

In a particular group of compounds of Formula Ia above, R₁ is as defined in paragraph (12) above and R₃ has any one of the definitions herein.

In a particular group of compounds of Formula Ia above, R₁ is as defined in paragraph (13) above and R₃ has any one of the definitions herein.

In a particular group of compounds of Formula Ia above, R₁ is as defined in paragraph (14) above and R₃ has any one of the definitions herein.

In a particular group of compounds of Formula Ia above, R₃ is as defined in any one of paragraphs (15) to (18), or (19) to (21) above and R₁ has any one of the definitions herein.

In a particular group of compounds of Formula Ia above, R₃ is as defined in paragraph (15) above and R₁ has any one of the definitions herein.

In a particular group of compounds of Formula Ia above, R₃ is as defined in paragraph (16) above and R₁ has any one of the definitions herein.

In a particular group of compounds of Formula Ia above, R₃ is as defined in paragraph (17) above and R₁ has any one of the definitions herein.

In a particular group of compounds of Formula Ia above, R₃ is as defined in paragraph (15a) above and R₁ has any one of the definitions herein.

In a particular group of compounds of Formula Ia above, R₃ is as defined in paragraph (16a) above and R₁ has any one of the definitions herein.

In a particular group of compounds of Formula Ia above, R₃ is as defined in paragraph (17a) above and R₁ has any one of the definitions herein.

In a particular group of compounds of Formula Ia above, R₃ is as defined in paragraph (18) above and R₁ has any one of the definitions herein.

In a particular group of compounds of Formula Ia above, R₃ is as defined in paragraph (19) above and R₁ has any one of the definitions herein.

In a particular group of compounds of Formula Ia above, R₃ is as defined in paragraph (20) above and R₁ has any one of the definitions herein.

In a particular group of compounds of Formula Ia above, R₃ is as defined in paragraph (21) above and R₁ has any one of the definitions herein.

In a particular group of compounds of Formula Ia above, R₃ is hydrogen and R₁ has any one of the definitions herein.

In a particular group of compounds of Formula Ia above:
R₁ is as defined in any one of paragraphs (1) to (14); and
R₃ is as defined in any one of paragraphs (15) to (21) above.

In a particular group of compounds of Formula Ia, Ib, Ic, Id, Ie, If, Ig, Ih, Ij, Ik, Im, In, Io or Ip above:
R₁ is as defined in any one of paragraphs (7) to (14);
R₃ is as defined in any one of paragraphs (18) to (21) above.

In a particular group of compounds of Formula Ia above:
R₁ is as defined in any one of paragraphs (9) to (14) above;
R₃ is as defined in any one of paragraphs (18) to (21) above.

In a particular group of compounds of Formula Ia above:
R₁ is as defined in any one of paragraphs (12), (13) or (14); and
R₃ is as defined in any one of paragraphs (19) to (21) above.

In a particular group of compounds of the invention, the compounds have the structural formula Ik, [a sub-definition of formula (I)] shown below, or a pharmaceutically acceptable salt, hydrate and/or solvate thereof:

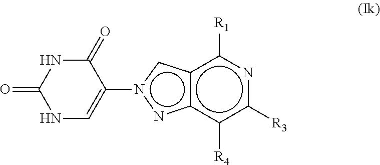

wherein R₁ and R₃ are as defined herein.

In a particular group of compounds of Formula Ik above, R₁ is as defined in paragraph (1) above and R₃ and R₄ have any one of the definitions herein.

In a particular group of compounds of Formula Ik above, R₁ is as defined in paragraph (2) above and R₃ and R₄ have any one of the definitions herein.

In a particular group of compounds of Formula Ik above, R₁ is as defined in paragraph (3) above and R₃ and R₄ have any one of the definitions herein.

In a particular group of compounds of Formula Ik above, R₁ is as defined in paragraph (4) above and R₃ and R₄ have any one of the definitions herein.

In a particular group of compounds of Formula Ik above, R₁ is as defined in paragraph (5) above and R₃ and R₄ have any one of the definitions herein.

In a particular group of compounds of Formula Ik above, R₁ is as defined in paragraph (6) above and R₃ and R₄ have any one of the definitions herein.

In a particular group of compounds of Formula Ik above, R₁ is as defined in paragraph (7) above and R₃ and R₄ have any one of the definitions herein.

In a particular group of compounds of Formula Ik above, R₁ is as defined in paragraph (8) above and R₃ and R₄ have any one of the definitions herein.

In a particular group of compounds of Formula Ik above, R₁ is as defined in paragraph (9) above and R₃ and R₄ have any one of the definitions herein.

In a particular group of compounds of Formula Ik above, R₁ is as defined in paragraph (10) above and R₃ and R₄ have any one of the definitions herein.

In a particular group of compounds of Formula Ik above, R₁ is as defined in paragraph (11) above and R₃ and R₄ have any one of the definitions herein.

In a particular group of compounds of Formula Ik above, R₁ is as defined in paragraph (12) above and R₃ and R₄ have any one of the definitions herein.

In a particular group of compounds of Formula Ik above, R₁ is as defined in paragraph (13) above and R₃ and R₄ have any one of the definitions herein.

In a particular group of compounds of Formula Ik above, R₁ is as defined in paragraph (14) above and R₃ and R₄ have any one of the definitions herein.

In a particular group of compounds of Formula Ik above, R₃ and R₄ are as defined in any one of paragraphs (15) to (18), or (19) to (21) above and R₁ has any one of the definitions herein.

In a particular group of compounds of Formula Ik above, R₃ and R₄ are as defined in any one of paragraphs (22) to (24) above and R₁ has any one of the definitions herein.

In a particular group of compounds of Formula Ik above, R₃ and R₄ are as defined in paragraph (15) above and R₁ has any one of the definitions herein.

In a particular group of compounds of Formula Ik above, R₃ and R₄ are as defined in paragraph (16) above and R₁ has any one of the definitions herein.

In a particular group of compounds of Formula Ik above, R₃ and R₄ are as defined in paragraph (17) above and R₁ has any one of the definitions herein.

In a particular group of compounds of Formula Ik above, R₃ and R₄ are as defined in paragraph (15a) above and R₁ has any one of the definitions herein.

In a particular group of compounds of Formula Ik above, R₃ and R₄ are as defined in paragraph (16a) above and R₁ has any one of the definitions herein.

In a particular group of compounds of Formula Ik above, R₃ and R₄ are as defined in paragraph (17a) above and R₁ has any one of the definitions herein.

In a particular group of compounds of Formula Ik above, R₃ and R₄ are as defined in paragraph (18) above and R₁ has any one of the definitions herein.

In a particular group of compounds of Formula Ik above, R₃ and R₄ are as defined in paragraph (19) above and R₁ has any one of the definitions herein.

In a particular group of compounds of Formula Ik above, R₃ and R₄ are as defined in paragraph (20) above and R₁ has any one of the definitions herein.

In a particular group of compounds of Formula Ik above, R₃ and R₄ are as defined in paragraph (21) above and R₁ has any one of the definitions herein.

In a particular group of compounds of Formula Im above, R₃ and R₄ are as defined in paragraph (22) above and R₁ has any one of the definitions herein.

In a particular group of compounds of Formula Im above, R₃ and R₄ are as defined in paragraph (23) above and R₁ has any one of the definitions herein.

In a particular group of compounds of Formula Im above, R₃ and R₄ are as defined in paragraph (24) above and R₁ has any one of the definitions herein.

In a particular group of compounds of Formula Ik above, R₃ is hydrogen, and R₄ is as defined in any one of paragraphs (15) to (20) above, and R₁ has any one of the definitions herein.

In a particular group of compounds of Formula Ik above, R₃ is hydrogen, and R₄ is as defined in paragraph (15) above and R₁ has any one of the definitions herein.

In a particular group of compounds of Formula Ik above, R₃ is hydrogen, and R₄ is as defined in paragraph (16) above and R₁ has any one of the definitions herein.

In a particular group of compounds of Formula Ik above, R₃ is hydrogen, and R₄ is as defined in paragraph (17) above and R₁ has any one of the definitions herein.

In a particular group of compounds of Formula Ik above, R₃ is hydrogen, and R₄ is as defined in paragraph (15a) above and R₁ has any one of the definitions herein.

In a particular group of compounds of Formula Ik above, R₃ is hydrogen, and R₄ is as defined in paragraph (16a) above and R₁ has any one of the definitions herein.

In a particular group of compounds of Formula Ik above, R₃ is hydrogen, and R₄ is as defined in paragraph (17a) above and R₁ has any one of the definitions herein.

In a particular group of compounds of Formula Ik above, R₃ is hydrogen, and R₄ is as defined in paragraph (18) above and R₁ has any one of the definitions herein.

In a particular group of compounds of Formula Ik above, R₃ is hydrogen, and R₄ is as defined in paragraph (19) above and R₁ has any one of the definitions herein.

In a particular group of compounds of Formula Ik above, R₃ is hydrogen, and R₄ is as defined in paragraph (20) above and R₁ has any one of the definitions herein.

In a particular group of compounds of Formula Ik above, R₃ is hydrogen, and R₄ is as defined in paragraph (21) above and R₁ has any one of the definitions herein.

In a particular group of compounds of Formula Ik above, R₃ and R₄ are as defined in paragraph (22) above and R₁ has any one of the definitions herein.

In a particular group of compounds of Formula Ik above, R₃ and R₄ are as defined in paragraph (23) above and R₁ has any one of the definitions herein.

In a particular group of compounds of Formula Ik above, R₃ and R₄ are as defined in paragraph (24) above and R₁ has any one of the definitions herein.

In a particular group of compounds of Formula Ik above:
R₁ is as defined in any one of paragraphs (1) to (14);
R₃ is as defined in any one of paragraphs (15) to (21) above; and
R₄ is as defined in any one of paragraphs (15) to (21) above.

In a particular group of compounds of Formula Ik above:
R₁ is as defined in any one of paragraphs (7) to (14);
R₃ is as defined in any one of paragraphs (18) to (21) above; and
R₄ is as defined in any one of paragraphs (18) to (21) above.

In a particular group of compounds of Formula Ik above:
R₁ is as defined in any one of paragraphs (9) to (14) above;
R₃ is as defined in any one of paragraphs (18) to (21) above; and
R₄ is as defined in any one of paragraphs (18) to (21) above.

In a particular group of compounds of Formula Ik above:
R₁ is as defined in any one of paragraphs (12), (13) or (14);
R₃ is hydrogen; and
R₄ is as defined in any one of paragraphs (19) to (21) above.

In a particular group of compounds of the invention, the compounds have the structural formula Im, [a sub-definition of formula (I)] shown below, or a pharmaceutically acceptable salt, hydrate and/or solvate thereof:

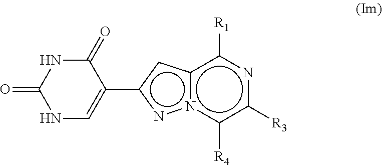

wherein R₁, R₃ and R₄ are as defined herein.

In a particular group of compounds of Formula Im above, R₁ is as defined in paragraph (1) above and R₃ and R₄ have any one of the definitions herein.

In a particular group of compounds of Formula Im above, R₁ is as defined in paragraph (2) above and R₃ and R₄ have any one of the definitions herein.

In a particular group of compounds of Formula Im above, R₁ is as defined in paragraph (3) above and R₃ and R₄ have any one of the definitions herein.

In a particular group of compounds of Formula Im above, R₁ is as defined in paragraph (4) above and R₃ and R₄ have any one of the definitions herein.

In a particular group of compounds of Formula Im above, R₁ is as defined in paragraph (5) above and R₃ and R₄ have any one of the definitions herein.

In a particular group of compounds of Formula Im above, R₁ is as defined in paragraph (6) above and R₃ and R₄ have any one of the definitions herein.

In a particular group of compounds of Formula Im above, R₁ is as defined in paragraph (7) above and R₃ and R₄ have any one of the definitions herein.

In a particular group of compounds of Formula Im above, R₁ is as defined in paragraph (8) above and R₃ and R₄ have any one of the definitions herein.

In a particular group of compounds of Formula Im above, R₁ is as defined in paragraph (9) above and R₃ and R₄ have any one of the definitions herein.

In a particular group of compounds of Formula Im above, R₁ is as defined in paragraph (10) above and R₃ and R₄ have any one of the definitions herein.

In a particular group of compounds of Formula Im above, R₁ is as defined in paragraph (11) above and R₃ and R₄ have any one of the definitions herein.

In a particular group of compounds of Formula Im above, R₁ is as defined in paragraph (12) above and R₃ and R₄ have any one of the definitions herein.

In a particular group of compounds of Formula Im above, R₁ is as defined in paragraph (13) above and R₃ and R₄ have any one of the definitions herein.

In a particular group of compounds of Formula Im above, R₁ is as defined in paragraph (14) above and R₃ and R₄ have any one of the definitions herein.

In a particular group of compounds of Formula Im above, R₃ and R₄ are as defined in any one of paragraphs (15) to (18), or (19) to (21) above and R₁ has any one of the definitions herein.

In a particular group of compounds of Formula Im above, R₃ and R₄ are as defined in any one of paragraphs (22) to (24) above and R₁ has any one of the definitions herein.

In a particular group of compounds of Formula Im above, R₃ and R₄ are as defined in paragraph (15) above and R₁ has any one of the definitions herein.

In a particular group of compounds of Formula Im above, R₃ and R₄ are as defined in paragraph (16) above and R₁ has any one of the definitions herein.

In a particular group of compounds of Formula Im above, R₃ and R₄ are as defined in paragraph (17) above and R₁ has any one of the definitions herein.

In a particular group of compounds of Formula Im above, R₃ and R₄ are as defined in paragraph (18) above and R₁ has any one of the definitions herein.

In a particular group of compounds of Formula Im above, R₃ and R₄ are as defined in paragraph (19) above and R₁ has any one of the definitions herein.

In a particular group of compounds of Formula Im above, R₃ and R₄ are as defined in paragraph (20) above and R₁ has any one of the definitions herein.

In a particular group of compounds of Formula Im above, R₃ and R₄ are as defined in paragraph (21) above and R₁ has any one of the definitions herein.

In a particular group of compounds of Formula Im above, R₃ and R₄ are as defined in paragraph (22) above and R₁ has any one of the definitions herein.

In a particular group of compounds of Formula Im above, R₃ and R₄ are as defined in paragraph (23) above and R₁ has any one of the definitions herein.

In a particular group of compounds of Formula Im above, R₃ and R₄ are as defined in paragraph (24) above and R₁ has any one of the definitions herein.

In a particular group of compounds of Formula Im above, R₃ is hydrogen, R₄ is as defined in paragraph (18) above and R₁ has any one of the definitions herein.

In a particular group of compounds of Formula Im above, R₃ is hydrogen, R₄ is as defined in paragraph (19) above and R₁ has any one of the definitions herein.

In a particular group of compounds of Formula Im above, R₃ is hydrogen, R₄ is as defined in paragraph (20) above and R₁ has any one of the definitions herein.

In a particular group of compounds of Formula Im above, R₃ is hydrogen, R₄ is as defined in paragraph (21) above and R₁ has any one of the definitions herein.

In a particular group of compounds of Formula Im above, R₃ is hydrogen, R₄ is as defined in paragraph (22) above and R₁ has any one of the definitions herein.

In a particular group of compounds of Formula Im above, R₃ is hydrogen, R₄ is as defined in paragraph (23) above and R₁ has any one of the definitions herein.

In a particular group of compounds of Formula Im above, R₃ is hydrogen, R₄ is as defined in paragraph (24) above and R₁ has any one of the definitions herein.

In a particular group of compounds of Formula Im above:
R₁ is as defined in any one of paragraphs (7) to (14);
R₃ is as defined in any one of paragraphs (18) to (21) above; and
R₄ is as defined in any one of paragraphs (18) to (21) above.

In a particular group of compounds of Formula Im above:
R₁ is as defined in any one of paragraphs (9) to (14) above;
R₃ is as defined in any one of paragraphs (18) to (21) above; and
R₄ is as defined in any one of paragraphs (18) to (21) above.

In a particular group of compounds of Formula Im above:
R₁ is as defined in any one of paragraphs (12), (13) or (14);
R₃ is hydrogen; and
R₄ is as defined in any one of paragraphs (19) to (21) above.

In a particular group of compounds of the invention, the compounds have the structural formula II, [a sub-definition of formula (I)] shown below, or a pharmaceutically acceptable salt, hydrate and/or solvate thereof:

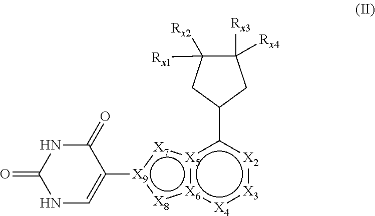

wherein X₂, X₃, X₄, X₅, Rₓ₁, Rₓ₂, Rₓ₃ and Rₓ₄ are as defined herein.

In a particular group of compounds of Formula I above X₂, X₃, X₄, X₅, X₆, X₇, X₈ and X₉ are as defined in paragraph (15) above, and Rₓ₁, Rₓ₂, Rₓ₃ and Rₓ₄ have any one of the definitions herein.

In a particular group of compounds of Formula I above, X₂, X₃, X₄, X₅, X₆, X₇, X₈ and X₉ are as defined in paragraph (16) above and Rₓ₁, Rₓ₂, Rₓ₃ and Rₓ₄ have any one of the definitions herein.

In a particular group of compounds of Formula I above, X₂, X₃, X₄, X₅, X₆, X₇, X₈ and X₉ are as defined in paragraph (17) above and Rₓ₁, Rₓ₂, Rₓ₃ and Rₓ₄ have any one of the definitions herein.

In a particular group of compounds of Formula I above, X₂, X₃, X₄, X₅, X₆, X₇, X₈ and X₉ are as defined in paragraph (18) above and Rₓ₁, Rₓ₂, Rₓ₃ and Rₓ₄ have any one of the definitions herein.

In a particular group of compounds of Formula I above, X₂, X₃, X₄, X₅, X₆, X₇, X₈ and X₉ are as defined in paragraph (19) above and Rₓ₁, Rₓ₂, Rₓ₃ and Rₓ₄ have any one of the definitions herein.

In a particular group of compounds of Formula I above, X₂, X₃, X₄, X₅, X₆, X₇, X₈ and X₉ are as defined in paragraph (20) above and Rₓ₁, Rₓ₂, Rₓ₃ and Rₓ₄ have any one of the definitions herein.

In a particular group of compounds of Formula I above, X₂, X₃, X₄, X₅, X₆, X₇, X₈ and X₉ are as defined in paragraph (21) above and Rₓ₁, Rₓ₂, Rₓ₃ and Rₓ₄ have any one of the definitions herein.

In a particular group of compounds of Formula I above X₂, X₃, X₄, X₅, X₆, X₇, X₈ and X₉ are as defined anywhere herein, and Rₓ₁, Rₓ₂, Rₓ₃ and Rₓ₄ are as defined in paragraph (10) above.

In a particular group of compounds of Formula I above X₂, X₃, X₄, X₅, X₆, X₇, X₈ and X₉ are as defined anywhere herein, and Rₓ₁, Rₓ₂, Rₓ₃ and Rₓ₄ are as defined in paragraph (11) above.

In a particular group of compounds of Formula I above X₂, X₃, X₄, X₅, X₆, X₇, X₈ and X₉ are as defined anywhere herein, and Rₓ₁, Rₓ₂, Rₓ₃ and Rₓ₄ are as defined in paragraph (12) above.

Particular compounds of the present invention include any of the compounds described in the example section of the present application, or a pharmaceutically acceptable salt or solvate thereof, and, in particular, any of the following:
5-[4-[3-(Hydroxymethyl)pyrrolidin-1-yl]thieno[2,3-d]pyrimidin-6-yl]-1H-pyrimidine-2,4-dione;
5-[4-[3-(2-Hydroxyethyl)pyrrolidin-1-yl]thieno[2,3-d]pyrimidin-6-yl]-1H-pyrimidine-2,4-dione;
5-[4-(3,3-Dimethylazetidin-1-yl)thieno[2,3-d]pyrimidin-6-yl]-1H-pyrimidine-2,4-dione
5-[4-[3-(2-Hydroxyethoxy)pyrrolidin-1-yl]thieno[2,3-d]pyrimidin-6-yl]-1H-pyrimidine-2,4-dione;
5-[4-[3-[2-(1-Piperidyl)ethoxy]pyrrolidin-1-yl]thieno[2,3-d]pyrimidin-6-yl]-1H-pyrimidine-2,4-dione;
5-[4-(3,3-Dimethylpyrrolidin-1-yl)thieno[2,3-d]pyrimidin-6-yl]-1H-pyrimidine-2,4-dione;
6-[1-[6-(2,4-Dioxo-1H-pyrimidin-5-yl)thieno[2,3-d]pyrimidin-4-yl]pyrrolidin-3-yl]oxypyridine-3-carbonitrile;
5-[4-(3,3-Difluoro-4,4-dimethyl-pyrrolidin-1-yl)thieno[2,3-d]pyrimidin-6-yl]-1H-pyrimidine-2,4-dione;
5-[4-[3-(2-pyridylmethoxy)pyrrolidine-1-yl]thieno[2,3-d]pyrimidin-6-yl]-1H-pyrimidine-2,4-dione;
5-[4-(3-Benzyloxypyrrolidin-1-yl)thieno[2,3-d]pyrimidin-6-yl]-1H-pyrimidine-2,4-dione;
5-[4-[3,3-Difluoro-4-[(4-methylmorpholin-2-yl)methoxy]pyrrolidine-1-yl]thieno[2,3-d]pyrimidin-6-yl]-1H-pyrimidine-2,4-dione;
5-[4-[3-[(4-Methylmorpholin-2-yl)methoxy]pyrrolidine-1-yl]thieno[2,3-d]pyrimidin-6-yl]-1H-pyrimidine-2,4-dione;
5-[4-[3,3-Difluoro-4-[2-(1-piperidyl)ethoxy]pyrrolidine-1-yl]thieno[2,3-d]pyrimidin-6-yl]-1H-pyrimidine-2,4-dione;
5-[7-[3-[2-(1-Piperidyl)ethoxy]pyrrolidin-1-yl]thiazolo[4,5-d]pyrimidin-2-yl]-1H-pyrimidine-2,4-dione;
5-[7-(3,3-difluoro-4,4-dimethyl-pyrrolidin-1-yl)thiazolo[4,5-d]pyrimidin-2-yl]-1H-pyrimidine-2,4-dione;
5-[4-[3-[2-(1-Piperidyl)ethoxy]pyrrolidin-1-yl]furo[2,3-d]pyrimidin-6-yl]-1H-pyrimidine-2,4-dione;
5-[4-(3,3-Difluoro-4,4-dimethyl-pyrrolidin-1-yl)furo[2,3-d]pyrimidin-6-yl]-1H-pyrimidine-2,4-dione;
5-[4-[3-[2-(1-Piperidyl)ethoxy]pyrrolidin-1-yl]thieno[3,2-d]pyrimidin-6-yl]-1H-pyrimidine-2,4-dione;
5-[4-[3-[2-(1-Piperidyl)ethoxy]pyrrolidin-1-yl]thieno[3,2-c]pyridin-2-yl]-1H-pyrimidine-2,4-dione;
5-[4-[3-[2-(1-Piperidyl)ethoxy]pyrrolidin-1-yl]pyrrolo[2,1-f][1,2,4]triazin-6-yl]pyrimidine-2,4-diol; 5-[4-[3-[2-(4,4-Difluoro-1-piperidyl)ethoxy]pyrrolidin-1-yl]pyrrolo[2,1-f][1,2,4]triazin-6-yl]-1H-pyrimidine-2,4-dione;
5-[4-(3,3-Difluoro-4,4-dimethyl-pyrrolidin-1-yl)pyrrolo[2,1-f][1,2,4]triazin-6-yl]-1H-pyrimidine-2,4-dione;
2-(2,4-Dioxo-1H-pyrimidin-5-yl)-4-[3-[2-(1-piperidyl)ethoxy]pyrrolidin-1-yl]thieno[2,3-b]pyridine-5-carbonitrile; 5-[4-[3-[2-(1-Piperidyl)ethoxy]pyrrolidin-1-yl]pyrazolo[3,4-d]pyrimidin-2-yl]-1H-pyrimidine-2,4-dione;
5-[4-(3,3-Difluoro-4,4-dimethyl-pyrrolidin-1-yl)pyrazolo[3,4-d]pyrimidin-2-yl]-1H-pyrimidine-2,4-dione;
5-[4-(7,7-Difluoro-5-azaspiro[2.4]heptan-5-yl)pyrazolo[3,4-d]pyrimidin-2-yl]-1H-pyrimidine-2,4-dione;
5-[4-(8,8-Difluoro-6-azaspiro[3.4]octan-6-yl)pyrazolo[3,4-d]pyrimidin-2-yl]-1H-pyrimidine-2,4-dione;
5-[4-(4,4-Difluoro-2-azaspiro[4.4]nonan-2-yl)pyrazolo[3,4-d]pyrimidin-2-yl]-1H-pyrimidine-2,4-dione;
5-[4-[(3aR,6aS)-3,3a,4,5,6,6a-Hexahydro-1H-cyclopenta[c]pyrrol-2-yl]pyrazolo[3,4-d]pyrimidin-2-yl]-1H-pyrimidine-2,4-dione;
5-[4-(3-Ethyl-3-methyl-pyrrolidin-1-yl)pyrazolo[3,4-d]pyrimidin-2-yl]-1H-pyrimidine-2,4-dione;
5-[4-(3,3-Dimethylpyrrolidin-1-yl)pyrazolo[3,4-d]pyrimidin-2-yl]-1H-pyrimidine-2,4-dione;
5-[4-(3,3-Diethylpyrrolidin-1-yl)pyrazolo[3,4-d]pyrimidin-2-yl]-1H-pyrimidine-2,4-dione;
5-[4-(3,3,4,4-Tetrafluoropyrrolidin-1-yl)pyrazolo[3,4-d]pyrimidin-2-yl]-1H-pyrimidine-2,4-dione;
5-[4-[(3aR,6aS)-1,3,3a,4,6,6a-Hexahydrofuro[3,4-c]pyrrol-5-yl]pyrazolo[3,4-d]pyrimidin-2-yl]-1H-pyrimidine-2,4-dione;
5-[4-[(3aR,6aR)-1,3,3a,4,6,6a-Hexahydrofuro[3,4-c]pyrrol-5-yl]pyrazolo[3,4-d]pyrimidin-2-yl]-5H-pyrimidine-2,4-dione;
5-[4-(3,3-Difluoro-4-methoxy-pyrrolidin-1-yl)pyrazolo[3,4-d]pyrimidin-2-yl]-1H-pyrimidine-2,4-dione;
5-[4-[3-[2-(1-Piperidyl)ethoxy]pyrrolidin-1-yl]thieno[2,3-b]pyridin-2-yl]-1H-pyrimidine-2,4-dione;
5-[4-(3,3-Difluoro-4,4-dimethyl-pyrrolidin-1-yl)thieno[2,3-b]pyridin-2-yl]-1H-pyrimidine-2,4-dione;
5-[6-[3-[2-(1-Piperidyl)ethoxy]pyrrolidin-1-yl]-9H-purin-8-yl]-1H-pyrimidine-2,4-dione;
5-[7-[3-[2-(1-Piperidyl)ethoxy]pyrrolidin-1-yl]thieno[3,2-b]pyridin-2-yl]-1H-pyrimidine-2,4-dione;
5-[4-[3,3-Difluoro-4-[2-(1-piperidyl)ethoxy]pyrrolidin-1-yl]pyrrolo[2,1-f][1,2,4]triazin-6-yl]-1H-pyrimidine-2,4-dione;
5-[2-Chloro-4-[3-[2-(1-piperidyl)ethoxy]pyrrolidin-1-yl]pyrrolo[2,1-f][1,2,4]triazin-6-yl]-5H-pyrimidine-2,4-dione;
5-[4-(8,8-Difluoro-6-azaspiro[3.4]octan-6-yl)furo[2,3-d]pyrimidin-6-yl]-1H-pyrimidine-2,4-dione;
5-[4-(4,4-Difluoro-2-azaspiro[4.4]nonan-2-yl)furo[2,3-d]pyrimidin-6-yl]-1H-pyrimidine-2,4-dione;
5-[4-[(4-Chlorophenyl)methylamino]furo[2,3-d]pyrimidin-6-yl]-1H-pyrimidine-2,4-dione;
5-[4-(7,7-Difluoro-5-azaspiro[2.4]heptan-5-yl)furo[2,3-d]pyrimidin-6-yl]-1H-pyrimidine-2,4-dione;
[3-[[6-(2,4-Dioxo-1H-pyrimidin-5-yl)furo[2,3-d]pyrimidin-4-yl]amino]-2,2-difluoro-propyl]N-isopropylcarbamate;
5-[4-[3-[2-[4-(Trifluoromethyl)-1-piperidyl]ethoxy]63yrrolidine-1-yl]furo[2,3-d]pyrimidin-6-yl]-1H-pyrimidine-2,4-dione;
[1-[6-(2,4-Dioxo-1H-pyrimidin-5-yl)furo[2,3-d]pyrimidin-4-yl]-4,4-difluoro-pyrrolidin-3-yl]morpholine-4-carboxylate;
[1-[6-(2,4-Dioxo-1H-pyrimidin-5-yl)furo[2,3-d]pyrimidin-4-yl]-4,4-difluoro-pyrrolidin-3-yl]8-oxa-3-azabicyclo[3.2.1]octane-3-carboxylate;
[1-[6-(2,4-Dioxo-1H-pyrimidin-5-yl)furo[2,3-d]pyrimidin-4-yl]-4,4-difluoro-pyrrolidin-3-yl]N-isopropylcarbamate;
5-[4-(3,3-Difluoro-4,4-dimethyl-pyrrolidin-1-yl)-6-methyl-pyrazolo[3,4-d]pyrimidin-2-yl]-1H-pyrimidine-2,4-dione;
6-[1-[2-(2,4-Dioxo-1H-pyrimidin-5-yl)pyrazolo[3,4-d]pyrimidin-4-yl]63yrrolidine-3-yl]oxypyridine-3-carbonitrile;
5-[4-(3,3-Difluoro-4,4-dimethyl-pyrrolidin-1-yl)pyrazolo[1,5-a]pyrazin-2-yl]-1H-pyrimidine-2,4-dione;
5-[4-(8,8-Difluoro-6-azaspiro[3.4]octan-6-yl)pyrazolo[1,5-a]pyrazin-2-yl]-1H-pyrimidine-2,4-dione;
5-[4-(7,7-Difluoro-5-azaspiro[2.4]heptan-5-yl)pyrazolo[1,5-a]pyrazin-2-yl]-1H-pyrimidine-2,4-dione;
5-[4-(3,3-difluoro-4-methoxy-pyrrolidin-1-yl)pyrazolo[1,5-a]pyrazin-2-yl]-1H-pyrimidine-2,4-dione;
5-[4-(2-oxa-7-azaspiro[3.4]octan-7-yl)pyrazolo[1,5-a]pyrazin-2-yl]-1H-pyrimidine-2,4-dione;
5-[4-(3,3-difluoro-4-hydroxy-pyrrolidin-1-yl)pyrazolo[1,5-a]pyrazin-2-yl]-1H-pyrimidine-2,4-dione;
5-[4-[3,3-difluoro-4-[(4-methylmorpholin-2-yl)methoxy]pyrrolidin-1-yl]pyrazolo[1,5-a]pyrazin-2-yl]-1H-pyrimidine-2,4-dione;
5-[4-[3,3-difluoro-4-(2-morpholinoethoxy)pyrrolidin-1-yl]pyrazolo[1,5-a]pyrazin-2-yl]-1H-pyrimidine-2,4-dione;
5-[7-chloro-4-(3,3-difluoro-4,4-dimethyl-pyrrolidin-1-yl)pyrazolo[1,5-a]pyrazin-2-yl]-1H-pyrimidine-2,4-dione;
5-[4-(3,3-difluoro-4,4-dimethyl-pyrrolidin-1-yl)-7-(trifluoromethyl)pyrazolo[1,5-a]pyrazin-2-yl]-1H-pyrimidine-2,4-dione;
5-[4-(3,3-difluoro-4,4-dimethyl-pyrrolidin-1-yl)-7-fluoro-pyrazolo[1,5-a]pyrazin-2-yl]-1H-pyrimidine-2,4-dione;
5-[4-(3,3-difluoro-4,4-dimethyl-pyrrolidin-1-yl)-7-methyl-pyrazolo[1,5-a]pyrazin-2-yl]-1H-pyrimidine-2,4-dione;
5-[4-(3,3-Difluoro-4,4-dimethyl-pyrrolidin-1-yl)-7-fluoro-pyrazolo[4,3-c]pyridin-2-yl]-1H-pyrimidine-2,4-dione;
5-[4-(8,8-difluoro-6-azaspiro[3.4]octan-6-yl)-7-fluoro-pyrazolo[4,3-c]pyridin-2-yl]-1H-pyrimidine-2,4-dione;
5-[4-(3,3-difluoro-4-methoxy-pyrrolidin-1-yl)-7-fluoro-pyrazolo[4,3-c]pyridin-2-yl]-1H-pyrimidine-2,4-dione;
5-[4-(3,3-difluoro-4-hydroxy-pyrrolidin-1-yl)-7-fluoro-pyrazolo[4,3-c]pyridin-2-yl]-1H-pyrimidine-2,4-dione;
5-[7-fluoro-4-(2-oxa-7-azaspiro[3.4]octan-7-yl)pyrazolo[4,3-c]pyridin-2-yl]-1H-pyrimidine-2,4-dione;
5-[4-(7,7-difluoro-5-azaspiro[2.4]heptan-5-yl)-7-fluoro-pyrazolo[4,3-c]pyridin-2-yl]-1H-pyrimidine-2,4-dione;
5-[4-[3,3-difluoro-4-[(4-methylmorpholin-2-yl)methoxy]pyrrolidin-1-yl]-7-fluoro-pyrazolo[4,3-c]pyridin-2-yl]-1H-pyrimidine-2,4-dione;
5-[4-(3,3-Difluoro-4,4-dimethyl-pyrrolidin-1-yl)pyrazolo[4,3-c]pyridin-2-yl]-1H-pyrimidine-2,4-dione;
5-[7-chloro-4-(3,3-difluoro-4,4-dimethyl-pyrrolidin-1-yl)pyrazolo[4,3-c]pyridin-2-yl]-1H-pyrimidine-2,4-dione;
5-[7-chloro-4-(7,7-difluoro-5-azaspiro[2.4]heptan-5-yl)pyrazolo[4,3-c]pyridin-2-yl]-1H-pyrimidine-2,4-dione;
5-[7-chloro-4-(8,8-difluoro-6-azaspiro[3.4]octan-6-yl)pyrazolo[4,3-c]pyridin-2-yl]-1H-pyrimidine-2,4-dione;
4-(3,3-difluoro-4,4-dimethyl-pyrrolidin-1-yl)-2-(2,4-dioxo-1H-pyrimidin-5-yl)pyrazolo[4,3-c]pyridine-7-carbonitrile; 5-[4-(3,3-difluoro-4,4-dimethyl-pyrrolidin-1-yl)pyrazolo[3,4-b]pyridine-2-yl]-1H-pyrimidine-2,4-dione;
5-[4-(3,3-difluoro-4,4-dimethyl-pyrrolidin-1-yl)pyrazolo[3,4-c]pyridine-2-yl]-1H-pyrimidine-2,4-dione;
5-4-(3,3-difluoro-4,4-dimethyl-pyrrolidin-1-yl)-7-iodo-pyrazolo[1,5-a]pyrazin-2-yl]-1H-pyrimidine-2,4-dione;
5-[7-Chloro-4-(3,3-difluoro-4-hydroxy-pyrrolidin-1-yl)pyrazolo[1,5-a]pyrazin-2-yl]-1H-pyrimidine-2,4-dione;
5-[7-Chloro-4-(3,3-difluoro-4-methoxy-pyrrolidin-1-yl)pyrazolo[1,5-a]pyrazin-2-yl]-1H-pyrimidine-2,4-dione;
5-[7-Chloro-4-(3,3-difluoro-4,4-dimethyl-pyrrolidin-1-yl)-6-methyl-pyrazolo[1,5-a]pyrazin-2-yl]-1H-pyrimidine-2,4-dione;
5-[7-chloro-4-(3,3-dimethylpyrrolidin-1-yl)pyrazolo[1,5-a]pyrazin-2-yl]-1H-pyrimidine-2,4-dione; and
5-[4-(3,3-dimethylpyrrolidin-1-yl)-7-fluoro-pyrazolo[1,5-a]pyrazin-2-yl]-1H-pyrimidine-2,4-dione.

Particular compounds of the present invention include any of the compounds described in the example section of the present application, or a pharmaceutically acceptable salt or solvate thereof, and, in particular, any of the following:
5-[4-[3-(Hydroxymethyl)pyrrolidin-1-yl]thieno[2,3-d]pyrimidin-6-yl]-1H-pyrimidine-2,4-dione;
5-[4-[3-(2-Hydroxyethyl)pyrrolidin-1-yl]thieno[2,3-d]pyrimidin-6-yl]-1H-pyrimidine-2,4-dione;
5-[4-(3,3-Dimethylazetidin-1-yl)thieno[2,3-d]pyrimidin-6-yl]-1H-pyrimidine-2,4-dione
5-[4-[3-(2-Hydroxyethoxy)pyrrolidin-1-yl]thieno[2,3-d]pyrimidin-6-yl]-1H-pyrimidine-2,4-dione;
5-[4-[3-[2-(1-Piperidyl)ethoxy]pyrrolidin-1-yl]thieno[2,3-d]pyrimidin-6-yl]-1H-pyrimidine-2,4-dione;
5-[4-(3,3-Dimethylpyrrolidin-1-yl)thieno[2,3-d]pyrimidin-6-yl]-1H-pyrimidine-2,4-dione;
6-[1-[6-(2,4-Dioxo-1H-pyrimidin-5-yl)thieno[2,3-d]pyrimidin-4-yl]pyrrolidin-3-yl]oxypyridine-3-carbonitrile;
5-[4-(3,3-Difluoro-4,4-dimethyl-pyrrolidin-1-yl)thieno[2,3-d]pyrimidin-6-yl]-1H-pyrimidine-2,4-dione;
5-[4-[3-(2-pyridylmethoxy)pyrrolidine-1-yl]thieno[2,3-d]pyrimidin-6-yl]-1H-pyrimidine-2,4-dione;
5-[4-(3-Benzyloxypyrrolidin-1-yl)thieno[2,3-d]pyrimidin-6-yl]-1H-pyrimidine-2,4-dione;
5-[4-[3,3-Difluoro-4-[(4-methylmorpholin-2-yl)methoxy]pyrrolidine-1-yl]thieno[2,3-d]pyrimidin-6-yl]-1H-pyrimidine-2,4-dione;
5-[4-[3-[(4-Methylmorpholin-2-yl)methoxy]pyrrolidine-1-yl]thieno[2,3-d]pyrimidin-6-yl]-1H-pyrimidine-2,4-dione;
5-[4-[3,3-Difluoro-4-[2-(1-piperidyl)ethoxy]pyrrolidine-1-yl]thieno[2,3-d]pyrimidin-6-yl]-1H-pyrimidine-2,4-dione;
5-[7-[3-[2-(1-Piperidyl)ethoxy]pyrrolidin-1-yl]thiazolo[4,5-d]pyrimidin-2-yl]-1H-pyrimidine-2,4-dione;
5-[7-(3,3-difluoro-4,4-dimethyl-pyrrolidin-1-yl)thiazolo[4,5-d]pyrimidin-2-yl]-1H-pyrimidine-2,4-dione;
5-[4-[3-[2-(1-Piperidyl)ethoxy]pyrrolidin-1-yl]furo[2,3-d]pyrimidin-6-yl]-1H-pyrimidine-2,4-dione;
5-[4-(3,3-Difluoro-4,4-dimethyl-pyrrolidin-1-yl)furo[2,3-d]pyrimidin-6-yl]-1H-pyrimidine-2,4-dione;
5-[4-[3-[2-(1-Piperidyl)ethoxy]pyrrolidin-1-yl]thieno[3,2-d]pyrimidin-6-yl]-1H-pyrimidine-2,4-dione;
5-[4-[3-[2-(1-Piperidyl)ethoxy]pyrrolidin-1-yl]thieno[3,2-c]pyridin-2-yl]-1H-pyrimidine-2,4-dione;
5-[4-[3-[2-(1-Piperidyl)ethoxy]pyrrolidin-1-yl]pyrrolo[2,1-f][1,2,4]triazin-6-yl]pyrimidine-2,4-diol; 5-[4-[3-[2-(4,4-Difluoro-1-piperidyl)ethoxy]pyrrolidin-1-yl]pyrrolo[2,1-f][1,2,4]triazin-6-yl]-1H-pyrimidine-2,4-dione;
5-[4-(3,3-Difluoro-4,4-dimethyl-pyrrolidin-1-yl)pyrrolo[2,1-f][1,2,4]triazin-6-yl]-1H-pyrimidine-2,4-dione;
2-(2,4-Dioxo-1H-pyrimidin-5-yl)-4-[3-[2-(1-piperidyl)ethoxy]pyrrolidin-1-yl]thieno[2,3-b]pyridine-5-carbonitrile;
5-[4-[3-[2-(1-Piperidyl)ethoxy]pyrrolidin-1-yl]pyrazolo[3,4-d]pyrimidin-2-yl]-1H-pyrimidine-2,4-dione;
5-[4-(3,3-Difluoro-4,4-dimethyl-pyrrolidin-1-yl)pyrazolo[3,4-d]pyrimidin-2-yl]-1H-pyrimidine-2,4-dione;
5-[4-(7,7-Difluoro-5-azaspiro[2.4]heptan-5-yl)pyrazolo[3,4-d]pyrimidin-2-yl]-1H-pyrimidine-2,4-dione;
5-[4-(8,8-Difluoro-6-azaspiro[3.4]octan-6-yl)pyrazolo[3,4-d]pyrimidin-2-yl]-1H-pyrimidine-2,4-dione;
5-[4-(4,4-Difluoro-2-azaspiro[4.4]nonan-2-yl)pyrazolo[3,4-d]pyrimidin-2-yl]-1H-pyrimidine-2,4-dione;
5-[4-[(3aR,6aS)-3,3a,4,5,6,6a-Hexahydro-1H-cyclopenta[c]pyrrol-2-yl]pyrazolo[3,4-d]pyrimidin-2-yl]-1H-pyrimidine-2,4-dione;
5-[4-(3-Ethyl-3-methyl-pyrrolidin-1-yl)pyrazolo[3,4-d]pyrimidin-2-yl]-1H-pyrimidine-2,4-dione;
5-[4-(3,3-Dimethylpyrrolidin-1-yl)pyrazolo[3,4-d]pyrimidin-2-yl]-1H-pyrimidine-2,4-dione;
5-[4-(3,3-Diethylpyrrolidin-1-yl)pyrazolo[3,4-d]pyrimidin-2-yl]-1H-pyrimidine-2,4-dione;
5-[4-(3,3,4,4-Tetrafluoropyrrolidin-1-yl)pyrazolo[3,4-d]pyrimidin-2-yl]-1H-pyrimidine-2,4-dione;
5-[4-[(3aR,6aS)-1,3,3a,4,6,6a-Hexahydrofuro[3,4-c]pyrrol-5-yl]pyrazolo[3,4-d]pyrimidin-2-yl]-1H-pyrimidine-2,4-dione;
5-[4-[(3aR,6aR)-1,3,3a,4,6,6a-Hexahydrofuro[3,4-c]pyrrol-5-yl]pyrazolo[3,4-d]pyrimidin-2-yl]-5H-pyrimidine-2,4-dione;
5-[4-(3,3-Difluoro-4-methoxy-pyrrolidin-1-yl)pyrazolo[3,4-d]pyrimidin-2-yl]-1H-pyrimidine-2,4-dione;
5-[4-[3-[2-(1-Piperidyl)ethoxy]pyrrolidin-1-yl]thieno[2,3-b]pyridin-2-yl]-1H-pyrimidine-2,4-dione;
5-[4-(3,3-Difluoro-4,4-dimethyl-pyrrolidin-1-yl)thieno[2,3-b]pyridin-2-yl]-1H-pyrimidine-2,4-dione;
5-[6-[3-[2-(1-Piperidyl)ethoxy]pyrrolidin-1-yl]-9H-purin-8-yl]-1H-pyrimidine-2,4-dione;
5-[7-[3-[2-(1-Piperidyl)ethoxy]pyrrolidin-1-yl]thieno[3,2-b]pyridin-2-yl]-1H-pyrimidine-2,4-dione;
5-[4-[3,3-Difluoro-4-[2-(1-piperidyl)ethoxy]pyrrolidin-1-yl]pyrrolo[2,1-f][1,2,4]triazin-6-yl]-1H-pyrimidine-2,4-dione;
5-[2-Chloro-4-[3-[2-(1-piperidyl)ethoxy]pyrrolidin-1-yl]pyrrolo[2,1-f][1,2,4]triazin-6-yl]-5H-pyrimidine-2,4-dione;
5-[4-(8,8-Difluoro-6-azaspiro[3.4]octan-6-yl)furo[2,3-d]pyrimidin-6-yl]-1H-pyrimidine-2,4-dione;
5-[4-(4,4-Difluoro-2-azaspiro[4.4]nonan-2-yl)furo[2,3-d]pyrimidin-6-yl]-1H-pyrimidine-2,4-dione;
5-[4-[(4-Chlorophenyl)methylamino]furo[2,3-d]pyrimidin-6-yl]-1H-pyrimidine-2,4-dione;
5-[4-(7,7-Difluoro-5-azaspiro[2.4]heptan-5-yl)furo[2,3-d]pyrimidin-6-yl]-1H-pyrimidine-2,4-dione;
[3-[[6-(2,4-Dioxo-1H-pyrimidin-5-yl)furo[2,3-d]pyrimidin-4-yl]amino]-2,2-difluoro-propyl]N-isopropylcarbamate;
5-[4-[3-[2-[4-(Trifluoromethyl)-1-piperidyl]ethoxy]68yrrolidine-1-yl]furo[2,3-d]pyrimidin-6-yl]-1H-pyrimidine-2,4-dione;
[1-[6-(2,4-Dioxo-1H-pyrimidin-5-yl)furo[2,3-d]pyrimidin-4-yl]-4,4-difluoro-pyrrolidin-3-yl]morpholine-4-carboxylate;
[1-[6-(2,4-Dioxo-1H-pyrimidin-5-yl)furo[2,3-d]pyrimidin-4-yl]-4,4-difluoro-pyrrolidin-3-yl]8-oxa-3-azabicyclo[3.2.1]octane-3-carboxylate;
[1-[6-(2,4-Dioxo-1H-pyrimidin-5-yl)furo[2,3-d]pyrimidin-4-yl]-4,4-difluoro-pyrrolidin-3-yl]N-isopropylcarbamate;
5-[4-(3,3-Difluoro-4,4-dimethyl-pyrrolidin-1-yl)-6-methyl-pyrazolo[3,4-d]pyrimidin-2-yl]-1H-pyrimidine-2,4-dione;
6-[1-[2-(2,4-Dioxo-1H-pyrimidin-5-yl)pyrazolo[3,4-d]pyrimidin-4-yl]68yrrolidine-3-yl]oxypyridine-3-carbonitrile;
5-[4-(3,3-Difluoro-4,4-dimethyl-pyrrolidin-1-yl)pyrazolo[1,5-a]pyrazin-2-yl]-1H-pyrimidine-2,4-dione;
5-[4-(8,8-Difluoro-6-azaspiro[3.4]octan-6-yl)pyrazolo[1,5-a]pyrazin-2-yl]-1H-pyrimidine-2,4-dione;
5-[4-(7,7-Difluoro-5-azaspiro[2.4]heptan-5-yl)pyrazolo[1,5-a]pyrazin-2-yl]-1H-pyrimidine-2,4-dione;
5-[4-(3,3-difluoro-4-methoxy-pyrrolidin-1-yl)pyrazolo[1,5-a]pyrazin-2-yl]-1H-pyrimidine-2,4-dione;
5-[4-(2-oxa-7-azaspiro[3.4]octan-7-yl)pyrazolo[1,5-a]pyrazin-2-yl]-1H-pyrimidine-2,4-dione;
5-[4-(3,3-difluoro-4-hydroxy-pyrrolidin-1-yl)pyrazolo[1,5-a]pyrazin-2-yl]-1H-pyrimidine-2,4-dione;
5-[4-[3,3-difluoro-4-[(4-methylmorpholin-2-yl)methoxy]pyrrolidin-1-yl]pyrazolo[1,5-a]pyrazin-2-yl]-1H-pyrimidine-2,4-dione;
5-[4-[3,3-difluoro-4-(2-morpholinoethoxy)pyrrolidin-1-yl]pyrazolo[1,5-a]pyrazin-2-yl]-1H-pyrimidine-2,4-dione;
5-[7-chloro-4-(3,3-difluoro-4,4-dimethyl-pyrrolidin-1-yl)pyrazolo[1,5-a]pyrazin-2-yl]-1H-pyrimidine-2,4-dione;
5-[4-(3,3-difluoro-4,4-dimethyl-pyrrolidin-1-yl)-7-(trifluoromethyl)pyrazolo[1,5-a]pyrazin-2-yl]-1H-pyrimidine-2,4-dione;
5-[4-(3,3-difluoro-4,4-dimethyl-pyrrolidin-1-yl)-7-fluoro-pyrazolo[1,5-a]pyrazin-2-yl]-1H-pyrimidine-2,4-dione;
5-[4-(3,3-difluoro-4,4-dimethyl-pyrrolidin-1-yl)-7-methyl-pyrazolo[1,5-a]pyrazin-2-yl]-1H-pyrimidine-2,4-dione;
5-[4-(3,3-Difluoro-4,4-dimethyl-pyrrolidin-1-yl)-7-fluoro-pyrazolo[4,3-c]pyridin-2-yl]-1H-pyrimidine-2,4-dione;
5-[4-(8,8-difluoro-6-azaspiro[3.4]octan-6-yl)-7-fluoro-pyrazolo[4,3-c]pyridin-2-yl]-1H-pyrimidine-2,4-dione;
5-[4-(3,3-difluoro-4-methoxy-pyrrolidin-1-yl)-7-fluoro-pyrazolo[4,3-c]pyridin-2-yl]-1H-pyrimidine-2,4-dione;
5-[4-(3,3-difluoro-4-hydroxy-pyrrolidin-1-yl)-7-fluoro-pyrazolo[4,3-c]pyridin-2-yl]-1H-pyrimidine-2,4-dione;
5-[7-fluoro-4-(2-oxa-7-azaspiro[3.4]octan-7-yl)pyrazolo[4,3-c]pyridin-2-yl]-1H-pyrimidine-2,4-dione;
5-[4-(7,7-difluoro-5-azaspiro[2.4]heptan-5-yl)-7-fluoro-pyrazolo[4,3-c]pyridin-2-yl]-1H-pyrimidine-2,4-dione;
5-[4-[3,3-difluoro-4-[(4-methylmorpholin-2-yl)methoxy]pyrrolidin-1-yl]-7-fluoro-pyrazolo[4,3-c]pyridin-2-yl]-1H-pyrimidine-2,4-dione;
5-[4-(3,3-Difluoro-4,4-dimethyl-pyrrolidin-1-yl)pyrazolo[4,3-c]pyridin-2-yl]-1H-pyrimidine-2,4-dione;
5-[7-chloro-4-(3,3-difluoro-4,4-dimethyl-pyrrolidin-1-yl)pyrazolo[4,3-c]pyridin-2-yl]-1H-pyrimidine-2,4-dione;
5-[7-chloro-4-(7,7-difluoro-5-azaspiro[2.4]heptan-5-yl)pyrazolo[4,3-c]pyridin-2-yl]-1H-pyrimidine-2,4-dione;
5-[7-chloro-4-(8,8-difluoro-6-azaspiro[3.4]octan-6-yl)pyrazolo[4,3-c]pyridin-2-yl]-1H-pyrimidine-2,4-dione;
4-(3,3-difluoro-4,4-dimethyl-pyrrolidin-1-yl)-2-(2,4-dioxo-1H-pyrimidin-5-yl)pyrazolo[4,3-c]pyridine-7-carbonitrile; 5-[4-(3,3-difluoro-4,4-dimethyl-pyrrolidin-1-yl)pyrazolo[3,4-b]pyridine-2-yl]-1H-pyrimidine-2,4-dione; and
5-[4-(3,3-difluoro-4,4-dimethyl-pyrrolidin-1-yl)pyrazolo[3,4-c]pyridine-2-yl]-1H-pyrimidine-2,4-dione.

Particular compounds of the present invention include any of the compounds described in the example section of the present application, or a pharmaceutically acceptable salt or solvate thereof, and, in particular, any of the following:
5-[4-[3-(Hydroxymethyl)pyrrolidin-1-yl]thieno[2,3-d]pyrimidin-6-yl]-1H-pyrimidine-2,4-dione;
5-[4-[3-(2-Hydroxyethyl)pyrrolidin-1-yl]thieno[2,3-d]pyrimidin-6-yl]-1H-pyrimidine-2,4-dione;
5-[4-(3,3-Dimethylazetidin-1-yl)thieno[2,3-d]pyrimidin-6-yl]-1H-pyrimidine-2,4-dione;
5-[4-[3-(2-Hydroxyethoxy)pyrrolidin-1-yl]thieno[2,3-d]pyrimidin-6-yl]-1H-pyrimidine-2,4-dione;
5-[4-[3-[2-(1-Piperidyl)ethoxy]pyrrolidin-1-yl]thieno[2,3-d]pyrimidin-6-yl]-1H-pyrimidine-2,4-dione;
5-[4-(3,3-Dimethylpyrrolidin-1-yl)thieno[2,3-d]pyrimidin-6-yl]-1H-pyrimidine-2,4-dione;
6-[1-[6-(2,4-Dioxo-1H-pyrimidin-5-yl)thieno[2,3-d]pyrimidin-4-yl]pyrrolidin-3-yl]oxypyridine-3-carbonitrile;
5-[4-(3,3-Difluoro-4,4-dimethyl-pyrrolidin-1-yl)thieno[2,3-d]pyrimidin-6-yl]-1H-pyrimidine-2,4-dione;
5-[4-[3-(2-pyridylmethoxy)pyrrolidine-1-yl]thieno[2,3-d]pyrimidin-6-yl]-1H-pyrimidine-2,4-dione;
5-[4-(3-Benzyloxypyrrolidin-1-yl)thieno[2,3-d]pyrimidin-6-yl]-1H-pyrimidine-2,4-dione;
5-[4-[3,3-Difluoro-4-[(4-methylmorpholin-2-yl)methoxy]pyrrolidine-1-yl]thieno[2,3-d]pyrimidin-6-yl]-1H-pyrimidine-2,4-dione;
5-[4-[3-[(4-Methylmorpholin-2-yl)methoxy]pyrrolidine-1-yl]thieno[2,3-d]pyrimidin-6-yl]-1H-pyrimidine-2,4-dione;
5-[4-[3,3-Difluoro-4-[2-(1-piperidyl)ethoxy]pyrrolidine-1-yl]thieno[2,3-d]pyrimidin-6-yl]-1H-pyrimidine-2,4-dione;
5-[7-[3-[2-(1-Piperidyl)ethoxy]pyrrolidin-1-yl]thiazolo[4,5-d]pyrimidin-2-yl]-1H-pyrimidine-2,4-dione;
5-[4-[3-[2-(1-Piperidyl)ethoxy]pyrrolidin-1-yl]furo[2,3-d]pyrimidin-6-yl]-1H-pyrimidine-2,4-dione;
5-[4-(3,3-Difluoro-4,4-dimethyl-pyrrolidin-1-yl)furo[2,3-d]pyrimidin-6-yl]-1H-pyrimidine-2,4-dione;
5-[4-[3-[2-(1-Piperidyl)ethoxy]pyrrolidin-1-yl]thieno[3,2-d]pyrimidin-6-yl]-1H-pyrimidine-2,4-dione;
5-[4-[3-[2-(1-Piperidyl)ethoxy]pyrrolidin-1-yl]thieno[3,2-c]pyridin-2-yl]-1H-pyrimidine-2,4-dione;
5-[4-[3-[2-(1-Piperidyl)ethoxy]pyrrolidin-1-yl]pyrrolo[2,1-f][1,2,4]triazin-6-yl]pyrimidine-2,4-diol; 5-[4-[3-[2-(4,4-Difluoro-1-piperidyl)ethoxy]pyrrolidin-1-yl]pyrrolo[2,1-f][1,2,4]triazin-6-yl]-1H-pyrimidine-2,4-dione;
5-[4-(3,3-Difluoro-4,4-dimethyl-pyrrolidin-1-yl)pyrrolo[2,1-f][1,2,4]triazin-6-yl]-1H-pyrimidine-2,4-dione;
2-(2,4-Dioxo-1H-pyrimidin-5-yl)-4-[3-[2-(1-piperidyl)ethoxy]pyrrolidin-1-yl]thieno[2,3-b]pyridine-5-carbonitrile;
5-[4-[3-[2-(1-Piperidyl)ethoxy]pyrrolidin-1-yl]pyrazolo[3,4-d]pyrimidin-2-yl]-1H-pyrimidine-2,4-dione hydrochloride;
5-[4-(3,3-Difluoro-4,4-dimethyl-pyrrolidin-1-yl)pyrazolo[3,4-d]pyrimidin-2-yl]-1H-pyrimidine-2,4-dione;
5-[4-(7,7-Difluoro-5-azaspiro[2.4]heptan-5-yl)pyrazolo[3,4-d]pyrimidin-2-yl]-1H-pyrimidine-2,4-dione;
5-[4-(8,8-Difluoro-6-azaspiro[3.4]octan-6-yl)pyrazolo[3,4-d]pyrimidin-2-yl]-1H-pyrimidine-2,4-dione;
5-[4-(4,4-Difluoro-2-azaspiro[4.4]nonan-2-yl)pyrazolo[3,4-d]pyrimidin-2-yl]-1H-pyrimidine-2,4-dione;
5-[4-[(3aR,6aS)-3,3a,4,5,6,6a-Hexahydro-1H-cyclopenta[c]pyrrol-2-yl]pyrazolo[3,4-d]pyrimidin-2-yl]-1H-pyrimidine-2,4-dione;
5-[4-(3-Ethyl-3-methyl-pyrrolidin-1-yl)pyrazolo[3,4-d]pyrimidin-2-yl]-1H-pyrimidine-2,4-dione;
5-[4-(3,3-Dimethylpyrrolidin-1-yl)pyrazolo[3,4-d]pyrimidin-2-yl]-1H-pyrimidine-2,4-dione;
5-[4-(3,3-Diethylpyrrolidin-1-yl)pyrazolo[3,4-d]pyrimidin-2-yl]-1H-pyrimidine-2,4-dione;
5-[4-(3,3,4,4-Tetrafluoropyrrolidin-1-yl)pyrazolo[3,4-d]pyrimidin-2-yl]-1H-pyrimidine-2,4-dione;
5-[4-[(3aR,6aS)-1,3,3a,4,6,6a-Hexahydrofuro[3,4-c]pyrrol-5-yl]pyrazolo[3,4-d]pyrimidin-2-yl]-1H-pyrimidine-2,4-dione;
5-[4-[(3aR,6aR)-1,3,3a,4,6,6a-Hexahydrofuro[3,4-c]pyrrol-5-yl]pyrazolo[3,4-d]pyrimidin-2-yl]-5H-pyrimidine-2,4-dione;
5-[4-(3,3-Difluoro-4-methoxy-pyrrolidin-1-yl)pyrazolo[3,4-d]pyrimidin-2-yl]-1H-pyrimidine-2,4-dione;
5-[4-[3-[2-(1-Piperidyl)ethoxy]pyrrolidin-1-yl]thieno[2,3-b]pyridin-2-yl]-1H-pyrimidine-2,4-dione;
5-[4-(3,3-Difluoro-4,4-dimethyl-pyrrolidin-1-yl)thieno[2,3-b]pyridin-2-yl]-1H-pyrimidine-2,4-dione;
5-[6-[3-[2-(1-Piperidyl)ethoxy]pyrrolidin-1-yl]-9H-purin-8-yl]-1H-pyrimidine-2,4-dione;
5-[7-[3-[2-(1-Piperidyl)ethoxy]pyrrolidin-1-yl]thieno[3,2-b]pyridin-2-yl]-1H-pyrimidine-2,4-dione;
5-[4-[3,3-Difluoro-4-[2-(1-piperidyl)ethoxy]pyrrolidin-1-yl]pyrrolo[2,1-f][1,2,4]triazin-6-yl]-1H-pyrimidine-2,4-dione;
5-[2-Chloro-4-[3-[2-(1-piperidyl)ethoxy]pyrrolidin-1-yl]pyrrolo[2,1-f][1,2,4]triazin-6-yl]-5H-pyrimidine-2,4-dione;
5-[4-(8,8-Difluoro-6-azaspiro[3.4]octan-6-yl)furo[2,3-d]pyrimidin-6-yl]-1H-pyrimidine-2,4-dione;
5-[4-(4,4-Difluoro-2-azaspiro[4.4]nonan-2-yl)furo[2,3-d]pyrimidin-6-yl]-1H-pyrimidine-2,4-dione;
5-[4-[(4-Chlorophenyl)methylamino]furo[2,3-d]pyrimidin-6-yl]-1H-pyrimidine-2,4-dione;
5-[4-(7,7-Difluoro-5-azaspiro[2.4]heptan-5-yl)furo[2,3-d]pyrimidin-6-yl]-1H-pyrimidine-2,4-dione;
[3-[[6-(2,4-Dioxo-1H-pyrimidin-5-yl)furo[2,3-d]pyrimidin-4-yl]amino]-2,2-difluoro-propyl]N-isopropylcarbamate;
5-[4-[3-[2-[4-(Trifluoromethyl)-1-piperidyl]ethoxy]pyrrolidin-1-yl]furo[2,3-d]pyrimidin-6-yl]-1H-pyrimidine-2,4-dione;
[1-[6-(2,4-Dioxo-1H-pyrimidin-5-yl)furo[2,3-d]pyrimidin-4-yl]-4,4-difluoro-pyrrolidin-3-yl]morpholine-4-carboxylate;
[1-[6-(2,4-Dioxo-1H-pyrimidin-5-yl)furo[2,3-d]pyrimidin-4-yl]-4,4-difluoro-pyrrolidin-3-yl]8-oxa-3-azabicyclo[3.2.1]octane-3-carboxylate;
[1-[6-(2,4-Dioxo-1H-pyrimidin-5-yl)furo[2,3-d]pyrimidin-4-yl]-4,4-difluoro-pyrrolidin-3-yl]N-isopropylcarbamate;
5-[4-(3,3-Difluoro-4,4-dimethyl-pyrrolidin-1-yl)-6-methyl-pyrazolo[3,4-d]pyrimidin-2-yl]-1H-pyrimidine-2,4-dione;
6-[1-[2-(2,4-Dioxo-1H-pyrimidin-5-yl)pyrazolo[3,4-d]pyrimidin-4-yl]pyrrolidin-3-yl]oxypyridine-3-carbonitrile;
5-[4-(3,3-Difluoro-4,4-dimethyl-pyrrolidin-1-yl)pyrazolo[1,5-a]pyrazin-2-yl]-1H-pyrimidine-2,4-dione;
5-[4-(8,8-Difluoro-6-azaspiro[3.4]octan-6-yl)pyrazolo[1,5-a]pyrazin-2-yl]-1H-pyrimidine-2,4-dione;
5-[4-(7,7-Difluoro-5-azaspiro[2.4]heptan-5-yl)pyrazolo[1,5-a]pyrazin-2-yl]-1H-pyrimidine-2,4-dione;
5-[4-(3,3-Difluoro-4,4-dimethyl-pyrrolidin-1-yl)-7-fluoro-pyrazolo[4,3-c]pyridin-2-yl]-1H-pyrimidine-2,4-dione; and
5-[4-(3,3-Difluoro-4,4-dimethyl-pyrrolidin-1-yl)pyrazolo[4,3-c]pyridin-2-yl]-1H-pyrimidine-2,4-dione.

The various functional groups and substituents making up the compounds of the formula (I) are typically chosen such that the molecular weight of the compound of the formula (I) does not exceed 1000. More usually, the molecular weight of the compound will be less than 900, for example less than 800, or less than 750, or less than 700, or less than 650. More preferably, the molecular weight is less than 600 and, for example, is 550 or less.

A suitable pharmaceutically acceptable salt of a compound of the invention is, for example, an acidaddition-salt of a compound of the invention which is sufficiently basic, for example, an acidaddition-salt with, for example, an inorganic or organic acid, for example hydrochloric, hydrobromic, sulfuric, phosphoric, trifluoroacetic, formic, citric methane sulfonate or maleic acid. In addition, a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a pharmaceutically acceptable cation, for example a salt with methylamine, dimethylamine, trimethylamine, piperidine, morpholine or tris-(2-hydroxyethyl)amine.

Compounds that have the same molecular formula but differ in the nature or sequence of bonding of their atoms or the arrangement of their atoms in space are termed “isomers”. Isomers that differ in the arrangement of their atoms in space are termed “stereoisomers”. Stereoisomers that are not mirror images of one another are termed “diastereomers” and those that are nonsuperimposable mirror images of each other are termed “enantiomers”. When a compound has an asymmetric center-, for example, it is bonded to four different groups, a pair of enantiomers is possible. An enantiomer can be characterized by the absolute configuration of its asymmetric center and is described by the R and S sequencing rules of Cahn and Prelog, or by the manner in which the molecule rotates the plane of polarized light and designated as dextrorotatory or -levorotatory- (i.e., as (+) or (−)-isomers respectively). A chiral compound can exist as either individual enantiomer or as a mixture thereof. A mixture containing equal proportions of the enantiomers is called a “racemic mixture”.

The compounds of this invention may possess one or more asymmetric centers; such compounds can therefore be produced as individual (R) or (S)stereoisomers or as mixtures thereof. Unless indicated otherwise, the description or naming of a particular compound in the specification and claims is intended to include both individual enantiomers and mixtures, racemic or otherwise, thereof. The methods for the determination of stereochemistry and the separation of stereoisomers are wellknown in the art (see discussion in Chapter 4 of “Advanced Organic Chemistry”, 4th edition J. March, John Wiley and Sons, New York, 2001), for example by synthesis from optically active starting materials or by resolution of a racemic form. Some of the compounds of the invention may have geometric isomeric centres (E and Z isomers). It is to be understood that the present invention encompasses all optical, diastereoisomers and geometric isomers and mixtures thereof that possess antiproliferative activity.

The present invention also encompasses compounds of the invention as defined herein which comprise one or more isotopic substitutions. For Example, H may be in any isotopic form, including 1H, 2H(D), and 3H (T); C may be in any isotopic form, including 12C, 13C, and 14C; and O may be in any isotopic form, including 160 and 180; and the like.

It is also to be understood that certain compounds of the formula (I) may exist in solvated as well as unsolvated forms such as, for example, hydrated forms. It is to be understood that the invention encompasses all such solvated forms that possess antiproliferative activity.

It is also to be understood that certain compounds of the formula I may exhibit polymorphism, and that the invention encompasses all such forms that possess antiproliferative activity.

Compounds of the formula I may exist in a number of different tautomeric forms and references to compounds of the formula I include all such forms. For the avoidance of doubt, where a compound can exist in one of several tautomeric forms, and only one is specifically described or shown, all others are nevertheless embraced by formula I. Examples of tautomeric forms include keto-, enol-, and enolate-forms, as in, for example, the following tautomeric pairs: keto/enol (illustrated below), imine/enamine, amide/imino alcohol, amidine/amidine, nitroso/oxime, thioketone/enethiol, and nitro/aci-nitro.

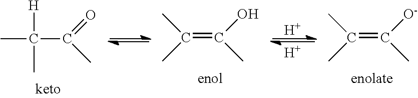

Compounds of the formula I containing an amine function may also form N-oxides. A reference herein to a compound of the formula I that contains an amine function also includes the N-oxide. Where a compound contains several amine functions, one or more than one nitrogen atom may be oxidised to form an N-oxide. Particular Examples of N-oxides are the N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g. a peroxycarboxylic acid), see for example Advanced Organic Chemistry, by Jerry March, 4th Edition, Wiley Interscience, pages. More particularly, N-oxides can be made by the procedure of L. W. Deady (Syn. Comm. 1977, 7, 509-514) in which the amine compound is reacted with m-chloroperoxybenzoic acid (mCPBA), for example, in an inert solvent such as dichloromethane.

The compounds of formula (I) may be administered in the form of a prodrug which is broken down in the human or animal body to release a compound of the invention. A pro-drug may be used to alter the physical properties and/or the pharmacokinetic properties of a compound of the invention. A pro-drug can be formed when the compound of the invention contains a suitable group or substituent to which a property-modifying group can be attached. Examples of pro-drugs include in vivo cleavable ester derivatives that may be formed at a carboxy group or a hydroxy group in a compound of the formula (I) and in-vivo cleavable amide derivatives that may be formed at a carboxy group or an amino group in a compound of the formula (I).

Accordingly, the present invention includes those compounds of the formula (I) as defined hereinbefore when made available by organic synthesis and when made available within the human or animal body by way of cleavage of a pro-drug thereof. Accordingly, the present invention includes those compounds of the formula I that are produced by organic synthetic means and also such compounds that are produced in the human or animal body by way of metabolism of a precursor compound, that is a compound of the formula (I) may be a synthetically-produced compound or a metabolically-produced compound.

A suitable pharmaceutically acceptable pro-drug of a compound of the formula (I) is one that is based on reasonable medical judgement as being suitable for administration to the human or animal body without undesirable pharmacological activities and without undue toxicity.

Various forms of pro-drug have been described, for example in the following documents:—
a) Methods in Enzymology, Vol. 42, p. 309-396, edited by K. Widder, et al. (Academic Press, 1985);
b) Design of Pro-drugs, edited by H. Bundgaard, (Elsevier, 1985);
c) A Textbook of Drug Design and Development, edited by Krogsgaard-Larsen and H. Bundgaard, Chapter 5 “Design and Application of Pro-drugs”, by H. Bundgaard p. 113-191 (1991);
d) H. Bundgaard, Advanced Drug Delivery Reviews, 8, 1-38 (1992);
e) H. Bundgaard, et al., Journal of Pharmaceutical Sciences, 77, 285 (1988);
f) N. Kakeya, et al., Chem. Pharm. Bull., 32, 692 (1984);
g) T. Higuchi and V. Stella, “Pro-Drugs as Novel Delivery Systems”, A.C.S. Symposium Series, Volume 14; and
h) E. Roche (editor), “Bioreversible Carriers in Drug Design”, Pergamon Press, 1987.

A suitable pharmaceutically acceptable pro-drug of a compound of the formula I that possesses a carboxy group is, for example, an in vivo cleavable ester thereof. An in vivo cleavable ester of a compound of the formula I containing a carboxy group is, for example, a pharmaceutically acceptable ester which is cleaved in the human or animal body to produce the parent acid. Suitable pharmaceutically acceptable esters for carboxy include C1-6alkyl esters such as methyl, ethyl and tert-butyl, C1-6alkoxymethyl esters such as methoxymethyl esters, C1-6alkanoyloxymethyl esters such as pivaloyloxymethyl esters, 3-phthalidyl esters, C3-8cycloalkylcarbonyloxy-C1-6alkyl esters such as cyclopentylcarbonyloxymethyl and 1-cyclohexylcarbonyloxyethyl esters, 2-oxo-1,3-dioxolenylmethyl esters such as 5-methyl-2-oxo-1,3-dioxolen-4-ylmethyl esters and C1-6alkoxycarbonyloxy-C1-6alkyl esters such as methoxycarbonyloxymethyl and 1-methoxycarbonyloxyethyl esters.

A suitable pharmaceutically acceptable pro-drug of a compound of the Formula (I) that possesses a hydroxy group is, for example, an in vivo cleavable ester or ether thereof.

An in vivo cleavable ester or ether of a compound of the formula I containing a hydroxy group is, for example, a pharmaceutically acceptable ester or ether which is cleaved in the human or animal body to produce the parent hydroxy compound. Suitable pharmaceutically acceptable ester forming groups for a hydroxy group include inorganic esters such as phosphate esters (including phosphoramidic cyclic esters). Further suitable pharmaceutically acceptable ester forming groups for a hydroxy group include C1-10alkanoyl groups such as acetyl, benzoyl, phenylacetyl and substituted benzoyl and phenylacetyl groups, C1-10alkoxycarbonyl groups such as ethoxycarbonyl, N,N—(C1-6)2carbamoyl, 2-dialkylaminoacetyl and 2-carboxyacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl. Suitable pharmaceutically acceptable ether forming groups for a hydroxy group include α-acyloxyalkyl groups such as acetoxymethyl and pivaloyloxymethyl groups.

A suitable pharmaceutically acceptable pro-drug of a compound of the formula (I) that possesses a carboxy group is, for example, an in vivo cleavable amide thereof, for example an amide formed with an amine such as ammonia, a C1-4alkylamine such as methylamine, a (C1-4alkyl)2amine such as dimethylamine, N-ethyl-N-methylamine or diethylamine, a C1-4alkoxy-C2-4alkylamine such as 2-methoxyethylamine, a phenyl-C1-4alkylamine such as benzylamine and amino acids such as glycine or an ester thereof.

A suitable pharmaceutically acceptable pro-drug of a compound of the formula I that possesses an amino group is, for example, an in vivo cleavable amide derivative thereof. Suitable pharmaceutically acceptable amides from an amino group include, for example an amide formed with C1-10alkanoyl groups such as an acetyl, benzoyl, phenylacetyl and substituted benzoyl and phenylacetyl groups. Examples of ring substituents on the phenylacetyl and benzoyl groups include aminomethyl, N-alkylaminomethyl, N,N-dialkylaminomethyl, morpholinomethyl, piperazin-1-ylmethyl and 4-(C1-4alkyl)piperazin-1-ylmethyl.

The in vivo effects of a compound of the formula (I) may be exerted in part by one or more metabolites that are formed within the human or animal body after administration of a compound of the formula (I). As stated hereinbefore, the in vivo effects of a compound of the formula (I) may also be exerted by way of metabolism of a precursor compound (a pro-drug).

Though the present invention may relate to any compound or particular group of compounds defined herein by way of optional, preferred or suitable features or otherwise in terms of particular embodiments, the present invention may also relate to any compound or particular group of compounds that specifically excludes said optional, preferred or suitable features or particular embodiments.

Suitably, the present invention excludes any individual compounds not possessing the biological activity defined herein.

### Synthesis

The compounds of the present invention can be prepared by any suitable technique known in the art. Particular processes for the preparation of the compounds of the invention are described in the Example section below.

In the description of the synthetic methods described herein and in any referenced synthetic methods that are used to prepare the starting materials, it is to be understood that all proposed reaction conditions, including choice of solvent, reaction atmosphere, reaction temperature, duration of the experiment and workup procedures, can be selected by a person skilled in the art.

It is understood by one skilled in the art of organic synthesis that the functionality present on various portions of the molecule must be compatible with the reagents and reaction conditions utilised.

It will be appreciated that during the synthesis of the compounds of the invention in the processes defined herein, or during the synthesis of certain starting materials, it may be desirable to protect certain substituent groups to prevent their undesired reaction. The skilled chemist will appreciate when such protection is required, and how such protecting groups may be put in place, and later removed.

For Examples of protecting groups see one of the many general texts on the subject, for example, ‘Protective Groups in Organic Synthesis’ by Theodora Green (publisher: John Wiley & Sons). Protecting groups may be removed by any convenient method described in the literature or known to the skilled chemist as appropriate for the removal of the protecting group in question, such methods being chosen so as to effect removal of the protecting group with the minimum disturbance of groups elsewhere in the molecule.

Thus, if reactants include, for example, groups such as amino, carboxy or hydroxy it may be desirable to protect the group in some of the reactions mentioned herein.

By way of Example, a suitable protecting group for an amino or alkylamino group is, for example, an acyl group, for example an alkanoyl group such as acetyl, an alkoxycarbonyl group, for example a methoxycarbonyl, ethoxycarbonyl or tbutoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed by, for example, hydrolysis with a suitable base such as an alkali metal hydroxide, for example lithium or sodium hydroxide. Alternatively an acyl group such as a tertbutoxycarbonyl group may be removed, for example, by treatment with a suitable acid as hydrochloric, sulfuric or phosphoric acid or trifluoroacetic acid and an arylmethoxycarbonyl group such as a benzyloxycarbonyl group may be removed, for example, by hydrogenation over a catalyst such as palladiumoncarbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium, sodium hydroxide or ammonia. Alternatively, an arylmethyl group such as a benzyl group may be removed, for example, by hydrogenation over a catalyst such as palladiumoncarbon.

A suitable protecting group for a carboxy group is, for example, an esterifying group, for example a methyl or an ethyl group which may be removed, for example, by hydrolysis with a base such as sodium hydroxide, or for example a tbutyl group which may be removed, for example, by treatment with an acid, for example an organic acid such as trifluoroacetic acid, or for example a benzyl group which may be removed, for example, by hydrogenation over a catalyst such as palladiumoncarbon.

Resins may also be used as a protecting group.

The methodology employed to synthesise a compound of formula (I) will vary depending on the nature of R₁ and R₂ and any substituent groups associated therewith. Suitable processes for their preparation are described further in the accompanying Examples.

Once a compound of formula (I) has been synthesised by any one of the processes defined herein, the processes may then further comprise one or more of the additional steps of:
(i) removing any protecting groups present;
(ii) converting the compound formula (I) into another compound of formula (I);
(iii) forming a pharmaceutically acceptable salt, hydrate or solvate of the compound of formula I; and/or
(iv) forming a prodrug of the compound of formula I.

An example of (ii) above is when a compound of formula (I) is synthesised and then one or more of the groups of R1 or R2 may be further reacted to change the nature of the group and provide an alternative compound of formula (I).

The resultant compounds of formula (I) can be isolated and purified using techniques well known in the art.

### Biological Activity

The biological assay described in the example section (Biological Examples A, B and C) may be used to measure the pharmacological effects of the compounds of the present invention.

Although the pharmacological properties of the compounds of formula I vary with structural change, as expected, the compounds of the invention were found to be active in the assay described in Biological Examples A, B and C.

In general, in terms of CD73 inhibitory activity, the compounds of the invention demonstrate an IC₅₀ of 3 μM or less in the assay described in Biological Example A, with preferred compounds of the invention demonstrating an IC₅₀ of 500 nM or less and the most preferred compounds of the invention demonstrating an IC₅₀ of 200 nM or less.

### Pharmaceutical Compositions

According to a further aspect of the invention there is provided a pharmaceutical composition which comprises a compound of the invention as defined hereinbefore, or a pharmaceutically acceptable salt, hydrate or solvate thereof, in association with a pharmaceutically acceptable diluent or carrier.

The compositions of the invention may be in a form suitable for oral use (for example as tablets, lozenges, hard or soft capsules, aqueous or oily suspensions, emulsions, dispersible powders or granules, syrups or elixirs), for topical use (for example as creams, ointments, gels, or aqueous or oily solutions or suspensions), for administration by inhalation (for example as a finely divided powder or a liquid aerosol), for administration by insufflation (for example as a finely divided powder) or for parenteral administration (for example as a sterile aqueous or oily solution for intravenous, subcutaneous, intramuscular, intraperitoneal or intramuscular dosing or as a suppository for rectal dosing).

The compositions of the invention may be obtained by conventional procedures using conventional pharmaceutical excipients, well known in the art. Thus, compositions intended for oral use may contain, for example, one or more colouring, sweetening, flavouring and/or preservative agents.

An effective amount of a compound of the present invention for use in therapy is an amount sufficient to treat or prevent a proliferative condition referred to herein, slow its progression and/or reduce the symptoms associated with the condition.

The amount of active ingredient that is combined with one or more excipients to produce a single dosage form will necessarily vary depending upon the individual treated and the particular route of administration. For Example, a formulation intended for oral administration to humans will generally contain, for example, from 0.5 mg to 0.5 g of active agent (more suitably from 0.5 to 100 mg, for example from 1 to 30 mg) compounded with an appropriate and convenient amount of excipients which may vary from about 5 to about 98 percent by weight of the total composition.

The size of the dose for therapeutic or prophylactic purposes of a compound of the formula I will naturally vary according to the nature and severity of the conditions, the age and sex of the animal or patient and the route of administration, according to well-known principles of medicine.

In using a compound of the invention for therapeutic or prophylactic purposes it will generally be administered so that a daily dose in the range, for example, 0.1 mg/kg to 75 mg/kg body weight is received, given if required in divided doses. In general lower doses will be administered when a parenteral route is employed. Thus, for example, for intravenous or intraperitoneal administration, a dose in the range, for example, 0.1 mg/kg to 30 mg/kg body weight will generally be used. Similarly, for administration by inhalation, a dose in the range, for example, 0.05 mg/kg to 25 mg/kg body weight will be used. Oral administration may also be suitable, particularly in tablet form. Typically, unit dosage forms will contain about 0.5 mg to 0.5 g of a compound of this invention.

### Therapeutic Uses and Applications

The present invention provides compounds that function as CD73 inhibitors.

According to a further aspect of the present invention, there is provided a method of inhibiting CD73 in vitro or in vivo, said method comprising contacting a cell with an effective amount of a compound or a pharmaceutically acceptable salt, hydrate or solvate thereof as defined herein.

According to a further aspect of the present invention, there is provided a method of selectively inhibiting CD73 in vitro or in vivo, said method comprising contacting a cell with an effective amount of a compound, or a pharmaceutically acceptable salt, hydrate or solvate thereof, as defined herein.

According to a further aspect of the present invention, there is provided a method of inhibiting cell proliferation, in vitro or in vivo, said method comprising contacting a cell with an effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating a disease or disorder associated CD73 activity in a patient in need of such treatment, said method comprising administering to said patient a therapeutically effective amount of a compound or a pharmaceutically acceptable salt, hydrate or solvate thereof as defined herein, or a pharmaceutical composition as defined herein.

According to a further aspect of the present invention, there is provided a method of treating a proliferative disorder in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional antiproliferative agents (e.g. checkpoint inhibitors and/or cytotoxic agents).

According to a further aspect of the present invention, there is provided a method of treating cancer in a patient in need of such treatment, said method comprising administering to said patient a therapeutically effective amount of a compound or a pharmaceutically acceptable salt, hydrate or solvate thereof as defined herein, or a pharmaceutical composition as defined herein. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein, for use in therapy.

According to a further aspect of the present invention, there is provided a compound or a pharmaceutically acceptable salt, hydrate or solvate thereof as defined herein, or a pharmaceutical composition as defined herein, for use in the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provided a compound, or a pharmaceutically acceptable salt, hydrate or solvate thereof, or a pharmaceutical composition as defined herein for use in the treatment of cancer. In a particular embodiment, the cancer is human cancer. Suitably, the compound or pharmaceutical composition is administered in combination with one or more additional anticancer agents (e.g. checkpoint inhibitors and/or cytotoxic agents).

According to a further aspect of the present invention, there is provided a compound, or a pharmaceutically acceptable salt, hydrate or solvate thereof, as defined herein for use as an inhibitor of CD73 activity. Certain compounds of the invention are selective CD73 inhibitors.

According to a further aspect of the present invention, there is provided a compound, or a pharmaceutically acceptable salt, hydrate or solvate thereof, as defined herein for use in the treatment of a disease or disorder in which CD73 activity is implicated.

According to a further aspect of the present invention, there is provided the use of a compound, or a pharmaceutically acceptable salt, hydrate or solvate thereof, as defined herein in the manufacture of a medicament for the treatment of a proliferative condition. Suitably, the compound or pharmaceutical composition is administered in combination with one or more additional antiproliferative agents (e.g. checkpoint inhibitors and/or cytotoxic agents).

According to a further aspect of the present invention, there is provide the use of a compound, or a pharmaceutically acceptable salt, hydrate or solvate thereof, as defined herein in the manufacture of a medicament for the treatment of cancer. Suitably, the cancer is a human cancer. Suitably, the compound or pharmaceutical composition is administered in combination with one or more additional anticancer agents (e.g. checkpoint inhibitors and/or cytotoxic agents).

According to a further aspect of the present invention, there is provided a use of a compound, or a pharmaceutically acceptable salt, hydrate or solvate thereof, as defined herein in the manufacture of a medicament for use as an inhibitor of CD73 activity.

According to a further aspect of the present invention, there is provided a use of a compound, or a pharmaceutically acceptable salt, hydrate or solvate thereof, as defined herein in the manufacture of a medicament for the treatment of a disease or disorder in which CD73 activity is implicated.

The term “proliferative disorder” are used interchangeably herein and pertain to an unwanted or uncontrolled cellular proliferation of excessive or abnormal cells which is undesired, such as, neoplastic or hyperplastic growth, whether in vitro or in vivo. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

The anti-proliferative effects of the compounds of the present invention have particular application in the treatment of human cancers.

More specifically, there is provided a compound of general formula (I) for use in the treatment of cancer, particularly solid tumours, for example non-small cell lung cancer, head and neck squamous cancer and urothelial cancer.

There is also provided the use of a compound of general formula (I) in the manufacture of a medicament for the treatment of cancer, particularly solid tumours, for example non-small cell lung cancer, head and neck squamous cancer and urothelial cancer.

The invention further provides a method for the treatment of cancer, particularly solid tumours, for example non-small cell lung cancer, head and neck squamous cancer and urothelial cancer, the method comprising administering to a patient in need of such treatment an effective amount of a compound of general formula (I).

The patient to be treated is suitably a mammal and more suitably a human.

### Routes of Administration

The compounds of the invention or pharmaceutical compositions comprising these compounds may be administered to a subject by any convenient route of administration, whether systemically/peripherally or topically (i.e., at the site of desired action).

Routes of administration include, but are not limited to, oral (e.g, by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eye drops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intra-arterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### Combination Therapies

The compounds of formula I are useful for the treatment and/or prophylaxis of proliferative disorders, such as, for example, cancer. A compound of formula I defined herein may be used in combination with one or more additional antiproliferative/anticancer therapies, such as, for example, chemotherapy with one or more additional antiproliferative/anticancer agents, radiotherapy and/or conventional surgery.

An additional antiproliferative/anticancer agent may be included in the pharmaceutical composition with a compound of formula (I) as defined herein or, alternatively, it may be administered separately, either at the same time as the compound of formula (I) or at an earlier or later time.

Therefore, in a further aspect of the present invention there is provided a product comprising a compound of general formula (I) and an additional agent useful in the treatment or prevention of cancer as a combined preparation for simultaneous, sequential or separate use in the treatment of cancer.

The present invention also provides a compound of general formula (I) in combination with one or more additional antiproliferative/anticancer agents for use in the treatment of cancer as a combined preparation for simultaneous, sequential or separate use in the treatment of treatment of cancer.

In particular, the combination therapy defined herein is suitable for the treatment of solid tumours for example non-small cell lung cancer, head and neck squamous cancer and urothelial cancer.

Suitable additional antiproliferative/anti-cancer agents that may be used in combination with a compound of formula I defined herein [either separately or as part of a combined pharmaceutical composition or a combined preparation with the compounds of general formula (I)] include:
1) other forms of cancer immunotherapy and anti-cancer chemotherapeutic agents;
2) A2b antagonists;
3) anti-PD-1 and PDL-1 antibodies including, but not limited to, pembrolizumab, nivolumab, durvalumab, avelumab and atezolizumab; and
4) anti-CTLA4 antibodies including, but not limited to, ipilimumab.

The compounds of of formula I defined herein are particularly suited to use in combination with anti-PD-1 and PDL-1 antibodies including, but not limited to, pembrolizumab, nivolumab, durvalumab, avelumab and atezolizumab.

The CD73 inhibitors of general formula (I) can also be used in combination with cell-based immunotherapy and cancer vaccines that include, but are not limited to CAR-T cell therapy.

Examples of the additional antiproliferative/anticancer chemotherapeutic agents include, but are not limited to, any one or more of the following: MEK (e.g. MEK1, MEK2, or MEK1 and MEK2) inhibitors (e.g. XL518, CI-1040, PD035901, selumetinib/AZD6244, GSK1 120212/trametinib, GDC-0973, ARRY-162, ARRY-300, AZD8330, PD0325901, U0126, PD98059, TAK-733, PD3 18088, AS703026, BAY 869766), alkylating agents (e.g., cyclophosphamide, ifosfamide, chlorambucil, busulfan, melphalan, mechlorethamine, uramustine, thiotepa, nitrosoureas, nitrogen mustards (e.g., mechloroethamine, cyclophosphamide, chlorambucil, meiphalan), ethylenimine and methylmelamines (e.g., hexamethlymelamine, thiotepa), alkyl sulfonates (e.g., busulfan), nitrosoureas (e.g., carmustine, lomusitne, semustine, streptozocin), triazenes (decarbazine)), anti-metabolites (e.g., 5-azathioprine, leucovorin, capecitabine, fludarabine, gemcitabine, pemetrexed, raltitrexed, folic acid analog (e.g., methotrexate), or pyrimidine analogs (e.g., fluorouracil, floxouridine, Cytarabine), purine analogs (e.g., mercaptopurine, thioguanine, pentostatin), etc.), plant alkaloids (e.g., vincristine, vinblastine, vinorelbine, vindesine, podophyllotoxin, paclitaxel, docetaxel, etc.), topoisomerase inhibitors (e.g., irinotecan, topotecan, amsacrine, etoposide (VP16), etoposide phosphate, teniposide, etc.), antitumor antibiotics (e.g., doxorubicin, adriamycin, daunorubicin, epirubicin, actinomycin, bleomycin, mitomycin, mitoxantrone, plicamycin, etc.), platinum-based compounds or platinum containing agents (e.g. cisplatin, oxaloplatin, arboplatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide), epipodophyllotoxins (e.g., etoposide), antibiotics (e.g., daunorubicin, doxorubicin, bleomycin), enzymes (e.g., L-asparaginase), inhibitors of mitogen-activated protein kinase signaling (e.g. U0126, PD98059, PD184352, PD0325901, ARRY-142886, SB239063, SP600125, BAY 43-9006, wortmannin, or LY294002, Syk inhibitors, mTOR inhibitors, antibodies (e.g., rituxan), gossyphol, genasense, polyphenol E, Chlorofusin, all trans-retinoic acid (ATRA), bryostatin, tumor necrosis factor-related apoptosis-inducing ligand (TRAIL), 5-aza-2′-deoxycytidine, all trans retinoic acid, doxorubicin, vincristine, etoposide, gemcitabine, imatinib (Gleevec®), geldanamycin, 17-N-Allylamino-17-Demethoxygeldanamycin (17-AAG), flavopiridol, LY294002, bortezomib, trastuzumab, BAY 11-7082, PKC412, PD184352, 20-epi-I, 25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin Ill derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cisporphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fiudarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatinA; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzyl guanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-basedimmune modulator; protein kinase C inhibitor; protein kinase C inhibitors, microalgal; proteintyrosine phosphatase inhibitors; purine nucleoside phosphorylase inhibitors; purpurins; pyrazoloacridine; pyridoxylated hemoglobin polyoxyethylerie conjugate; raf antagonists; raltitrexed; ramosetron; ras farnesyl protein transferase inhibitors; ras inhibitors; ras-GAPinhibitor; retelliptine demethylated; rhenium Re 186 etidronate; rhizoxin; ribozymes; RII retinamide; rogletimide; rohitukine; romurtide; roquinimex; rubiginone 1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stemcell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; zinostatin stimalamer, Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; iimofosine; interleukin 2 (including recombinant interleukin 2, or rIL.sub.2), interferon alfa-2a; interferon alfa-2b: interferon alfa-nl; interferon alfa-n3; interferon beta-la; interferon gamma-lb; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazoie; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride, agents that arrest cells in the G2-M phases and/or modulate the formation or stability of microtubules, (e.g. Taxol™ (i.e. paclitaxel), Taxotere™, compounds comprising the taxane skeleton, Erbulozole (i.e. R-55104), Dolastatin 10 (i.e. DLS-10 and NSC-376128), Mivobulin isethionate (i.e. as CI-980), Vincristine, NSC-639829, Discodermolide (i.e. as NVP-XX-A-296), ABT-751 (Abbott, i.e. E-7010), Altorhyrtins (e.g. Altorhyrtin A and Altorhyrtin C), Spongistatins (e.g. Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (i.e. LU-103793 and NSC-D-669356), Epothilones (e.g. Epothilone A, Epothilone B, Epothilone C (i.e. desoxyepothilone A or dEpoA), Epothilone D (i.e. KOS-862, dEpoB, and desoxyepothilone B), Epothilone E, Epothilone F, Epothilone B N-oxide, Epothilone A N-oxide, 16-aza-epothilone B, 21-aminoepothilone B (i.e. BMS-3 10705), 21-hydroxyepothilone D (i.e. Desoxyepothilone F and dEpoF), 26-fluoroepothilone, Auristatin PE (i.e. NSC-654663), Soblidotin (i.e. TZT-1027), Vincristine sulfate, Cryptophycin 52 (i.e. LY-355703), Vitilevuamide, Tubulysin A, Canadensol, Centaureidin (i.e. NSC-106969), Oncocidin AI (i.e. BTO-956 and DF E), Fijianolide B, Laulimalide, Narcosine (also known as NSC-5366), Nascapine, Hemiasterlin, Vanadocene acetylacetonate, Monsatrol, Inanocine (i.e. NSC-698666), Eleutherobins (such as Desmethyleleutherobin, Desaetyleleutherobin, Isoeleutherobin A, and ZEleutherobin), Caribaeoside, Caribaeolin, Halichondrin B, Diazonamide A, Taccalonolide A, Diozostatin, (−)-Phenylahistin (i.e. NSCL-96F037), Myoseverin B, Resverastatin phosphate sodium, steroids (e.g., dexamethasone), finasteride, aromatase inhibitors, gonadotropin-releasing hormone agonists (GnRH) such as goserelin or leuprolide, adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), immunostimulants (e.g., Bacillus Calmette-Guerin (BCG), levamisole, interleukin-2, alpha-interferon, etc.), monoclonal antibodies (e.g., anti-CD20, anti-F£ER2, anti-CD52, anti-ULA-DR, and anti-VEGF monoclonal antibodies), immunotoxins (e.g., anti-CD33 monoclonal antibody-calicheamicin conjugate, anti-CD22 monoclonal antibody-pseudomonas exotoxin conjugate, etc.), radioimmunotherapy (e.g., anti-CD20 monoclonal antibody conjugated to In, 0Y, or I, etc.), triptolide, homoharringtonine, dactinomycin, doxorubicin, epirubicin, topotecan, itraconazole, vindesine, cerivastatin, vincristine, deoxyadenosine, sertraline, pitavastatin, irinotecan, clofazimine, 5-nonyloxytryptamine, vemurafenib, dabrafenib, erlotinib, gefitinib, EGFR inhibitors, epidermal growth factor receptor (EGFR)-targeted therapy or therapeutic (e.g. gefitinib (Iressa™), erlotinib (Tarceva™), cetuximab (Erbitux™), lapatinib (Tykerb™) panitumumab (Vectibix™), vandetanib (Caprelsa™), afatinib/BIBW2992, CI-1033/canertinib, neratinib/HKI-272, CP-724714, TAK-285, AST-1306, ARRY334543, ARRY-380, AG-1478, dacomitinib/PF299804, OSI-420/desmethyl erlotinib, AZD8931, AEE788, pelitinib/EKB-569, CUDC-101, WZ8040, WZ4002, WZ3146, AG-490, XL647, PD153035, BMS-599626), sorafenib, imatinib, sunitinib, dasatinib, hormonal therapies, or the like.

As indicated above, the combination therapy of the present invention may be achieved by way of the simultaneous, sequential or separate dosing of the individual components of the treatment. Such combination products employ the compounds of this invention within the dosage range described hereinbefore and the other pharmaceutically-active agent within its approved dosage range.

According to this aspect of the invention there is provided a combination for use in the treatment of a cancer (for example a cancer involving a solid tumour) comprising a compound of the invention as defined hereinbefore, or a pharmaceutically acceptable salt, hydrate or solvate thereof, and one or more additional antiproliferative/anticancer agents.

According to this aspect of the invention there is also provided a combination for use in the treatment of a proliferative condition, such as cancer (for example a cancer involving a solid tumour), comprising a compound of the invention as defined hereinbefore, or a pharmaceutically acceptable salt, hydrate or solvate thereof, and one or more additional antiproliferative/anticancer agents selected from those listed above.

In a further aspect of the invention there is provided a compound of the invention or a pharmaceutically acceptable salt, hydrate or solvate thereof, for use in the treatment of cancer in combination with another anti-tumour agent, optionally selected from one listed herein above.

Herein, where the term “combination” is used it is to be understood that this refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

According to a further aspect of the invention there is provided a pharmaceutical composition which comprises a compound of the invention, or a pharmaceutically acceptable salt, hydrate or solvate thereof, in combination with an anti-tumour agent (optionally selected from one listed herein above), in association with a pharmaceutically acceptable diluent or carrier.

### EXAMPLES

### General Conditions:

Mass spectra were run on LC-MS systems using electrospray ionization. These were run using either a Waters Acquity H-Class UPLC with PDA and QDa mass detection, an Acquity UPLC (binary pump/PDA detector)+ZQ Mass Spectrometer or Acquity i-Class (quaternary pump/PDA detector)+Quattro Micro Mass Spectrometer, a Waters Acquity uPLC system with Waters PDA and ELS detectors or a Shimadzu LC-MS-2010EV system. [M+H]+ refers to mono-isotopic molecular weights.

NMR spectra were run on either a Bruker Ultrashield 500 MHz NMR spectrometer or a Bruker Avance III HD 400 MHz NMR spectrometer. Spectra were recorded at 298K and were referenced using the solvent peak.

The following examples are intended to illustrate the invention and are not to be construed as being limitations thereon. Temperatures are given in degrees centigrade. If not mentioned otherwise, all evaporations are performed in vacuo, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS and NMR. Abbreviations used are those conventional in the art. If not defined, the terms have their generally accepted meanings.

### Abbreviation

| | |
|---|---|
| br | broad |
| CDI | 1,1′-carbonyldiimidazole |
| d | doubletDCM dichloromethane |
| dd | doublet of doubletsDIPEA diisopropylethylamine |
| DMA | dimethylacetamide |
| DMAP | 4-dimethylaminopyridine |
| DMF | N,N-dimethylformamide |
| DMSO | dimethyl sulfoxide |
| EtOAc | ethyl acetate |
| EtOH | ethanol |
| HATU | N-[(Dimethylamino)-1H-1,2,3-triazolo-[4,5-b]pyridin-1-ylmethylene]- |
| | N-methylmethanaminium hexafluorophosphate N-oxide |
| HPLC | high pressure liquid chromatography |
| LC-MS | liquid chromatography and mass spectrometry |
| LDA | Lithium diisopropylamide |
| m | multiplet |
| mCPBA | meta-chloroperbenzoic acid |
| MeCN | acetonitrileMeOH MeOH |
| MS | mass spectrometry |
| min(s) | minute(s) |
| mL | milliliter(s) |
| m/z | mass to charge ratio |
| NBS | N-bromosuccinimide |
| NMP | N-methyl-2-pyrrolidone |
| NMR | nuclear magnetic resonance |
| Pd(dppf)Cl₂ | [1,1′-bis(diphenylphosphino)ferrocene]palladium(II) dichloride |
| pTSA | para-toluenesulfonic acid |
| Rt | retention time |
| s | singlet |
| t | triplet |
| TBAB | tetrabutylammonium bromide |
| TFA | trifluoroacetic acid |
| TFAA | trifluoroacetic anhydride |
| THF | tetrahydrofuran |
| X-phos | 2-dicyclohexylphosphino-2′,4′,6′-triisopropylbiphenyl |

Referring to the examples that follow, compounds of the preferred embodiments were synthesized using the methods described herein, or other methods, which are known in the art.

The various starting materials, intermediates, and compounds of the preferred embodiments may be isolated and purified, where appropriate, using conventional techniques such as precipitation, filtration, crystallization, evaporation, distillation, and chromatography. Unless otherwise stated, all starting materials are obtained from commercial suppliers and used without further purification. Salts may be prepared from compounds by known salt-forming procedures.

If not indicated otherwise, the analytical HPLC conditions are as follows:

### Instrument: LC-MS-1:

### Method 2A

Column: Acquity UPLC BEH C18 2.1×50 mm 1.7 μm
Column Temp: 50° C.
Flow rate: 0.8 mL/min.
Eluents: A: H₂O, 0.1% formic acid, B: MeCN
Gradient: 0.0-1.8 min 2-98% B, 1.8-2.1 min 98% B, 2.1-2.5 98% A.

### Method 2B

Column: Acquity UPLC BEH C18 2.1×50 mm 1.7 μm
Column Temp: 50° C.
Flow rate: 0.8 mL/min.
Eluents: A: H₂O, 0.1% ammonia B: MeCN
Gradient: 0.0-1.8 min 2-98% B, 1.8-2.1 min 98% B, 2.1-2.5 98% A.

### Method 8A

Column: Acquity UPLC BEH C18 2.1×50 mm 1.7 μm
Column Temp: 50° C.
Eluents: A: H₂O, 0.1% formic acid, B: MeCN
Flow Rate: 0.6 mL/min
Gradient: 0.5-6.5 min 2-98% B, 6.5-7.6 min 98% B, 7.6-8.0 98% A.

### Method 8B

Column: Acquity UPLC BEH C18 2.1×50 mm 1.7 μm
Column Temp: 50° C.
Eluents: A: H₂O, 0.1% ammonia, B: MeCN
Flow Rate: 0.6 mL/min
Gradient: 0.5-6.5 min 2-98% B, 6.5-7.6 min 98% B, 7.6-8.0 98% A.

### Method 8B [0-10%]

Column: Acquity UPLC BEH C18 2.1×50 mm 1.7 μm
Column Temp: 50° C.
Eluents: A: H₂O, 0.1% ammonia, B: MeCN
Flow Rate: 0.6 mL/min
Gradient: 0.0-0.5 mins 2% B; 0.5-6.5 mins 2-10% B 6.5-7.5 mins 98% B; 7.5-7.6 mins 2% B; 7.6-8.0 2% B

### Instrument: LC-MS-2:

### Method 2A

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% formic acid
Gradient: 0.0-1.7 mins 0-95% B, 5% C; 1.7-2.1 mins 95% B, 5% C 2.1-2.5 mins 95% A, 5% C.

### Method 2B

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% ammonia (aq.)
Gradient: 0.0-1.7 mins 0-95% B, 5% D; 1.7-2.1 mins 95% B, 5% D 2.1-2.5 mins 95% A, 5% D.

### Method 8A

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% formic acid
Gradient: 0.0-0.5 mins 95% A, 5% C; 0.5-6.5 mins 0-95% B, 5% C; 6.6-7.5 mins 95% B, 5% C; 7.5-7.6 mins 0-95% A, 5% C; 7.6-8.0 mins 95% A, 5% C.

### Method 8B

Column: Acquity UPLC BEH C18 2.1×50 mm 1.7 μm
Column Temp: 50° C.
Flow rate: 0.8 mL/min.
Eluents: A: H₂O, B: MeCN, C: 50% H₂O/50% MeCN+2.0% ammonia (aq.)
Gradient: 0.0-0.5 mins 95% A, 5% D; 0.5-6.5 mins 0-95% B, 5% D; 6.6-7.5 mins 95% B, 5% D; 7.5-7.6 mins 0-95% A, 5% D; 7.6-8.0 mins 95% A, 5% D.

### Instrument: LC-MS

### Method 7A

Column: Phenomenex Kinetix-XB C18 2.1×100 mm, 1.7 μm
Column Temp 40° C.
Eluents: A: H₂O 0.1% formic acid, B: acetonitrile, 0.1% formic acid
Flow Rate: 0.6 mL/mins
Gradient: 0-5.3 mins 5-100% B, 5.3-5.8 mins 100% B, 5.8-5.82 mins 100-5% B, 5.82-7.00 mins 5% B.

### Method 7B

Column: Waters UPLC® BEH™ 018, 2.1 mm×100 mm, 1.7 μm column
Column Temp: 55° C.
Eluents: A: 2 mM ammonium bicarbonate buffered to pH10, B: acetonitrile
Flow Rate: 0.6 mL/mins
Gradient: 0-5.3 mins 5-100% B, 5.3-5.8 mins 100% B, 5.8-5.82 mins 100-5% B, 5.82-7.00 mins 5% B.

### Method 2A

Column: Waters UPLC™ BEH™ C18 2.1×50 mm, 1.7 μm
Column Temp 40° C.
Eluents: A: H₂O 0.1% formic acid, B: acetonitrile, 0.1% formic acid
Flow Rate: 0.9 mL/mins
Gradient: 0-1.10 mins 5-100% B, 1.10-1.35 mins 100% B, 1.35-1.40 mins 100-5% B, 1.40-1.50 mins 5% B.

### Method 2B:

Column: Waters UPLC™ BEH™ C18 2.1×30 mm, 1.7 μm
Column Temp 55° C. from 21 Sep. 2020, before that 40C
Eluents: A: 2 mM ammonium bicarbonate, buffered to pH10, B: acetonitrile
Flow Rate: 1 mL/mins
Gradient: 0-1.10 mins 1-100% B, 1.10-1.35 mins 100% B, 1.35-1.40 mins 100-% B, 1.40-1.80 mins 1% B

### Example 1

### 5-[4-[3-(Hydroxymethyl)pyrrolidin-1-yl]thieno[2,3-d]pyrimidin-6-yl]-1H-pyrimidine-2,4-dione

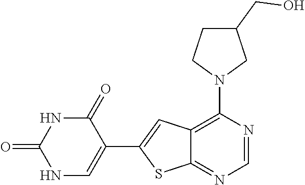 [SMILES: O=c1[nH]cc(-c2cc3c(N4CCC(CO)C4)ncnc3s2)c(=O)[nH]1]

5-(4-Chlorothieno[2,3-d]pyrimidin-6-yl)-1H-pyrimidine-2,4-dione (Intermediate D) (80 mg, 0.24 mmol), pyrrolidin-3-ylmethanol (37 mg, 0.36 mmol) and DIPEA (0.13 mL, 0.73 mmol) were dissolved in MeCN (3 mL) and heated at 70° C. overnight. The resulting mixture was allowed to cool to room temperature before being filtered. The collected solid was washed with MeCN (5 mL) and diethyl ether (5 mL) and purified by C18 reverse phase chromatography eluting with a gradient of 5 to 35% MeCN in water (+0.1% formic acid). The product was freeze dried and then azeotroped with MeCN to afford the title compound as a yellow solid.

LC-MS-1 (Method 8A): Rt 1.52 mins; MS m/z 346.2=[M+H]+

1H NMR (400 MHz, DMSO-d6) δ 11.52 (s, 2H), 8.28 (s, 1H), 8.26 (s, 1H), 7.90 (s, 1H), 4.76 (t, J=5.3 Hz, 1H), 3.98-3.72 (m, 3H), 3.56 (s, 1H), 3.50-3.43 (m, 2H), 2.07 (s, 1H), 1.86-1.75 (m, 1H).

### Example 1.1

### 5-[4-[3-(2-Hydroxyethyl)pyrrolidin-1-yl]thieno[2,3-d]pyrimidin-6-yl]-1H-pyrimidine-2,4-dione

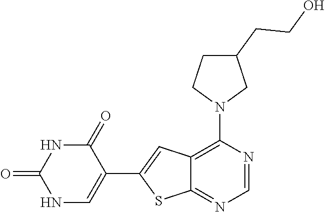 [SMILES: O=c1[nH]cc(-c2cc3c(N4CCC(CCO)C4)ncnc3s2)c(=O)[nH]1]

The title compound was prepared from 5-(4-chlorothieno[2,3-d]pyrimidin-6-yl)-1H-pyrimidine-2,4-dione (Intermediate D) and 2-pyrrolidin-3-ylethanol analogously to Example 1.

LC-MS-2 (Method 8A): Rt 1.91 mins; MS m/z 360.1=[M+H]+

1H NMR (400 MHz, DMSO-d6 @70° C.) δ 11.53 (s, 2H), 8.29 (s, 1H), 8.25 (s, 1H), 7.90 (s, 1H), 4.51 (t, J=5.1 Hz, 1H), 4.06-3.86 (m, 2H), 3.75 (s, 1H), 3.56-3.47 (m, 2H), 2.35 (s, 1H), 2.16 (s, 1H), 1.69-1.53 (m, 3H).

### Example 1.2

### 5-[4-(3,3-Dimethylazetidin-1-yl)thieno[2,3-d]pyrimidin-6-yl]-1H-pyrimidine-2,4-dione

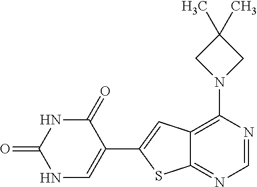 [SMILES: CC1(C)CN(c2ncnc3sc(-c4c[nH]c(=O)[nH]c4=O)cc23)C1]

The title compound was prepared from 5-(4-chlorothieno[2,3-d]pyrimidin-6-yl)-1H-pyrimidine-2,4-dione (Intermediate D) and 3,3-dimethylazetidine hydrochloride analogously to Example 1.

LC-MS-1 (Method 8A): Rt 1.93 mins; MS m/z 330.2=[M+H]+

1H NMR (400 MHz, DMSO-d6) δ 11.53 (s, 2H), 8.31 (s, 1H), 8.26 (s, 1H), 7.65 (s, 1H), 4.08 (s, 4H), 1.34 (s, 6H).

### Example 1.3

### 5-[4-[3-(2-Hydroxyethoxy)pyrrolidin-1-yl]thieno[2,3-d]pyrimidin-6-yl]-1H-pyrimidine-2,4-dione

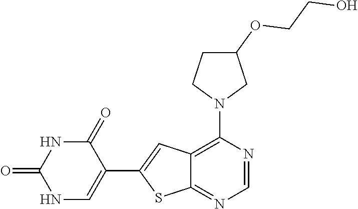 [SMILES: O=c1[nH]cc(-c2cc3c(N4CCC(OCCO)C4)ncnc3s2)c(=O)[nH]1]

The title compound was prepared from 5-(4-chlorothieno[2,3-d]pyrimidin-6-yl)-1H-pyrimidine-2,4-dione (Intermediate D) and 2-pyrrolidin-3-yloxyethanol hydrochloride analogously to Example 1.

LC-MS-1 (Method 8A): Rt 1.53 mins; MS m/z 376.2=[M+H]+

1H NMR (400 MHz, DMSO-d6) δ 11.54 (s, 2H), 8.34 (s, 1H), 8.27 (s, 1H), 7.91 (s, 1H), 4.67-4.56 (m, 1H), 4.26 (s, 1H), 3.88 (s, 4H), 3.55-3.44 (m, 4H), 2.21-2.00 (m, 2H).

### Example 2

### 5-[4-[3-[2-(1-Piperidyl)ethoxy]pyrrolidin-1-yl]thieno[2,3-d]pyrimidin-6-yl]-1H-pyrimidine-2,4-dione

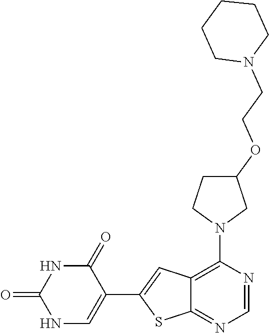 [SMILES: O=c1[nH]cc(-c2cc3c(N4CCC(OCCN5CCCCC5)C4)ncnc3s2)c(=O)[nH]1]

### Step 1: 6-Bromo-4-[3-[2-(1-piperidyl)ethoxy]pyrrolidin-1-yl]thieno[2,3-d]pyrimidine

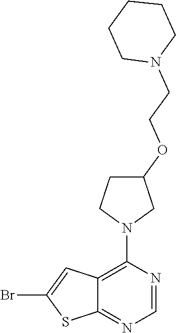 [SMILES: Brc1cc2c(N3CCC(OCCN4CCCCC4)C3)ncnc2s1]

DIPEA (0.92 mL, 5.29 mmol) was added to a stirred solution of 6-bromo-4-chloro-thieno[2,3-d]pyrimidine (330 mg, 1.32 mmol) and 1-(2-pyrrolidin-3-yloxyethyl)piperidine dihydrochloride (430 mg, 1.59 mmol) in 1,4-dioxane (7 mL) and the solution was heated at 80° C. for 3 h. Additional DIPEA (0.46 mL, 2.65 mmol) was added and stirring continued at 80° C. for 30 mins. The resulting mixture was allowed to cool to room temperature and the solution was concentrated in vacuo before being partitioned between DCM (20 mL) and water (20 mL). The layers were separated and the aqueous layer was further extracted with DCM (2×20 mL). The combined organic extracts were dried over magnesium sulfate and concentrated in vacuo. Purification by chromatography on silica eluting with a gradient of 2 to 10% MeOH in DCM afforded the title compound as a pink solid.

LC-MS-2 (Method 2B): Rt 1.66 mins; MS m/z 411.0/413.0=[M+H]+

1H NMR (500 MHz, Methanol-d4) δ 8.27 (s, 1H), 7.73 (s, 1H), 4.33 (s, 1H), 4.06-3.93 (m, 2H), 3.93-3.74 (m, 4H), 3.09-2.83 (m, 6H), 2.31 (s, 1H), 2.17 (s, 1H), 1.71 (s, 4H), 1.56 (s, 2H).

### Step 2: 6-(2,4-Dimethoxypyrimidin-5-yl)-4-[3-[2-(1-piperidyl)ethoxy]pyrrolidin-1-yl]thieno[2,3-d]pyrimidine

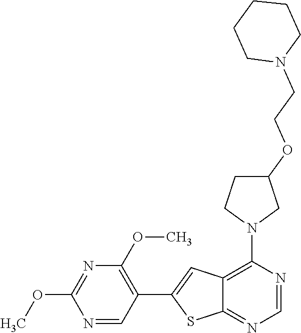 [SMILES: COc1ncc(-c2cc3c(N4CCC(OCCN5CCCCC5)C4)ncnc3s2)c(OC)n1]

Pd(dppf)Cl₂ (12 mg, 0.02 mmol) and (2,4-dimethoxypyrimidin-5-yl)boronic acid (47 mg, 0.26 mmol) were added to a degassed solution of 6-bromo-4-[3-[2-(1-piperidyl)ethoxy]pyrrolidin-1-yl]thieno[2,3-d]pyrimidine (step 1) (70 mg, 0.17 mmol) and cesium carbonate (78 mg, 0.51 mmol) in 1,4-dioxane (1 mL) and water (0.25 mL). The flask was evacuated and backfilled with nitrogen (3× cycles) and the solution was heated at 100° C. for 1 h. The resulting mixture was allowed to cool to room temperature and was partitioned between DCM (30 mL) and water (30 mL). The layers were separated and the aqueous layer was further extracted with DCM (2×30 mL). The combined organic extracts were dried over magnesium sulfate and concentrated in vacuo. Purification by chromatography on silica eluting with a gradient of 2 to 8% 1M NH₃ in MeOH in DCM afforded the title compound as a brown solid.

LC-MS-1 (Method 2B): Rt 1.47 mins; MS m/z 471.3=[M+H]+

1H NMR (500 MHz, Methanol-d4) δ 8.56 (s, 1H), 8.17 (s, 1H), 7.81 (s, 1H), 4.19 (s, 1H), 4.06 (s, 3H), 3.95 (s, 3H), 3.92 (s, 1H), 3.80 (s, 3H), 3.65-3.53 (m, 2H), 2.49 (t, J=5.7 Hz, 2H), 2.38 (s, 4H), 2.19 (s, 1H), 2.10-2.03 (m, 1H), 1.45 (p, J=5.6 Hz, 4H), 1.36-1.27 (m, 2H).

### Step 3: 5-[4-[3-[2-(1-Piperidyl)ethoxy]pyrrolidin-1-yl]thieno[2,3-d]pyrimidin-6-yl]-1H-pyrimidine-2,4-dione

A stirred solution of 6-(2,4-dimethoxypyrimidin-5-yl)-4-[3-[2-(1-piperidyl)ethoxy]pyrrolidin-1-yl]thieno[2,3-d]pyrimidine (step 2) (58 mg, 0.12 mmol) in 1M HCl (2 mL, 2 mmol) and MeOH (1 mL) was heated at 70° C. overnight. After cooling to room temperature and stirring for 2 days, the mixture was concentrated in vacuo. Purification by C18 reverse phase chromatography eluting with a gradient of 2 to 30% MeCN in water (+0.1% NH4OH) afforded the title compound as a beige solid.

LC-MS-2 (Method 8B): Rt 2.21 mins; MS m/z 443.1=[M+H]+

1H NMR 70° C. (500 MHz, DMSO-d6) δ 11.25 (s, 2H), 8.28 (s, 1H), 8.20-8.15 (m, 1H), 7.91 (s, 1H), 4.25 (p, J=3.8 Hz, 1H), 4.00-3.76 (m, 4H), 3.66-3.51 (m, 2H), 2.46 (t, J=6.1 Hz, 2H), 2.40-2.34 (m, 4H), 2.16-2.07 (m, 2H), 1.46 (p, J=5.6 Hz, 4H), 1.38-1.31 (m, 2H).

### Example 2.1

### 5-[4-(3,3-Dimethylpyrrolidin-1-yl)thieno[2,3-d]pyrimidin-6-yl]-1H-pyrimidine-2,4-dione

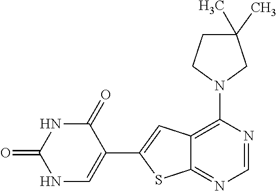 [SMILES: CC1(C)CCN(c2ncnc3sc(-c4c[nH]c(=O)[nH]c4=O)cc23)C1]

### Step 1: 6-Bromo-4-(3,3-dimethylpyrrolidin-1-yl)thieno[2,3-d]pyrimidine

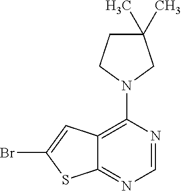 [SMILES: CC1(C)CCN(c2ncnc3sc(Br)cc23)C1]

The title compound was prepared from 6-bromo-4-chloro-thieno[2,3-d]pyrimidine and 3,3-dimethylpyrrolidine hydrochloride analogously to Example 2 step 1.

LC-MS-2 (Method 2A): Rt 1.70 mins; MS m/z 311.8/313.8=[M+H]+

1H NMR (500 MHz, DMSO-d6) δ 8.29 (s, 1H), 7.79 (s, 1H), 3.82 (m, 2H), 3.52 (m, 2H), 1.78 (m, 2H), 1.12 (s, 6H).

### Step 2: 6-(2,4-Dimethoxypyrimidin-5-yl)-4-(3,3-dimethylpyrrolidin-1-yl)thieno[2,3-d]pyrimidine

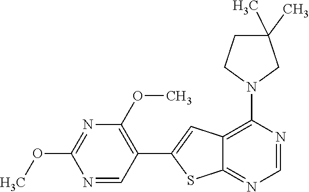

The title compound was prepared from 6-bromo-4-(3,3-dimethylpyrrolidin-1-yl)thieno[2,3-d]pyrimidine (step 1) and (2,4-dimethoxypyrimidin-5-yl)boronic acid analogously to Example 2 step 2.

LC-MS-2 (Method 2A): Rt 1.53 mins; MS m/z 372.0=[M+H]+

1H NMR (500 MHz, DMSO-d6) δ 8.91 (s, 1H), 8.30 (s, 1H), 7.98 (s, 1H), 4.02 (m, 8H), 3.58 (s, 2H), 1.82 (s, 2H), 1.14 (s, 6H).

### Step 3: 5-[4-(3,3-Dimethylpyrrolidin-1-yl)thieno[2,3-d]pyrimidin-6-yl]-1H-pyrimidine-2,4-dione

The title compound was prepared from 6-(2,4-dimethoxypyrimidin-5-yl)-4-(3,3-dimethylpyrrolidin-1-yl)thieno[2,3-d]pyrimidine (step 2) and 1M HCl analogously to Example 2 step 3.

LC-MS-2 (Method 8A): Rt 2.32 mins; MS m/z 344.0=[M+H]+

1H NMR (500 MHz, DMSO-d6) δ 11.51 (s, 1H), 11.44 (s, 1H), 8.46 (d, J=1.5 Hz, 1H), 8.34 (d, J=5.6 Hz, 1H), 7.97 (s, 1H), 4.07-3.96 (m, 2H), 3.69 (s, 2H), 1.88 (t, J=7.1 Hz, 2H), 1.18 (s, 6H).

### Example 3

### 6-[1-[6-(2,4-Dioxo-1H-pyrimidin-5-yl)thieno[2,3-d]pyrimidin-4-yl]pyrrolidin-3-yl]oxypyridine-3-carbonitrile

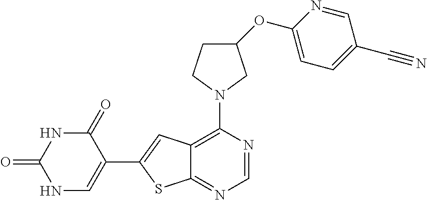 [SMILES: N#Cc1ccc(OC2CCN(c3ncnc4sc(-c5c[nH]c(=O)[nH]c5=O)cc34)C2)nc1]

### Step 1: 1-(6-Bromothieno[2,3-d]pyrimidin-4-yl)pyrrolidin-3-ol

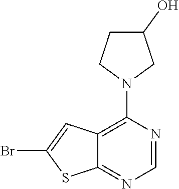 [SMILES: OC1CCN(c2ncnc3sc(Br)cc23)C1]

A stirred suspension of 6-bromo-4-chloro-thieno[2,3-d]pyrimidine (3.00 g, 12.02 mmol) and pyrrolidin-3-ol (1.26 g, 14.43 mmol) in MeCN (50 mL) under nitrogen was treated with DIPEA (8.38 mL, 48.09 mmol) and the reaction mixture was heated at 70° C. for 30 mins. After cooling to room temperature, the resulting suspension was filtered and the collected solid washed with diethyl ether (5 mL) then dried under vacuum to afford the title compound as a cream solid.

LC-MS-2 (Method 2A): Rt 1.12 mins; MS m/z 299.9/301.9=[M+H]+

1H NMR (400 MHz, DMSO-d6) δ 8.30 (s, 1H), 7.79 (s, 1H), 5.08-5.01 (m, 1H), 4.41 (s, 1H), 3.99-3.59 (m, 4H), 2.06-1.86 (m, 2H).

### Step 2: 6-[1-(6-Bromothieno[2,3-d]pyrimidin-4-yl)pyrrolidin-3-yl]oxypyridine-3-carbonitrile

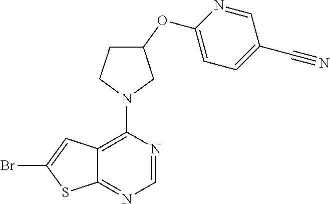 [SMILES: N#Cc1ccc(OC2CCN(c3ncnc4sc(Br)cc34)C2)nc1]

To a stirred suspension of 1-(6-bromothieno[2,3-d]pyrimidin-4-yl)pyrrolidin-3-ol (step 1) (300 mg, 1.0 mmol) in DMF (3 mL) was added sodium hydride (60% dispersion in mineral oil) (44 mg, 1.1 mmol) and the mixture was stirred at room temperature for 30 mins. 6-Fluoropyridine-3-carbonitrile (146 mg, 1.2 mmol) was added and stirring continued for 20 mins. The reaction was quenched with water (15 mL) and the mixture was extracted with EtOAc (3×20 mL). The combined organic extracts were washed with water (4×10 mL), brine (10 mL), dried over magnesium sulfate and concentrated in vacuo. Purification by chromatography on silica eluting with a gradient of 0 to 75% EtOAc in petroleum ether afforded the title compound as a colourless solid.

LC-MS-2 (Method 2A): Rt 1.60 mins; MS m/z 402.0/404.0=[M+H]+

1H NMR (400 MHz, CDCl3) δ 8.51 (d, J=2.3 Hz, 1H), 8.41 (s, 1H), 7.81 (dd, J=8.7, 2.3 Hz, 1H), 7.46 (s, 1H), 6.81 (d, J=8.7 Hz, 1H), 5.83 (s, 1H), 4.18-3.95 (m, 4H), 2.50-2.31 (m, 2H).

### Step 3: 6-[1-[6-(2,4-Dimethoxypyrimidin-5-yl)thieno[2,3-d]pyrimidin-4-yl]pyrrolidine-3-yl]oxypyridine-3-carbonitrile

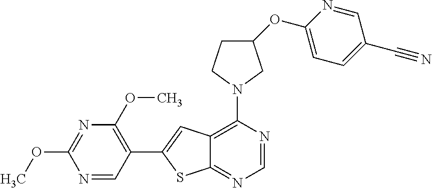 [SMILES: COc1ncc(-c2cc3c(N4CCC(Oc5ccc(C#N)cn5)C4)ncnc3s2)c(OC)n1]

A stirred mixture of 6-[1-(6-bromothieno[2,3-d]pyrimidin-4-yl)pyrrolidine-3-yl]oxypyridine-3-carbonitrile (step 2) (306 mg, 0.76 mmol), (2,4-dimethoxypyrimidin-5-yl)boronic acid (210 mg, 1.14 mmol) and cesium carbonate (347 mg, 2.28 mmol) was degassed with nitrogen for 10 mins. Pd(dppf)Cl₂ (56 mg, 0.08 mmol) was added and the reaction mixture was heated at 100° C. for 10 mins. The resulting mixture was cooled to room temperature and partitioned between EtOAc (20 mL) and water (15 mL). The layers were separated and the aqueous portion was further extracted with EtOAc (20 mL). The combined organic extracts were washed with brine (50 mL), dried over magnesium sulfate and concentrated in vacuo. Purification by chromatography on silica eluting with a gradient of 25 to 100% EtOAc in DCM afforded the title compound as a light brown solid.

LC-MS-1 (Method 2A): Rt 1.40 mins; MS m/z 462.3=[M+H]+

1H NMR (500 MHz, DMSO-d6 @343K) δ 8.85-8.78 (m, 1H), 8.71 (d, J=2.4 Hz, 1H), 8.34 (s, 1H), 8.13 (dd, J=8.7, 2.3 Hz, 1H), 7.98 (s, 1H), 7.01 (dd, J=8.7, 0.8 Hz, 1H), 5.84-5.79 (m, 1H), 4.25-4.18 (m, 1H), 4.15-4.10 (m, 1H), 4.09 (s, 3H), 4.07-4.01 (m, 2H), 3.99 (s, 3H), 2.46-2.41 (m, 1H), 2.36-2.31 (m, 1H).

### Step 4: 6-[1-[6-(2,4-Dioxo-1H-pyrimidin-5-yl)thieno[2,3-d]pyrimidin-4-yl]pyrrolidine-3-yl]oxypyridine-3-carbonitrile

A stirred solution of 6-[1-[6-(2,4-dimethoxypyrimidin-5-yl)thieno[2,3-d]pyrimidin-4-yl]pyrrolidine-3-yl]oxypyridine-3-carbonitrile (step 3) (303 mg, 0.66 mmol) in 1M HCl (6.57 mL, 6.57 mmol) and MeOH (6 mL) was heated at 70° C. for 7 h. The mixture was allowed to cool to room temperature and the resulting suspension was collected by filtration. The solid was washed with water (20 mL), MeCN (20 mL), diethyl ether (20 mL) and dried under vacuum to afford the title compound as a yellow solid.

LC-MS-2 (Method 8A): Rt 2.46 mins; MS m/z 434.1=[M+H]+

1H NMR (500 MHz, Acetic Acid-d4 @343K) δ 8.67-8.56 (m, 2H), 8.38 (s, 1H), 8.19 (s, 1H), 7.97 (dd, J=8.8, 2.3 Hz, 1H), 6.96 (d, J=8.7 Hz, 1H), 5.97 (s, 1H), 4.58-4.24 (m, 4H), 2.58 (s, 2H).

### Example 4

### 5-[4-(3,3-Difluoro-4,4-dimethyl-pyrrolidin-1-yl)thieno[2,3-d]pyrimidin-6-yl]-1H-pyrimidine-2,4-dione

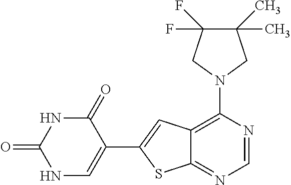 [SMILES: CC1(C)CN(c2ncnc3sc(-c4c[nH]c(=O)[nH]c4=O)cc23)CC1(F)F]

### Step 1: 4-(3,3-Difluoro-4,4-dimethyl-pyrrolidin-1-yl)-6-(2,4-dimethoxypyrimidin-5-yl)thieno[2,3-d]pyrimidine

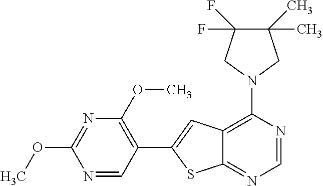

A suspension of 4-chloro-6-(2,4-dimethoxypyrimidin-5-yl)thieno[2,3-d]pyrimidine (Intermediate A) (200 mg, 0.65 mmol), 3,3-difluoro-4,4-dimethyl-pyrrolidine hydrochloride (133 mg, 0.78 mmol) and DIPEA (0.45 mL, 2.59 mmol) in MeCN (3.5 mL) was stirred at 80° C. for 16 h. The solution was allowed to cool to room temperature resulting in the formation of a suspension. The solids were removed by filtration and washed with MeCN (3×3 mL). The filtrate was concentrated in vacuo and purified by chromatography on silica eluting with a gradient of 20 to 60% EtOAc in petroleum ether afforded the title compound as a yellow solid.

LC-MS-1 (Method 2A): Rt 1.57 mins; MS m/z 408.3=[M+H]+

¹H NMR (400 MHz, DMSO-d6) δ 9.00 (s, 1H), 8.39 (s, 1H), 8.00 (s, 1H), 4.54-4.20 (m, 2H), 4.08 (s, 3H), 3.97 (s, 3H), 3.95-3.82 (m, 2H), 1.21 (s, 6H).

### Step 2: 5-[4-(3,3-Difluoro-4,4-dimethyl-pyrrolidin-1-yl)thieno[2,3-d]pyrimidin-6-yl]-1H-pyrimidine-2,4-dione

A suspension of 4-(3,3-difluoro-4,4-dimethyl-pyrrolidin-1-yl)-6-(2,4-dimethoxypyrimidin-5-yl)thieno[2,3-d]pyrimidine (step 1) (141 mg, 0.35 mmol) in 1M HCl (3.5 mL, 3.5 mmol) and EtOH (3.5 mL) was stirred at 70° C. for 3 h. The solution was allowed to cool to room temperature resulting in the formation of a suspension. The solids were collected by filtration, washed with water (3×3 mL), MeCN (3×3 mL) and diethyl ether (3×3 mL) and dried in vacuo. Purification by C18 reverse phase chromatography eluting with a gradient of 15 to 40% MeCN in water (+0.1% formic acid) afforded the title compound as a colourless solid.

LC-MS-2 (Method 8A): Rt 2.87 mins; MS m/z 380.1=[M+H]+

1H NMR (400 MHz, DMSO-d6) δ 11.59 (s, 2H), 8.40 (s, 1H), 8.34 (s, 1H), 7.86 (s, 1H), 4.45-4.27 (m, 2H), 3.94-3.76 (m, 2H), 1.21 (s, 6H).

### Example 4.1

### 5-[4-[3-(2-pyridylmethoxy)pyrrolidine-1-yl]thieno[2,3-d]pyrimidin-6-yl]-1H-pyrimidine-2,4-dione formate salt

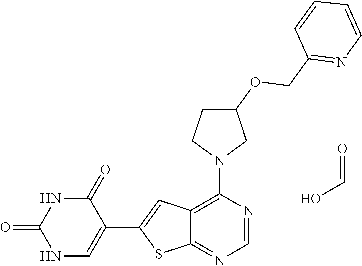 [SMILES: O=c1[nH]cc(-c2cc3c(N4CCC(OCc5ccccn5)C4)ncnc3s2)c(=O)[nH]1 ; O=CO]

### Step 1: 6-(2,4-Dimethoxypyrimidin-5-yl)-4-[3-(2-pyridylmethoxy)pyrrolidine-1-yl]thieno[2,3-d]pyrimidine

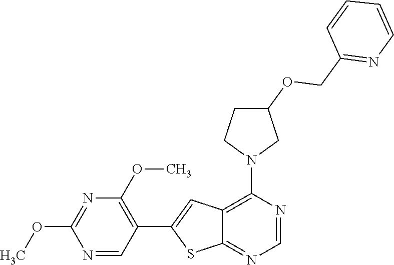 [SMILES: COc1ncc(-c2cc3c(N4CCC(OCc5ccccn5)C4)ncnc3s2)c(OC)n1]

The title compound was prepared from 4-chloro-6-(2,4-dimethoxypyrimidin-5-yl)thieno[2,3-d]pyrimidine (Intermediate A) and 2-(pyrrolidine-3-yloxymethyl)pyridine dihydrochloride analogously to Example 4 step 1.

LC-MS-2 (Method 2A): Rt 1.15 mins; MS m/z 451.1=[M+H]+

¹H NMR (400 MHz, DMSO-d₆) δ 8.92 (s, 1H), 8.50 (d, J=4.3 Hz, 1H), 8.33 (s, 1H), 8.01 (s, 1H), 7.77 (td, J=7.7, 1.8 Hz, 1H), 7.44 (d, J=7.8 Hz, 1H), 7.28 (dd, J=7.6, 4.9 Hz, 1H), 4.67 (s, 2H), 4.42 (br s, 1H), 4.20-3.70 (m, 10H), 2.27 (br s, 1H), 2.15 (br s, 1H).

### Step 2: 5-[4-[3-(2-Pyridylmethoxy)pyrrolidine-1-yl]thieno[2,3-d]pyrimidin-6-yl]-1H-pyrimidine-2,4-dione formate salt

The title compound was prepared from 6-(2,4-dimethoxypyrimidin-5-yl)-4-[3-(2-pyridylmethoxy)pyrrolidine-1-yl]thieno[2,3-d]pyrimidine (step 1) and 1M HCl analogously to Example 4 step 2.

LC-MS-2 (Method 8A): Rt 1.78 mins; MS m/z 423.1=[M+H]+

¹H NMR (400 MHz, DMSO-d₆) δ 11.97-11.25 (m, 2H), 8.51 (dd, J=5.0, 1.8 Hz, 1H), 8.34 (s, 1H), 8.28 (s, 1H), 8.15 (s, 1H), 7.92 (s, 1H), 7.77 (td, J=7.8, 1.8 Hz, 1H), 7.43 (d, J=7.8 Hz, 1H), 7.28 (dd, J=7.5, 4.8 Hz, 1H), 4.74-4.59 (m, 2H), 4.42 (s, 1H), 3.97 (br s, 4H), 2.31-2.21 (m, 1H), 2.14 (br s, 1H).

### Example 4.2

### 5-[4-(3-Benzyloxypyrrolidin-1-yl)thieno[2,3-d]pyrimidin-6-yl]-1H-pyrimidine-2,4-dione

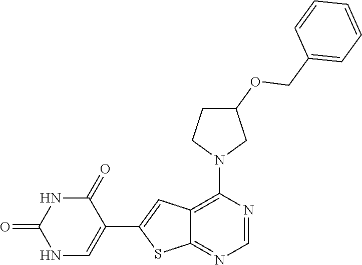 [SMILES: O=c1[nH]cc(-c2cc3c(N4CCC(OCc5ccccc5)C4)ncnc3s2)c(=O)[nH]1]

### Step 1: 4-(3-Benzyloxypyrrolidin-1-yl)-6-(2,4-dimethoxypyrimidin-5-yl)thieno[2,3-d]pyrimidine

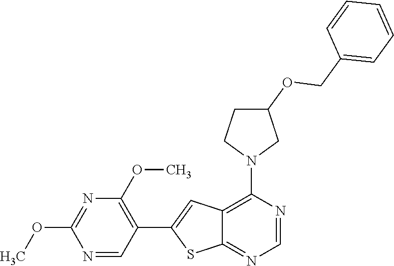 [SMILES: COc1ncc(-c2cc3c(N4CCC(OCc5ccccc5)C4)ncnc3s2)c(OC)n1]

The title compound was prepared from 4-chloro-6-(2,4-dimethoxypyrimidin-5-yl)thieno[2,3-d]pyrimidine (Intermediate A) and 3-benzyloxypyrrolidine analogously to Example 4 step 1.

LC-MS-1 (Method 2A): Rt 1.51 mins; MS m/z 450.3=[M+H]+

1H NMR (400 MHz, DMSO-d6) δ 8.92 (s, 1H), 8.33 (s, 1H), 8.00 (s, 1H), 7.54-7.14 (m, 4H), 7.32-7.15 (m, 1H), 4.60 (s, 2H), 4.46-4.25 (m, 1H), 4.14-4.03 (m, 4H), 3.97 (s, 6H), 2.30-2.20 (m, 1H), 2.19-2.10 (m, 1H).

### Step 2: 5-[4-(3-Benzyloxypyrrolidin-1-yl)thieno[2,3-d]pyrimidin-6-yl]-1H-pyrimidine-2,4-dione

The title compound was prepared from 4-(3-benzyloxypyrrolidin-1-yl)-6-(2,4-dimethoxypyrimidin-5-yl)thieno[2,3-d]pyrimidine (step 1) and 1M HCl analogously to Example 4 step 2.

LC-MS-1 (Method 8A): Rt 2.57 mins; MS m/z 422.3=[M+H]+

1H NMR (500 MHz, DMSO-d6) δ 11.28 (s, 2H), 8.28 (s, 1H), 8.18-8.15 (m, 1H), 7.90 (s, 1H), 7.40-7.22 (m, 5H), 4.64-4.57 (m, 2H), 4.39-4.32 (m, 1H), 3.99-3.86 (m, 4H), 2.27-2.19 (m, 1H), 2.19-2.11 (m, 1H).

### Example 4.3

### 5-[4-[3,3-Difluoro-4-[(4-methylmorpholin-2-yl)methoxy]pyrrolidine-1-yl]thieno[2,3-d]pyrimidin-6-yl]-1H-pyrimidine-2,4-dione formate salt

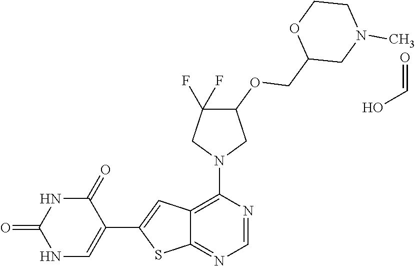

### Step 1: 2-[[1-[6-(2,4-Dimethoxypyrimidin-5-yl)thieno[2,3-d]pyrimidin-4-yl]-4,4-difluoro-pyrrolidin-3-yl]oxymethyl]-4-methyl-morpholine

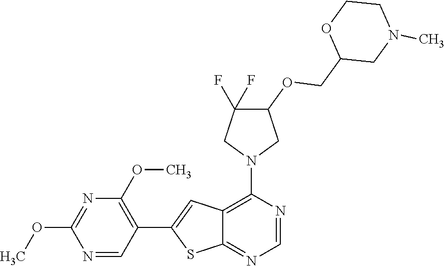

The title compound was prepared from 4-chloro-6-(2,4-dimethoxypyrimidin-5-yl)thieno[2,3-d]pyrimidine (Intermediate A) and 2-[(4,4-difluoropyrrolidin-3-yl)oxymethyl]-4-methyl-morpholine dihydrochloride (Intermediate H) analogously to Example 4 step 1.

LC-MS-2 (Method 2A): Rt 1.16 mins; MS m/z 509.1=[M+H]+

¹H NMR (400 MHz, DMSO-d₆) δ 9.02 (s, 1H), 8.41 (s, 1H), 8.02 (s, 1H), 4.44 (br s, 1H), 4.38-4.13 (m, 3H), 4.08 (s, 3H), 4.05-3.91 (m, 4H), 3.78-3.56 (m, 4H), 3.53-3.42 (m, 1H), 2.71-2.63 (m, 1H), 2.60-2.54 (m, 1H), 2.17-2.12 (m, 3H), 1.99-1.86 (m, 1H), 1.79-1.70 (m, 1H).

### Step 2: 5-[4-[3,3-Difluoro-4-[(4-methylmorpholin-2-yl)methoxy]pyrrolidine-1-yl]thieno[2,3-d]pyrimidin-6-yl]-1H-pyrimidine-2,4-dione formate salt

The title compound was prepared from 2-[[1-[6-(2,4-dimethoxypyrimidin-5-yl)thieno[2,3-d]pyrimidin-4-yl]-4,4-difluoro-pyrrolidin-3-yl]oxymethyl]-4-methyl-morpholine (step 1) and 1M HCl analogously to Example 4 step 2.

LC-MS-2 (Method 8A): Rt 2.02 mins; MS m/z 481.0=[M+H]+

1H NMR (400 MHz, Acetic Acid-d4) δ 8.46 (s, 1H), 8.10 (d, J=3.8 Hz, 1H), 8.06 (s, 1H), 7.92-7.85 (m, 1H), 4.41-4.02 (m, 7H), 4.02-3.79 (m, 3H), 3.65 (t, J=14.1 Hz, 1H), 3.54 (d, J=12.4 Hz, 1H), 3.12-2.91 (m, 2H), 2.87 (s, 3H).

### Example 4.4

### 5-[4-[3-[(4-Methylmorpholin-2-yl)methoxy]pyrrolidine-1-yl]thieno[2,3-d]pyrimidin-6-yl]-1H-pyrimidine-2,4-dione

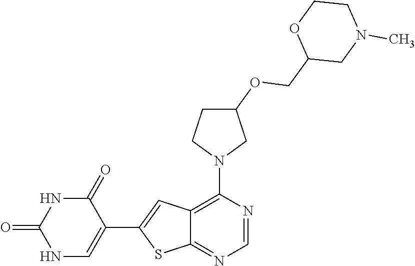 [SMILES: CN1CCOC(COC2CCN(c3ncnc4sc(-c5c[nH]c(=O)[nH]c5=O)cc34)C2)C1]

### Step 1: 2-[[1-[6-(2,4-Dimethoxypyrimidin-5-yl)thieno[2,3-d]pyrimidin-4-yl]pyrrolidine-3-yl]oxymethyl]-4-methyl-morpholine

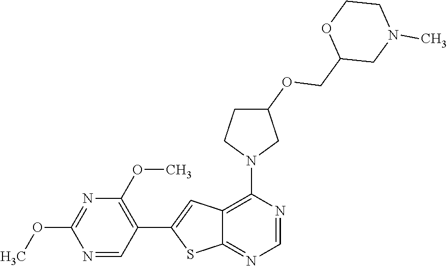

The title compound was prepared from 4-chloro-6-(2,4-dimethoxypyrimidin-5-yl)thieno[2,3-d]pyrimidine (Intermediate A) and 4-methyl-2-(pyrrolidine-3-yloxymethyl)morpholine (Intermediate I) analogously to Example 4 step 1.

LC-MS-1 (Method 2A): Rt 0.87 mins; MS m/z 473.3=[M+H]+

1H NMR (400 MHz, DMSO-d6) δ 8.93 (s, 1H), 8.33 (s, 1H), 8.00 (s, 1H), 4.26 (s, 1H), 4.12-3.62 (m, 10H), 3.61-3.40 (m, 4H), 2.72-2.63 (m, 1H), 2.59-2.53 (m, 2H), 2.18-2.04 (m, 5H), 1.98-1.86 (m, 1H), 1.72 (q, J=9.8 Hz, 1H).

### Step 2: 5-[4-[3-[(4-Methylmorpholin-2-yl)methoxy]pyrrolidine-1-yl]thieno[2,3-d]pyrimidin-6-yl]-1H-pyrimidine-2,4-dione formate salt

The title compound was prepared from 2-[[1-[6-(2,4-dimethoxypyrimidin-5-yl)thieno[2,3-d]pyrimidin-4-yl]pyrrolidine-3-yl]oxymethyl]-4-methyl-morpholine (step 1) and 1M HCl analogously to Example 4 step 2.

LC-MS-1 (Method 8B): Rt 1.47 mins; MS m/z 445.3=[M+H]+

1H NMR (400 MHz, Acetic Acid-d4) δ 8.49 (s, 1H), 8.15 (s, 1H), 8.09 (s, 1H), 8.01 (d, J=3.2 Hz, 1H), 4.39 (s, 1H), 4.24-3.81 (m, 7H), 3.82-3.60 (m, 3H), 3.60-3.48 (m, 1H), 3.20-2.91 (m, 2H), 2.88 (s, 3H), 2.47-2.30 (m, 1H), 2.28-2.15 (m, 1H).

### Example 5

### 5-[4-[3,3-Difluoro-4-[2-(1-piperidyl)ethoxy]pyrrolidine-1-yl]thieno[2,3-d]pyrimidin-6-yl]-1H-pyrimidine-2,4-dione formate salt

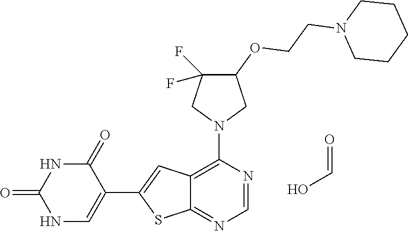 [SMILES: O=c1[nH]cc(-c2cc3c(N4CC(OCCN5CCCCC5)C(F)(F)C4)ncnc3s2)c(=O)[nH]1 ; O=CO]

### Step 1: 1-(6-Bromothieno[2,3-d]pyrimidin-4-yl)-4,4-difluoro-pyrrolidin-3-ol

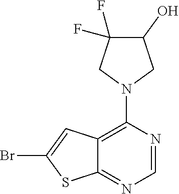 [SMILES: OC1CN(c2ncnc3sc(Br)cc23)CC1(F)F]

The title compound was prepared from 6-bromo-4-chloro-thieno[2,3-d]pyrimidine and 4,4-difluoropyrrolidin-3-ol hydrochloride analogously to Example 3 step 1.

LC-MS-1 (Method 2A): Rt 1.20 mins; MS m/z 336.1/338.1=[M+H]+

1H NMR (400 MHz, DMSO-d6) δ 8.40 (s, 1H), 7.87 (s, 1H), 6.26 (m, 1H), 4.57-4.31 (m, 1H), 4.31-3.96 (m, 3H), 3.90-3.58 (m, 1H).

### Step 2: 6-Bromo-4-[3,3-difluoro-4-[2-(1-piperidyl)ethoxy]pyrrolidine-1-yl]thieno[2,3-d]pyrimidine

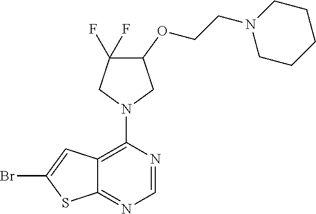 [SMILES: FC1(F)CN(c2ncnc3sc(Br)cc23)CC1OCCN1CCCCC1]

A mixture of 1-(6-bromothieno[2,3-d]pyrimidin-4-yl)-4,4-difluoro-pyrrolidin-3-ol (step 1) (200 mg, 0.59 mmol), 1-(2-chloroethyl)piperidine (Intermediate J step 1) (108 mg, 0.73 mmol), TBAB (108 mg, 0.34 mmol) and 5M aq. sodium hydroxide (2 mL, 10 mmol) in toluene (6 mL) was heated at 80° C. for 100 mins. The reaction mixture was allowed to cool to room temperature before being partitioned between EtOAc (50 mL) and water (50 mL). The phases were separated and the aqueous portion was further extracted with EtOAc (2×50 mL). The combined organic extracts were dried over magnesium sulfate and concentrated in vacuo. Purification by chromatography on silica eluting with a gradient of 0 to 10% MeOH in DCM afforded the title compound as a yellow oil.

LC-MS-2 (Method 2A): Rt 1.17 mins; MS m/z 447.0/449.0=[M+H]+

1H NMR (400 MHz, DMSO-d6) δ 8.40 (s, 1H), 7.88 (s, 1H), 4.41 (p, J=6.2 Hz, 1H), 4.27-4.13 (m, 3H), 4.09 (q, J=5.3 Hz, 2H), 4.00-3.89 (m, 1H), 3.79 (t, J=5.9 Hz, 2H), 2.46-2.34 (m, 4H), 1.51-1.44 (m, 4H), 1.38-1.32 (m, 2H).

### Step 3: 4-[3,3-Difluoro-4-[2-(1-piperidyl)ethoxy]pyrrolidin-1-yl]-6-(2,4-dimethoxypyrimidin-5-yl)thieno[2,3-d]pyrimidine

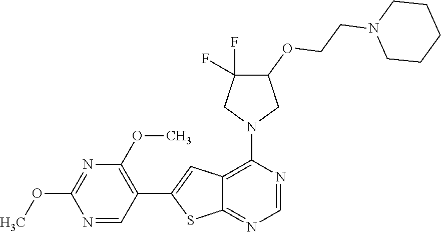 [SMILES: COc1ncc(-c2cc3c(N4CC(OCCN5CCCCC5)C(F)(F)C4)ncnc3s2)c(OC)n1]

The title compound was prepared from 4-[2-[1-(6-bromothieno[2,3-d]pyrimidin-4-yl)-4,4-difluoro-pyrrolidin-3-yl]oxyethyl]morpholine (step 2) and 2,4-dimethoxypyrimidin-5-yl)boronic acid analogously to Example 3 step 3.

LC-MS-2 (Method 2A): Rt 1.18 mins; MS m/z 507.2=[M+H]+

1H NMR (400 MHz, DMSO-d6) δ 9.01 (s, 1H), 8.41 (s, 1H), 8.01 (s, 1H), 4.45 (s, 1H), 4.39-4.15 (m, 3H), 4.08 (s, 3H), 4.05-3.99 (m, 1H), 3.97 (s, 3H), 3.83 (s, 2H), 2.63-2.53 (m, 2H), 2.42 (s, 4H), 1.56-1.42 (m, 4H), 1.41-1.30 (m, 2H).

### Step 4: 5-[4-[3,3-Difluoro-4-[2-(1-piperidyl)ethoxy]pyrrolidin-1-yl]thieno[2,3-d]pyrimidin-6-yl]-1H-pyrimidine-2,4-dione formate salt

The title compound was prepared from 4-[3,3-difluoro-4-[2-(1-piperidyl)ethoxy]pyrrolidin-1-yl]-6-(2,4-dimethoxypyrimidin-5-yl)thieno[2,3-d]pyrimidine (step 3) and 1M HCl analogously to Example 3 step 4.

LC-MS-2 (Method 8A): Rt 2.04 mins; MS m/z 479.1=[M+H]+

1H NMR (400 MHz, DMSO-d6) δ 11.59 (s, 2H), 8.42 (s, 1H), 8.36 (s, 1H), 8.15 (s, 1H), 7.88 (s, 1H), 4.44 (d, J=7.3 Hz, 1H), 4.28 (s, 2H), 4.18 (s, 1H), 3.97 (d, J=12.1 Hz, 1H), 3.83-3.76 (m, 2H), 2.58-2.56 (m, 2H), 2.46-2.35 (m, 4H), 1.52-1.41 (m, 4H), 1.39-1.29 (m, 2H).

### Example 6

### 5-[7-[3-[2-(1-Piperidyl)ethoxy]pyrrolidin-1-yl]thiazolo[4,5-d]pyrimidin-2-yl]-1H-pyrimidine-2,4-dione

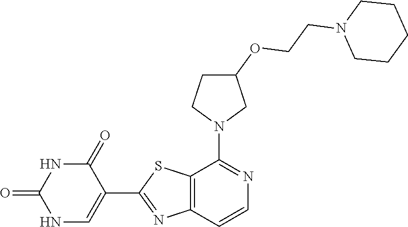 [SMILES: O=c1[nH]cc(-c2nc3ccnc(N4CCC(OCCN5CCCCC5)C4)c3s2)c(=O)[nH]1]

### Step 1: 7-Chloro-2-methylsulfanyl-thiazolo[4,5-d]pyrimidine

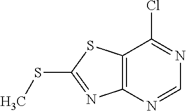

N,N-Dimethylaniline (0.74 mL, 5.88 mmol) was added to a solution of 2-methylsulfanyl-6H-thiazolo[4,5-d]pyrimidin-7-one (500 mg, 2.51 mmol) in phosphorus (V) oxychloride (5.0 mL, 68.55 mmol) and the reaction mixture was stirred at 80° C. for 2 h. The resulting mixture was concentrated in vacuo, azeotroping with chloroform (2×15 mL). The residue was triturated with MeCN (˜3 mL) and the resulting solid was collected by filtration and washed with MeCN (2×1 mL) to afford the title compound as an off-white solid.

LC-MS (Method 2A): Rt 0.74 mins; MS m/z 218.5=[M+H]+

1H NMR (500 MHz, DMSO) δ 9.01 (s, 1H), 2.89 (s, 3H).

### Step 2: 2-Methylsulfanyl-7-[3-[2-(1-piperidyl)ethoxy]pyrrolidin-1-yl]thiazolo[4,5-d]pyrimidine

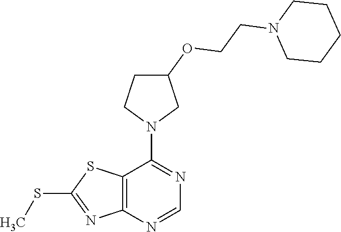 [SMILES: CSc1nc2ncnc(N3CCC(OCCN4CCCCC4)C3)c2s1]

The title compound was prepared from 7-chloro-2-methylsulfanyl-thiazolo[4,5-d]pyrimidine (step 1) and 1-(2-pyrrolidin-3-yloxyethyl)piperidine (Intermediate J) analogously to Example 2 step 1.

LC-MS (Method 2A): Rt 0.46 mins; MS m/z 380.1=[M+H]+

1H NMR (500 MHz, DMSO-d6) δ 8.39 (s, 1H), 4.26-4.18 (m, 1H), 3.92-3.62 (m, 4H), 3.61-3.50 (m, 2H), 2.80 (s, 3H), 2.47-2.41 (m, 2H), 2.41-2.23 (m, 4H), 2.18-1.99 (m, 2H), 1.51-1.38 (m, 4H), 1.38-1.26 (m, 2H).

### Step 3: 2-(2,4-Dimethoxypyrimidin-5-yl)-7-[3-[2-(1-piperidyl)ethoxy]pyrrolidin-1-yl]thiazolo[4,5-d]pyrimidine

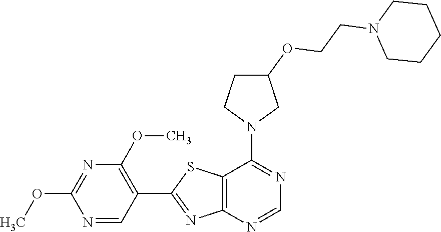 [SMILES: COc1ncc(-c2nc3ncnc(N4CCC(OCCN5CCCCC5)C4)c3s2)c(OC)n1]

A solution of 2-methylsulfanyl-7-[3-[2-(1-piperidyl)ethoxy]pyrrolidin-1-yl]thiazolo[4,5-d]pyrimidine (step 2) (100 mg, 0.25 mmol), copper (I) thiophene-2-carboxylate (117 mg, 0.61 mmol) and Pd(PPh₃)₄ (28 mg, 0.02 mmol) in THF (2 mL) was stirred under nitrogen at room temperature for 10 mins. (2,4-Dimethoxypyrimidin-5-yl)boronic acid (90 mg, 0.49 mmol) was added and the reaction mixture was heated at 60° C. overnight. Additional Pd(PPh₃)₄ (28 mg, 0.02 mmol) was added and heating continued at 60° C. for 4 h. The mixture was allowed to cool to room temperature before being partitioned between EtOAc (5 mL) and water (1 mL). The mixture was filtered through glass fibre filter paper and the precipitate was washed with water (3 mL) and EtOAc (10 mL). The filtrate layers were separated and the organic layer was washed further with 10% aqueous NH₄OH (20 mL). The combined aqueous layers were extracted with EtOAc (30 mL) and the organic extracts were combined and washed with 10% aq. NH₄OH (3×20 mL) until the aqueous layer was no longer blue. The combined organic extracts were dried over magnesium sulfate and concentrated in vacuo. The crude material was purified by C18 reverse phase chromatography eluting with a MeCN in water (+0.1% NH₄OH) and the column was then flushed with 100% MeOH. A fraction of the MeOH flush containing desired product was concentrated in vacuo to afford the title compound as a yellow oil

LC-MS (Method 7B): Rt 3.07 mins; MS m/z 472.3=[M+H]+

### Step 4: 5-[7-[3-[2-(1-Piperidyl)ethoxy]pyrrolidin-1-yl]thiazolo[4,5-d]pyrimidin-2-yl]-1H-pyrimidine-2,4-dione

The title compound was prepared from 2-(2,4-dimethoxypyrimidin-5-yl)-7-[3-[2-(1-piperidyl)ethoxy]pyrrolidin-1-yl]thiazolo[4,5-d]pyrimidine (step 3) and 1M HCl analogously to Example 2 step 3.

LC-MS (Method 7B): Rt 1.51 mins; MS m/z 444.2=[M+H]+

1H NMR (400 MHz, DMSO-d6) δ 11.60 (s, 2H), 8.62 (s, 1H), 8.40 (s, 1H), 4.28-4.23 (m, 1H), 3.96-3.88 (m, 1H), 3.88-3.82 (m, 2H), 3.80-3.72 (m, 1H), 3.65-3.55 (m, 2H), 2.55 (t, J=6.0 Hz, 2H), 2.48-2.41 (m, 4H), 2.20-2.05 (m, 2H), 1.52-1.42 (m, 4H), 1.38-1.30 (m, 2H).

### Example 6.1

### 5-[7-(3,3-difluoro-4,4-dimethyl-pyrrolidin-1-yl)thiazolo[4,5-d]pyrimidin-2-yl]-1H-pyrimidine-2,4-dione

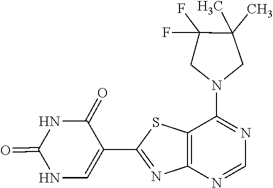 [SMILES: CC1(C)CN(c2ncnc3nc(-c4c[nH]c(=O)[nH]c4=O)sc23)CC1(F)F]

### Step 1: 7-(3,3-difluoro-4,4-dimethyl-pyrrolidin-1-yl)-2-methylsulfanyl-thiazolo[4,5-d]pyrimidine

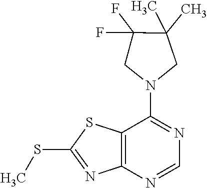

The title compound was prepared from 7-chloro-2-methylsulfanyl-thiazolo[4,5-d]pyrimidine (Example 6 step 1) and 3,3-difluoro-4,4-dimethyl-pyrrolidine hydrochloride analogously the Example 6 step 2.

LC-MS-1 (Method 2A): Rt 1.37 mins; MS m/z 317.5=[M+H]+

1H NMR (400 MHz, DMSO-d6) δ 8.46 (s, 1H), 4.23 (t, J=13.6 Hz, 2H), 3.80 (s, 2H), 2.82 (s, 3H), 1.19 (s, 6H).

### Step 2: 7-(3,3-difluoro-4,4-dimethyl-pyrrolidin-1-yl)-2-(2,4-dimethoxypyrimidin-5-yl)thiazolo[4,5-d]pyrimidine

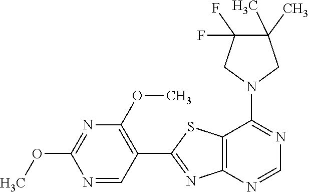

The title compound was prepared from 7-(3,3-difluoro-4,4-dimethyl-pyrrolidin-1-yl)-2-methylsulfanyl-thiazolo[4,5-d]pyrimidine (step 1) and (2,4-Dimethoxypyrimidin-5-yl)boronic acid analogously to Example 6 step 3.

LC-MS-2 (Method 2A): Rt 1.45 mins; MS m/z 409.0=[M+H]+

1H NMR (400 MHz, DMSO-d6) δ 9.32 (s, 1H), 8.55 (s, 1H), 4.39-4.28 (m, 2H), 4.22 (s, 3H), 4.04 (s, 3H), 3.92-3.84 (m, 2H), 1.23 (s, 6H).

### Step 3: 5-[7-(3,3-difluoro-4,4-dimethyl-pyrrolidin-1-yl)thiazolo[4,5-d]pyrimidin-2-yl]-1H-pyrimidine-2,4-dione

The title compound was prepared from 7-(3,3-difluoro-4,4-dimethyl-pyrrolidin-1-yl)-2-(2,4-dimethoxypyrimidin-5-yl)thiazolo[4,5-d]pyrimidine (step 2) and 1M HCl analogously to Example 6 step 4.

LC-MS-2 (Method 8B): Rt 2.48 mins; MS m/z 381.0=[M+H]+

1H NMR (400 MHz, DMSO-d6) δ 12.04 (br s, 1H), 11.92 (s, 1H), 8.63 (br s, 1H), 8.50 (s, 1H), 4.28 (t, J=11.8 Hz, 2H), 3.89 (s, 2H), 1.23 (s, 6H).

### Example 7

### 5-[4-[3-[2-(1-Piperidyl)ethoxy]pyrrolidin-1-yl]furo[2,3-d]pyrimidin-6-yl]-1H-pyrimidine-2,4-dione formate salt

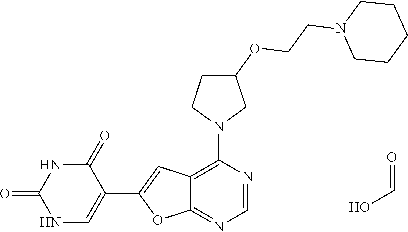 [SMILES: O=c1[nH]cc(-c2cc3c(N4CCC(OCCN5CCCCC5)C4)ncnc3o2)c(=O)[nH]1 ; O=CO]

### Step 1: 6-(2,4-Dimethoxypyrimidin-5-yl)-4-[3-[2-(1-piperidyl)ethoxy]pyrrolidin-1-yl]furo[2,3-d]pyrimidine

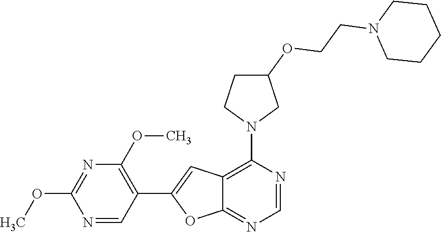 [SMILES: COc1ncc(-c2cc3c(N4CCC(OCCN5CCCCC5)C4)ncnc3o2)c(OC)n1]

To a stirred suspension of 4-chloro-6-(2,4-dimethoxypyrimidin-5-yl)furo[2,3-d]pyrimidine (Intermediate C) (200 mg, 0.68 mmol) and 1-(2-pyrrolidin-3-yloxyethyl)piperidine (Intermediate J) (163 mg, 0.82 mmol) in MeCN (5 mL) was added DIPEA (0.48 mL, 2.73 mmol) and the reaction mixture was heated at 70° C. for 90 mins. The resulting mixture was allowed to cool to room temperature, filtered and the solid washed with MeCN (50 mL). The filtrate was concentrated in vacuo and purified by chromatography on silica eluting with a gradient of 0 to 10% 1 M NH3 in MeOH in DCM afforded the title compound as a yellow solid.

LC-MS-1 (Method 2A): Rt 0.90 mins; MS m/z 455.4=[M+H]+

1H NMR (500 MHz, Acetic Acid-d4) δ 8.83 (s, 1H), 8.42 (s, 1H), 7.25 (s, 1H), 4.53-4.33 (m, 1H), 4.21 (s, 3H), 4.10-3.87 (m, 8H), 3.83-3.59 (m, 3H), 3.46-3.35 (m, 2H), 3.00-2.86 (m, 2H), 2.49-2.08 (m, 2H), 1.93-1.70 (m, 5H), 1.52-1.37 (m, 1H).

### Step 2: 5-[4-[3-[2-(1-Piperidyl)ethoxy]pyrrolidin-1-yl]furo[2,3-d]pyrimidin-6-yl]-1H-pyrimidine-2,4-dione formate salt

To a stirred solution of 6-(2,4-dimethoxypyrimidin-5-yl)-4-[3-[2-(1-piperidyl)ethoxy]pyrrolidin-1-yl]furo[2,3-d]pyrimidine (step 1) (170 mg, 0.37 mmol) in MeOH (4 mL) was added 1M HCl (3.74 mL, 3.74 mmol) and the mixture was heated at 70° C. for 6 h. The resulting mixture was allowed to cool to room temperature before being concentrated in vacuo. Purification by C18 reverse phase chromatography eluting with a gradient of 5 to 30% MeCN in water (+0.1% formic acid) afforded the title compound as an off-white solid.

LC-MS-2 (Method 8A): Rt 1.73 mins; MS m/z 427.1=[M+H]+

1H NMR (500 MHz, DMSO-d6+D₂O, @343K) δ 8.28 (s, 1H), 8.17 (d, J=2.3 Hz, 1H), 7.90 (d, J=1.9 Hz, 1H), 7.36 (d, J=2.3 Hz, 1H), 4.29 (s, 1H), 3.87-3.76 (m, 3H), 3.75-3.65 (m, 3H), 2.95-2.88 (m, 2H), 2.86-2.76 (m, 4H), 2.21-2.08 (m, 2H), 1.62-1.54 (m, 4H), 1.46-1.37 (m, 2H).

### Example 7.1

### 5-[4-(3,3-Difluoro-4,4-dimethyl-pyrrolidin-1-yl)furo[2,3-d]pyrimidin-6-yl]-1H-pyrimidine-2,4-dione

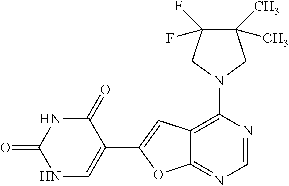 [SMILES: CC1(C)CN(c2ncnc3oc(-c4c[nH]c(=O)[nH]c4=O)cc23)CC1(F)F]

### Step 1: 4-(3,3-Difluoro-4,4-dimethyl-pyrrolidin-1-yl)-6-(2,4-dimethoxypyrimidin-5-yl)furo[2,3-d]pyrimidine

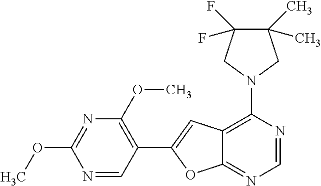

The title compound was prepared from 4-chloro-6-(2,4-dimethoxypyrimidin-5-yl)furo[2,3-d]pyrimidine (Intermediate C) and 3,3-difluoro-4,4-dimethyl-pyrrolidine hydrochloride analogously to Example 7 step 1.

LC-MS-1 (Method 2A): Rt 1.54 mins; MS m/z 392.3=[M+H]+

1H NMR (400 MHz, DMSO-d6) δ 8.75 (s, 1H), 8.31 (s, 1H), 7.28 (s, 1H), 4.48-4.17 (m, 2H), 4.11 (s, 3H), 3.96 (s, 3H), 3.87-3.66 (m, 2H), 1.22 (s, 6H).

### Step 2: 5-[4-(3,3-Difluoro-4,4-dimethyl-pyrrolidin-1-yl)furo[2,3-d]pyrimidin-6-yl]-1H-pyrimidine-2,4-dione

The title compound was prepared from 4-(3,3-difluoro-4,4-dimethyl-pyrrolidin-1-yl)-6-(2,4-dimethoxypyrimidin-5-yl)furo[2,3-d]pyrimidine (step 1) and 1M HCl analogously to Example 7 step 2.

LC-MS-2 (Method 8A): Rt 2.99 mins; MS m/z 364.0=[M+H]+

1H NMR (400 MHz, DMSO-d6) δ 11.61-11.43 (m, 2H), 8.29 (s, 1H), 7.95 (s, 1H), 7.37 (s, 1H), 4.23 (s, 2H), 3.91-3.70 (m, 2H), 1.21 (s, 6H).

### Example 8

### 5-[4-[3-[2-(1-Piperidyl)ethoxy]pyrrolidin-1-yl]thieno[3,2-d]pyrimidin-6-yl]-1H-pyrimidine-2,4-dione

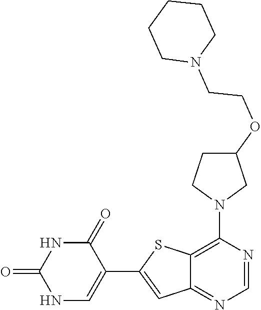 [SMILES: O=c1[nH]cc(-c2cc3ncnc(N4CCC(OCCN5CCCCC5)C4)c3s2)c(=O)[nH]1]

### Step 1: 4-Chloro-6-(2,4-dimethoxypyrimidin-5-yl)thieno[3,2-d]pyrimidine

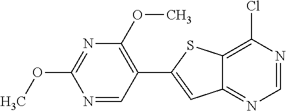 [SMILES: COc1ncc(-c2cc3ncnc(Cl)c3s2)c(OC)n1]

The title compound was prepared from 6-bromo-4-chloro-thieno[3,2-d]pyrimidine and (2,4-dimethoxypyrimidin-5-yl)boronic acid analogously to Example 2 step 2.

LC-MS (Method 7A): Rt 3.29 mins; MS m/z 309.5/311.4=[M+H]+

1H NMR (500 MHz, CDCl3) δ 8.96 (s, 1H), 8.73 (s, 1H), 7.91 (s, 1H), 4.21 (s, 3H), 4.09 (s, 3H).

### Step 2: 5-[4-[3-[2-(1-Piperidyl)ethoxy]pyrrolidin-1-yl]thieno[3,2-d]pyrimidin-6-yl]-1H-pyrimidine-2,4-dione

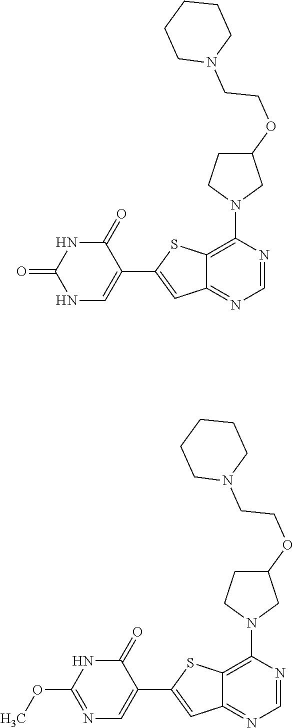 [SMILES: O=c1[nH]cc(-c2cc3ncnc(N4CCC(OCCN5CCCCC5)C4)c3s2)c(=O)[nH]1 ; COc1ncc(-c2cc3ncnc(N4CCC(OCCN5CCCCC5)C4)c3s2)c(=O)[nH]1]

The title compound was prepared from 4-chloro-6-(2,4-dimethoxypyrimidin-5-yl)thieno[3,2-d]pyrimidine (step 1) and 1-(2-pyrrolidin-3-yloxyethyl)piperidine (Intermediate J) analogously to Example 2 step 1. The reaction was carried out in NMP at 130° C. and afforded a 3:7 mixture of 2-methoxy-5-[4-[3-[2-(1-piperidyl)ethoxy]pyrrolidin-1-yl]thieno[3,2-d]pyrimidin-6-yl]-1H-pyrimidin-6-one and 5-[4-[3-[2-(1-piperidyl)ethoxy]pyrrolidin-1-yl]thieno[3,2-d]pyrimidin-6-yl]-1H-pyrimidine-2,4-dione.

The aforementioned mixture of 2-methoxy-5-[4-[3-[2-(1-piperidyl)ethoxy]pyrrolidin-1-yl]thieno[3,2-d]pyrimidin-6-yl]-1H-pyrimidin-6-one and 5-[4-[3-[2-(1-piperidyl)ethoxy]pyrrolidin-1-yl]thieno[3,2-d]pyrimidin-6-yl]-1H-pyrimidine-2,4-dione was treated with 1M HCl analogously to Example 2 step 3.

LC-MS (Method 7B): Rt 1.56 mins; MS m/z 443.2=[M+H]+

1H NMR (400 MHz, DMSO-d6) δ 11.36 (s, 2H), 8.39 (s, 1H), 8.31 (s, 1H), 7.72 (s, 1H), 4.26-4.17 (m, 1H), 3.97-3.90 (m, 1H), 3.90-3.82 (m, 2H), 3.80-3.73 (m, 1H), 3.61-3.50 (m, 2H), 2.42 (t, J=6.1 Hz, 2H), 2.37-2.29 (m, 4H), 2.18-2.02 (m, 2H), 1.48-1.38 (m, 4H), 1.34-1.26 (m, 2H).

### Example 9

### 5-[4-[3-[2-(1-Piperidyl)ethoxy]pyrrolidin-1-yl]thieno[3,2-c]pyridin-2-yl]-1H-pyrimidine-2,4-dione

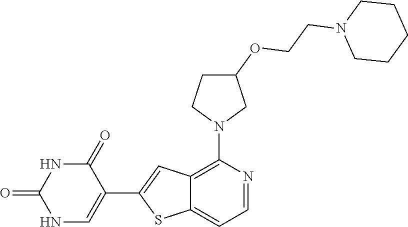 [SMILES: O=c1[nH]cc(-c2cc3c(N4CCC(OCCN5CCCCC5)C4)nccc3s2)c(=O)[nH]1]

### Step 1: 4-Chloro-2-(2,4-dimethoxypyrimidin-5-yl)thieno[3,2-c]pyridine

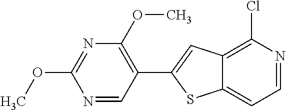 [SMILES: COc1ncc(-c2cc3c(Cl)nccc3s2)c(OC)n1]

The title compound was prepared from 2-bromo-4-chloro-thieno[3,2-c]pyridine and (2,4-dimethoxypyrimidin-5-yl)boronic acid analogously to Example 2 step 2.

LC-MS-2 (Method 2A): Rt 1.59 mins; MS m/z 308.0=[M+H]+

1H NMR (400 MHz, DMSO-d6) δ 8.98 (s, 1H), 8.23 (d, J=5.5 Hz, 1H), 8.11 (d, J=5.5 Hz, 1H), 7.99 (s, 1H), 4.11 (s, 3H), 3.99 (s, 3H).

### Step 2: 5-(4-Chlorothieno[3,2-c]pyridin-2-yl)-1H-pyrimidine-2,4-dione

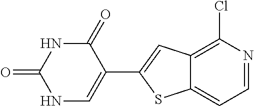 [SMILES: O=c1[nH]cc(-c2cc3c(Cl)nccc3s2)c(=O)[nH]1]

The title compound was prepared from 4-chloro-2-(2,4-dimethoxypyrimidin-5-yl)thieno[3,2-c]pyridine (step 1) and 1M HCl analogously to Example 2 step 3.

LC-MS-2 (Method 2A): Rt 1.05 mins; MS m/z 279.9=[M+H]+

1H NMR (400 MHz, DMSO-d6) δ 11.71 (d, J=5.8 Hz, 1H), 11.66 (s, 1H), 8.52 (d, J=5.9 Hz, 1H), 8.15 (d, J=5.4 Hz, 1H), 8.03 (d, J=5.5 Hz, 1H), 7.98 (s, 1H).

### Step 3: 5-[4-[3-[2-(1-Piperidyl)ethoxy]pyrrolidin-1-yl]thieno[3,2-c]pyridin-2-yl]-1H-pyrimidine-2,4-dione

The title compound was prepared from 4-chloro-2-(2,4-dimethoxypyrimidin-5-yl)thieno[3,2-c]pyridine (step 2) and 1-(2-pyrrolidin-3-yloxyethyl)piperidine (Intermediate J) analogously to Example 2 step 1. The reaction was carried out in NMP at 120° C.

LC-MS-2 (Method 8B): Rt 2.29 mins; MS m/z 442.1=[M+H]+

1H NMR (400 MHz, Acetic Acid-d4) δ 8.47 (s, 1H), 8.31 (s, 1H), 7.70 (d, J=6.9 Hz, 1H), 7.40 (d, J=6.9 Hz, 1H), 4.51 (s, 1H), 4.27-4.09 (m, 4H), 4.02-3.91 (m, 2H), 3.65 (d, J=12.2 Hz, 2H), 3.45-3.34 (m, 2H), 3.01-2.86 (m, 2H), 2.50-2.39 (m, 1H), 2.31-2.21 (m, 1H), 1.87-1.78 (m, 4H), 1.78-1.68 (m, 1H), 1.55-1.38 (m, 1H).

### Example 10

### 5-[4-[3-[2-(1-Piperidyl)ethoxy]pyrrolidin-1-yl]pyrrolo[2,1-f][1,2,4]triazin-6-yl]pyrimidine-2,4-diol

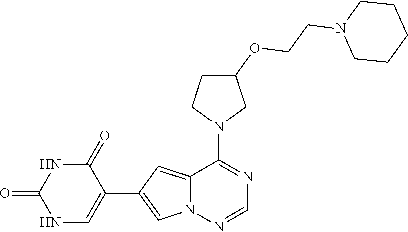 [SMILES: O=c1[nH]cc(-c2cc3c(N4CCC(OCCN5CCCCC5)C4)ncnn3c2)c(=O)[nH]1]

### Step 1: 6-Bromo-4-[3-[2-(1-piperidyl)ethoxy]pyrrolidin-1-yl]pyrrolo[2,1-f][1,2,4]triazine

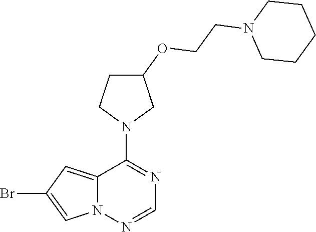 [SMILES: Brc1cc2c(N3CCC(OCCN4CCCCC4)C3)ncnn2c1]

A solution of 6-bromo-4-chloro-pyrrolo[2,1-f][1,2,4]triazine (75 mg, 0.32 mmol), 1-(2-pyrrolidin-3-yloxyethyl)piperidine (Intermediate J) (70 mg, 0.35 mmol) and DIPEA (112 μL, 0.65 mmol) in EtOH (0.5 mL) was stirred at room temperature for 1 h. The resulting solid was removed by vacuum filtration and the filtrate was concentrated in vacuo. Purification by chromatography on silica eluting with a gradient of 0 to 15% MeOH in DCM afforded the title compound as a colourless glass.

LC-MS (Method 2B): Rt 0.81 mins; MS m/z 394.2/396.2=[M+H]+

1H NMR (500 MHz, CDCl3) δ 7.80 (s, 1H), 7.52 (d, J=1.6 Hz, 1H), 6.74 (s, 1H), 4.38-4.20 (m, 1H), 4.05-3.93 (m, 4H), 3.88-3.81 (m, 1H), 3.78-3.67 (m, 1H), 2.94-2.75 (m, 6H), 2.41-1.97 (m, 2H), 1.88-1.78 (m, 4H), 1.58-1.47 (m, 2H).

### Step 2: 6-(2,4-Dimethoxypyrimidin-5-yl)-4-[3-[2-(1-piperidyl)ethoxy]pyrrolidin-1-yl]pyrrolo[2,1-f][1,2,4]triazine

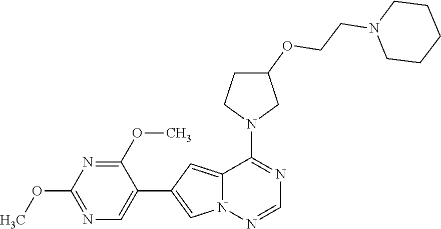 [SMILES: COc1ncc(-c2cc3c(N4CCC(OCCN5CCCCC5)C4)ncnn3c2)c(OC)n1]

A solution of 6-bromo-4-[3-[2-(1-piperidyl)ethoxy]pyrrolidin-1-yl]pyrrolo[2,1-f][1,2,4]triazine (step 1) (30 mg, 0.08 mmol), (2,4-dimethoxypyrimidin-5-yl)boronic acid (21 mg, 0.11 mmol) and cesium carbonate (74 mg, 0.23 mmol) in 1,4-dioxane (1 mL) and water (0.1 mL) was degassed for 5 mins and treated with Pd(dppf)Cl₂-DCM (3 mg, 3.8 μmol). The reaction mixture was heated at 100° C. overnight and then allowed to cool to room temperature. The resulting mixture was diluted with EtOAc and filtered through a magnesium sulfate plug. The filtrate was concentrated in vacuo and purification by chromatography on silica eluting with a gradient of 0 to 20% 1M NH3 in MeOH in DCM afforded the title compound as a brown oil.

LC-MS (Method 7B): Rt 3.33 mins; MS m/z 454.7=[M+H]+

1H NMR (400 MHz, CDCl3) δ 8.50 (s, 1H), 7.96 (d, J=1.7 Hz, 1H), 7.85 (s, 1H), 7.00 (s, 1H), 4.32-4.13 (m, 3H), 4.11 (s, 3H), 4.03 (s, 3H), 4.01-3.96 (m, 2H), 3.67-3.59 (m, 2H), 2.58-2.51 (m, 2H), 2.46-2.37 (m, 4H), 2.34-2.00 (m, 2H), 1.60-1.52 (m, 4H), 1.45-1.34 (m, 2H).

### Step 3: 5-[4-[3-[2-(1-Piperidyl)ethoxy]pyrrolidin-1-yl]pyrrolo[2,1-f][1,2,4]triazin-6-yl]pyrimidine-2,4-diol

A solution of 6-(2,4-dimethoxypyrimidin-5-yl)-4-[3-[2-(1-piperidyl)ethoxy]pyrrolidin-1-yl]pyrrolo[2,1-f][1,2,4]triazine (step 2) (37 mg, 0.04 mmol) in 1M HCl (500 μL, 0.5 mmol) and MeOH (0.5 mL) was heated at 70° C. overnight. The resulting mixture was allowed to cool to room temperature before being concentrated in vacuo. The crude product was dissolved in 3:1:1 DMSO:MeCN:H₂O (1.4 mL) and purified by preparative HPLC under basic conditions (details below) to afford the title compound as a colourless powder.

LC-MS (Method 7B): Rt 1.87 mins; MS m/z 426.3=[M+H]+

1H NMR (500 MHz, Methanol-d4) δ 8.10 (d, J=1.7 Hz, 1H), 7.88 (s, 1H), 7.71 (s, 1H), 7.27 (d, J=1.7 Hz, 1H), 4.60-4.56 (m, 1H), 4.28-4.04 (m, 4H), 3.72-3.66 (m, 2H), 2.60 (t, J=5.7 Hz, 2H), 2.56-2.42 (m, 4H), 2.25-2.16 (m, 1H), 2.14-2.02 (m, 1H), 1.60-1.53 (m, 4H), 1.47-1.39 (m, 2H).

### Purification Conditions:

Waters XBridge C18 column (30 mm×100 mm, 5 μm; temperature: room temperature). Injection volume of 1500 μL at a flow rate of 40 mL/min. 10% B (A=0.2% ammonium hydroxide in water; B=0.2% ammonium hydroxide in acetonitrile) for 2.0 minutes then a gradient of 10-95% B over 14.0 minutes and held for 2.0.minutes. A second gradient of 95-10% B was then applied over 0.2 minutes and held for 0.9 min. UV spectra were recorded at 215 nm using a Gilson detector.

### Example 10.1

### 5-[4-[3-[2-(4,4-Difluoro-1-piperidyl)ethoxy]pyrrolidin-1-yl]pyrrolo[2,1-f][1,2,4]triazin-6-yl]-1H-pyrimidine-2,4-dione

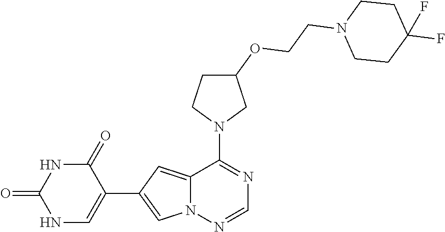 [SMILES: O=c1[nH]cc(-c2cc3c(N4CCC(OCCN5CCC(F)(F)CC5)C4)ncnn3c2)c(=O)[nH]1]

### Step 1: 6-Bromo-4-[3-[2-(4,4-difluoro-1-piperidyl)ethoxy]pyrrolidin-1-yl]pyrrolo[2,1-f][1,2,4]triazine

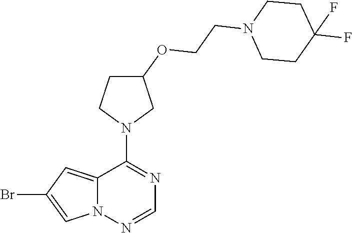 [SMILES: FC1(F)CCN(CCOC2CCN(c3ncnn4cc(Br)cc34)C2)CC1]

The title compound was prepared from 6-bromo-4-chloro-pyrrolo[2,1-f][1,2,4]triazine and 4,4-difluoro-1-(2-pyrrolidin-3-yloxyethyl)piperidine dihydrochloride (Intermediate B) analogously to Example 10 step 1.

LC-MS-2 (Method 2B): Rt 1.59 mins; MS m/z 430.0/431.9=[M+H]+

1H NMR (400 MHz, Methanol-d4) δ 7.74 (s, 1H), 7.62 (d, J=1.7 Hz, 1H), 7.00 (d, J=1.7 Hz, 1H), 4.41-4.19 (m, 1H), 4.15-3.80 (m, 3H), 3.77-3.60 (m, 3H), 2.70-2.55 (m, 6H), 2.44-2.00 (m, 2H), 2.00-1.85 (m, 4H).

### Step 2: 4-[3-[2-(4,4-Difluoro-1-piperidyl)ethoxy]pyrrolidine-1-yl]-6-(2,4-dimethoxypyrimidin-5-yl)pyrrolo[2,1-f][1,2,4]triazine

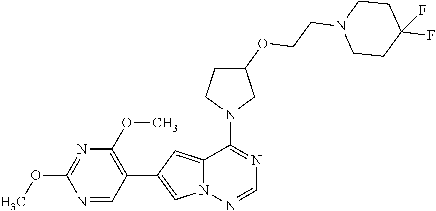 [SMILES: COc1ncc(-c2cc3c(N4CCC(OCCN5CCC(F)(F)CC5)C4)ncnn3c2)c(OC)n1]

The title compound was prepared from 6-bromo-4-[3-[2-(4,4-difluoro-1-piperidyl)ethoxy]pyrrolidine-1-yl]pyrrolo[2,1-f][1,2,4]triazine (step 1) and (2,4-dimethoxypyrimidin-5-yl)boronic acid analogously to Example 10 step 2.

LC-MS-1 (Method 2A): Rt 0.95 mins; MS m/z 490.4=[M+H]+

1H NMR (400 MHz, Methanol-d4) δ 8.67 (br s, 1H), 8.04-7.99 (m, 1H), 7.75 (s, 1H), 7.35-7.28 (m, 1H), 4.40-4.11 (m, 6H), 4.06-3.83 (m, 4H), 3.81-3.63 (m, 3H), 2.43-2.04 (m, 2H), 2.70-2.57 (m, 6H), 2.02-1.85 (m, 4H).

### Step 3: 5-[4-[3-[2-(4,4-Difluoro-1-piperidyl)ethoxy]pyrrolidine-1-yl]pyrrolo[2,1-f][1,2,4]triazin-6-yl]-1H-pyrimidine-2,4-dione

The title compound was prepared from 4-[3-[2-(4,4-difluoro-1-piperidyl)ethoxy]pyrrolidine-1-yl]-6-(2,4-dimethoxypyrimidin-5-yl)pyrrolo[2,1-f][1,2,4]triazine (step 2) and 1M HCl analogously to Example 10 step 3.

LC-MS-1 (Method 8A): Rt 1.48 mins; MS m/z 462.3=[M+H]+

1H NMR (400 MHz, Acetic Acid-d4) δ 8.35-8.27 (m, 1H), 8.07 (d, J=3.5 Hz, 1H), 7.98 (d, J=8.3 Hz, 1H), 7.58 (d, J=5.5 Hz, 1H), 4.55-4.40 (m, 1H), 4.35-4.14 (m, 2H), 4.14-3.85 (m, 4H), 3.78-3.35 (m, 6H), 2.53-2.22 (m, 6H).

### Example 10.2

### 5-[4-(3,3-Difluoro-4,4-dimethyl-pyrrolidin-1-yl)pyrrolo[2,1-f][1,2,4]triazin-6-yl]-1H-pyrimidine-2,4-dione

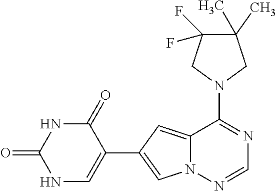 [SMILES: CC1(C)CN(c2ncnn3cc(-c4c[nH]c(=O)[nH]c4=O)cc23)CC1(F)F]

### Step 1: 6-Bromo-4-(3,3-difluoro-4,4-dimethyl-pyrrolidin-1-yl)pyrrolo[2,1-f][1,2,4]triazine

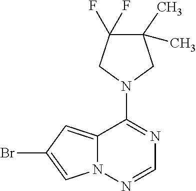 [SMILES: CC1(C)CN(c2ncnn3cc(Br)cc23)CC1(F)F]

The title compound was prepared from 6-bromo-4-chloro-pyrrolo[2,1-f][1,2,4]triazine and 3,3-difluoro-4,4-dimethyl-pyrrolidine hydrochloride analogously to Example 10 step 1.

LC-MS-1 (Method 2A): Rt 1.46 mins; MS m/z 331.1/333.1=[M+H]+

1H NMR (400 MHz, DMSO-d6) δ 7.97 (d, J=1.7 Hz, 1H), 7.90 (s, 1H), 7.12 (d, J=1.7 Hz, 1H), 4.50 (m, 1H), 4.17-4.10 (m, 1H), 4.01 (s, 1H), 3.67 (s, 1H), 1.18 (s, 6H).

### Step 2: 4-(3,3-Difluoro-4,4-dimethyl-pyrrolidin-1-yl)-6-(2,4-dimethoxypyrimidin-5-yl)pyrrolo[2,1-f][1,2,4]triazine

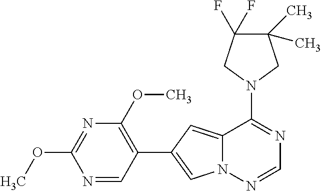

The title compound was prepared from 6-bromo-4-(3,3-difluoro-4,4-dimethyl-pyrrolidin-1-yl)pyrrolo[2,1-f][1,2,4]triazine (step 1) and (2,4-dimethoxypyrimidin-5-yl)boronic acid analogously to Example 10 step 2.

LC-MS-2 (Method 2A): Rt 1.69 mins; MS m/z 391.1=[M+H]+

1H NMR (400 MHz, DMSO-d6) δ 8.90 (s, 1H), 8.13 (d, J=1.6 Hz, 1H), 7.89 (s, 1H), 7.45 (d, J=1.7 Hz, 1H), 4.58 (s, 1H), 4.06 (m, 5H), 3.94 (s, 3H), 3.73 (s, 1H), 1.21 (s, 6H).

### Step 3: 5-[4-(3,3-Difluoro-4,4-dimethyl-pyrrolidin-1-yl)pyrrolo[2,1-f][1,2,4]triazin-6-yl]-1H-pyrimidine-2,4-dione

The title compound was prepared from 4-(3,3-difluoro-4,4-dimethyl-pyrrolidin-1-yl)-6-(2,4-dimethoxypyrimidin-5-yl)pyrrolo[2,1-f][1,2,4]triazine (step 2) and 1M HCl analogously to Example 10 step 3.

LC-MS-2 (Method 8A): Rt 2.79 mins; MS m/z 363.1=[M+H]+

1H NMR (400 MHz, DMSO-d6) δ 11.04 (s, 2H), 8.18 (d, J=1.6 Hz, 1H), 7.98 (s, 1H), 7.82 (s, 1H), 7.28 (d, J=1.6 Hz, 1H), 4.41-4.25 (m, 2H), 3.88 (s, 2H), 1.22 (s, 6H).

### Example 11

### 2-(2,4-Dioxo-1H-pyrimidin-5-yl)-4-[3-[2-(1-piperidyl)ethoxy]pyrrolidin-1-yl]thieno[2,3-b]pyridine-5-carbonitrile

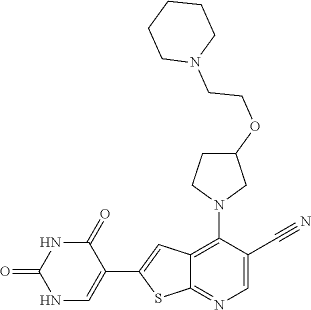 [SMILES: N#Cc1cnc2sc(-c3c[nH]c(=O)[nH]c3=O)cc2c1N1CCC(OCCN2CCCCC2)C1]

### Step 1: 4-Chloro-2-(2,4-dimethoxypyrimidin-5-yl)thieno[2,3-b]pyridine-5-carbonitrile

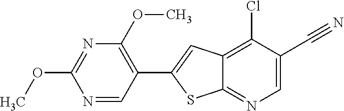 [SMILES: COc1ncc(-c2cc3c(Cl)c(C#N)cnc3s2)c(OC)n1]

The title compound was prepared from 4-chloro-2-iodo-thieno[2,3-b]pyridine-5-carbonitrile and (2,4-dimethoxypyrimidin-5-yl)boronic acid analogously to Example 2 step 2.

LC-MS (Method 2A): Rt 1.01 mins; MS m/z 332.9=[M+H]+

1H NMR (500 MHz, DMSO-d6) δ 9.06 (s, 1H), 8.98 (s, 1H), 8.09 (s, 1H), 4.12 (s, 3H), 4.00 (s, 3H).

### Step 2: 2-(2,4-Dimethoxypyrimidin-5-yl)-4-[3-[2-(1-piperidyl)ethoxy]pyrrolidin-1-yl]thieno[2,3-b]pyridine-5-carbonitrile

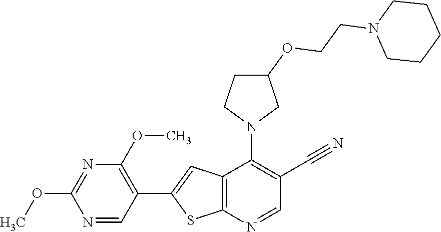 [SMILES: COc1ncc(-c2cc3c(N4CCC(OCCN5CCCCC5)C4)c(C#N)cnc3s2)c(OC)n1]

The title compound was prepared from 4-chloro-2-(2,4-dimethoxypyrimidin-5-yl)thieno[2,3-b]pyridine-5-carbonitrile (step 1) and 1-(2-pyrrolidin-3-yloxyethyl)piperidine (Intermediate J) analogously to Example 2 step 1. The reaction was carried out in DMA at 80° C.

LC-MS (Method 2A): Rt 0.71 mins; MS m/z 495.5=[M+H]+

1H NMR (500 MHz, DMSO-d6) δ 8.93 (s, 1H), 8.33 (s, 1H), 8.15 (s, 1H), 4.38-4.34 (m, 1H), 4.29 (dd, J=11.4, 3.8 Hz, 1H), 4.25-4.17 (m, 1H), 4.10-4.03 (m, 5H), 3.97 (s, 3H), 3.90-3.82 (m, 2H), 3.39 (s, 2H), 3.25 (s, 2H), 2.93-2.83 (m, 2H), 2.30-2.22 (m, 1H), 2.14-2.04 (m, 1H), 1.81-1.59 (m, 6H).

### Step 3: 2-(2,4-Dioxo-1H-pyrimidin-5-yl)-4-[3-[2-(1-piperidyl)ethoxy]pyrrolidin-1-yl]thieno[2,3-b]pyridine-5-carbonitrile

The title compound was prepared from 2-(2,4-dimethoxypyrimidin-5-yl)-4-[3-[2-(1-piperidyl)ethoxy]pyrrolidin-1-yl]thieno[2,3-b]pyridine-5-carbonitrile (step 2) and 1M HCl analogously to Example 2 step 3.

LC-MS (Method 7A): Rt 1.38 mins; MS m/z 467.5=[M+H]+

1H NMR (500 MHz, DMSO-d6) δ 9.71 (s, 2H), 8.33 (s, 1H), 8.10 (s, 1H), 7.83 (s, 1H), 4.27-4.18 (m, 2H), 4.18-4.04 (m, 1H), 4.02-3.95 (m, 1H), 3.94-3.87 (m, 1H), 3.63-3.52 (m, 2H), 2.45-2.39 (m, 2H), 2.36-2.25 (m, 4H), 2.17-2.10 (m, 1H), 2.08-1.98 (m, 1H), 1.48-1.36 (m, 4H), 1.35-1.26 (m, 2H).

### Example 12

### 5-[4-[3-[2-(1-Piperidyl)ethoxy]pyrrolidin-1-yl]pyrazolo[3,4-d]pyrimidin-2-yl]-1H-pyrimidine-2,4-dione hydrochloride

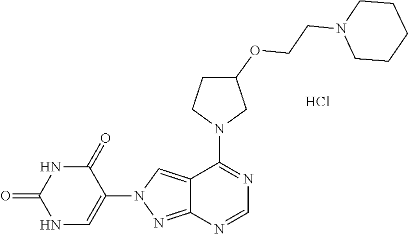 [SMILES: O=c1[nH]cc(-n2cc3c(N4CCC(OCCN5CCCCC5)C4)ncnc3n2)c(=O)[nH]1 ; Cl]

### Step 1: 2-(2,4-Dimethoxypyrimidin-5-yl)-4-[3-[2-(1-piperidyl)ethoxy]pyrrolidin-1-yl]pyrazolo[3,4-d]pyrimidine

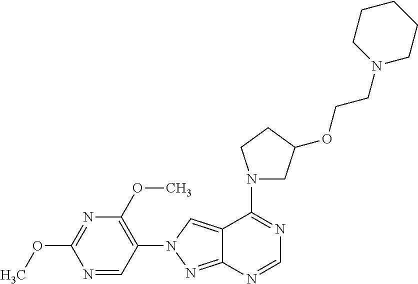 [SMILES: COc1ncc(-n2cc3c(N4CCC(OCCN5CCCCC5)C4)ncnc3n2)c(OC)n1]

A solution of 4-chloro-2-(2,4-dimethoxypyrimidin-5-yl)pyrazolo[3,4-d]pyrimidine (Intermediate E) (100 mg, 0.34 mmol), 1-(2-pyrrolidin-3-yloxyethyl)piperidine (Intermediate J) (75 mg, 0.38 mmol) and DIPEA (0.18 mL, 1.03 mmol) in MeCN (1.5 mL) was stirred at 80° C. for 5 h 45 mins. After cooling to room temperature, the resulting suspension was filtered and the collected solid dried under vacuum to afford the title compound as a colourless solid.

LC-MS (Method 7B): Rt 2.56 mins; MS m/z 455.6=[M+H]+

1H NMR (500 MHz, CDCl3) δ 9.03-8.87 (m, 1H), 8.50-8.39 (m, 2H), 4.40-4.21 (m, 1H), 4.15 (s, 3H), 4.11-4.04 (m, 3H), 4.06-3.58 (m, 6H), 2.65-2.02 (m, 8H), 1.60-1.52 (m, 4H), 1.46-1.36 (m, 2H).

### Step 2: 5-[4-[3-[2-(1-Piperidyl)ethoxy]pyrrolidin-1-yl]pyrazolo[3,4-d]pyrimidin-2-yl]-1H-pyrimidine-2,4-dione hydrochloride

2-(2,4-Dimethoxypyrimidin-5-yl)-4-[3-[2-(1-piperidyl)ethoxy]pyrrolidin-1-yl]pyrazolo[3,4-d]pyrimidine (step 1) (47 mg, 0.1 mmol) was dissolved in MeOH (1 mL). 1M HCl (1 mL, 1.03 mmol) was added and the mixture was stirred at 70° C. overnight. After cooling to room temperature, the resulting mixture was concentrated in vacuo to afford the title compound as an orange solid.

LC-MS (Method 7B): Rt 1.37 mins; MS m/z 427.4=[M+H]+

1H NMR (400 MHz, DMSO-d6) δ 11.96-11.85 (m, 1H), 11.86-11.68 (m, 1H), 10.67-10.39 (m, 1H), 9.37-9.17 (m, 1H), 8.75-8.56 (m, 1H), 8.32-8.16 (m, 1H), 4.54-4.33 (m, 1H), 4.21-4.04 (m, 3H), 4.02-3.77 (m, 3H), 3.42-3.30 (obscured m, 2H), 3.26-3.14 (m, 2H), 2.95-2.80 (m, 2H), 2.43-2.09 (m, 2H), 1.89-1.54 (m, 5H), 1.44-1.21 (m, 1H).

### Example 13

### 5-[4-(3,3-Difluoro-4,4-dimethyl-pyrrolidin-1-yl)pyrazolo[3,4-d]pyrimidin-2-yl]-1H-pyrimidine-2,4-dione

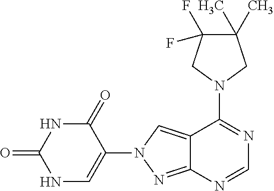 [SMILES: CC1(C)CN(c2ncnc3nn(-c4c[nH]c(=O)[nH]c4=O)cc23)CC1(F)F]

### Step 1: 4-(3,3-Difluoro-4,4-dimethyl-pyrrolidin-1-yl)-2-(2,4-dimethoxypyrimidin-5-yl)pyrazolo[3,4-d]pyrimidine

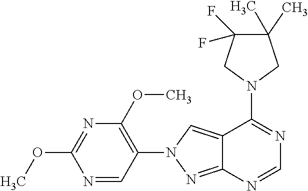

To a stirred suspension of 4-chloro-2-(2,4-dimethoxypyrimidin-5-yl)pyrazolo[3,4-d]pyrimidine (Intermediate E) (150 mg, 0.51 mmol) and 3,3-difluoro-4,4-dimethyl-pyrrolidine hydrochloride (106 mg, 0.62 mmol) in MeCN (3 mL) under nitrogen was added DIPEA (0.27 mL, 1.54 mmol) and the reaction mixture was heated at 70° C. for 5 mins. The mixture was allowed to cool to room temperature resulting in the precipitation of a solid. The solid was collected by filtration and washed with water (10 mL). Purification of the crude material by chromatography on silica eluting with a gradient of 0 to 10% MeOH in DCM afforded the title compound as an off-white solid.

LC-MS-1 (Method 2A): Rt 1.09 mins; MS m/z 392.3=[M+H]+

1H NMR (400 MHz, DMSO-d6) δ 8.94 (s, 1H), 8.72 (s, 1H), 8.32 (s, 1H), 4.40 (t, J=13. Hz, 1H), 4.17 (t, J=13.5 Hz, 1H), 4.05-3.98 (m, 6H), 3.89 (s, 1H), 3.74 (s, 1H), 1.28-1.13 (m, 6H).

### Step 2: 5-[4-(3,3-Difluoro-4,4-dimethyl-pyrrolidin-1-yl)pyrazolo[3,4-d]pyrimidin-2-yl]-1H-pyrimidine-2,4-dione

A stirred solution of 4-(3,3-difluoro-4,4-dimethyl-pyrrolidin-1-yl)-2-(2,4-dimethoxypyrimidin-5-yl)pyrazolo[3,4-d]pyrimidine (step 1) (182 mg, 0.47 mmol) in MeOH (3 mL) and 1M HCl (4.65 mL, 4.65 mmol) was heated at 70° C. for 4.5 h. The resulting mixture was concentrated in vacuo and the crude material was treated with water (30 mL) causing a solid to precipitate. The solid was collected by filtration, washed with water (20 mL), MeCN (2×20 mL) and diethyl ether (2×20 mL) to afford the title compound as a pink solid.

LC-MS-1 (Method 8A): Rt 1.94 mins; MS m/z 364.3=[M+H]+

1H NMR (400 MHz, DMSO-d6) δ 11.91 (s, 1H), 11.76-11.65 (m, 1H), 9.22 (d, J=9.8 Hz, 1H), 8.66 (d, J=3.6 Hz, 1H), 8.25-8.17 (m, 1H), 4.57 (t, J=13.1 Hz, 1H), 4.36 (t, J=13.1 Hz, 1H), 4.01 (s, 1H), 3.90 (s, 1H), 1.25 (s, 3H), 1.21 (s, 3H).

### Example 13.1

### 5-[4-(7,7-Difluoro-5-azaspiro[2.4]heptan-5-yl)pyrazolo[3,4-d]pyrimidin-2-yl]-1H-pyrimidine-2,4-dione

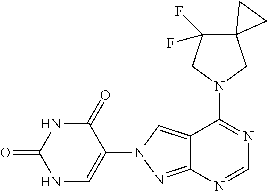 [SMILES: O=c1[nH]cc(-n2cc3c(N4CC(F)(F)C5(CC5)C4)ncnc3n2)c(=O)[nH]1]

### Step 1: 4-(7,7-Difluoro-5-azaspiro[2.4]heptan-5-yl)-2-(2,4-dimethoxypyrimidin-5-yl)pyrazolo[3,4-d]pyrimidine

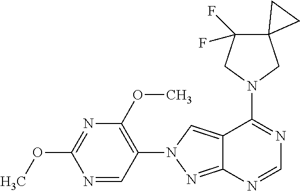 [SMILES: COc1ncc(-n2cc3c(N4CC(F)(F)C5(CC5)C4)ncnc3n2)c(OC)n1]

The title compound was prepared from 4-chloro-2-(2,4-dimethoxypyrimidin-5-yl)pyrazolo[3,4-d]pyrimidine (Intermediate E) and 7,7-difluoro-5-azaspiro[2.4]heptane hydrochloride analogously to Example 13 step 1.

LC-MS-2 (Method 2A): Rt 1.17 mins; MS m/z 390.1=[M+H]+

1H NMR (400 MHz, DMSO-d6) δ 9.01-8.82 (m, 1H), 8.73 (s, 1H), 8.34 (s, 1H), 4.54-4.44 (m, 1H), 4.33-4.23 (m, 1H), 4.12 (s, 1H), 4.05-3.99 (m, 6H), 3.94 (s, 1H), 1.12-1.03 (m, 2H), 1.02-0.96 (m, 2H).

### Step 2: 5-[4-(7,7-Difluoro-5-azaspiro[2.4]heptan-5-yl)pyrazolo[3,4-d]pyrimidin-2-yl]-1H-pyrimidine-2,4-dione

The title compound was prepared from 4-(7,7-difluoro-5-azaspiro[2.4]heptan-5-yl)-2-(2,4-dimethoxypyrimidin-5-yl)pyrazolo[3,4-d]pyrimidine (step 1) and 1M HCl analogously to Example 13 step 2.

LC-MS-2 (Method 8A): Rt 1.89 mins; MS m/z 362.0=[M+H]+

1H NMR (400 MHz, DMSO-d6) δ 11.62 (s, 1H), 11.36 (s, 1H), 8.98 (s, 1H), 8.37 (s, 1H), 8.14 (d, J=5.4 Hz, 1H), 4.38 (t, J=11.9 Hz, 2H), 4.06 (s, 2H), 1.12-1.05 (m, 2H), 1.05-0.98 (m, 2H).

### Example 13.2

### 5-[4-(8,8-Difluoro-6-azaspiro[3.4]octan-6-yl)pyrazolo[3,4-d]pyrimidin-2-yl]-1H-pyrimidine-2,4-dione

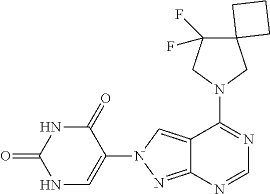 [SMILES: O=c1[nH]cc(-n2cc3c(N4CC(F)(F)C5(CCC5)C4)ncnc3n2)c(=O)[nH]1]

### Step 1: 4-(8,8-Difluoro-6-azaspiro[3.4]octan-6-yl)-2-(2,4-dimethoxypyrimidin-5-yl)pyrazolo[3,4-d]pyrimidine

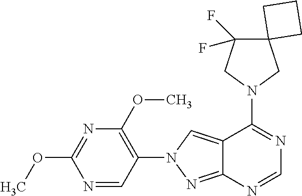 [SMILES: COc1ncc(-n2cc3c(N4CC(F)(F)C5(CCC5)C4)ncnc3n2)c(OC)n1]

The title compound was prepared from 4-chloro-2-(2,4-dimethoxypyrimidin-5-yl)pyrazolo[3,4-d]pyrimidine (Intermediate E) and 8,8-difluoro-6-azaspiro[3.4]octane hydrochloride analogously to Example 13 step 1.

LC-MS-1 (Method 2B): Rt 1.29 mins; MS m/z 404.3=[M+H]+

1H NMR (400 MHz, DMSO-d6) δ 9.05-8.88 (m, 1H), 8.72 (d, J=3.6 Hz, 1H), 8.32 (d, J=5.8 Hz, 1H), 4.31 (t, J=12.8 Hz, 1H), 4.23-3.92 (m, 9H), 2.41-2.28 (m, 2H), 2.14-1.94 (m, 3H), 1.94-1.75 (m, 1H).

### Step 2: 5-[4-(8,8-Difluoro-6-azaspiro[3.4]octan-6-yl)pyrazolo[3,4-d]pyrimidin-2-yl]-1H-pyrimidine-2,4-dione

The title compound was prepared from 4-(8,8-difluoro-6-azaspiro[3.4]octan-6-yl)-2-(2,4-dimethoxypyrimidin-5-yl)pyrazolo[3,4-d]pyrimidine (step 1) and 1M HCl analogously to Example 13 step 2.

LC-MS-1 (Method 8A): Rt 2.05 mins; MS m/z 376.2=[M+H]+

1H NMR (400 MHz, DMSO-d6) δ 11.81 (s, 1H), 11.55 (s, 1H), 9.06-8.92 (m, 1H), 8.29 (s, 1H), 8.19 (s, 1H), 4.35-4.23 (m, 1H), 4.17-3.93 (m, 3H), 2.37-2.30 (m, 2H), 2.16-1.95 (m, 3H), 1.93-1.82 (m, 1H).

### Example 13.3

### 5-[4-(4,4-Difluoro-2-azaspiro[4.4]nonan-2-yl)pyrazolo[3,4-d]pyrimidin-2-yl]-1H-pyrimidine-2,4-dione

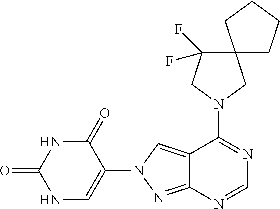 [SMILES: O=c1[nH]cc(-n2cc3c(N4CC(F)(F)C5(CCCC5)C4)ncnc3n2)c(=O)[nH]1]

### Step 1: 4-(4,4-Difluoro-2-azaspiro[4.4]nonan-2-yl)-2-(2,4-dimethoxypyrimidin-5-yl)pyrazolo[3,4-d]pyrimidine

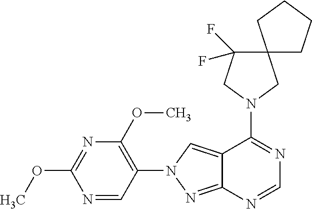 [SMILES: COc1ncc(-n2cc3c(N4CC(F)(F)C5(CCCC5)C4)ncnc3n2)c(OC)n1]

The title compound was prepared from 4-chloro-2-(2,4-dimethoxypyrimidin-5-yl)pyrazolo[3,4-d]pyrimidine (Intermediate E) and 4,4-difluoro-2-azaspiro[4.4]nonane hydrochloride analogously to example 13 step 1.

LC-MS-1 (Method 2A): Rt 1.20 mins; MS m/z 418.5=[M+H]+

1H NMR (400 MHz, DMSO-d6) δ 9.00-8.91 (m, 1H), 8.74-8.65 (m, 1H), 8.32 (s, 1H), 4.36 (t, J=13.0 Hz, 1H), 4.12 (t, J=13.0 Hz, 1H), 4.06-3.99 (m, 6H), 3.93 (s, 1H), 3.80 (s, 1H), 2.02-1.87 (m, 2H), 1.77-1.58 (m, 6H).

### Step 2: 5-[4-(4,4-Difluoro-2-azaspiro[4.4]nonan-2-yl)pyrazolo[3,4-d]pyrimidin-2-yl]-1H-pyrimidine-2,4-dione

The title compound was prepared from 4-(4,4-difluoro-2-azaspiro[4.4]nonan-2-yl)-2-(2,4-dimethoxypyrimidin-5-yl)pyrazolo[3,4-d]pyrimidine (step 1) and 1M HCl analogously to Example 13 step 2.

LC-MS-1 (Method 8A): Rt 2.27 mins; MS m/z 390.3=[M+H]+

1H NMR (400 MHz, DMSO-d6) δ 11.88 (s, 1H), 11.67 (d, J=5.9 Hz, 1H), 9.19 (s, 1H), 8.60 (s, 1H), 8.21 (d, J=5.7 Hz, 1H), 4.49 (t, J=12.6 Hz, 1H), 4.28 (t, J=12.9 Hz, 1H), 4.02 (s, 1H), 3.93 (s, 1H), 2.02-1.86 (m, 2H), 1.79-1.61 (m, 6H).

### Example 13.4

### 5-[4-[(3aR,6aS)-3,3a,4,5,6,6a-Hexahydro-1H-cyclopenta[c]pyrrol-2-yl]pyrazolo[3,4-d]pyrimidin-2-yl]-1H-pyrimidine-2,4-dione

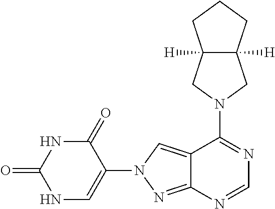 [SMILES: O=c1[nH]cc(-n2cc3c(N4C[C@H]5CCC[C@H]5C4)ncnc3n2)c(=O)[nH]1]

### Step 1: 4-[(3aR,6aS)-3,3a,4,5,6,6a-Hexahydro-1H-cyclopenta[c]pyrrol-2-yl]-2-(2,4-dimethoxypyrimidin-5-yl)pyrazolo[3,4-d]pyrimidine

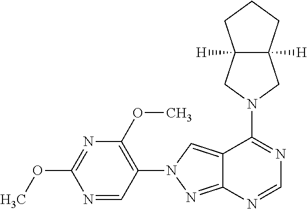 [SMILES: COc1ncc(-n2cc3c(N4C[C@H]5CCC[C@H]5C4)ncnc3n2)c(OC)n1]

The title compound was prepared from 4-chloro-2-(2,4-dimethoxypyrimidin-5-yl)pyrazolo[3,4-d]pyrimidine (Intermediate E) and (3aR,6aS)-1,2,3,3a,4,5,6,6a-octahydrocyclopenta[c]pyrrole hydrochloride analogously to Example 13 step 1.

LC-MS-1 (Method 2A): Rt 1.01 mins; MS m/z 368.5=[M+H]+

1H NMR (400 MHz, Methanol-d4) δ 8.78 (s, 1H), 8.71 (s, 1H), 8.24 (s, 1H), 4.22-4.10 (m, 4H), 4.09 (s, 3H), 4.07-3.97 (m, 1H), 3.74-3.59 (m, 2H), 3.06-2.95 (m, 1H), 2.95-2.80 (m, 1H), 2.09-1.94 (m, 2H), 1.94-1.84 (m, 1H), 1.80-1.72 (m, 1H), 1.72-1.57 (m, 2H).

### Step 2: 5-[4-[(3aR,6aS)-3,3a,4,5,6,6a-Hexahydro-1H-cyclopenta[c]pyrrol-2-yl]pyrazolo[3,4-d]pyrimidin-2-yl]-1H-pyrimidine-2,4-dione

The title compound was prepared from 4-[(3aR,6aS)-3,3a,4,5,6,6a-hexahydro-1H-cyclopenta[c]pyrrol-2-yl]-2-(2,4-dimethoxypyrimidin-5-yl)pyrazolo[3,4-d]pyrimidine (step 1) and 1M HCl analogously to Example 13 step 2.

LC-MS-1 (Method 8A): Rt 1.70 mins; MS m/z 340.2=[M+H]+

1H NMR (400 MHz, DMSO-d6) δ 11.84 (s, 1H), 11.57 (d, J=6.4 Hz, 1H), 9.03 (s, 1H), 8.36 (s, 1H), 8.19 (d, J=6.2 Hz, 1H), 4.12-4.02 (m, 1H), 4.02-3.92 (m, 1H), 3.67-3.51 (m, 2H), 2.98-2.88 (m, 1H), 2.84-2.74 (m, 1H), 1.94-1.73 (m, 3H), 1.68-1.46 (m, 3H).

### Example 13.5

### 5-[4-(3-Ethyl-3-methyl-pyrrolidin-1-yl)pyrazolo[3,4-d]pyrimidin-2-yl]-1H-pyrimidine-2,4-dione

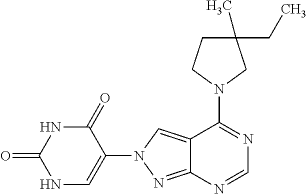 [SMILES: CCC1(C)CCN(c2ncnc3nn(-c4c[nH]c(=O)[nH]c4=O)cc23)C1]

### Step 1: 2-(2,4-Dimethoxypyrimidin-5-yl)-4-(3-ethyl-3-methyl-pyrrolidin-1-yl)pyrazolo[3,4-d]pyrimidine

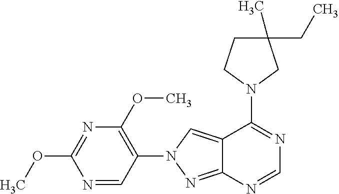 [SMILES: CCC1(C)CCN(c2ncnc3nn(-c4cnc(OC)nc4OC)cc23)C1]

The title compound was prepared from 4-chloro-2-(2,4-dimethoxypyrimidin-5-yl)pyrazolo[3,4-d]pyrimidine (Intermediate E) and 3-ethyl-3-methyl-pyrrolidine analogously to Example 13 step 1.

LC-MS-2 (Method 2A): Rt 1.18 mins; MS m/z 370.1=[M+H]+

1H NMR (400 MHz, DMSO-d6) δ 8.80 (s, 1H), 8.70 (s, 1H), 8.24 (s, 1H), 4.05 (s, 3H), 4.03 (s, 3H), 3.93-3.69 (m, 2H), 3.64-3.43 (m, 2H), 1.87 (m, 2H), 1.51 (q, J=7.5 Hz, 2H), 1.09 (s, 3H), 0.94 (t, J=7.5 Hz, 3H).

### Step 2: 5-[4-(3-Ethyl-3-methyl-pyrrolidin-1-yl)pyrazolo[3,4-d]pyrimidin-2-yl]-1H-pyrimidine-2,4-dione

The title compound was prepared from 2-(2,4-dimethoxypyrimidin-5-yl)-4-(3-ethyl-3-methyl-pyrrolidin-1-yl)pyrazolo[3,4-d]pyrimidine (step 1) and 1M HCl to analogously to Example 13 step 2.

LC-MS-2 (Method 8A): Rt 2.01 mins; MS m/z 342.0=[M+H]+

1H NMR (400 MHz, DMSO-d6) δ 11.68 (s, 1H), 11.53 (s, 1H), 9.19 (s, 1H), 8.51 (s, 1H), 8.15 (s, 1H), 4.08-3.88 (m, 2H), 3.74-3.61 (m, 2H), 2.11-1.80 (m, 2H), 1.61-1.45 (m, 2H), 1.18-1.05 (m, 3H), 1.02-0.88 (m, 3H).

### Example 13.6

### 5-[4-(3,3-Dimethylpyrrolidin-1-yl)pyrazolo[3,4-d]pyrimidin-2-yl]-1H-pyrimidine-2,4-dione

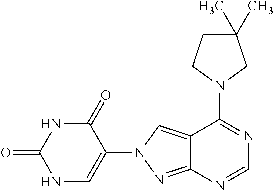 [SMILES: CC1(C)CCN(c2ncnc3nn(-c4c[nH]c(=O)[nH]c4=O)cc23)C1]

### Step 1: 2-(2,4-Dimethoxypyrimidin-5-yl)-4-(3,3-dimethylpyrrolidin-1-yl)pyrazolo[3,4-d]pyrimidine

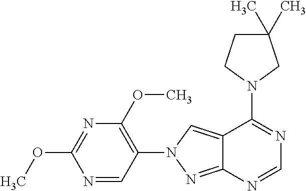

The title compound was prepared from 4-chloro-2-(2,4-dimethoxypyrimidin-5-yl)pyrazolo[3,4-d]pyrimidine (Intermediate E) and 3,3-dimethylpyrrolidine hydrochloride analogously to Example 13 step 1.

LC-MS-2 (Method 2A): Rt 1.10 mins; MS m/z 356.1=[M+H]+

1H NMR (400 MHz, DMSO-d6) δ 8.79 (s, 1H), 8.70 (s, 1H), 8.24 (s, 1H), 4.05 (s, 3H), 4.03 (s, 3H), 3.95-3.73 (m, 2H), 3.54 (s, 2H), 1.87 (m, 2H), 1.16 (s, 6H).

### Step 2: 5-[4-(3,3-Dimethylpyrrolidin-1-yl)pyrazolo[3,4-d]pyrimidin-2-yl]-1H-pyrimidine-2,4-dione

The title compound was prepared from 2-(2,4-dimethoxypyrimidin-5-yl)-4-(3,3-dimethylpyrrolidin-1-yl)pyrazolo[3,4-d]pyrimidine (step 1) and 1M HCl analogously to Example 13 step 2.

LC-MS-2 (Method 8A): Rt 1.80 mins; MS m/z 328.0=[M+H]+

1H NMR (400 MHz, DMSO-d6) δ 11.57 (s, 1H), 11.29 (s, 1H), 8.90 (s, 1H), 8.21 (d, J=5.0 Hz, 1H), 8.12 (s, 1H), 3.87-3.71 (m, 2H), 3.52 (s, 2H), 2.01-1.76 (m, 2H), 1.15 (s, 6H).

### Example 13.7

### 5-[4-(3,3-Diethylpyrrolidin-1-yl)pyrazolo[3,4-d]pyrimidin-2-yl]-1H-pyrimidine-2,4-dione

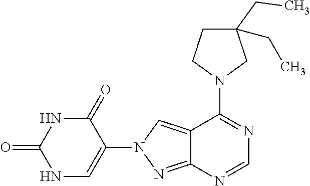 [SMILES: CCC1(CC)CCN(c2ncnc3nn(-c4c[nH]c(=O)[nH]c4=O)cc23)C1]

### Step 1: 4-(3,3-Diethylpyrrolidin-1-yl)-2-(2,4-dimethoxypyrimidin-5-yl)pyrazolo[3,4-d]pyrimidine

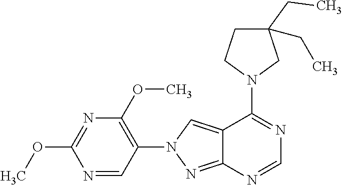 [SMILES: CCC1(CC)CCN(c2ncnc3nn(-c4cnc(OC)nc4OC)cc23)C1]

The title compound was prepared from 4-chloro-2-(2,4-dimethoxypyrimidin-5-yl)pyrazolo[3,4-d]pyrimidine (Intermediate E) and 3,3-diethylpyrrolidine analogously to Example 13 step 1.

LC-MS-2 (Method 2A): Rt 1.24 mins; MS m/z 384.1=[M+H]+

1H NMR (400 MHz, DMSO-d6) δ 8.81 (s, 1H), 8.71 (s, 1H), 8.25 (s, 1H), 4.05 (s, 3H), 4.04 (s, 3H), 3.91-3.69 (m, 2H), 3.56 (s, 2H), 1.99-1.78 (m, 2H), 1.50 (q, J=7.6 Hz, 4H), 0.90 (t, J=7.6 Hz, 6H).

### Step 2: 5-[4-(3,3-Diethylpyrrolidin-1-yl)pyrazolo[3,4-d]pyrimidin-2-yl]-1H-pyrimidine-2,4-dione

The title compound was prepared from 4-(3,3-diethylpyrrolidin-1-yl)-2-(2.4-dimethoxypyrimidin-5-yl)pyrazolo[3,4-d]pyrimidine (step 1) and 1M HCl analogously to Example 13 step 2.

LC-MS-2 (Method 8A): Rt 2.29 mins; MS m/z 356.1=[M+H]+

1H NMR (400 MHz, DMSO-d6) δ 11.41 (s, 2H), 8.91 (s, 1H), 8.21 (s, 1H), 8.11 (s, 1H), 3.86-3.75 (m, 2H), 3.53 (s, 2H), 1.99-1.75 (m, 2H), 1.48 (q, J=7.6 Hz, 4H), 0.89 (t, J=7.6 Hz, 6H).

### Example 13.8

### 5-[4-(3,3,4,4-Tetrafluoropyrrolidin-1-yl)pyrazolo[3,4-d]pyrimidin-2-yl]-1H-pyrimidine-2,4-dione

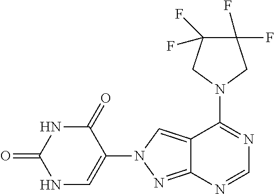 [SMILES: O=c1[nH]cc(-n2cc3c(N4CC(F)(F)C(F)(F)C4)ncnc3n2)c(=O)[nH]1]

### Step 1: 2-(2,4-Dimethoxypyrimidin-5-yl)-4-(3,3,4,4-tetrafluoropyrrolidin-1-yl)pyrazolo[3,4-d]pyrimidine

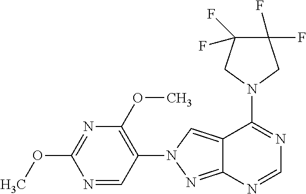 [SMILES: COc1ncc(-n2cc3c(N4CC(F)(F)C(F)(F)C4)ncnc3n2)c(OC)n1]

The title compound was prepared from 4-chloro-2-(2,4-dimethoxypyrimidin-5-yl)pyrazolo[3,4-d]pyrimidine (Intermediate E) and 3,3,4,4-tetrafluoropyrrolidine hydrochloride analogously to Example 13 step 1.

LC-MS-2 (Method 2B): Rt 1.36 mins; MS m/z 400.0=[M+H]+

1H NMR (400 MHz, DMSO-d6) δ 9.02 (s, 1H), 8.74 (s, 1H), 8.41 (s, 1H), 4.78-4.57 (m, 2H), 4.57-4.35 (m, 2H), 4.04 (s, 3H), 4.02 (s, 3H).

### Step 2: 5-[4-(3,3,4,4-Tetrafluoropyrrolidin-1-yl)pyrazolo[3,4-d]pyrimidin-2-yl]-1H-pyrimidine-2,4-dione

The title compound was prepared from 2-(2,4-dimethoxypyrimidin-5-yl)-4-(3,3,4,4-tetrafluoropyrrolidin-1-yl)pyrazolo[3,4-d]pyrimidine (step 1) and 1M HCl analogously to Example 13 step 2.

LC-MS-2 (Method 8A): Rt 2.09 mins; MS m/z 371.9=[M+H]+

1H NMR (400 MHz, DMSO-d6) δ 11.65 (s, 1H), 11.45-11.34 (m, 1H), 9.09 (s, 1H), 8.43 (s, 1H), 8.15 (d, J=5.9 Hz, 1H), 4.57 (t, J=13.9 Hz, 4H).

### Example 13.9

### 5-[4-[(3aR,6aS)-1,3,3a,4,6,6a-Hexahydrofuro[3,4-c]pyrrol-5-yl]pyrazolo[3,4-d]pyrimidin-2-yl]-1H-pyrimidine-2,4-dione

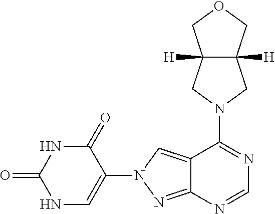 [SMILES: O=c1[nH]cc(-n2cc3c(N4C[C@H]5COC[C@H]5C4)ncnc3n2)c(=O)[nH]1]

### Step 1: (3aR,6aS)-5-[2-(2,4-Dimethoxypyrimidin-5-yl)pyrazolo[3,4-d]pyrimidin-4-yl]-1,3,3a,4,6,6a-hexahydrofuro[3,4-c]pyrrole

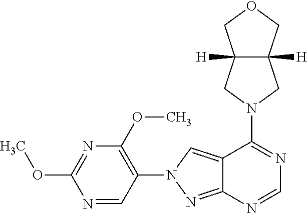 [SMILES: COc1ncc(-n2cc3c(N4C[C@H]5COC[C@H]5C4)ncnc3n2)c(OC)n1]

The title compound was prepared from 4-chloro-2-(2,4-dimethoxypyrimidin-5-yl)pyrazolo[3,4-d]pyrimidine (Intermediate E) and (3aS,6aR)-3,3a,4,5,6,6a-hexahydro-1H-furo[3,4-c]pyrrole hydrochloride analogously to Example 13 step 1.

LC-MS-2 (Method 2A): Rt 0.93 mins; MS m/z 370.1=[M+H]+

1H NMR (400 MHz, DMSO-d6) δ 8.80 (s, 1H), 8.71 (s, 1H), 8.26 (s, 1H), 4.04 (m, 8H), 3.91-3.84 (m, 2H), 3.74 (dd, J=12.2, 3.3 Hz, 2H), 3.68 (dd, J=9.0, 2.9 Hz, 2H), 3.17-3.12 (m, 2H).

### Step 2: 5-[4-[(3aR,6aS)-1,3,3a,4,6,6a-Hexahydrofuro[3,4-c]pyrrol-5-yl]pyrazolo[3,4-d]pyrimidin-2-yl]-1H-pyrimidine-2,4-dione

The title compound was prepared from (3aR,6aS)-5-[2-(2,4-dimethoxypyrimidin-5-yl)pyrazolo[3,4-d]pyrimidin-4-yl]-1,3,3a,4,6,6a-hexahydrofuro[3,4-c]pyrrole (step 1) and 1M HCl analogously to Example 13 step 2.

LC-MS-1 (Method 8B): Rt 1.14 mins; MS m/z 342.2=[M+H]+

1H NMR (400 MHz, DMSO-d6) δ 11.44 (s, 2H), 8.92 (s, 1H), 8.23 (s, 1H), 8.12 (s, 1H), 4.06-3.96 (m, 2H), 3.91-3.83 (m, 2H), 3.75-3.62 (m, 4H), 3.18-3.12 (m, 2H).

### Example 13.10

### 5-[4-[(3aR,6aR)-1,3,3a,4,6,6a-Hexahydrofuro[3,4-c]pyrrol-5-yl]pyrazolo[3,4-d]pyrimidin-2-yl]-5H-pyrimidine-2,4-dione

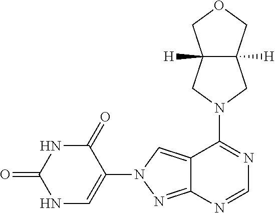 [SMILES: O=c1[nH]cc(-n2cc3c(N4C[C@@H]5COC[C@H]5C4)ncnc3n2)c(=O)[nH]1]

### Step 1: (3aR,6aR)-5-[2-(2,4-Dimethoxypyrimidin-5-yl)pyrazolo[3,4-d]pyrimidin-4-yl]-1,3,3a,4,6,6a-hexahydrofuro[3,4-c]pyrrole

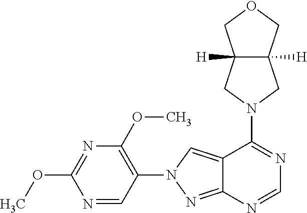 [SMILES: COc1ncc(-n2cc3c(N4C[C@@H]5COC[C@H]5C4)ncnc3n2)c(OC)n1]

The title compound was prepared from 4-chloro-2-(2,4-dimethoxypyrimidin-5-yl)pyrazolo[3,4-d]pyrimidine (Intermediate E) and (3aR,6aR)-3,3a,4,5,6,6a-hexahydro-1H-furo[3,4-c]pyrrole hydrochloride analogously to Example 13 step 1.

LC-MS-1 (Method 2A): Rt 0.73 mins; MS m/z 370.2=[M+H]+

1H NMR (400 MHz, CDCl3) δ 8.93 (s, 1H), 8.60 (s, 1H), 8.49 (s, 1H), 4.48-4.39 (m, 1H), 4.18 (s, 3H), 4.13 (dt, J=7.0, 3.8 Hz, 2H), 4.09 (s, 3H), 3.78 (t, J=10.1 Hz, 1H), 3.69-3.62 (m, 3H), 3.13-3.05 (m, 1H), 2.87-2.77 (m, 1H), 2.75-2.66 (m, 1H).

### Step 2: 5-[4-[(3aR,6aR)-1,3,3a,4,6,6a-Hexahydrofuro[3,4-c]pyrrol-5-yl]pyrazolo[3,4-d]pyrimidin-2-yl]-5H-pyrimidine-2,4-dione

The title compound was prepared from (3aR,6aR)-5-[2-(2,4-dimethoxypyrimidin-5-yl)pyrazolo[3,4-d]pyrimidin-4-yl]-1,3,3a,4,6,6a-hexahydrofuro[3,4-c]pyrrole (step 1) and 1M HCl analogously to Example 13 step 2.

LC-MS-1 (method 8B [0-10%]): Rt 0.38 mins; MS m/z 342.2=[M+H]+

1H NMR (400 MHz, Acetic Acid-d4) δ 9.36 (s, 1H), 8.63 (s, 1H), 8.56 (s, 1H), 4.49-4.42 (m, 1H), 4.31 (dd, J=10.2, 6.9 Hz, 1H), 4.21 (q, J=6.7 Hz, 2H), 3.93 (t, J=10.7 Hz, 1H), 3.79-3.60 (m, 3H), 2.95-2.86 (m, 1H), 2.84-2.76 (m, 1H).

### Example 13.11

### 5-[4-(3,3-Difluoro-4-methoxy-pyrrolidin-1-yl)pyrazolo[3,4-d]pyrimidin-2-yl]-1H-pyrimidine-2,4-dione

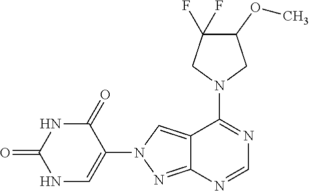 [SMILES: COC1CN(c2ncnc3nn(-c4c[nH]c(=O)[nH]c4=O)cc23)CC1(F)F]

### Step 1: 4-(3,3-Difluoro-4-methoxy-pyrrolidin-1-yl)-2-(2,4-dimethoxypyrimidin-5-yl)pyrazolo[3,4-d]pyrimidine

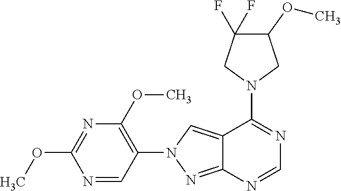

The title compound was prepared from 4-chloro-2-(2,4-dimethoxypyrimidin-5-yl)pyrazolo[3,4-d]pyrimidine (Intermediate E) and 3,3-difluoro-4-methoxy-pyrrolidine hydrochloride analogously to Example 13 step 1.

LC-MS-1 (Method 2A): Rt 0.95 mins; MS m/z 394.4=[M+H]+

1H NMR (400 MHz, DMSO-d6) δ 8.98 (d, J=11.9 Hz, 1H), 8.72 (s, 1H), 8.33 (s, 1H), 4.52-4.22 (m, 3H), 4.22-4.10 (m, 1H), 4.07-3.89 (m, 7H), 3.29 (s, 3H).

### Step 2: 5-[4-(3,3-Difluoro-4-methoxy-pyrrolidin-1-yl)pyrazolo[3,4-d]pyrimidin-2-yl]-1H-pyrimidine-2,4-dione

The title compound was prepared from 4-(3,3-difluoro-4-methoxy-pyrrolidin-1-yl)-2-(2,4-dimethoxypyrimidin-5-yl)pyrazolo[3,4-d]pyrimidine (step 1) and 1M HCl analogously to Example 13 step 2.

LC-MS-2 (Method 8A): Rt 1.70 mins; MS m/z 366.0=[M+H]+

1H NMR (400 MHz, DMSO-d6) δ 11.55 (s, 2H), 8.99 (s, 1H), 8.30 (s, 1H), 8.13 (s, 1H), 4.39-4.30 (m, 1H), 4.27-4.04 (m, 3H), 3.93-3.83 (m, 1H), 3.51 (s, 3H).

### Example 14

### 5-[4-[3-[2-(1-Piperidyl)ethoxy]pyrrolidin-1-yl]thieno[2,3-b]pyridin-2-yl]-1H-pyrimidine-2,4-dione

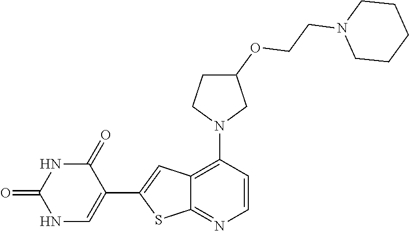 [SMILES: O=c1[nH]cc(-c2cc3c(N4CCC(OCCN5CCCCC5)C4)ccnc3s2)c(=O)[nH]1]

The title compound was prepared from 5-(4-chlorothieno[2,3-b]pyridin-2-yl)-1H-pyrimidine-2,4-dione hydrochloride (Intermediate F) and 1-(2-pyrrolidin-3-yloxyethyl)piperidine (Intermediate J) analogously to Example 13 step 1. The reaction was carried out in NMP at 120° C.

LC-MS-2 (Method 8B): Rt 2.29 mins; MS m/z 442.1=[M+H]+

1H NMR (400 MHz, Acetic Acid-d4) δ 8.39 (s, 1H), 8.21 (d, J=7. Hz, 1H), 8.05 (s, 1H), 6.68 (d, J=7.2 Hz, 1H), 4.51 (s, 1H), 4.33-3.86 (m, 6H), 3.76-3.63 (m, 2H), 3.50-3.40 (m, 2H), 3.05-2.88 (m, 2H), 2.51-2.39 (m, 1H), 2.33-2.20 (m, 1H), 1.94-1.72 (m, 5H), 1.55-1.40 (m, 1H).

### Example 14.1

### 5-[4-(3,3-Difluoro-4,4-dimethyl-pyrrolidin-1-yl)thieno[2,3-b]pyridin-2-yl]-1H-pyrimidine-2,4-dione

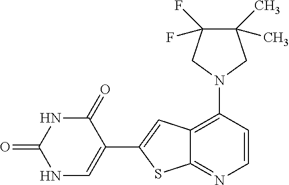 [SMILES: CC1(C)CN(c2ccnc3sc(-c4c[nH]c(=O)[nH]c4=O)cc23)CC1(F)F]

The title compound was prepared from 5-(4-chlorothieno[2,3-b]pyridin-2-yl)-1H-pyrimidine-2,4-dione hydrochloride (Intermediate F) and 3,3-difluoro-4,4-dimethyl-pyrrolidine hydrochloride analogously to Example 13 step 1. The reaction was carried out in NMP at 120° C.

LC-MS-1 (Method 8A): Rt 2.05 mins; MS m/z 379.2=[M+H]+

1H NMR (400 MHz, DMSO-d6) δ 11.52 (s, 2H), 8.29 (s, 1H), 8.06 (d, J=5.7 Hz, 1H), 7.85 (s, 1H), 6.38 (d, J=5.7 Hz, 1H), 4.26 (t, J=13.8 Hz, 2H), 3.65 (s, 2H), 1.21 (s, 6H).

### Example 15

### 5-[6-[3-[2-(1-Piperidyl)ethoxy]pyrrolidin-1-yl]-9H-purin-8-yl]-1H-pyrimidine-2,4-dione

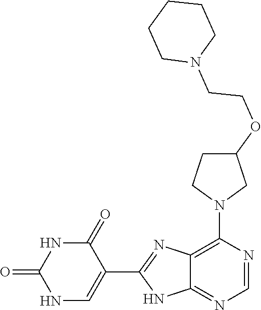 [SMILES: O=c1[nH]cc(-c2nc3c(N4CCC(OCCN5CCCCC5)C4)ncnc3[nH]2)c(=O)[nH]1]

### Step 1: 8-Bromo-6-chloro-9-tetrahydropyran-2-yl-purine

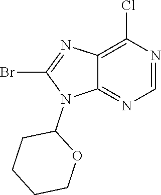 [SMILES: Clc1ncnc2c1nc(Br)n2C1CCCCO1]

A solution of 6-chloro-9-tetrahydropyran-2-yl-purine (1.50 g, 6.28 mmol) in THF (10 mL) was added to a solution of 2M LDA in THF/n-heptane/ethylbenzene (4.71 mL, 9.43 mmol) in THF (10 mL) at −78° C. and the resulting mixture stirred at this temperature for 1 h. To this mixture was added 1,2-dibromo-1,1,2,2-tetrachloro-ethane (4.09 g, 12.57 mmol) in THF (10 mL) and the reaction mixture was stirred at −78° C. for a further 1 h. Saturated aqueous NH₄Cl (10 mL) was added and the reaction mixture was allowed to warm to room temperature, stirring for 30 mins. The resulting mixture was concentrated in vacuo to remove THF and the aqueous mixture was extracted with DCM (3×15 mL). The combined organic extracts were passed through a hydrophobic frit and concentrated in vacuo. Purification by chromatography on silica eluting with a gradient of 0 to 50% EtOAc in heptane afforded the title compound as an orange oil.

LC-MS (Method 2A): Rt 0.78 mins; MS m/z 319.4=[M+H]+

1H NMR (400 MHz, DMSO-d6) δ 8.80 (s, 1H), 5.73 (dd, J=11.1, 2.3 Hz, 1H), 4.12-4.02 (m, 1H), 3.76-3.63 (m, 1H), 3.05-2.88 (m, 1H), 2.06-1.92 (m, 2H), 1.83-1.56 (m, 3H).

### Step 2: 6-Chloro-8-(2,4-dimethoxypyrimidin-5-yl)-9-tetrahydropyran-2-yl-purine

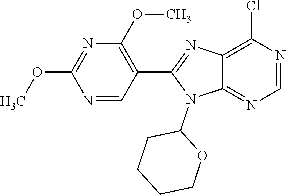 [SMILES: COc1ncc(-c2nc3c(Cl)ncnc3n2C2CCCCO2)c(OC)n1]

A suspension of 8-bromo-6-chloro-9-tetrahydropyran-2-yl-purine (step 1) (500 mg, 1.57 mmol), (2,4-dimethoxypyrimidin-5-yl)boronic acid (319 mg, 1.73 mmol) and Pd(dppf)Cl₂ (115 mg, 0.16 mmol) in 1M aq. sodium carbonate (3.94 mL, 3.94 mmol) and 1,4-dioxane (10 mL) was degassed with nitrogen for 5 mins and heated at 60° C. for 2 h. The mixture was diluted with water (20 mL) and the resulting suspension was collected by filtration. The solid was washed with water (2×10 mL) to afford an orange solid. The filtrate was extracted with EtOAc (2×25 mL) and the combined organic extracts were washed with brine (30 mL), dried over magnesium sulfate and concentrated in vacuo to afford an orange oil. The oil was combined with the previously isolated orange solid and purified by chromatography on silica eluting with a gradient of 0 to 100% EtOAc in heptane to afford the title compound as a colourless solid.

LC-MS (Method 2A): Rt 0.73 mins; MS m/z 377.4=[M+H]+

1H NMR (500 MHz, DMSO-d6) δ 8.84 (s, 1H), 8.63 (s, 1H), 5.35 (dd, J=10.9, 2.2 Hz, 1H), 4.04 (s, 3H), 3.99 (s, 3H), 3.97-3.92 (m, 1H), 3.60-3.53 (m, 1H), 2.92-2.81 (m, 1H), 1.97-1.84 (m, 2H), 1.72-1.46 (m, 3H).

### Step 3: 8-(2,4-Dimethoxypyrimidin-5-yl)-6-[3-[2-(1-piperidyl)ethoxy]pyrrolidin-1-yl]-9-tetrahydropyran-2-yl-purine

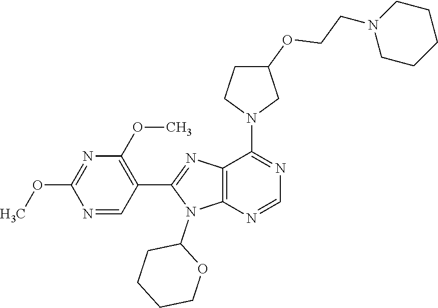 [SMILES: COc1ncc(-c2nc3c(N4CCC(OCCN5CCCCC5)C4)ncnc3n2C2CCCCO2)c(OC)n1]

The title compound was prepared from 6-chloro-8-(2,4-dimethoxypyrimidin-5-yl)-9-tetrahydropyran-2-yl-purine (step 2) and 1-(2-pyrrolidin-3-yloxyethyl)piperidine (Intermediate J) analogously to Example 7 step 1. The reaction was carried out in DMA at 70° C.

LC-MS (Method 2A): Rt 0.62 mins; MS m/z 539.6=[M+H]+

1H NMR (500 MHz, DMSO-d6) δ 8.50 (d, J=5.2 Hz, 1H), 8.25 (s, 1H), 5.22 (dd, J=10.9, 2.2 Hz, 1H), 4.42-4.19 (m, 2H), 4.14-4.06 (m, 1H), 4.01 (s, 3H), 3.96 (s, 3H), 3.94-3.88 (m, 2H), 3.86-3.55 (m, 6H), 3.54-3.46 (m, 2H), 2.88-2.80 (m, 2H), 2.23-1.97 (m, 2H), 1.93-1.86 (m, 1H), 1.79-1.73 (m, 1H), 1.69-1.55 (m, 6H), 1.49-1.43 (m, 2H), 1.29-1.25 (m, 2H).

### Step 4: 5-[6-[3-[2-(1-Piperidyl)ethoxy]pyrrolidin-1-yl]-9H-purin-8-yl]-1H-pyrimidine-2,4-dione

The title compound was prepared from 8-(2,4-dimethoxypyrimidin-5-yl)-6-[3-[2-(1-piperidyl)ethoxy]pyrrolidin-1-yl]-9-tetrahydropyran-2-yl-purine (step 3) and 1M HCl analogously to Example 7 step 2.

LC-MS (Method 7A): Rt 0.74 mins; MS m/z 427.6=[M+H]+

1H NMR (400 MHz, DMSO-d6) δ 12.11-11.75 (m, 1H), 8.52 (s, 1H), 8.07 (s, 1H), 4.21 (s, 1H), 3.92-3.50 (m, 6H), 2.45-2.38 (m, 2H), 2.37-2.29 (m, 4H), 2.16-1.96 (m, 2H), 1.53-1.37 (m, 4H), 1.37-1.26 (m, 2H).

### Example 16

### 5-[7-[3-[2-(1-Piperidyl)ethoxy]pyrrolidin-1-yl]thieno[3,2-b]pyridin-2-yl]-1H-pyrimidine-2,4-dione

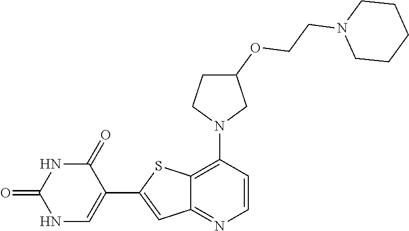 [SMILES: O=c1[nH]cc(-c2cc3nccc(N4CCC(OCCN5CCCCC5)C4)c3s2)c(=O)[nH]1]

### Step 1: 7-Chloro-2-(2,4-dimethoxypyrimidin-5-yl)thieno[3,2-b]pyridine

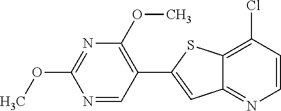 [SMILES: COc1ncc(-c2cc3nccc(Cl)c3s2)c(OC)n1]

The title compound was prepared from 2-bromo-7-chloro-thieno[3,2-b]pyridine and (2,4-dimethoxypyrimidin-5-yl)boronic acid analogously to Example 15 step 2.

LC-MS (Method 7A): Rt 3.49 mins; MS m/z 308.0=[M+H]+

1H NMR (500 MHz, CDCl3) δ 8.67 (s, 1H), 8.58 (d, J=5.1 Hz, 1H), 7.94 (s, 1H), 7.27 (m, 1H), 4.18 (s, 3H), 4.08 (s, 3H).

### Step 2: 5-(7-Chlorothieno[3,2-b]pyridin-2-yl)-1H-pyrimidine-2,4-dione

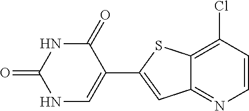 [SMILES: O=c1[nH]cc(-c2cc3nccc(Cl)c3s2)c(=O)[nH]1]

The title compound was prepared from 7-chloro-2-(2,4-dimethoxypyrimidin-5-yl)thieno[3,2-b]pyridine (step 1) and 1M HCl analogously to Example 9 step 2.

LC-MS (Method 2A): Rt 0.56 mins; MS m/z 280.2=[M+H]+

1H NMR (400 MHz, DMSO-d6) δ 11.82 (d, J=6.2 Hz, 1H), 11.73 (d, J=1.9 Hz, 1H), 8.63 (d, J=5.3 Hz, 1H), 8.55 (d, J=6.2 Hz, 1H), 8.10 (s, 1H), 7.54 (d, J=5.3 Hz, 1H).

### Step 3: 5-[7-[3-[2-(1-Piperidyl)ethoxy]pyrrolidin-1-yl]thieno[3,2-b]pyridin-2-yl]-1H-pyrimidine-2,4-dione

The title compound was prepared from 5-(7-chlorothieno[3,2-b]pyridin-2-yl)-1H-pyrimidine-2,4-dione (step 2) and 1-(2-pyrrolidin-3-yloxyethyl)piperidine (Intermediate J) analogously to Example 7 step 1. The reaction was carried out in NMP at 120° C.

LC-MS (Method 7B): Rt 1.95 mins; MS m/z 442.3=[M+H]+

1H NMR (500 MHz, DMSO-d6) δ 8.22 (s, 1H), 8.10 (d, J=5.6 Hz, 1H), 7.73 (s, 1H), 6.22 (d, J=5.6 Hz, 1H), 4.27-4.20 (m, 1H), 3.83 (dd, J=10.5, 4.7 Hz, 1H), 3.74 (td, J=8.6, 3.9 Hz, 1H), 3.71-3.65 (m, 2H), 3.59-3.52 (m, 2H), 2.42 (td, J=6.1, 1.3 Hz, 2H), 2.35-2.31 (m, 4H), 2.14-2.06 (m, 2H), 1.45-1.40 (m, 4H), 1.35-1.28 (m, 2H).

### Example 17

### 5-[4-[3,3-Difluoro-4-[2-(1-piperidyl)ethoxy]pyrrolidin-1-yl]pyrrolo[2,1-f][1,2,4]triazin-6-yl]-1H-pyrimidine-2,4-dione formate salt

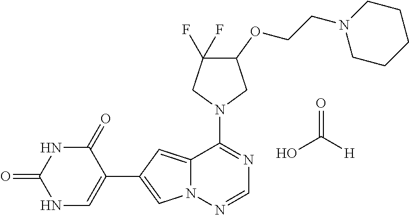

### Step 1: 1-(6-Bromopyrrolo[2,1-f][1,2,4]triazin-4-yl)-4,4-difluoro-pyrrolidin-3-ol

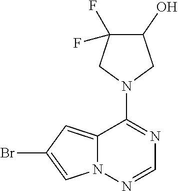 [SMILES: OC1CN(c2ncnn3cc(Br)cc23)CC1(F)F]

The title compound was prepared from 6-bromo-4-chloro-pyrrolo[2,1-f][1,2,4]triazine and 4,4-difluoropyrrolidin-3-ol hydrochloride analogously to Example 10 step 1.

LC-MS-2 (Method 2A): Rt 1.33 mins; MS m/z 319.0/320.9=[M+H]+

¹H NMR (400 MHz, DMSO-d₆) δ 7.97 (s, 1H), 7.91 (s, 1H), 7.13 (s, 1H), 6.45-6.21 (m, 1H), 1H), 4.38 (s, 2H), 4.15-3.58 (m, 3H).

### Step 2: 6-Bromo-4-[3,3-difluoro-4-[2-(1-piperidyl)ethoxy]pyrrolidin-1-yl]pyrrolo[2,1-f][1,2,4]triazine

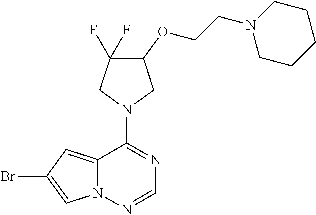 [SMILES: FC1(F)CN(c2ncnn3cc(Br)cc23)CC1OCCN1CCCCC1]

A mixture of 1-(6-bromopyrrolo[2,1-f][1,2,4]triazin-4-yl)-4,4-difluoro-pyrrolidin-3-ol (step 1) (150 mg, 0.47 mmol), 1-(2-chloroethyl)piperidine (Intermediate J step 1) (85 mg, 0.58 mmol), TBAB (85 mg, 0.26 mmol) and 5M aq. sodium hydroxide (1.6 mL, 7.9 mmol) in toluene (9 mL) was heated at 80° C. for 90 mins. The resulting mixture was allowed to cool to room temperature before being partitioned between EtOAc (50 mL) and water (50 mL). The phases were separated and the aqueous portion was further extracted with EtOAc (2×50 mL). The combined organic extracts were dried over magnesium sulfate and concentrated in vacuo. Purification by chromatography on silica eluting with a gradient of 0 to 10% MeOH in DCM afforded the title compound as a clear oil.

LC-MS-2 (Method 2A): Rt 1.12 mins; MS m/z 430.1, 432.0=[M+H]+

1H NMR (400 MHz, DMSO-d6) δ 7.98 (d, J=1.7 Hz, 1H), 7.91 (s, 1H), 7.16 (d, J=1.7 Hz, 1H), 4.42 (br s, 3H), 4.14-3.90 (br m, 3H), 3.81 (br s, 3H), 2.59 (br s, 2H), 2.40 (br s, 2H), 1.57-1.47 (br m, 4H), 1.39-1.32 (br m, 2H).

### Step 3: 4-[3,3-Difluoro-4-[2-(1-piperidyl)ethoxy]pyrrolidin-1-yl]-6-(2,4-dimethoxypyrimidin-5-yl)pyrrolo[2,1-f][1,2,4]triazine

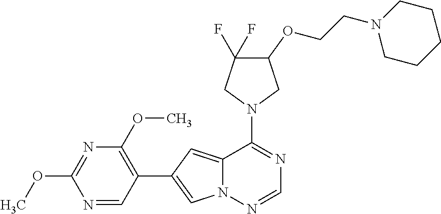 [SMILES: COc1ncc(-c2cc3c(N4CC(OCCN5CCCCC5)C(F)(F)C4)ncnn3c2)c(OC)n1]

The title compound was prepared from 6-bromo-4-[3,3-difluoro-4-[2-(1-piperidyl)ethoxy]pyrrolidin-1-yl]pyrrolo[2,1-f][1,2,4]triazine (step 2) and (2,4-dimethoxypyrimidin-5-yl)boronic acid analogously to Example 10 step 2.

LC-MS-2 (Method 2A): Rt 1.15 mins; MS m/z 490.1=[M+H]+

1H NMR (400 MHz, DMSO-d6) δ 8.90 (s, 1H), 8.15 (d, J=1.6 Hz, 1H), 7.91 (s, 1H), 7.47 (d, J=1.7 Hz, 1H), 4.63-4.31 (m, 3H), 4.27-3.96 (m, 6H), 3.94 (s, 3H), 3.92-3.70 (m, 3H), 2.67-2.54 (m, 2H), 2.45-2.34 (m, 2H), 1.60-1.43 (m, 4H), 1.43-1.29 (m, 2H).

### Step 4: 5-[4-[3,3-Difluoro-4-[2-(1-piperidyl)ethoxy]pyrrolidin-1-yl]pyrrolo[2,1-f][1,2,4]triazin-6-yl]-1H-pyrimidine-2,4-dione formate salt

The title compound was prepared from 4-[3,3-difluoro-4-[2-(1-piperidyl)ethoxy]pyrrolidin-1-yl]-6-(2,4-dimethoxypyrimidin-5-yl)pyrrolo[2,1-f][1,2,4]triazine (step 3) and 2M HCl analogously to Example 7 step 2.

LC-MS-2 (Method 8A): Rt 2.02 mins; MS m/z 462.1=[M+H]+

1H NMR (400 MHz, Acetic Acid-d4) δ 8.27 (d, J=1.5 Hz, 1H), 8.08 (s, 1H), 8.03 (s, 1H), 7.97 (s, 1H), 7.35 (d, J=1.5 Hz, 1H), 4.63-3.98 (br m, 8H), 3.71 (d, J=12.0 Hz, 2H), 3.44 (apr t, J=4.8 Hz, 2H), 3.01-2.84 (m, 2H), 1.93-1.78 (br m, 4H), 1.52-1.37 (br m, 1H).

### Example 18

### 5-[2-Chloro-4-[3-[2-(1-piperidyl)ethoxy]pyrrolidin-1-yl]pyrrolo[2,1-f][1,2,4]triazin-6-yl]-5H-pyrimidine-2,4-dione

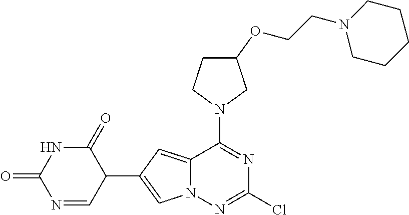 [SMILES: O=C1N=CC(c2cc3c(N4CCC(OCCN5CCCCC5)C4)nc(Cl)nn3c2)C(=O)N1]

### Step 1: 6-Bromo-2-chloro-4-[3-[2-(1-piperidyl)ethoxy]pyrrolidin-1-yl]pyrrolo[2,1-f][1,2,4]triazine

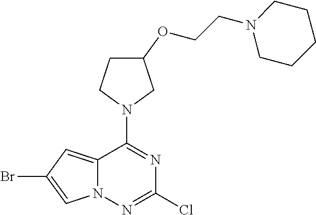 [SMILES: Clc1nc(N2CCC(OCCN3CCCCC3)C2)c2cc(Br)cn2n1]

The title compound was prepared from 6-bromo-2,4-dichloro-pyrrolo[2,1-f][1,2,4]triazine and 1-(2-pyrrolidin-3-yloxyethyl)piperidine (Intermediate J) analogously to Example 10 step 1.

LC-MS-1 (Method 2A): Rt 1.06 mins; MS m/z 428.2, 430.2=[M+H]+

1H NMR (400 MHz, DMSO-d6) δ 7.95 (d, J=1.7 Hz, 1H), 7.21-7.12 (m, 1H), 4.29-4.14 (m, 1H), 4.07-3.74 (m, 3H), 3.67-3.47 (m, 3H), 2.43-2.37 (m, 2H), 2.37-2.29 (m, 4H), 2.20-1.92 (m, 2H), 1.47-1.39 (m, 4H), 1.36-1.26 (m, 2H).

### Step 2: 2-Chloro-6-(2,4-dimethoxypyrimidin-5-yl)-4-[3-[2-(1-piperidyl)ethoxy]pyrrolidin-1-yl]pyrrolo[2,1-f][1,2,4]triazine

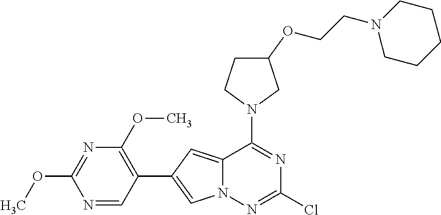 [SMILES: COc1ncc(-c2cc3c(N4CCC(OCCN5CCCCC5)C4)nc(Cl)nn3c2)c(OC)n1]

The title compound was prepared from 6-bromo-2-chloro-4-[3-[2-(1-piperidyl)ethoxy]pyrrolidin-1-yl]pyrrolo[2,1-f][1,2,4]triazine (step 1) and (2,4-dimethoxypyrimidin-5-yl)boronic acid analogously to Example 10 step 2.

LC-MS-2 (Method 2B): Rt 1.88 mins; MS m/z 488.1=[M+H]+

### Step 3: 5-[2-Chloro-4-[3-[2-(1-piperidyl)ethoxy]pyrrolidin-1-yl]pyrrolo[2,1-f][1,2,4]triazin-6-yl]-5H-pyrimidine-2,4-dione

The title compound was prepared from 2-chloro-6-(2,4-dimethoxypyrimidin-5-yl)-4-[3-[2-(1-piperidyl)ethoxy]pyrrolidin-1-yl]pyrrolo[2,1-f][1,2,4]triazine (step 2) and 2M HCl analogously to Example 10 step 3.

LC-MS-2 (Method 8B): Rt 2.53 mins; MS m/z 460.1=[M+H]+

1H NMR (400 MHz, DMSO-d6) δ 11.24 (s, 2H), 8.14 (s, 1H), 8.09 (d, J=8.6 Hz, 1H), 7.41 (dd, J=4.0, 1.7 Hz, 1H), 4.33-4.15 (m, 1H), 4.14-3.90 (m, 2H), 3.86-3.77 (m, 1H), 3.68-3.50 (m, 3H), 2.45-2.37 (m, 2H), 2.36-2.29 (m, 4H), 2.24-2.12 (m, 1H), 2.11-1.96 (m, 1H), 1.48-1.38 (m, 4H), 1.37-1.26 (m, 2H).

### Example 19

### 5-[4-(8,8-Difluoro-6-azaspiro[3.4]octan-6-yl)furo[2,3-d]pyrimidin-6-yl]-1H-pyrimidine-2,4-dione

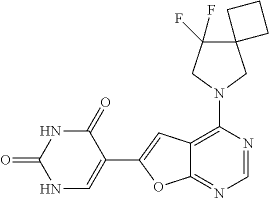 [SMILES: O=c1[nH]cc(-c2cc3c(N4CC(F)(F)C5(CCC5)C4)ncnc3o2)c(=O)[nH]1]

A mixture of 5-(4-chlorofuro[2,3-d]pyrimidin-6-yl)-1H-pyrimidine-2,4-dione (Intermediate G) (80 mg, 0.30 mmol), 8,8-difluoro-6-azaspiro[3.4]octane hydrochloride (57 mg, 0.31 mmol) and DIPEA (0.26 mL, 1.51 mmol) in MeCN (3 mL) was heated at 80° C. overnight. The resulting mixture was allowed to cool to room temperature and concentrated in vacuo. The residue was purified by C18 reverse phase chromatography eluting with a gradient of 15 to 55% MeCN in water (+0.1% formic acid) followed by a second chromatographic purification by C18 reverse phase chromatography eluting with a gradient of 20 to 55% MeCN in water (+0.1% formic acid) to afford the title compound as a colourless solid.

LC-MS-1 (Method 8A): Rt 2.55 mins; MS m/z 376.3=[M+H]+

1H NMR (400 MHz, DMSO-d6) δ 11.48 (s, 2H), 8.28 (s, 1H), 7.96 (s, 1H), 7.38 (s, 1H), 4.08 (m, 4H), 2.38-2.27 (m, 2H), 2.15-1.81 (m, 4H).

### Example 19.1

### 5-[4-(4,4-Difluoro-2-azaspiro[4.4]nonan-2-yl)furo[2,3-d]pyrimidin-6-yl]-1H-pyrimidine-2,4-dione

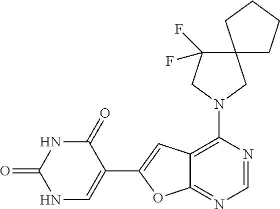 [SMILES: O=c1[nH]cc(-c2cc3c(N4CC(F)(F)C5(CCCC5)C4)ncnc3o2)c(=O)[nH]1]

The title compound was prepared from 5-(4-chlorofuro[2,3-d]pyrimidin-6-yl)-1H-pyrimidine-2,4-dione (Intermediate G) and 4,4-difluoro-2-azaspiro[4.4]nonane hydrochloride analogously to Example 19.

LC-MS-2 (Method 8A): Rt 3.38 mins; MS m/z 390.1=[M+H]+

1H NMR (400 MHz, DMSO-d6) δ 11.50 (s, 2H), 8.28 (s, 1H), 7.95 (s, 1H), 7.36 (s, 1H), 4.20 (s, 2H), 3.84 (s, 2H), 2.01-1.87 (m, 2H), 1.77-1.58 (m, 6H).

### Example 19.2

### 5-[4-[(4-Chlorophenyl)methylamino]furo[2,3-d]pyrimidin-6-yl]-1H-pyrimidine-2,4-dione

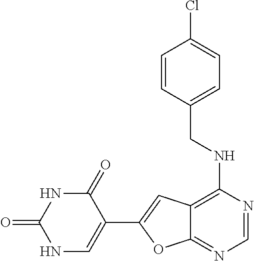 [SMILES: O=c1[nH]cc(-c2cc3c(NCc4ccc(Cl)cc4)ncnc3o2)c(=O)[nH]1]

The title compound was prepared from 5-(4-chlorofuro[2,3-d]pyrimidin-6-yl)-1H-pyrimidine-2,4-dione (Intermediate G) and (4-chlorophenyl)methanamine analogously to Example 19.

LC-MS-1 (Method 8A): Rt 2.00 mins; MS m/z 370.2=[M+H]+

1H NMR (400 MHz, DMSO-d6) δ 11.64-11.22 (m, 2H), 8.54 (t, J=6.1 Hz, 1H), 8.21 (s, 1H), 7.92 (s, 1H), 7.52 (s, 1H), 7.43-7.31 (m, 4H), 4.68 (d, J=5.7 Hz, 2H).

### Example 19.3

### 5-[4-(7,7-Difluoro-5-azaspiro[2.4]heptan-5-yl)furo[2,3-d]pyrimidin-6-yl]-1H-pyrimidine-2,4-dione

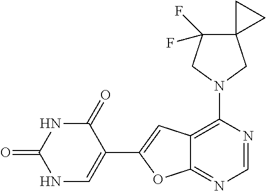 [SMILES: O=c1[nH]cc(-c2cc3c(N4CC(F)(F)C5(CC5)C4)ncnc3o2)c(=O)[nH]1]

The title compound was prepared from 5-(4-chlorofuro[2,3-d]pyrimidin-6-yl)-1H-pyrimidine-2,4-dione (Intermediate G) and 7,7-difluoro-5-azaspiro[2.4]heptane hydrochloride analogously to Example 19.

LC-MS-2 (Method 8A): Rt 2.87 mins; MS m/z 362.0=[M+H]+

1H NMR (400 MHz, DMSO-d6 @343K) δ 11.28 (s, 2H), 8.29 (s, 1H), 7.90 (s, 1H), 7.36 (s, 1H), 4.31 (t, J=12.5 Hz, 2H), 4.02 (s, 2H), 1.09-0.99 (m, 4H).

### Example 20

### [3-[[6-(2,4-Dioxo-1H-pyrimidin-5-yl)furo[2,3-d]pyrimidin-4-yl]amino]-2,2-difluoro-propyl]N-isopropylcarbamate

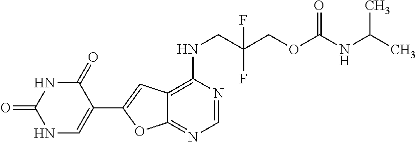 [SMILES: CC(C)NC(=O)OCC(F)(F)CNc1ncnc2oc(-c3c[nH]c(=O)[nH]c3=O)cc12]

### Step 1: 3-[[6-(2,4-Di-tert-butoxypyrimidin-5-yl)furo[2,3-d]pyrimidin-4-yl]amino]-2,2-difluoro-propan-1-ol

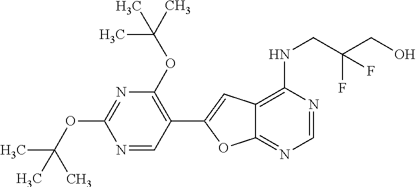 [SMILES: CC(C)(C)Oc1ncc(-c2cc3c(NCC(F)(F)CO)ncnc3o2)c(OC(C)(C)C)n1]

A mixture of 4-chloro-6-(2,4-di-tert-butoxypyrimidin-5-yl)furo[2,3-d]pyrimidine (Intermediate G step 1) (283 mg, 0.75 mmol), 3-amino-2,2-difluoro-propan-1-ol (125 mg, 1.13 mmol) and DIPEA (0.65 mL, 3.75 mmol) in NMP (3 mL) was heated at 120° C. in a sealed vial for 3.5 h. The reaction was cooled to room temperature and added dropwise to stirring water (50 mL). The precipitate was collected by filtration before being concentrated in vacuo from MeCN (20 mL) to afford the title compound as a beige solid.

LC-MS-2 (Method 2A): Rt 1.71 mins; MS m/z 452.1=[M+H]+

1H NMR (400 MHz, DMSO-d6) δ 8.72 (s, 1H), 8.39 (m, 1H), 8.29 (s, 1H), 7.33 (s, 1H), 5.62 (t, J=6.3 Hz, 1H), 4.09 (td, J=15.1, 6.1 Hz, 2H), 3.67 (td, J=13.6, 6.3 Hz, 2H), 1.73 (s, 9H), 1.60 (s, 9H).

### Step 2: [3-[[6-(2,4-Di-tert-butoxypyrimidin-5-yl)furo[2,3-d]pyrimidin-4-yl]amino]-2,2-difluoro-propyl]N-isopropylcarbamate

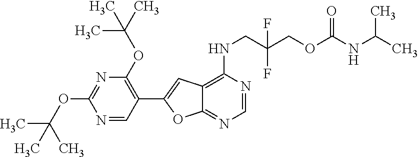 [SMILES: CC(C)NC(=O)OCC(F)(F)CNc1ncnc2oc(-c3cnc(OC(C)(C)C)nc3OC(C)(C)C)cc12]

3-[[6-(2,4-di-tert-butoxypyrimidin-5-yl)furo[2,3-d]pyrimidin-4-yl]amino]-2,2-difluoro-propan-1-ol (step 1) (200 mg, 0.44 mmol), 2-isocyanatopropane (0.44 mL, 4.43 mmol) and DIPEA (0.39 mL, 2.21 mmol) were dispersed in DCM (8 mL) and heated at 50° C. for 5 h. The reaction was cooled to room temperature then concentrated in vacuo. The residue was suspended in DCM (8 mL) before DIPEA (0.39 mL, 2.21 mmol) and 2-isocyanatopropane (0.24 mL, 2.44 mmol) were added. The reaction was heated at 50° C. for 6 h before cooling to room temperature and concentrating in vacuo. The residue was purified by chromatography on silica eluting with a gradient of 20 to 40% EtOAc in petroleum ether, followed by a second chromatographic purification on silica eluting with a gradient of 20 to 30% EtOAc in petroleum ether to afford the title compound as an off-white solid.

LC-MS-1 (Method 2B): Rt 1.77 mins; MS m/z 537.3=[M+H]+

### Step 3: [3-[[6-(2,4-Dioxo-1H-Pyrimidin-5-yl)furo[2,3-d]pyrimidin-4-yl]amino]-2,2-difluoro-propyl]N-isopropylcarbamate

3-[[6-(2,4-di-tert-butoxypyrimidin-5-yl)furo[2,3-d]pyrimidin-4-yl]amino]-2,2-difluoro-propyl]N-isopropylcarbamate (step 2) (60 mg, 0.09 mmol) was dispersed in DCM (1 mL) and cooled to O ° C. before TFA (0.07 mL, 0.89 mmol) was added. The reaction mixture was warmed to room temperature and stirred for 3 h before concentrating in vacuo. The residue was recrystalised from DMSO (2 mL) to afford the title compound as a colourless solid.

LC-MS-2 (Method 8A): Rt 2.77 mins; MS m/z 425.1=[M+H]+

1H NMR (400 MHz, DMSO-d6) δ 11.55-11.38 (m, 2H), 8.34 (t, J=6.3 Hz, 1H), 8.25 (s, 1H), 7.94 (s, 1H), 7.60 (s, 1H), 7.40 (d, J=7.7 Hz, 1H), 4.33 (t, J=14.2 Hz, 2H), 4.09 (td, J=14.8, 6.0 Hz, 2H), 3.63-3.53 (m, 1H), 1.05 (d, J=6.5 Hz, 6H).

### Example 21

### 5-[4-[3-[2-[4-(Trifluoromethyl)-1-piperidyl]ethoxy]149yrrolidine-1-yl]furo[2,3-d]pyrimidin-6-yl]-1H-pyrimidine-2,4-dione

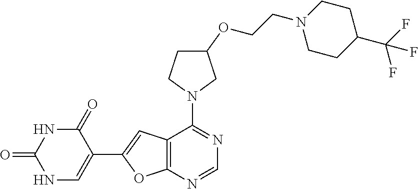 [SMILES: O=c1[nH]cc(-c2cc3c(N4CCC(OCCN5CCC(C(F)(F)F)CC5)C4)ncnc3o2)c(=O)[nH]1]

### Step 1: 2-[1-[6-(2,4-Dimethoxypyrimidin-5-yl)furo[2,3-d]pyrimidin-4-yl]149yrrolidine-3-yl]oxyethanol

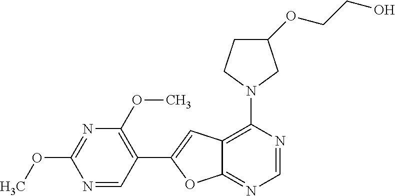 [SMILES: COc1ncc(-c2cc3c(N4CCC(OCCO)C4)ncnc3o2)c(OC)n1]

A mixture of 4-chloro-6-(2,4-dimethoxypyrimidin-5-yl)furo[2,3-d]pyrimidine (Intermediate C) (250 mg, 0.85 mmol), 2-pyrrolidin-3-yloxyethanol hydrochloride (186 mg, 1.11 mmol) and DIPEA (0.74 mL, 4.27 mmol) in MeCN (5 mL) was heated at 70° C. for 1 h. The reaction mixture was cooled to room temperature and the solid was collected by filtration, washing with MeCN (2×5 mL). The solid was dried in vacuo to afford the title compound as a grey solid.

LC-MS-1 (Method 2A): Rt 1.00 mins; MS m/z 388.3=[M+H]+

1H NMR (400 MHz, DMSO-d6) δ 8.77 (s, 1H), 8.26 (s, 1H), 7.32 (s, 1H), 4.42-4.37 (m, 1H), 4.31 (m, 1H), 4.15 (s, 3H), 4.01 (s, 3H), 3.94-3.75 (m, 4H), 3.60-3.48 (m, 4H), 2.26-2.10 (m, 2H).

### Step 2: 2-[1-[6-(2,4-Dimethoxypyrimidin-5-yl)furo[2,3-d]pyrimidin-4-yl]150yrrolidine-3-yl]oxyethyl 4-methylbenzenesulfonate

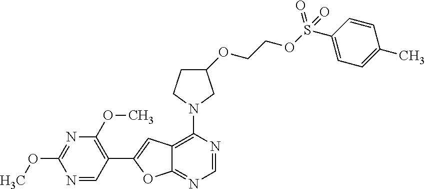

To a mixture of 2-[1-[6-(2,4-dimethoxypyrimidin-5-yl)furo[2,3-d]pyrimidin-4-yl]150yrrolidine-3-yl]oxyethanol (step 1) (260 mg, 0.67 mmol) and triethylamine (0.37 mL, 2.68 mmol) in DCM (4 mL) was added tosyl chloride (128 mg, 0.67 mmol). The suspension was stirred at room temperature for 16 h after which time additional tosyl chloride (64 mg, 0.34 mmol) was added and the suspension was stirred for a further 4 h. The resulting suspension was filtered before adding MeOH (0.4 mL) and water (10 mL) to the filtrate. The organics were separated and the aqueous was back extracted with DCM containing 10% MeOH (2×5 mL). The organic fractions were combined, dried over magnesium sulfate and concentrated in vacuo. The residue was purified by chromatography on silica eluting with a gradient of 0 to 5% MeOH in DCM to afford the title compound as a yellow solid.

LC-MS-1 (Method 2A): Rt 1.47 mins; MS m/z 542.2=[M+H]+

1H NMR (400 MHz, DMSO-d6) δ 8.78 (s, 1H), 8.27 (s, 1H), 7.78-7.72 (m, 2H), 7.38 (d, J=8.1 Hz, 2H), 7.30 (s, 1H), 4.35-4.13 (m, 3H), 4.12 (s, 3H), 3.97 (s, 3H), 3.91-3.74 (m, 2H), 3.72-3.59 (m, 4H), 2.32 (s, 3H), 2.20-2.00 (m, 2H).

### Step 3: 6-(2,4-Dimethoxypyrimidin-5-yl)-4-[3-[2-[4-(trifluoromethyl)-1-piperidyl]ethoxy]150yrrolidine-1-yl]furo[2,3-d]pyrimidine

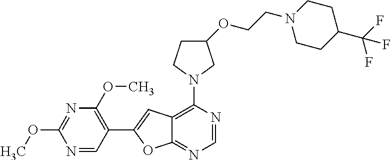 [SMILES: COc1ncc(-c2cc3c(N4CCC(OCCN5CCC(C(F)(F)F)CC5)C4)ncnc3o2)c(OC)n1]

A mixture of 2-[1-[6-(2,4-dimethoxypyrimidin-5-yl)furo[2,3-d]pyrimidin-4-yl]151yrrolidine-3-yl]oxyethyl 4-methylbenzenesulfonate (step 2) (200 mg, 0.37 mmol), potassium carbonate (153 mg, 1.11 mmol) and 4-(trifluoromethyl)piperidine (113 mg, 0.74 mmol) in MeCN (3 mL) was heated at 75° C. for 24 h before the mixture was concentrated in vacuo. The residue was triturated with MeCN (5 mL) and the solid was collected by filtration and dried in vacuo. The residue was purified by chromatography on silica eluting with a gradient of 1 to 10% 1M NH₃ in MeOH in DCM to afford the title compound as a pale yellow solid.

LC-MS-2 (Method 2A): Rt 1.12 mins; MS m/z 523.1=[M+H]+

1H NMR (400 MHz, DMSO-d6) δ 8.83 (s, 1H), 8.32 (s, 1H), 7.37 (s, 1H), 4.42-4.26 (m, 1H), 4.17 (s, 3H), 4.04 (s, 3H), 4.00-3.83 (m, 3H), 3.79-3.52 (m, 4H), 3.02-2.95 (m, 2H), 2.68-2.60 (m, 1H), 2.33-2.11 (m, 3H), 2.09-1.95 (m, 2H), 1.80-1.69 (m, 2H), 1.52-1.34 (m, 2H).

### Step 4: 5-[4-[3-[2-[4-(Trifluoromethyl)-1-piperidyl]ethoxy]pyrrolidine-1-yl]furo[2,3-d]pyrimidin-6-yl]-1H-pyrimidine-2,4-dione

A solution of 6-(2,4-dimethoxypyrimidin-5-yl)-4-[3-[2-[4-(trifluoromethyl)-1-piperidyl]ethoxy]pyrrolidine-1-yl]furo[2,3-d]pyrimidine (step 3) (140 mg, 0.27 mmol) in 1M HCl (2.68 mL, 2.68 mmol) and Methanol (2 mL) was heated at 70° C. for 7.5 h. The reaction was cooled to room temperature and concentrated in vacuo. The residue was purified by C18 reverse phase chromatography eluting with a gradient of 5 to 25% MeCN in water (+0.1% formic acid) to afford the title compound as a colourless solid.

LC-MS-1 (Method 8A): Rt 1.88 mins; MS m/z 495.3=[M+H]+

1H NMR (400 MHz, DMSO-d6) δ 11.37-11.13 (m, 2H), 8.21 (s, 1H), 7.86 (s, 1H), 7.38 (s, 1H), 4.30-4.22 (m, 1H), 3.89-3.76 (m, 3H), 3.76-3.68 (m, 1H), 3.66-3.52 (m, 2H), 2.93 (d, J=11.7 Hz, 2H), 2.48-2.39 (m, 2H), 2.20-2.08 (m, 3H), 2.07-1.98 (m, 2H), 1.75-1.65 (m, 2H), 1.48-1.34 (m, 2H).

### Example 22

### [1-[6-(2,4-Dioxo-1H-pyrimidin-5-yl)furo[2,3-d]pyrimidin-4-yl]-4,4-difluoro-pyrrolidin-3-yl]morpholine-4-carboxylate

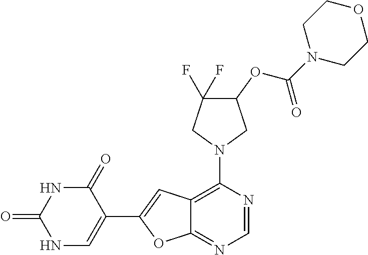 [SMILES: O=C(OC1CN(c2ncnc3oc(-c4c[nH]c(=O)[nH]c4=O)cc23)CC1(F)F)N1CCOCC1]

### Step 1: [1-[6-(2,4-Dimethoxypyrimidin-5-yl)furo[2,3-d]pyrimidin-4-yl]-4,4-difluoro-pyrrolidin-3-yl]morpholine-4-carboxylate

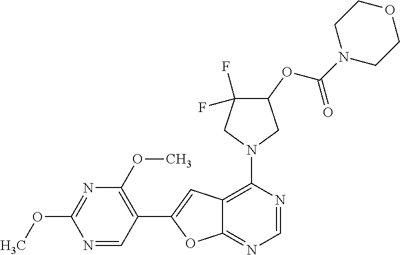 [SMILES: COc1ncc(-c2cc3c(N4CC(OC(=O)N5CCOCC5)C(F)(F)C4)ncnc3o2)c(OC)n1]

A mixture of DIPEA (0.14 mL, 0.79 mmol), 1-[6-(2,4-dimethoxypyrimidin-5-yl)furo[2,3-d]pyrimidin-4-yl]-4,4-difluoro-pyrrolidin-3-ol (Intermediate K) (100 mg, 0.26 mmol) and CDI mmol) in THF (5 mL) was heated at 50° C. for 2 h. Morpholine (0.09 mL, 1.05 mmol) was then added at this temperature and the reaction was stirred for 1 h 20 mins before cooling to room temperature and stirring overnight. The reaction mixture was diluted with EtOAc (30 mL) and washed with water (2×10 mL). The aqueous phase was extracted with 10% MeOH in DCM (2×10 mL) and the combined organic fractions dried over magnesium sulfate and concentrated in vacuo. Purification by chromatography on silica eluting with a gradient of 0 to 12% 1 M NH3 in MeOH in DCM followed by a second purification by chromatography on silica eluting with a gradient of 0 to 100% EtOAc in DCM afforded the title compound as an off-white solid.

LC-MS-1 (Method 2A): Rt 1.32 mins; MS m/z 493.4=[M+H]+

1H NMR (400 MHz, DMSO-d6) δ 8.81 (s, 1H), 8.36 (s, 1H), 7.38 (s, 1H), 5.52 (s, 1H), 4.45-4.26 (m, 3H), 4.12 (s, 3H), 4.06-4.02 (m, 1H), 3.98 (s, 3H), 3.63-3.51 (m, 4H), 3.45-3.36 (m, 4H).

### Step 2: [1-[6-(2,4-Dioxo-1H-pyrimidin-5-yl)furo[2,3-d]pyrimidin-4-yl]-4,4-difluoro-pyrrolidin-3-yl]morpholine-4-carboxylate

Trimethylsilyl iodide (0.2 mL, 1.43 mmol) was added to a solution of [1-[6-(2,4-dimethoxypyrimidin-5-yl)furo[2,3-d]pyrimidin-4-yl]-4,4-difluoro-pyrrolidin-3-yl]morpholine-4-carboxylate (step 1) (141 mg, 0.29 mmol) in chloroform (10 mL) and the reaction was stirred at room temperature for 90 mins. Water (2 mL) was added before concentrating in vacuo. The residue was dissolved in DMSO (2 mL) and water (5 mL) was added. The solid was collected by filtration and purified by C18 reverse phase chromatography eluting with a gradient of 10 to 60% MeCN in water (+0.1% NH₄OH) to afford the title compound as a colourless solid.

LC-MS-1 (Method 8B): Rt 1.81 mins; MS m/z 465.2=[M+H]+

1H NMR (400 MHz, DMSO-d6) δ 11.58 (s, 1H), 11.49 (s, 1H), 8.33 (s, 1H), 7.97 (s, 1H), 7.39 (s, 1H), 5.57-5.47 (m, 1H), 4.39-4.22 (m, 3H), 3.99 (d, J=12.3 Hz, 1H), 3.63-3.51 (m, 4H), 3.47-3.35 (m, 4H).

### Example 22.1

### [1-[6-(2,4-Dioxo-1H-pyrimidin-5-yl)furo[2,3-d]pyrimidin-4-yl]-4,4-difluoro-pyrrolidin-3-yl]8-oxa-3-azabicyclo[3.2.1]octane-3-carboxylate

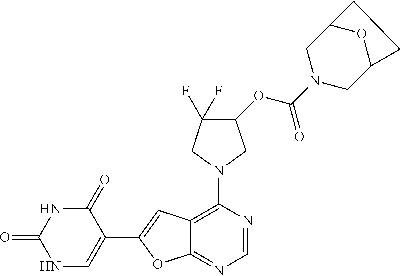 [SMILES: O=C(OC1CN(c2ncnc3oc(-c4c[nH]c(=O)[nH]c4=O)cc23)CC1(F)F)N1CC2CCC(C1)O2]

### Step 1: [1-[6-(2,4-Dimethoxypyrimidin-5-yl)furo[2,3-d]pyrimidin-4-yl]-4,4-difluoro-pyrrolidin-3-yl]8-oxa-3-azabicyclo[3.2.1]octane-3-carboxylate

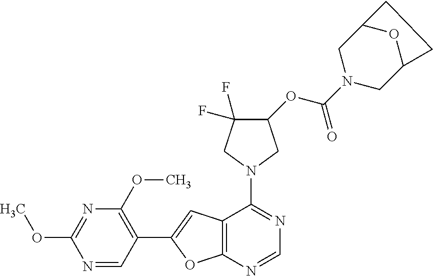 [SMILES: COc1ncc(-c2cc3c(N4CC(OC(=O)N5CC6CCC(C5)O6)C(F)(F)C4)ncnc3o2)c(OC)n1]

The title compound was prepared from 1-[6-(2,4-dimethoxypyrimidin-5-yl)furo[2,3-d]pyrimidin-4-yl]-4,4-difluoro-pyrrolidin-3-ol (Intermediate K) and 8-oxa-3-azabicyclo[3.2.1]octane hydrochloride analogously to Example 22 step 1.

LC-MS-2 (Method 2A): Rt 1.53 mins; MS m/z 519.0=[M+H]+

1H NMR (400 MHz, DMSO-d6) δ 8.81 (s, 1H), 8.36 (s, 1H), 7.38 (s, 1H), 4.45-4.20 (m, 5H), 4.13 (s, 3H), 3.98 (s, 3H), 3.67-3.56 (m, 2H), 3.14-3.00 (m, 2H), 2.45-2.42 (m, 2H), 1.87-1.72 (m, 2H), 1.69-1.60 (m, 2H).

### Step 2: [1-[6-(2,4-Dioxo-1H-pyrimidin-5-yl)furo[2,3-d]pyrimidin-4-yl]-4,4-difluoro-pyrrolidin-3-yl]8-oxa-3-azabicyclo[3.2.1]octane-3-carboxylate

The title compound was prepared from [1-[6-(2,4-dimethoxypyrimidin-5-yl)furo[2,3-d]pyrimidin-4-yl]-4,4-difluoro-pyrrolidin-3-yl]8-oxa-3-azabicyclo[3.2.1]octane-3-carboxylate (step 1) and trimethylsilyl iodide analogously to Example 22 step 2.

LC-MS-2 (Method 8A): Rt 2.27 mins; MS m/z 490.9=[M+H]+

1H NMR (400 MHz, DMSO-d6) δ 11.57 (s, 1H), 11.48 (s, 1H), 8.32 (s, 1H), 7.96 (s, 1H), 7.39 (s, 1H), 5.52 (s, 1H), 4.37-4.29 (m, 2H), 4.28-4.19 (m, 2H), 4.05-3.93 (m, 1H), 3.66-3.56 (m, 2H), 3.14-3.00 (m, 2H), 2.45-2.38 (m, 1H), 1.84-1.73 (m, 2H), 1.68-1.62 (m, 2H).

### Example 22.2

### [1-[6-(2,4-Dioxo-1H-pyrimidin-5-yl)furo[2,3-d]pyrimidin-4-yl]-4,4-difluoro-pyrrolidin-3-yl]N-isopropylcarbamate

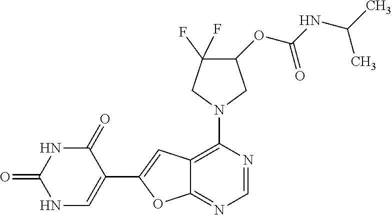 [SMILES: CC(C)NC(=O)OC1CN(c2ncnc3oc(-c4c[nH]c(=O)[nH]c4=O)cc23)CC1(F)F]

### Step 1: [1-[6-(2,4-Dimethoxypyrimidin-5-yl)furo[2,3-d]pyrimidin-4-yl]-4,4-difluoro-pyrrolidin-3-yl]N-isopropylcarbamate

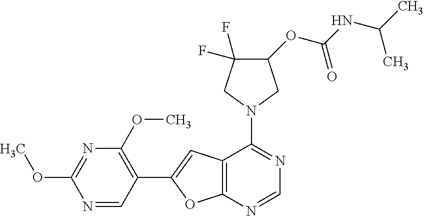 [SMILES: COc1ncc(-c2cc3c(N4CC(OC(=O)NC(C)C)C(F)(F)C4)ncnc3o2)c(OC)n1]

The title compound was prepared from 1-[6-(2,4-dimethoxypyrimidin-5-yl)furo[2,3-d]pyrimidin-4-yl]-4,4-difluoro-pyrrolidin-3-ol (Intermediate K) and propan-2-amine analogously to Example 22 step 1.

LC-MS-1 (Method 2A): Rt 1.41 mins; MS m/z 465.3=[M+H]+

1H NMR (400 MHz, DMSO-d6) δ 8.80 (s, 1H), 8.36 (s, 1H), 7.38 (s, 1H), 4.45-4.17 (m, 4H), 4.14-4.11 (m, 4H), 4.00-3.96 (m, 4H), 3.67-3.58 (m, 1H), 1.09-1.04 (m, 6H).

### Step 2: [1-[6-(2,4-Dioxo-1H-pyrimidin-5-yl)furo[2,3-d]pyrimidin-4-yl]-4,4-difluoro-pyrrolidin-3-yl]N-isopropylcarbamate

The title compound was prepared from [1-[6-(2,4-dimethoxypyrimidin-5-yl)furo[2,3-d]pyrimidin-4-yl]-4,4-difluoro-pyrrolidin-3-yl]N-isopropylcarbamate and trimethylsilyl iodide analogously to Example 22 step 2.

LC-MS-2 (Method 8B): Rt 2.05 mins; MS m/z 437.0=[M+H]+

1H NMR (400 MHz, DMSO-d6) δ 11.51-11.12 (m, 2H), 8.31 (s, 1H), 7.91 (s, 1H), 7.39 (s, 1H), 7.26 (s, 1H), 5.54-5.37 (m, 1H), 4.37-4.24 (m, 2H), 4.21-4.09 (m, 1H), 3.97-3.87 (m, 1H), 3.68-3.57 (m, 1H), 1.14-1.00 (m, 6H).

### Example 23

### 5-[4-(3,3-Difluoro-4,4-dimethyl-pyrrolidin-1-yl)-6-methyl-pyrazolo[3,4-d]pyrimidin-2-yl]-1H-pyrimidine-2,4-dione

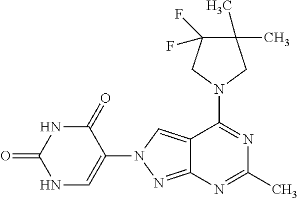 [SMILES: Cc1nc(N2CC(C)(C)C(F)(F)C2)c2cn(-c3c[nH]c(=O)[nH]c3=O)nc2n1]

### Step 1: 4-Chloro-2-(2,4-dimethoxypyrimidin-5-yl)-6-methyl-pyrazolo[3,4-d]pyrimidine

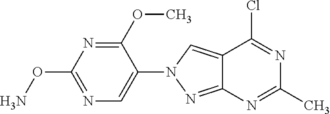 [SMILES: COc1nc(ON)ncc1-n1cc2c(Cl)nc(C)nc2n1]

(2,4-Dimethoxypyrimidin-5-yl)hydrazine hydrochloride (Intermediate E step 1a) (1.5 g, 5.44 mmol) and 4,6-dichloro-2-methyl-pyrimidine-5-carbaldehyde (988 mg, 5.17 mmol) were dispersed in THF (30 mL) and triethylamine (2.85 mL, 20.42 mmol) was added. The resulting suspension was heated at 65° C. for 1 h. The reaction was cooled to room temperature, concentrated in vacuo and the residue was triturated with DCM (20 mL) and cooled to 0° C. before filtering. The filtrate was concentrated and purified by chromatography on silica eluting with a gradient of 0 to 100% EtOAc in DCM to afford a solid which was triturated with diethyl ether (10 mL) to afford the title compound as a yellow solid.

LC-MS-2 (Method 2A): Rt 1.33 mins; MS m/z 306.9=[M+H]+

1H NMR (400 MHz, DMSO-d6) δ 9.16 (s, 1H), 8.82 (s, 1H), 4.05 (s, 3H), 4.02 (s, 3H), 2.69 (s, 3H).

### Step 2: 4-(3,3-Difluoro-4,4-dimethyl-pyrrolidin-1-yl)-2-(2,4-dimethoxypyrimidin-5-yl)-6-methyl-pyrazolo[3,4-d]pyrimidine

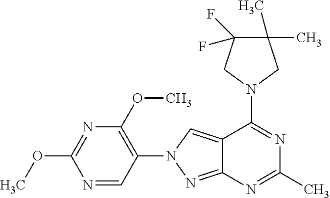

To a stirred suspension of 4-chloro-2-(2,4-dimethoxypyrimidin-5-yl)-6-methyl-pyrazolo[3,4-d]pyrimidine (step 1) (94 mg, 0.31 mmol) and 3,3-difluoro-4,4-dimethyl-pyrrolidine hydrochloride (63 mg, 0.37 mmol) in MeCN (2 mL) under nitrogen was added DIPEA (0.27 mL, 1.53 mmol). The reaction mixture was then heated at 70° C. for 20 mins before being cooled to room temperature. Water (10 mL) was added and the mixture extracted with DCM (2×15 mL). The combined organic extracts were washed with brine, dried over magnesium sulfate and concentrated in vacuo. Purification by column chromatography on silica eluting with a gradient of 0 to 5% MeOH in DCM afforded the title compound as a yellow solid.

LC-MS-2 (Method 2A): Rt 1.27 mins; MS m/z 406.0=[M+H]+

1H NMR (400 MHz, DMSO-d6) δ 8.85 (s, 1H), 8.69 (s, 1H), 4.37 (t, J=13.4 Hz, 1H), 4.21-4.11 (m, 1H), 4.02 (d, J=3.7 Hz, 3H), 4.00 (s, 3H), 3.86 (s, 1H), 3.72 (s, 1H), 2.41 (s, 3H), 1.21 (s, 3H), 1.19 (s, 3H).

### Step 3: 5-[4-(3,3-Difluoro-4,4-dimethyl-pyrrolidin-1-yl)-6-methyl-pyrazolo[3,4-d]pyrimidin-2-yl]-1H-pyrimidine-2,4-dione

2M HCl (0.79 mL, 1.58 mmol) was added to a suspension of 4-(3,3-difluoro-4,4-dimethyl-pyrrolidin-1-yl)-2-(2,4-dimethoxypyrimidin-5-yl)-6-methyl-pyrazolo[3,4-d]pyrimidine (step 2) (64 mg, 0.16 mmol) in MeOH (2 mL). The resulting solution was heated at 70° C. for 165 mins before cooling the reaction to room temperature and concentrating in vacuo. The residue was purified by C18 reverse phase chromatography eluting with a gradient of 2 to 45% MeCN in water (+0.1% formic acid) to afford the title compound as a pale yellow solid.

LC-MS-1 (Method 8A): Rt 2.04 mins; MS m/z 378.3=[M+H]+

1H NMR (400 MHz, DMSO-d6) δ 11.47 (s, 2H), 8.90 (s, 1H), 8.14 (s, 1H), 4.42-4.28 (m, 1H), 4.19-4.08 (m, 1H), 3.84 (s, 1H), 3.70 (s, 1H), 2.40 (s, 3H), 1.26-1.14 (m, 6H).

### Example 24

### 6-[1-[2-(2,4-Dioxo-1H-pyrimidin-5-yl)pyrazolo[3,4-d]pyrimidin-4-yl]157yrrolidine-3-yl]oxypyridine-3-carbonitrile

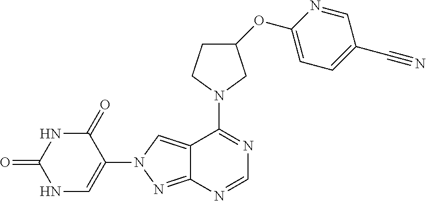 [SMILES: N#Cc1ccc(OC2CCN(c3ncnc4nn(-c5c[nH]c(=O)[nH]c5=O)cc34)C2)nc1]

### Step 1: 1-[2-(2,4-Dimethoxypyrimidin-5-yl)pyrazolo[3,4-d]pyrimidin-4-yl]158yrrolidine-3-ol

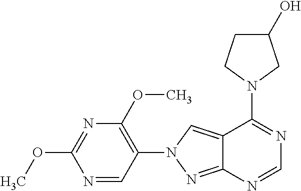 [SMILES: COc1ncc(-n2cc3c(N4CCC(O)C4)ncnc3n2)c(OC)n1]

The title compound was prepared from 4-chloro-2-(2,4-dimethoxypyrimidin-5-yl)pyrazolo[3,4-d]pyrimidine (Intermediate E) and pyrrolidine-3-ol analogously to Example 13 step 1.

LC-MS-1 (Method 2A): Rt 0.68 mins; MS m/z 344.2=[M+H]+

1H NMR (400 MHz, DMSO-d6) δ 8.80 (s, 1H), 8.72 (s, 1H), 8.25 (s, 1H), 5.07-4.82 (m, 1H), 4.57-4.36 (m, 1H), 4.06 (s, 3H), 4.03 (s, 3H), 3.95-3.81 (m, 2H), 3.81-3.60 (m, 2H), 2.18-1.96 (m, 2H).

### Step 2: 6-[1-[2-(2,4-Dimethoxypyrimidin-5-yl)pyrazolo[3,4-d]pyrimidin-4-yl]158yrrolidine-3-yl]oxypyridine-3-carbonitrile

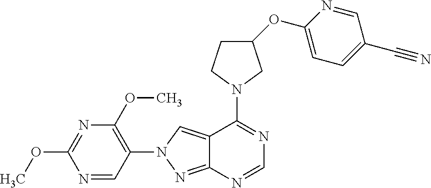 [SMILES: COc1ncc(-n2cc3c(N4CCC(Oc5ccc(C#N)cn5)C4)ncnc3n2)c(OC)n1]

To a stirred suspension of 1-[2-(2,4-dimethoxypyrimidin-5-yl)pyrazolo[3,4-d]pyrimidin-4-yl]pyrrolidine-3-ol (step 1) (160 mg, 0.47 mmol) in DMF (2.5 mL) was added sodium hydride (60% dispersion in mineral oil) (210 mg, 0.51 mmol). The reaction was stirred at room temperature for 30 mins before 6-fluoropyridine-3-carbonitrile (68.28 mg, 0.56 mmol) was added. The reaction was stirred at room temperature for 2 h before water (10 mL) was added and the mixture extracted with EtOAc (3×20 mL). The combined organic extracts were washed with brine (10 mL), dried over magnesium sulfate, filtered and concentrated in vacuo. Purification by chromatography on silica eluting with a gradient of 0 to 10% MeOH in DCM afforded the title compound as a yellow oil.

LC-MS-2 (Method 2A): Rt 1.16 mins; MS m/z 445.9=[M+H]+

1H NMR (400 MHz, DMSO-d6) δ 8.94-8.82 (m, 1H), 8.76-8.70 (m, 2H), 8.30-8.23 (m, 1H), 8.20-8.14 (m, 1H), 7.07-7.00 (m, 1H), 5.94-5.72 (m, 1H), 4.30-4.20 (m, 1H), 4.08-3.91 (m, 8H), 3.79-3.69 (m, 1H), 2.50-2.45 (m, 2H).

### Step 3: 6-[1-[2-(2,4-Dioxo-1H-pyrimidin-5-yl)pyrazolo[3,4-d]pyrimidin-4-yl]pyrrolidine-3-yl]oxypyridine-3-carbonitrile

The title compound was prepared from 6-[1-[2-(2,4-dimethoxypyrimidin-5-yl)pyrazolo[3,4-d]pyrimidin-4-yl]pyrrolidine-3-yl]oxypyridine-3-carbonitrile (step 2) and 1M HCl analogously to Example 13 step 2.

LC-MS-2 (Method 8B): Rt 1.72 mins; MS m/z 418.0=[M+H]+

1H NMR (400 MHz, Acetic Acid-d4) δ 9.40-9.30 (m, 1H), 8.67-8.50 (m, 3H), 8.02-7.95 (m, 1H), 6.98-6.89 (m, 1H), 6.05-5.90 (m, 1H), 4.56-4.05 (m, 4H), 2.70-2.49 (m, 2H).

### Example 25

### 5-[4-(3,3-Difluoro-4,4-dimethyl-pyrrolidin-1-yl)pyrazolo[1,5-a]pyrazin-2-yl]-1H-pyrimidine-2,4-dione

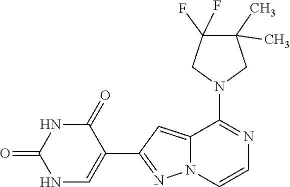 [SMILES: CC1(C)CN(c2nccn3nc(-c4c[nH]c(=O)[nH]c4=O)cc23)CC1(F)F]

### Step 1: 2-Bromo-4-(3,3-difluoro-4,4-dimethyl-pyrrolidin-1-yl)pyrazolo[1,5-a]pyrazine

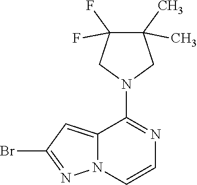 [SMILES: CC1(C)CN(c2nccn3nc(Br)cc23)CC1(F)F]

A mixture of 2-bromo-4-chloro-pyrazolo[1,5-a]pyrazine (Intermediate L) (110 mg, 0.47 mmol), 3,3-difluoro-4,4-dimethyl-pyrrolidine hydrochloride (97 mg, 0.57 mmol) and DIPEA (0.25 mL, 1.42 mmol) in MeCN (3 mL) was heated at 70° C. for 3 h after which time additional 3,3,4,4-tetramethylpyrrolidine hydrochloride (50 mg, 0.31 mmol) was added. After a further 6 h at 70° C., a third addition of 3,3-difluoro-4,4-dimethyl-pyrrolidine hydrochloride (98 mg, 0.57 mmol) was made and the reaction was heated at 70° C. for a further 18 h. The reaction was cooled to room temperature, diluted with DCM (50 mL) and washed with sat. aq. NH₄Cl (40 mL). The organic phase was dried over magnesium sulfate and concentrated in vacuo to afford the title compound as an orange solid.

LC-MS-2 (Method 2B): Rt 1.70 mins; MS m/z 330.9/332.9=[M+H]+

1H NMR (400 MHz, DMSO-d6) δ 8.02 (d, J=4.7 Hz, 1H), 7.37 (d, J=4.7 Hz, 1H), 7.26 (s, 1H), 4.23 (t, J=13.8 Hz, 2H), 3.79 (s, 2H), 1.18 (s, 6H).

### Step 2: 4-(3,3-Difluoro-4,4-dimethyl-pyrrolidin-1-yl)-2-(2,4-dimethoxypyrimidin-5-yl)pyrazolo[1,5-a]pyrazine

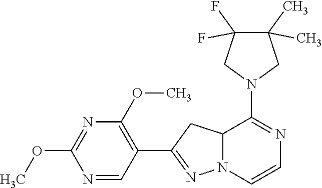

A mixture of 2-bromo-4-(3,3-difluoro-4,4-dimethyl-pyrrolidin-1-yl)pyrazolo[1,5-a]pyrazine (step 1) (170 mg, 0.51 mmol), (2,4-dimethoxypyrimidin-5-yl)boronic acid (113 mg, 0.62 mmol) and cesium carbonate (502 mg, 1.54 mmol) in dioxane (4 mL) and water (1 mL) was degassed under a flow of nitrogen. Pd(dppf)Cl₂ (38 mg, 0.05 mmol) was added and the reaction was heated at 100° C. for 10 mins. The mixture was cooled to room temperature, diluted with DCM (50 mL) and washed with water (40 mL) before being dried over magnesium sulfate and the solvent removed in vacuo. Purification by chromatography on silica eluting with a gradient of 0 to 100% EtOAc in petroleum ether afforded the title compound as a beige solid.

LC-MS-2 (Method 2B): Rt 1.69 mins; MS m/z 391.0=[M+H]+

1H NMR (400 MHz, DMSO-d6) δ 8.88 (s, 1H), 8.09 (d, J=4.7 Hz, 1H), 7.38 (d, J=4.7 Hz, 1H), 7.34 (s, 1H), 4.31 (t, J=13.7 Hz, 2H), 4.08 (s, 3H), 3.97 (s, 3H), 3.83 (s, 2H), 1.21 (s, 6H).

### Step 3: 5-[4-(3,3-Difluoro-4,4-dimethyl-pyrrolidin-1-yl)pyrazolo[1,5-a]pyrazin-2-yl]-1H-pyrimidine-2,4-dione

A mixture of 4-(3,3-difluoro-4,4-dimethyl-pyrrolidin-1-yl)-2-(2,4-dimethoxypyrimidin-5-yl)pyrazolo[1,5-a]pyrazine (step 2) (165 mg, 0.42 mmol) in 1M HCl (4.2 mL, 4.2 mmol) and MeOH (2 mL) was heated at 70° C. for 14 h. The reaction was cooled to room temperature and concentrated in vacuo. Purification by C18 reverse phase chromatography eluting with a gradient of 5 to 35% MeCN in water (+0.1% formic acid) afforded the title compound as a colourless solid.

LC-MS-2 (Method 8A): Rt 2.50 mins; MS m/z 363.0=[M+H]+

1H NMR (400 MHz, DMSO-d6) δ 11.40 (s, 1H), 11.29 (s, 1H), 8.02 (s, 1H), 8.00 (d, J=4.7 Hz, 1H), 7.43 (s, 1H), 7.34 (d, J=4.7 Hz, 1H), 4.24 (t, J=13.7 Hz, 2H), 3.82 (s, 2H), 1.21 (s, 6H).

### Example 25.1

### 5-[4-(8,8-Difluoro-6-azaspiro[3.4]octan-6-yl)pyrazolo[1,5-a]pyrazin-2-yl]-1H-pyrimidine-2,4-dione

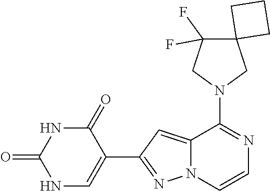 [SMILES: O=c1[nH]cc(-c2cc3c(N4CC(F)(F)C5(CCC5)C4)nccn3n2)c(=O)[nH]1]

### Step 1: 2-Bromo-4-(8,8-difluoro-6-azaspiro[3.4]octan-6-yl)pyrazolo[1,5-a]pyrazine

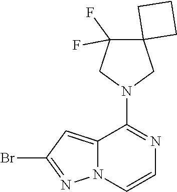 [SMILES: FC1(F)CN(c2nccn3nc(Br)cc23)CC12CCC2]

The title compound was prepared from 2-bromo-4-chloro-pyrazolo[1,5-a]pyrazine (Intermediate L) and 8,8-difluoro-6-azaspiro[3.4]octane hydrochloride analogously to Example 25 step 1.

LC-MS-2 (Method 2A): Rt 1.75 mins; MS m/z 342.6/344.9=[M+H]+

1H NMR (400 MHz, DMSO-d6) δ 8.02 (d, J=4.7 Hz, 1H), 7.36 (d, J=4.7 Hz, 1H), 7.29 (s, 1H), 4.11 (t, J=13.1 Hz, 2H), 4.05 (s, 2H), 2.35-2.27 (m, 2H), 2.07-2.00 (m, 2H), 1.92-1.72 (m, 2H).

### Step 2: 4-(8,8-Difluoro-6-azaspiro[3.4]octan-6-yl)-2-(2,4-dimethoxypyrimidin-5-yl)pyrazolo[1,5-a]pyrazine

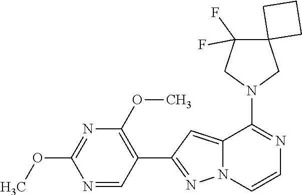 [SMILES: COc1ncc(-c2cc3c(N4CC(F)(F)C5(CCC5)C4)nccn3n2)c(OC)n1]

The title compound was prepared from 2-bromo-4-(8,8-difluoro-6-azaspiro[3.4]octan-6-yl)pyrazolo[1,5-a]pyrazine (step 1) and (2,4-dimethoxypyrimidin-5-yl)boronic acid analogously to Example 25 step 2.

LC-MS-2 (Method 2A): Rt 1.65 mins; MS m/z 403.0=[M+H]+

1H NMR (400 MHz, DMSO-d6) δ 8.88 (s, 1H), 8.09 (d, J=4.7 Hz, 1H), 7.37 (d, J=4.7 Hz, 1H), 7.35 (s, 1H), 4.19 (t, J=13.1 Hz, 2H), 4.09 (s, 3H), 4.07 (s, 2H), 3.97 (s, 3H), 2.37-2.29 (m, 2H), 2.14-2.04 (m, 2H), 2.03-1.83 (m, 2H).

### Step 3: 5-[4-(8,8-Difluoro-6-azaspiro[3.4]octan-6-yl)pyrazolo[1,5-a]pyrazin-2-yl]-1H-pyrimidine-2,4-dione

The title compound was prepared from 4-(8,8-difluoro-6-azaspiro[3.4]octan-6-yl)-2-(2,4-dimethoxypyrimidin-5-yl)pyrazolo[1,5-a]pyrazine (step 2) and 1M HCl analogously to Example 25 step 3.

LC-MS-2 (Method 8A): Rt 2.62 mins; MS m/z 375.0=[M+H]+

1H NMR (400 MHz, DMSO-d6) δ 11.38 (s, 1H), 11.23 (s, 1H), 8.02 (s, 1H), 8.00 (d, J=4.8 Hz, 1H), 7.44 (s, 1H), 7.34 (d, J=4.8 Hz, 1H), 4.14 (t, J=13.0 Hz, 2H), 4.06 (s, 2H), 2.37-2.28 (m, 2H), 2.13-2.03 (m, 2H), 2.02-1.94 (m, 1H), 1.93-1.84 (m, 1H).

### Example 25.2

### 5-[4-(7,7-Difluoro-5-azaspiro[2.4]heptan-5-yl)pyrazolo[1,5-a]pyrazin-2-yl]-1H-pyrimidine-2,4-dione

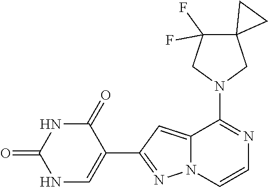 [SMILES: O=c1[nH]cc(-c2cc3c(N4CC(F)(F)C5(CC5)C4)nccn3n2)c(=O)[nH]1]

### Step 1: 2-Bromo-4-(7,7-difluoro-5-azaspiro[2.4]heptan-5-yl)pyrazolo[1,5-a]pyrazine

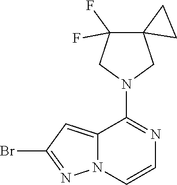 [SMILES: FC1(F)CN(c2nccn3nc(Br)cc23)CC12CC2]

The title compound was prepared from 2-bromo-4-chloro-pyrazolo[1,5-a]pyrazine (Intermediate L) and 7,7-difluoro-5-azaspiro[2.4]heptane hydrochloride analogously to Example 25 step 1.

LC-MS-1 (Method 2A): Rt 1.51 mins; MS m/z 329.4/331.4=[M+H]+

1H NMR (400 MHz, CDCl3) δ 7.75 (d, J=4.7 Hz, 1H), 7.34 (d, J=4.7 Hz, 1H), 6.72 (s, 1H), 4.28 (t, J=12.2 Hz, 2H), 4.00 (s, 2H), 1.22-1.16 (m, 2H), 0.87-0.81 (m, 2H).

### Step 2: 4-(7,7-Difluoro-5-azaspiro[2.4]heptan-5-yl)-2-(2,4-dimethoxypyrimidin-5-yl)pyrazolo[1,5-a]pyrazine

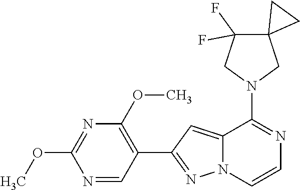 [SMILES: COc1ncc(-c2cc3c(N4CC(F)(F)C5(CC5)C4)nccn3n2)c(OC)n1]

The title compound was synthesised from 2-bromo-4-(7,7-difluoro-5-azaspiro[2.4]heptan-5-yl)pyrazolo[1,5-a]pyrazine (step 1) and (2,4-dimethoxypyrimidin-5-yl)boronic acid analogously to Example 25 step 2.

LC-MS-2 (Method 2A): Rt 1.57 mins; MS m/z 389.0=[M+H]+

1H NMR (400 MHz, DMSO-d6) δ 8.88 (s, 1H), 8.11 (d, J=4.7 Hz, 1H), 7.39 (d, J=4.7 Hz, 1H), 7.33 (s, 1H), 4.36 (t, J=12.5 Hz, 2H), 4.07 (d, J=4.4 Hz, 5H), 3.97 (s, 3H), 1.10-0.95 (m, 4H).

### Step 3: 5-[4-(7,7-Difluoro-5-azaspiro[2.4]heptan-5-yl)pyrazolo[1,5-a]pyrazin-2-yl]-1H-pyrimidine-2,4-dione

The title compound was synthesised from 4-(7,7-difluoro-5-azaspiro[2.4]heptan-5-yl)-2-(2,4-dimethoxypyrimidin-5-yl)pyrazolo[1,5-a]pyrazine and 1M HCl analogously to Example 25 step 3.

LC-MS-2 (Method 8A): Rt 2.40 mins; MS m/z 361.0=[M+H]+

1H NMR (400 MHz, DMSO-d6) δ 11.38 (s, 2H), 8.02 (d, J=5.5 Hz, 2H), 7.41 (s, 1H), 7.35 (d, J=4.7 Hz, 1H), 4.30 (t, J=12.5 Hz, 2H), 4.05 (s, 2H), 1.09-0.93 (m, 4H).

### Example 25.3

### 5-[4-(3,3-difluoro-4-methoxy-pyrrolidin-1-yl)pyrazolo[1,5-a]pyrazin-2-yl]-1H-pyrimidine-2,4-dione

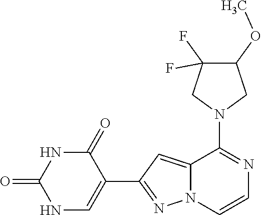 [SMILES: COC1CN(c2nccn3nc(-c4c[nH]c(=O)[nH]c4=O)cc23)CC1(F)F]

### Step 1: 2-bromo-4-(3,3-difluoro-4-methoxy-pyrrolidin-1-yl)pyrazolo[1,5-a]pyrazine

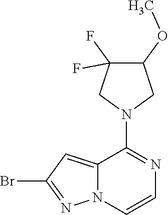 [SMILES: COC1CN(c2nccn3nc(Br)cc23)CC1(F)F]

The title compound was prepared from 2-bromo-4-chloro-pyrazolo[1,5-a]pyrazine (Intermediate L) and 3,3-difluoro-4-methoxy-pyrrolidine hydrochloride analogously to Example 25 step 1.

LC-MS-2 (Method 2A): Rt 1.46 mins; MS m/z 334.8=[M+H]+

1H NMR (400 MHz, Chloroform-d) δ 7.77 (d, J=4.8 Hz, 1H), 7.36 (d, J=4.8 Hz, 1H), 6.78 (s, 1H), 4.28-3.95 (m, 5H), 3.58 (s, 3H).

### Step 2: 4-(3,3-difluoro-4-methoxy-pyrrolidin-1-yl)-2-(2,4-dimethoxypyrimidin-5-yl)pyrazolo[1,5-a]pyrazine

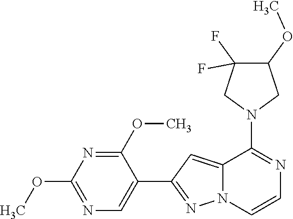 [SMILES: COc1ncc(-c2cc3c(N4CC(OC)C(F)(F)C4)nccn3n2)c(OC)n1]

The title compound was synthesised from 2-bromo-4-(3,3-difluoro-4-methoxy-pyrrolidin-1-yl)pyrazolo[1,5-a]pyrazine (step 1) and (2,4-dimethoxypyrimidin-5-yl)boronic acid analogously to Example 25 step 2.

LC-MS-2 (Method 2A): Rt 1.43 mins; MS m/z 393.0=[M+H]+

1H NMR (400 MHz, DMSO-d6) δ 8.88 (s, 1H), 8.11 (d, J=4.7 Hz, 1H), 7.39 (d, J=4.7 Hz, 1H), 7.35 (s, 1H), 4.34-4.15 (m, 4H), 4.08 (s, 3H), 3.97 (s, 3H), 3.94-3.86 (m, 1H), 3.50 (s, 3H).

### Step 3: 5-[4-(3,3-difluoro-4-methoxy-pyrrolidin-1-yl)pyrazolo[1,5-a]pyrazin-2-yl]-1H-pyrimidine-2,4-dione

The title compound was synthesised from 4-(3,3-difluoro-4-methoxy-pyrrolidin-1-yl)-2-(2,4-dimethoxypyrimidin-5-yl)pyrazolo[1,5-a]pyrazine (step 2) and 1 M HCl analogously to Example 25 step 3.

LC-MS-1 (Method 8A): Rt 2.02 mins; MS m/z 365.2=[M+H]+

1H NMR (400 MHz, DMSO-d6) δ 11.43 (s, 1H), 11.32 (d, J=6.1 Hz, 1H), 8.06 (d, J=4.9 Hz, 1H), 8.04 (d, J=6.0 Hz, 1H), 7.51 (s, 1H), 7.35 (d, J=4.8 Hz, 1H), 4.39-4.10 (m, 4H), 3.94-3.86 (m, 1H), 3.47 (s, 3H).

### Example 25.4

### 5-[4-(2-oxa-7-azaspiro[3.4]octan-7-yl)pyrazolo[1,5-a]pyrazin-2-yl]-1H-pyrimidine-2,4-dione

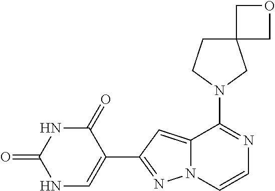 [SMILES: O=c1[nH]cc(-c2cc3c(N4CCC5(COC5)C4)nccn3n2)c(=O)[nH]1]

### Step 1: 7-(2-bromopyrazolo[1,5-a]pyrazin-4-yl)-2-oxa-7-azaspiro[3.4]octane

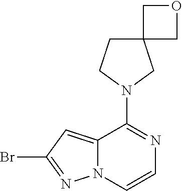 [SMILES: Brc1cc2c(N3CCC4(COC4)C3)nccn2n1]

The title compound was prepared from 2-bromo-4-chloro-pyrazolo[1,5-a]pyrazine (Intermediate L) and 2-oxa-7-azaspiro[3.4]octane oxalic acid analogously to Example 25 step 1.

LC-MS-2 (Method 2A): Rt 0.91 mins; MS m/z 310.9=[M+H]+

1H NMR (400 MHz, Chloroform-d) δ 7.70 (d, J=4.7 Hz, 1H), 7.33 (d, J=4.7 Hz, 1H), 6.82 (s, 1H), 4.74 (d, J=6.2 Hz, 2H), 4.67 (d, J=6.2 Hz, 2H), 4.06 (s, 2H), 3.88 (t, J=7.1 Hz, 2H), 2.37 (t, J=7.0 Hz, 2H).

### Step 2: 7-[2-(2,4-ditert-butoxypyrimidin-5-yl)pyrazolo[1,5-a]pyrazin-4-yl]-2-oxa-7-azaspiro[3.4]octane

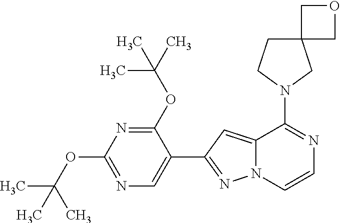 [SMILES: CC(C)(C)Oc1ncc(-c2cc3c(N4CCC5(COC5)C4)nccn3n2)c(OC(C)(C)C)n1]

The title compound was prepared from 7-(2-bromopyrazolo[1,5-a]pyrazin-4-yl)-2-oxa-7-azaspiro[3.4]octane (step 1) and (2,4-ditert-butoxypyrimidin-5-yl)boronic acid analogously to Example 25 step 2.

LC-MS-1 (Method 2A): Rt 1.27 mins; MS m/z 453.3=[M+H]+

1H NMR (400 MHz, DMSO-d6) δ 8.87 (s, 1H), 7.95 (d, J=4.7 Hz, 1H), 7.40 (s, 1H), 7.32 (d, J=4.7 Hz, 1H), 4.61 (d, J=6.1 Hz, 2H), 4.55 (d, J=6.1 Hz, 2H), 4.06 (s, 2H), 3.79 (t, J=6.9 Hz, 2H), 2.29 (t, J=6.8 Hz, 2H), 1.71 (s, 9H), 1.60 (s, 9H).

### Step 3: 5-[4-(2-oxa-7-azaspiro[3.4]octan-7-yl)pyrazolo[1,5-a]pyrazin-2-yl]-1H-pyrimidine-2,4-dione

7-[2-(2,4-ditert-butoxypyrimidin-5-yl)pyrazolo[1,5-a]pyrazin-4-yl]-2-oxa-7-azaspiro[3.4]octane (step 2) (103 mg, 0.23 mmol) was dispersed in DCM (2.5 mL) and cooled in to 0° C. TFA (0.17 mL, 2.28 mmol) was added dropwise. The reaction was stirred at 0° C. for 20 mins, then at room temperature for 40 mins. The reaction mixture was then concentrated in vacuo. The residue was purified by C18 reverse phase chromatography eluting with a gradient of 5 to 20% MeCN in water (+0.1% NH4OH) affording the title compound as a white solid.

LC-MS-2 (Method 8B): Rt 1.99 mins; MS m/z 341.0=[M+H]+

1H NMR (400 MHz, DMSO-d6) δ 11.52-11.04 (m, 2H), 8.02 (s, 1H), 7.89 (d, J=4.6 Hz, 1H), 7.46 (s, 1H), 7.30 (d, J=4.7 Hz, 1H), 4.64 (d, J=6.1 Hz, 2H), 4.54 (d, J=6.1 Hz, 2H), 4.00 (s, 2H), 3.78 (t, J=6.9 Hz, 2H), 2.29 (t, J=6.9 Hz, 2H).

### Example 25.5

### 5-[4-(3,3-difluoro-4-hydroxy-pyrrolidin-1-yl)pyrazolo[1,5-a]pyrazin-2-yl]-1H-pyrimidine-2,4-dione

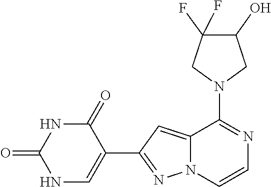 [SMILES: O=c1[nH]cc(-c2cc3c(N4CC(O)C(F)(F)C4)nccn3n2)c(=O)[nH]1]

### Step 1: 1-(2-bromopyrazolo[1,5-a]pyrazin-4-yl)-4,4-difluoro-pyrrolidin-3-ol

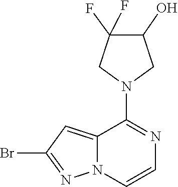 [SMILES: OC1CN(c2nccn3nc(Br)cc23)CC1(F)F]

The title compound was prepared from 2-bromo-4-chloro-pyrazolo[1,5-a]pyrazine (Intermediate L) and 4,4-difluoropyrrolidin-3-ol hydrochloride analogously to Example 25 step 1.

LC-MS-2 (Method 2A): Rt 1.19 mins; MS m/z 320.8=[M+H]+

1H NMR (400 MHz, DMSO-d6) δ 8.03 (d, J=4.7 Hz, 1H), 7.38 (d, J=4.7 Hz, 1H), 7.27 (s, 1H), 6.22 (d, J=5.1 Hz, 1H), 4.36 (m, 1H), 4.23-4.03 (m, 3H), 3.80-3.70 (m, 1H).

### Step 2: 1-[2-(2,4-dimethoxypyrimidin-5-yl)pyrazolo[1,5-a]pyrazin-4-yl]-4,4-difluoro-pyrrolidin-3-ol

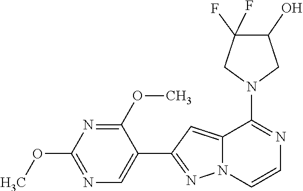 [SMILES: COc1ncc(-c2cc3c(N4CC(O)C(F)(F)C4)nccn3n2)c(OC)n1]

The title compound was synthesised from 1-(2-bromopyrazolo[1,5-a]pyrazin-4-yl)-4,4-difluoro-pyrrolidin-3-ol (step 1) and (2,4-dimethoxypyrimidin-5-yl)boronic acid analogously to Example 25 step 2.

LC-MS-2 (Method 2A): Rt 1.21 mins; MS m/z 379.0=[M+H]+

1H NMR (400 MHz, DMSO-d6) δ 8.90 (s, 1H), 8.11 (d, J=4.7 Hz, 1H), 7.40 (d, J=4.7 Hz, 1H), 7.35 (s, 1H), 6.25 (d, J=5.0 Hz, 1H), 4.47-4.37 (m, 1H), 4.24-4.15 (m, 3H), 4.09 (s, 3H), 3.98 (s, 3H), 3.88-3.79 (m, 1H).

### Step 3: 5-[4-(3,3-difluoro-4-hydroxy-pyrrolidin-1-yl)pyrazolo[1,5-a]pyrazin-2-yl]-1H-pyrimidine-2,4-dione

The title compound was synthesised from 1-[2-(2,4-dimethoxypyrimidin-5-yl)pyrazolo[1,5-a]pyrazin-4-yl]-4,4-difluoro-pyrrolidin-3-ol (step 2) and 1M HCl analogously to Example 25 step 3.

LC-MS-1 (Method 8A): Rt 1.53 mins; MS m/z 351.2=[M+H]+

1H NMR (500 MHz, DMSO-d6) δ 11.21 (s, 1H), 11.11 (s, 1H), 8.02 (d, J=6.0 Hz, 1H), 7.99 (d, J=4.7 Hz, 1H), 7.49 (s, 1H), 7.34 (d, J=4.7 Hz, 1H), 4.46-4.40 (m, 1H), 4.22-4.14 (m, 3H), 3.87-3.83 (m, 1H).

### Example 25.6

### 5-[4-[3,3-difluoro-4-[(4-methylmorpholin-2-yl)methoxy]pyrrolidin-1-yl]pyrazolo[1,5-a]pyrazin-2-yl]-1H-pyrimidine-2,4-dione

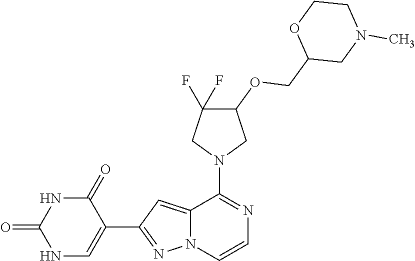 [SMILES: CN1CCOC(COC2CN(c3nccn4nc(-c5c[nH]c(=O)[nH]c5=O)cc34)CC2(F)F)C1]

### Step 1: 2-[[1-(2-bromopyrazolo[1,5-a]pyrazin-4-yl)-4,4-difluoro-pyrrolidin-3-yl]oxymethyl]-4-methyl-morpholine

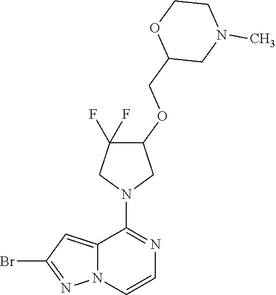 [SMILES: CN1CCOC(COC2CN(c3nccn4nc(Br)cc34)CC2(F)F)C1]

To a stirred solution of 1-(2-bromopyrazolo[1,5-a]pyrazin-4-yl)-4,4-difluoro-pyrrolidin-3-ol (Example 25.5 step 1) (300 mg, 0.94 mmol), (4-methylmorpholin-2-yl)methyl 4-methylbenzenesulfonate (intermediate H step 1) (295 mg, 1.03 mmol) and TBAB (90 mg, 0.2800 mmol) in toluene (6 mL) was added 50% aq. NaOH (1.13 mL, 14.1 mmol). The reaction was heated to 80° C. for 18 h. 50% aq. NaOH (1.13 mL, 14.1 mmol), TBAB (90.92 mg, 0.28 mmol) and (4-methylmorpholin-2-yl)methyl 4-methylbenzenesulfonate (Intermediate H step 1) (295 mg, 1.03 mmol) were added to the reaction and heated at 80° C. for 22 h. The reaction was cooled to room temperature, diluted with EtOAc (50 mL) and washed with water (30 mL). The aqueous phase was re-extracted with EtOAc (30 mL), and the combined organic extracts were washed with brine (20 mL), dried over magnesium sulfate, and concentrated in vacuo. The residue was purified by chromatography on silica eluting with a gradient of 0 to 10% MeOH in DCM, affording the title compound as a yellow oil.

LC-MS-2 (Method 2A): Rt 1.03 mins; MS m/z 431.9=[M+H]+.

1H NMR (400 MHz, Chloroform-d) δ 7.76 (d, J=4.7 Hz, 1H), 7.34 (d, J=4.7 Hz, 1H), 6.74 (s, 1H), 4.22-4.10 (m, 5H), 4.04-3.90 (m, 4H), 3.86-3.80 (m, 1H), 3.73-3.67 (m, 1H), 3.06-2.77 (m, 2H), 2.60-2.39 (m, 4H).

### Step 2: 2-[[1-[2-(2,4-dimethoxypyrimidin-5-yl)pyrazolo[1,5-a]pyrazin-4-yl]-4,4-difluoro-pyrrolidin-3-yl]oxymethyl]-4-methyl-morpholine

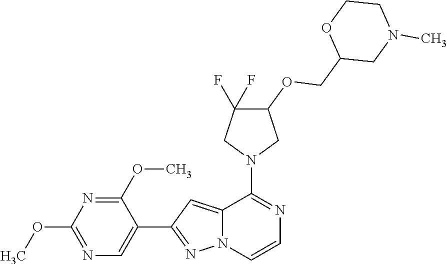

The title compound was synthesised from 2-[[1-(2-bromopyrazolo[1,5-a]pyrazin-4-yl)-4,4-difluoro-pyrrolidin-3-yl]oxymethyl]-4-methyl-morpholine (step 1) and (2,4-dimethoxypyrimidin-5-yl)boronic acid analogously to Example 25 step 2.

LC-MS-2 (Method 2A): Rt 1.07 mins; MS m/z 492.1=[M+H]+

1H NMR (400 MHz, DMSO) δ 8.88 (s, 1H), 8.11 (d, J=4.7 Hz, 1H), 7.39 (d, J=4.7 Hz, 1H), 7.35 (s, 1H), 4.47-4.38 (m, 1H), 4.28-4.13 (m, 3H), 4.08 (s, 3H), 4.01-3.85 (m, 4H), 3.81-3.55 (m, 4H), 3.53-3.43 (m, 1H), 2.70-2.64 (m, 2H), 2.14 (d, J=6.0 Hz, 3H), 1.99-1.86 (m, 1H), 1.81-1.67 (m, 1H).

### Step 3: 5-[4-[3,3-difluoro-4-[(4-methylmorpholin-2-yl)methoxy]pyrrolidin-1-yl]pyrazolo[1,5-a]pyrazin-2-yl]-1H-pyrimidine-2,4-dione

The title compound was synthesised from 2-[[1-[2-(2,4-dimethoxypyrimidin-5-yl)pyrazolo[1,5-a]pyrazin-4-yl]-4,4-difluoro-pyrrolidin-3-yl]oxymethyl]-4-methyl-morpholine (step 2) and 1M HCl analogously to Example 25 step 3.

LC-MS-2 (Method 8A): Rt 1.85 mins; MS m/z 464.0=[M+H]+

1H NMR (400 MHz, Acetic Acid-d4) δ 8.40 (s, 1H), 7.98 (d, J=5.1 Hz, 1H), 7.75 (s, 1H), 7.47 (d, J=5.1 Hz, 1H), 4.41-4.28 (m, 4H), 4.23-4.05 (m, 3H), 4.02-3.80 (m, 3H), 3.70-3.52 (m, 2H), 3.08-2.93 (m, 2H), 2.88 (s, 3H).

### Example 25.7

### 5-[4-[3,3-difluoro-4-(2-morpholinoethoxy)pyrrolidin-1-yl]pyrazolo[1,5-a]pyrazin-2-yl]-1H-pyrimidine-2,4-dione

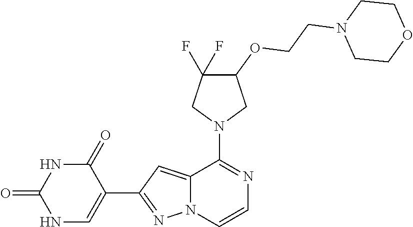 [SMILES: O=c1[nH]cc(-c2cc3c(N4CC(OCCN5CCOCC5)C(F)(F)C4)nccn3n2)c(=O)[nH]1]

### Step 1: 4-[2-[1-(2-bromopyrazolo[1,5-a]pyrazin-4-yl)-4,4-difluoro-pyrrolidin-3-yl]oxyethyl]morpholine

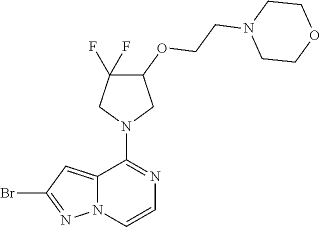 [SMILES: FC1(F)CN(c2nccn3nc(Br)cc23)CC1OCCN1CCOCC1]

To a mixture of 1-(2-bromopyrazolo[1,5-a]pyrazin-4-yl)-4,4-difluoro-pyrrolidin-3-ol (Example 25.5 step 1) (300 mg, 0.94 mmol) in THF (4 mL) was added sodium hydride (60% dispersion in mineral oil) (75 mg, 1.88 mmol) portion wise, and then stirred at room temperature for 45 mins. A solution of 4-(2-chloroethyl)morpholine (211 mg, 1.41 mmol) in THF (4 mL) was then added and the reaction was heated to 65° C. for 90 mins. The reaction was cooled to rt and sodium hydride (60% dispersion in mineral oil) (75 mg, 1.88 mmol) was added. The reaction was heated to 65° C. for 3 h. Further sodium hydride (60% dispersion in mineral oil) (75 mg, 1.88 mmol) and 4-(2-chloroethyl)morpholine (211 mg, 1.41 mmol) were added and the reaction heated at 65° C. for 16 h. Further sodium hydride (60% dispersion in mineral oil) (75 mg, 1.88 mmol) was added and the reaction heated for 4 h. The reaction was cooled to room temperature, water (20 mL) was added and the aqueous phase was extracted with EtOAc (3×20 mL). The combined organic extracts were dried over magnesium sulfate and concentrated in vacuo. The residue was purified by chromatography on silica eluting with a gradient of 0 to 10% MeOH in DCM, affording impure material. The residue was dissolved in EtOAc (20 mL) and extracted with 2M HCl (3×20 mL). The aqueous phase was neutralised with NaHCO₃ and extracted with DCM (40 mL) before concentrating in vacuo. The residue was purified by chromatography on silica eluting with a gradient of 20 to 100% EtOAc/IMS (3:1) in petroleum ether, affording the title compound as a yellow oil.

LC-MS-2 (Method 2A): Rt 1.01 mins; MS m/z 433.9=[M+H]+

1H NMR (400 MHz, Chloroform-d) δ 7.77-7.72 (m, 1H), 7.34-7.30 (m, 1H), 6.74-6.70 (m, 1H), 4.22-4.07 (m, 4H), 4.05-3.91 (m, 2H), 3.84-3.74 (m, 1H), 3.74-3.66 (m, 4H), 2.71-2.60 (m, 2H), 2.59-2.45 (m, 4H).

### Step 2: 4-[2-[1-[2-(2,4-dimethoxypyrimidin-5-yl)pyrazolo[1,5-a]pyrazin-4-yl]-4,4-difluoro-pyrrolidin-3-yl]oxyethyl]morpholine

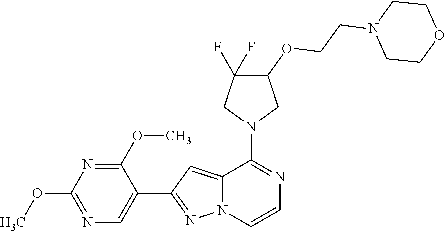 [SMILES: COc1ncc(-c2cc3c(N4CC(OCCN5CCOCC5)C(F)(F)C4)nccn3n2)c(OC)n1]

The title compound was synthesised from 4-[2-[1-(2-bromopyrazolo[1,5-a]pyrazin-4-yl)-4,4-difluoro-pyrrolidin-3-yl]oxyethyl]morpholine (step 1) and (2,4-dimethoxypyrimidin-5-yl)boronic acid analogously to Example 25 step 2.

LC-MS-2 (Method 2A): Rt 1.06 mins; MS m/z 492.1=[M+H]+

1H NMR (400 MHz, DMSO-d6) δ 8.88 (s, 1H), 8.11 (d, J=4.7 Hz, 1H), 7.39 (d, J=4.7 Hz, 1H), 7.35 (s, 1H), 4.45-4.37 (m, 1H), 4.30-4.12 (m, 4H), 4.07 (s, 3H), 3.97 (s, 3H), 3.97-3.86 (m, 2H), 3.87-3.74 (m, 2H), 3.52 (t, J=4.7 Hz, 4H), 2.44-2.36 (m, 4H).

### Step 3: 5-[4-[3,3-difluoro-4-(2-morpholinoethoxy)pyrrolidin-1-yl]pyrazolo[1,5-a]pyrazin-2-yl]-1H-pyrimidine-2,4-dione

The title compound was synthesised from 4-[2-[1-[2-(2,4-dimethoxypyrimidin-5-yl)pyrazolo[1,5-a]pyrazin-4-yl]-4,4-difluoro-pyrrolidin-3-yl]oxyethyl]morpholine (step 2) and 1M HCl analogously to Example 25 step 3.

LC-MS-1 (Method 8B): Rt 1.71 mins; MS m/z 464.3=[M+H]+

1H NMR (400 MHz, DMSO-d6) δ 10.88 (m, 2H), 8.01 (s, 1H), 7.97 (d, J=4.7 Hz, 1H), 7.44 (s, 1H), 7.35 (d, J=4.7 Hz, 1H), 4.46-4.38 (m, 1H), 4.27-4.20 (m, 1H), 4.20-4.05 (m, 2H), 3.94-3.87 (m, 1H), 3.86-3.73 (m, 2H), 3.54 (t, J=4.6 Hz, 4H), 2.57-2.53 (m, 2H), 2.45-2.40 (m, 4H).

### Example 25.8

### 5-[7-chloro-4-(3,3-difluoro-4,4-dimethyl-pyrrolidin-1-yl)pyrazolo[1,5-a]pyrazin-2-yl]-1H-pyrimidine-2,4-dione

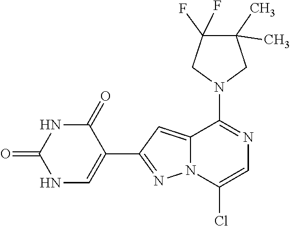 [SMILES: CC1(C)CN(c2ncc(Cl)n3nc(-c4c[nH]c(=O)[nH]c4=O)cc23)CC1(F)F]

### Step 1: 7-chloro-4-(3,3-difluoro-4,4-dimethyl-pyrrolidin-1-yl)-2-(2,4-dimethoxypyrimidin-5-yl)pyrazolo[1,5-a]pyrazine

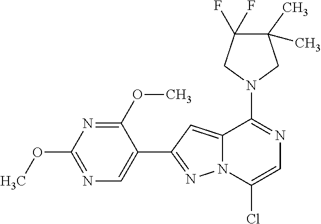

N-Chlorosuccinimide (171.02 mg, 1.28 mmol) was added to a suspension of 4-(3,3-difluoro-4,4-dimethyl-pyrrolidin-1-yl)-2-(2,4-dimethoxypyrimidin-5-yl)pyrazolo[1,5-a]pyrazine (Example 25 step 2) (500 mg, 1.28 mmol) in DMF (10 mL) and stirred at 50° C. for 3 h. The suspension was then filtered and washed with diethyl ether (15 mL) to afford the title compound as a beige solid.

LC-MS-2 (Method 8A): Rt 4.37 mins; MS m/z 425.0=[M+H]+.

1H NMR (400 MHz, DMSO) δ 8.89 (s, 1H), 7.59 (s, 1H), 7.49 (s, 1H), 4.31 (t, J=13.7 Hz, 2H), 4.09 (s, 3H), 3.98 (s, 3H), 3.82 (s, 2H), 1.21 (s, 6H).

### Step 2: 5-[7-chloro-4-(3,3-difluoro-4,4-dimethyl-pyrrolidin-1-yl)pyrazolo[1,5-a]pyrazin-2-yl]-1H-pyrimidine-2,4-dione

The title compound was synthesised from 7-chloro-4-(3,3-difluoro-4,4-dimethyl-pyrrolidin-1-yl)-2-(2,4-dimethoxypyrimidin-5-yl)pyrazolo[1,5-a]pyrazine (step 1) and 1M HCl analogously to Example 25 step 3.

LC-MS-2 (Method 8B): Rt 2.28 mins; MS m/z 397.0=[M+H]+.

1H NMR (400 MHz, DMSO) δ 11.38 (m, 2H), 8.07 (s, 1H), 7.58 (s, 1H), 7.54 (s, 1H), 4.25 (t, J=13.7 Hz, 2H), 3.82 (s, 2H), 1.20 (s, 6H).

### Example 25.9

### 5-[4-(3,3-difluoro-4,4-dimethyl-pyrrolidin-1-yl)-7-(trifluoromethyl)pyrazolo[1,5-a]pyrazin-2-yl]-1H-pyrimidine-2,4-dione

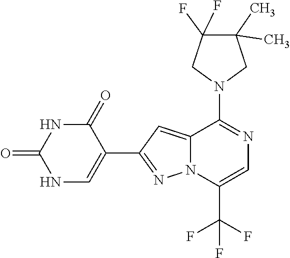 [SMILES: CC1(C)CN(c2ncc(C(F)(F)F)n3nc(-c4c[nH]c(=O)[nH]c4=O)cc23)CC1(F)F]

### Step 1: 4-(3,3-difluoro-4,4-dimethyl-pyrrolidin-1-yl)-2-(2,4-dimethoxypyrimidin-5-yl)-7-iodo-pyrazolo[1,5-a]pyrazine

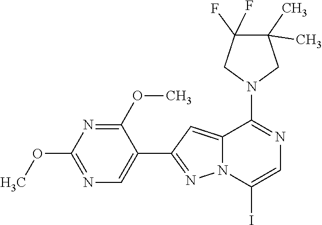

N-Iodosuccinimide (288 mg, 1.28 mmol) was added to a solution of 4-(3,3-difluoro-4,4-dimethyl-pyrrolidin-1-yl)-2-(2,4-dimethoxypyrimidin-5-yl)pyrazolo[1,5-a]pyrazine (Example 25 step 2) (500 mg, 1.28 mmol) in DMF (10 mL) and stirred at 50° C. for 8 h. Further N-iodosuccinimide (58 mg, 0.26 mmol) was added, and the reaction heated for a further 1 h. The reaction mixture was cooled to room temperature before addition of Et2O (20 mL). The resulting suspension was filtered and washed with MeCN (3 mL) and diethyl ether (3×5 mL). The residue was concentrated in vacuo to afford the title compound as an off white solid.

LC-MS-2 (Method 2B): Rt 1.86 mins; MS m/z 517.0=[M+H]+.

1H NMR (400 MHz) δ 9.09 (s, 1H), 7.64 (s, 1H), 7.34 (s, 1H), 4.23 (t, J=13.1 Hz, 2H), 4.15 (s, 3H), 4.07 (s, 3H), 3.81 (s, 2H), 1.27 (s, 6H).

### Step 2: 4-(3,3-difluoro-4,4-dimethyl-pyrrolidin-1-yl)-2-(2,4-dimethoxypyrimidin-5-yl)-7-(trifluoromethyl)pyrazolo[1,5-a]pyrazine

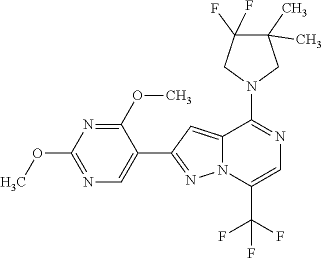

A sealed tube of 4-(3,3-difluoro-4,4-dimethyl-pyrrolidin-1-yl)-2-(2,4-dimethoxypyrimidin-5-yl)-7-iodo-pyrazolo[1,5-a]pyrazine (step 1) (400 mg, 0.77 mmol), potassium fluoride (135 mg, 2.32 mmol), CuI (30 mg, 0.15 mmol) and 1,10-phenanthroline (28 mg, 0.15 mmol) was purged with N2. Dry DMSO (8 mL) was added then trimethylborate (0.26 mL, 2.32 mmol) and trifluoromethyl trimethylsilane (0.34 mL, 2.32 mmol), before heating to 60° C. for 4 h. Additional trimethylborate (0.26 mL, 2.32 mmol), trifluoromethyl trimethylsilane (0.34 mL, 2.32 mmol), potassium fluoride (135 mg, 2.32 mmol), CuI (30 mg, 0.15 mmol) and 1,10-phenanthroline (28 mg, 0.15 mmol) were added and the reaction was stirred at 60° C. for a further 8 h. Additional trimethylborate (0.26 mL, 2.32 mmol), trifluoromethyl trimethylsilane (0.34 mL, 2.32 mmol), potassium fluoride (135 mg, 2.32 mmol), CuI (30 mg, 0.15 mmol) and 1,10-phenanthroline (28 mg, 0.15 mmol) were added and reaction was stirred at 60° C. for a further 12 h. Additional trimethylborate (0.26 mL, 2.32 mmol), trifluoromethyl trimethylsilane (0.34 mL, 2.32 mmol), potassium fluoride (135 mg, 2.32 mmol), CuI (30 mg, 0.15 mmol) and 1,10-phenanthroline (28 mg, 0.15 mmol) were added and reaction was stirred at 60° C. for a further 20 h. The reaction was cooled to room temperature, and the precipitate was collected by filtration. The solid was washed with water (3 mL) and MeCN (2 mL). Purification by chromatography on silica eluting with a gradient of 0 to 10% MeOH in DCM afforded the title compound as a white solid.

LC-MS-2 (Method 2A): Rt 1.81 mins; MS m/z 459.0=[M+H]+.

1H NMR (400 MHz, Acetic) δ 9.09 (s, 1H), 7.88 (s, 1H), 7.49 (s, 1H), 4.41 (s, 2H), 4.19 (s, 3H), 4.07 (s, 3H), 3.98 (s, 2H), 1.29 (s, 6H).

### Step 3: 5-[4-(3,3-difluoro-4,4-dimethyl-pyrrolidin-1-yl)-7-(trifluoromethyl)pyrazolo[1,5-a]pyrazin-2-yl]-1H-pyrimidine-2,4-dione

The title compound was synthesised from 4-(3,3-difluoro-4,4-dimethyl-pyrrolidin-1-yl)-2-(2,4-dimethoxypyrimidin-5-yl)-7-(trifluoromethyl)pyrazolo[1,5-a]pyrazine (step 2) and 2 M HCl analogously to Example 25 step 3.

LC-MS-1 (Method 8A): Rt 3.68 mins; MS m/z 431.3=[M+H]+.

1H NMR (400 MHz, Acetic Acid-d4) δ 8.53 (s, 1H), 8.01 (s, 1H), 7.96 (s, 1H), 4.58 (s, 2H), 4.18 (s, 2H), 1.37 (s, 6H).

### Example 25.10

### 5-[4-(3,3-difluoro-4,4-dimethyl-pyrrolidin-1-yl)-7-fluoro-pyrazolo[1,5-a]pyrazin-2-yl]-1H-pyrimidine-2,4-dione

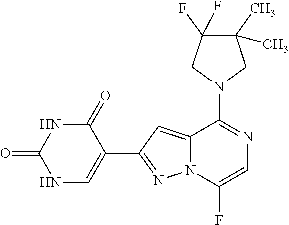 [SMILES: CC1(C)CN(c2ncc(F)n3nc(-c4c[nH]c(=O)[nH]c4=O)cc23)CC1(F)F]

### Step 1: 2-bromo-4-(3,3-difluoro-4,4-dimethyl-pyrrolidin-1-yl)-7-fluoro-6-methoxy-6,7-dihydropyrazolo[1,5-a]pyrazine

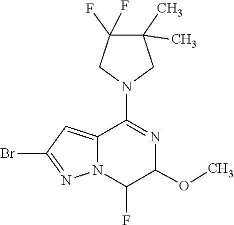 [SMILES: COC1N=C(N2CC(C)(C)C(F)(F)C2)c2cc(Br)nn2C1F]

A mixture of 2-bromo-4-(3,3-difluoro-4,4-dimethyl-pyrrolidin-1-yl)pyrazolo[1,5-a]pyrazine (Example 25 step 1) (928 mg, 2.8 mmol) and selectfluor (1.09 g, 3.08 mmol) in DMF (8 mL) and MeOH (8 mL) was stirred at room temperature for 10 min. The reaction was concentrated in vacuo to ˜¼ volume. The residue was diluted in EtOAc (100 mL) and washed with brine (50%, 3×100 mL). The organic phase was dried over magnesium sulfate and concentrated in vacuo to afford the title compound as a beige solid.

LC-MS-2 (Method 2A): Rt 1.23 mins; MS m/z 383.0=[M+H]+

### Step 2: 2-bromo-4-(3,3-difluoro-4,4-dimethyl-pyrrolidin-1-yl)-7-fluoro-pyrazolo[1,5-a]pyrazine

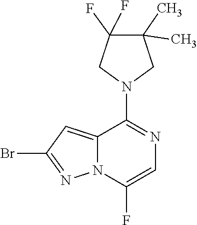 [SMILES: CC1(C)CN(c2ncc(F)n3nc(Br)cc23)CC1(F)F]

A mixture of 2-bromo-4-(3,3-difluoro-4,4-dimethyl-pyrrolidin-1-yl)-7-fluoro-6-methoxy-6,7-dihydropyrazolo[1,5-a]pyrazine (step 1) (889 mg, 2.33 mmol) and 4 M HCl in dioxane (10. mL, 40 mmol) in MeCN (10 mL) was heated to 80° C. for 20 h. The reaction was concentrated in vacuo. The residue was triturated with water (30 mL) and the solid collected by filtration. The solid was taken into CH3CN (10 mL) and concentrated in vacuo to afford the title compound as a brown solid.

LC-MS-2 (Method 2A): Rt 1.69 mins; MS m/z 350.9=[M+H]+.

1H NMR (400 MHz, DMSO) δ 7.48 (d, J=1.6 Hz, 1H), 7.45 (d, J=3.7 Hz, 1H), 4.22 (t, J=13.8 Hz, 2H), 3.76 (s, 2H), 1.18 (s, 6H).

### Step 3: 4-(3,3-difluoro-4,4-dimethyl-pyrrolidin-1-yl)-2-(2,4-dimethoxypyrimidin-5-yl)-7-fluoro-pyrazolo[1,5-a]pyrazine

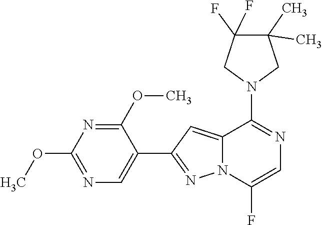

A mixture of 2-bromo-4-(3,3-difluoro-4,4-dimethyl-pyrrolidin-1-yl)-7-fluoro-pyrazolo[1,5-a]pyrazine (step 2) (647 mg, 1.85 mmol), (2,4-dimethoxypyrimidin-5-yl)boronic acid (682 mg, 3.71 mmol) and cesium carbonate (1.81 g, 5.56 mmol) in dioxane (14 mL) and water (3.5 mL) was degassed under a flow of N2. Pd(dppf)Cl2 (136 mg, 0.19 mmol) was added and the reaction was heated to 100° C. for 25 min. The reaction was cooled to rt and concentrated in vacuo. The residue was taken into DCM (100 mL) and washed with water (100 mL). The aq. phase was re-extracted with DCM (100 mL) and the combined organic extracts were dried over magnesium sulfate and concentrated in vacuo. Purification by chromatography on silica eluting with a gradient of 0 to 30% EtOAc in DCM afforded the title compound as a light brown solid.

LC-MS-2 (Method 2A): Rt 1.66 mins; MS m/z 409.1=[M+H]+.

1H NMR (400 MHz, DMSO) δ 8.91 (s, 1H), 7.52-7.42 (m, 2H), 4.28 (t, J=13.7 Hz, 2H), 4.09 (s, 3H), 3.98 (s, 3H), 3.79 (s, 2H), 1.21 (s, 6H).

### Step 4: 5-[4-(3,3-difluoro-4,4-dimethyl-pyrrolidin-1-yl)-7-fluoro-pyrazolo[1,5-a]pyrazin-2-yl]-1H-pyrimidine-2,4-dione

The title compound was synthesised from 4-(3,3-difluoro-4,4-dimethyl-pyrrolidin-1-yl)-2-(2,4-dimethoxypyrimidin-5-yl)-7-fluoro-pyrazolo[1,5-a]pyrazine (step 3) and 1M HCl analogously to Example 25 step 3.

LC-MS-2 (Method 8A): Rt 3.05 mins; MS m/z 381.0=[M+H]+.

1H NMR (400 MHz, DMSO) δ 11.47 (s, 1H), 11.40-11.34 (m, 1H), 8.09 (d, J=6.1 Hz, 1H), 7.55 (d, J=4.0 Hz, 1H), 7.44 (d, J=1.5 Hz, 1H), 4.22 (t, J=13.7 Hz, 2H), 3.79 (s, 2H), 1.20 (s, 6H).

### Example 25.11

### 5-[4-(3,3-difluoro-4,4-dimethyl-pyrrolidin-1-yl)-7-methyl-pyrazolo[1,5-a]pyrazin-2-yl]-1H-pyrimidine-2,4-dione

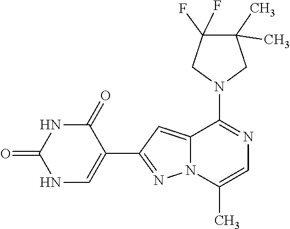 [SMILES: Cc1cnc(N2CC(C)(C)C(F)(F)C2)c2cc(-c3c[nH]c(=O)[nH]c3=O)nn12]

### Step 1: 4-(3,3-difluoro-4,4-dimethyl-pyrrolidin-1-yl)-2-(2,4-dimethoxypyrimidin-5-yl)-7-methyl-pyrazolo[1,5-a]pyrazine

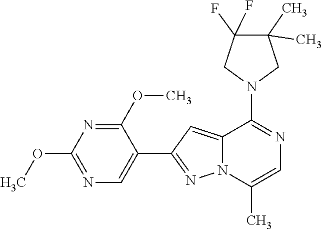

A stirred suspension of 4-(3,3-difluoro-4,4-dimethyl-pyrrolidin-1-yl)-2-(2,4-dimethoxypyrimidin-5-yl)-7-iodo-pyrazolo[1,5-a]pyrazine (Example 25.9 step 1) (400 mg, 0.77 mmol), 2,4,6-trimethyl-1,3,5,2,4,6-trioxatriborinane (3.5 M in THF, 314 uL, 1.16 mmol), and cesium carbonate (757 mg, 2.32 mmol) in 1,4-Dioxane (10 mL) and Water (2.5 mL) was degassed with N2. Pd(dppf)Cl2 (57 mg, 0.08 mmol) was added and the reaction was heated to 100° C. for 2.5 h. The reaction was cooled to room temperature and concentrated in vacuo. The residue was taken into DCM (80 mL) and washed with water (40 mL) and brine (40 mL). The organic phase was dried over magnesium sulfate and concentrated in vacuo. Purification by chromatography on silica eluting with a gradient of 0 to 100% EtOAc in DCM afforded the title compound as an off white solid.

LC-MS-2 (Method 2A): Rt 1.65 mins; MS m/z 405.1=[M+H]+.

1H NMR (400 MHz, DMSO) δ 8.93 (s, 1H), 7.36 (s, 1H), 7.29 (d, J=1.2 Hz, 1H), 4.27 (t, J=13.8 Hz, 2H), 4.09 (s, 3H), 3.97 (s, 3H), 3.79 (s, 2H), 1.20 (s, 6H).

### Step 2: 5-[4-(3,3-difluoro-4,4-dimethyl-pyrrolidin-1-yl)-7-methyl-pyrazolo[1,5-a]pyrazin-2-yl]-1H-pyrimidine-2,4-dione

The title compound was synthesised from 4-(3,3-difluoro-4,4-dimethyl-pyrrolidin-1-yl)-2-(2,4-dimethoxypyrimidin-5-yl)-7-methyl-pyrazolo[1,5-a]pyrazine (step 1) and 1M HCl analogously to Example 25 step 3.

LC-MS-1 (Method 8A): Rt 2.33 mins: MS m/z 375.4=[M−H]−.

1H NMR (400 MHz, DMSO) δ 11.38 (s, 1H), 8.06 (s, 1H), 7.47 (s, 1H), 7.25 (s, 1H), 4.21 (t, J=13.7 Hz, 2H), 3.79 (s, 2H), 2.49 (s, 3H), 1.20 (s, 6H).

### Example 25.12

### 5-[4-(3,3-difluoro-4,4-dimethyl-pyrrolidin-1-yl)-7-iodo-pyrazolo[1,5-a]pyrazin-2-yl]-1H-pyrimidine-2,4-dione

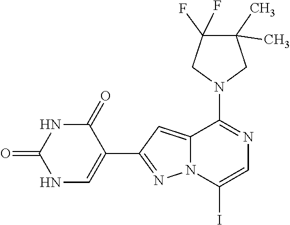 [SMILES: CC1(C)CN(c2ncc(I)n3nc(-c4c[nH]c(=O)[nH]c4=O)cc23)CC1(F)F]

### Step 1: 2-(2,4-ditert-butoxypyrimidin-5-yl)-4-(3,3-difluoro-4,4-dimethyl-pyrrolidin-1-yl)pyrazolo[1,5-a]pyrazine

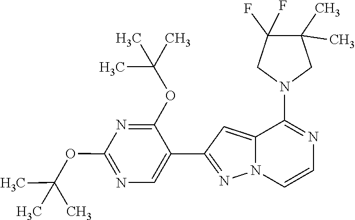 [SMILES: CC(C)(C)Oc1ncc(-c2cc3c(N4CC(C)(C)C(F)(F)C4)nccn3n2)c(OC(C)(C)C)n1]

The title compound was synthesised from 2-bromo-4-(3,3-difluoro-4,4-dimethyl-pyrrolidin-1-yl)pyrazolo[1,5-a]pyrazine (Example 25 step 1) and (2,4-ditert-butoxypyrimidin-5-yl)boronic acid analogously to Example 25 step 2.

LC-MS-2 (Method 2A): Rt 2.01 mins; MS m/z 475.2=[M+H]+

1H NMR (400 MHz, DMSO) δ 8.88 (s, 1H), 8.05 (dd, J=4.6, 0.9 Hz, 1H), 7.40 (d, J=1.0 Hz, 1H), 7.36 (d, J=4.6 Hz, 1H), 4.27 (t, J=13.5 Hz, 2H), 3.86 (s, 2H), 1.70 (s, 9H), 1.60 (s, 9H), 1.20 (s, 6H).

### Step 2: -[4-(3,3-difluoro-4,4-dimethyl-pyrrolidin-1-yl)-7-iodo-pyrazolo[1,5-a]pyrazin-2-yl]-1H-pyrimidine-2,4-dione

N-Iodosuccinimide (1.07 g, 4.75 mmol) was added to a suspension of 2-(2,4-ditert-butoxypyrimidin-5-yl)-4-(3,3-difluoro-4,4-dimethyl-pyrrolidin-1-yl)pyrazolo[1,5-a]pyrazine (step 1) (1.88 g, 3.96 mmol) in DMF (40 mL) and stirred at 50° C. for 5 h.

Additional N-iodosuccinimide (1.07 g, 4.75 mmol) was added and the reaction mixture stirred at 50° C. for 18 h. Additional N-iodosuccinimide (1.07 g, 4.75 mmol) was added and the reaction mixture was stirred at 50° C. for a further 25 h before additional N-iodosuccinimide (1.07 g, 4.75 mmol) was added. After a further 1 h at 50° C. the reaction was cooled to room temperature before adding dropwise to ice cooled water (40 mL). The precipitate was collected by filtration, washing with water (20 mL) and Et2O (100 mL). The solid was the triturated with MeCN (15 mL), and the solid was collected by filtration washing with water (15 mL) and Et2O (30 mL). The residue was purified by C18 reverse phase chromatography eluting with a gradient of 5 to 70% MeCN in water (+0.1% formic acid). The residue was then repurified by C18 reverse phase chromatography eluting with a gradient of 5 to 70% MeCN in water (+0.1% formic acid) affording the title compound as a light brown solid.

LC-MS-2A (Method 8A): Rt 3.30 mins; MS m/z 488.9=[M+H]+

1H NMR (500 MHz, DMSO) δ 11.46 (s, 1H), 11.30 (d, J=6.6 Hz, 1H), 8.02 (d, J=5.6 Hz, 1H), 7.64 (s, 2H), 4.23 (t, J=13.5 Hz, 2H), 3.80 (s, 2H), 1.20 (s, 6H).

### Example 25.13

### 5-[7-Chloro-4-(3,3-difluoro-4-hydroxy-pyrrolidin-1-yl)pyrazolo[1,5-a]pyrazin-2-yl]-1H-pyrimidine-2,4-dione

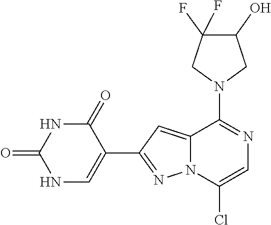 [SMILES: O=c1[nH]cc(-c2cc3c(N4CC(O)C(F)(F)C4)ncc(Cl)n3n2)c(=O)[nH]1]

### Step 1: 1-[7-Chloro-2-(2,4-dimethoxypyrimidin-5-yl)pyrazolo[1,5-a]pyrazin-4-yl]-4,4-difluoro-pyrrolidin-3-ol

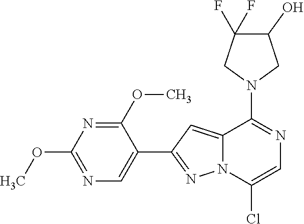 [SMILES: COc1ncc(-c2cc3c(N4CC(O)C(F)(F)C4)ncc(Cl)n3n2)c(OC)n1]

N-Chlorosuccinimide (260 mg, 1.95 mmol) was added to a solution of 1-[2-(2,4-dimethoxypyrimidin-5-yl)pyrazolo[1,5-a]pyrazin-4-yl]-4,4-difluoro-pyrrolidin-3-ol (Example 25.5 step 2) (670 mg, 1.77 mmol) in THF (12 mL) and the resulting suspension was heated at 50° C. for 48 h. Further N-chlorosuccinimide (12 mg, 0.09 mmol) was added before cooling to room temperature. The solid was collected by filtration, washing with THF (10 mL). The solid was suspended in THF (12 mL) and N-chlorosuccinimide (12 mg, 0.09 mmol) was added, and the reaction was heated at 50° C. for 19 h. Further N-chlorosuccinimide (12 mg, 0.09 mmol) was added and the reaction was heated at 50° C. for 4 h before addition of further N-chlorosuccinimide (12 mg, 0.09 mmol). The reaction was stirred at 50° C. for 24 h before cooling to room temperature. The solid was collected by filtration, washing with MeCN (3×2 mL) and Et2O (3×2 mL), and dried in vacuo to afford the title compound as a white solid.

LC-MS-2 (Method 2A): Rt 1.42 mins; MS m/z 413.0=[M+H]+

1H NMR (400 MHz, DMSO-d6) δ 8.89 (s, 1H), 7.58 (s, 1H), 7.47 (s, 1H), 6.27 (br s, 1H), 4.46-4.37 (m, 1H), 4.24-4.13 (m, 3H), 4.08 (s, 3H), 3.98 (s, 3H), 3.85-3.78 (m, 1H).

### Step 2: 5-[7-Chloro-4-(3,3-difluoro-4-hydroxy-pyrrolidin-1-yl)pyrazolo[1,5-a]pyrazin-2-yl]-1H-pyrimidine-2,4-dione

The title compound was synthesised from 1-[7-chloro-2-(2,4-dimethoxypyrimidin-5-yl)pyrazolo[1,5-a]pyrazin-4-yl]-4,4-difluoro-pyrrolidin-3-ol (step 1) and 1M HCl analogously to Example 25 step 3.

LC-MS-2 (Method 8A): Rt 2.35 mins; MS m/z 384.9=[M+H]+

1H NMR (400 MHz, DMSO-d6) δ 11.46 (s, 1H), 11.35 (s, 1H), 8.06 (s, 1H), 7.59 (s, 1H), 7.55 (s, 1H), 6.25 (d, J=5.2 Hz, 1H), 4.50-4.37 (m, 1H), 4.24-4.04 (m, 3H), 3.86-3.74 (m, 1H).

### Example 25.14

### 5-[7-Chloro-4-(3,3-difluoro-4-methoxy-pyrrolidin-1-yl)pyrazolo[1,5-a]pyrazin-2-yl]-1H-pyrimidine-2,4-dione

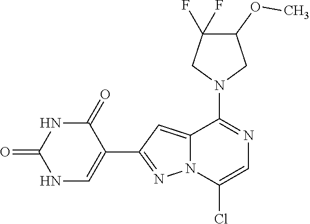 [SMILES: COC1CN(c2ncc(Cl)n3nc(-c4c[nH]c(=O)[nH]c4=O)cc23)CC1(F)F]

### Step 1: 7-Chloro-4-(3,3-difluoro-4-methoxy-pyrrolidin-1-yl)-2-(2,4-dimethoxypyrimidin-5-yl)pyrazolo[1,5-a]pyrazine

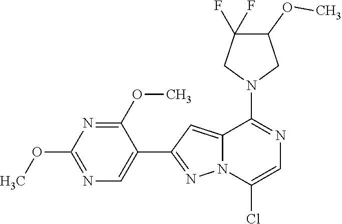

A mixture of 4-(3,3-difluoro-4-methoxy-pyrrolidin-1-yl)-2-(2,4-dimethoxypyrimidin-5-yl)pyrazolo[1,5-a]pyrazine (640 mg, 1.63 mmol) (Example 25.3 step 2) and N-chlorosuccinimide (240 mg, 1.80 mmol) in THF (1 mL) was heated to 50° C. for 48 h. The reaction was cooled to room temperature and the solid was collected by filtration. Purification by chromatography on silica eluting with a gradient of 0 to 40% EtOAc in DCM afforded the title compound as a white solid.

LC-MS-2 (Method 2A): Rt 1.64 mins; MS m/z 427.0=[M+H]+.

1H NMR (400 MHz, DMSO) δ 8.89 (s, 1H), 7.60 (s, 1H), 7.51 (s, 1H), 4.33-4.15 (m, 4H), 4.09 (s, 3H), 3.98 (s, 3H), 3.92-3.86 (m, 1H), 3.50 (s, 3H).

### Step 2: 5-[7-Chloro-4-(3,3-difluoro-4-methoxy-pyrrolidin-1-yl)pyrazolo[1,5-a]pyrazin-2-yl]-1H-pyrimidine-2,4-dione

The title compound was synthesised from 7-chloro-4-(3,3-difluoro-4-methoxy-pyrrolidin-1-yl)-2-(2,4-dimethoxypyrimidin-5-yl)pyrazolo[1,5-a]pyrazine (step 1) and 1M HCl analogously to Example 25 step 3.

LC-MS-1 (Method 8A): Rt 2.86 mins; MS m/z 399.2=[M+H]+

1H NMR (400 MHz, DMSO) δ 11.46 (s, 1H), 11.35 (s, 1H), 8.06 (s, 1H), 7.59 (s, 1H), 7.56 (s, 1H), 4.38-4.27 (m, 1H), 4.23-4.09 (m, 3H), 3.89-3.82 (m, 1H), 3.49 (s, 3H).

### Example 25.15

### 5-[7-Chloro-4-(3,3-difluoro-4,4-dimethyl-pyrrolidin-1-yl)-6-methyl-pyrazolo[1,5-a]pyrazin-2-yl]-1H-pyrimidine-2,4-dione

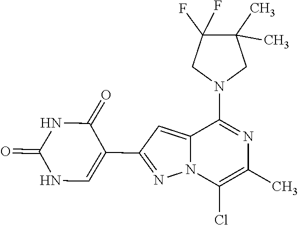 [SMILES: Cc1nc(N2CC(C)(C)C(F)(F)C2)c2cc(-c3c[nH]c(=O)[nH]c3=O)nn2c1Cl]

### Step 1: Ethyl 2-acetonyl-5-bromo-pyrazole-3-carboxylate

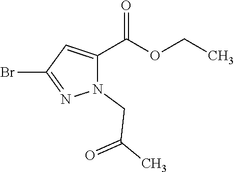

K₂CO₃ (1.39 g, 10.04 mmol) was added to a solution of ethyl 3-bromo-1H-pyrazole-5-carboxylate (2.00 g, 9.13 mmol) and 1-chloropropan-2-one (0.73 mL, 9.13 mmol) in anhydrous MeCN (75 mL). The resulting mixture was heated at 30° C. for 18 h before concentrating in vacuo. Water (150 mL) was added and extracted with EtOAc (3×100 mL). The combined organic extracts were dried over magnesium sulfate and concentrated in vacuo. Purification by chromatography on silica eluting with a gradient of 0 to 50% EtOAc in petroleum ether afforded the title compound as a white solid.

LC-MS-2 (Method 2A): Rt 1.42 mins; MS m/z 276.8=[M+H]+

1H NMR (400 MHz, Chloroform-d) δ 6.89 (s, 1H), 5.31 (s, 2H), 4.30 (q, J=7.2 Hz, 2H), 2.21 (s, 3H), 1.34 (t, J=7.2 Hz, 3H).

### Step 2: 2-bromo-6-methyl-5H-pyrazolo[1,5-a]pyrazin-4-one

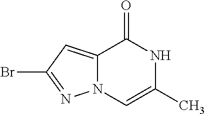

A solution of ethyl 2-acetonyl-5-bromo-pyrazole-3-carboxylate (step 1) (1.29 g, 4.67 mmol) and ammonium acetate (6.29 g, 81.57 mmol) in acetic acid (80 mL) was heated at 110° C. for 3 days. The reaction mixture was concentrated in vacuo and the residue was suspended in water (100 mL). The solid was collected by filtration and dried in vacuo to afford the title compound as a cream solid.

LC-MS-1 (Method 2A): Rt 0.93 mins; MS m/z 230.0=[M+H]+

1H NMR (400 MHz, DMSO-d6) δ 11.19 (s, 1H), 7.55-7.51 (m, 1H), 7.07 (d, J=0.7 Hz, 1H), 2.10 (d, J=1.2 Hz, 3H).

### Step 3: 2-bromo-4-chloro-6-methyl-pyrazolo[1,5-a]pyrazine

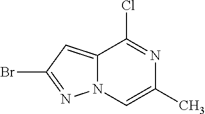

The title compound was synthesised from 2-bromo-6-methyl-5H-pyrazolo[1,5-a]pyrazin-4-one (step 2) and phosphorous (V) oxychloride analogously to Intermediate L step 4.

LC-MS-1 (Method 2A): Rt 1.45 mins; MS m/z 247.9=[M+H]+

1H NMR (400 MHz, DMSO-d6) δ 8.73-8.70 (m, 1H), 7.22 (d, J=0.9 Hz, 1H), 2.42 (d, J=1.0 Hz, 3H).

### Step 4: 2-bromo-4-(3,3-difluoro-4,4-dimethyl-pyrrolidin-1-yl)-6-methyl-pyrazolo[1,5-a]pyrazine

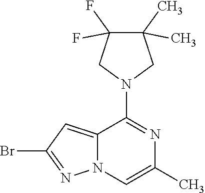 [SMILES: Cc1cn2nc(Br)cc2c(N2CC(C)(C)C(F)(F)C2)n1]

The title compound was synthesised from 2-bromo-4-chloro-6-methyl-pyrazolo[1,5-a]pyrazine (step 3) and 3,3-difluoro-4,4-dimethyl-pyrrolidine; hydrochloride analogously to Example 25 step 1.

LC-MS-1 (Method 2A): Rt 1.77 mins; MS m/z 344.9=[M+H]+

1H NMR (400 MHz, DMSO-d6) δ 7.90-7.86 (m, 1H), 7.18 (d, J=1.0 Hz, 1H), 4.23 (t, J=13.7 Hz, 2H), 3.77 (s, 2H), 2.21 (d, J=1.0 Hz, 3H), 1.18 (s, 6H).

### Step 5: 4-(3,3-difluoro-4,4-dimethyl-pyrrolidin-1-yl)-2-(2,4-dimethoxypyrimidin-5-yl)-6-methyl-pyrazolo[1,5-a]pyrazine

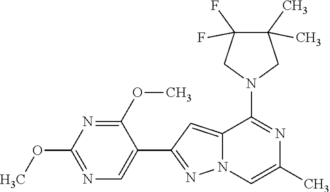

The title compound was synthesised from 2-bromo-4-(3,3-difluoro-4,4-dimethyl-pyrrolidin-1-yl)-6-methyl-pyrazolo[1,5-a]pyrazine (step 4) and (2,4-dimethoxypyrimidin-5-yl)boronic acid analogously to Example 25 step 2.

LC-MS-1 (Method 2A): Rt 1.70 mins; MS m/z 405.0=[M+H]+

1H NMR (400 MHz, DMSO-d6) δ 8.85 (s, 1H), 7.95-7.93 (m, 1H), 7.26 (d, J=1.0 Hz, 1H), 4.31 (t, J=13.7 Hz, 2H), 4.07 (s, 3H), 3.96 (s, 3H), 3.80 (s, 2H), 2.24 (d, J=1.0 Hz, 3H), 1.21 (s, 6H).

### Step 6: 7-chloro-4-(3,3-difluoro-4,4-dimethyl-pyrrolidin-1-yl)-2-(2,4-dimethoxypyrimidin-5-yl)-6-methyl-pyrazolo[1,5-a]pyrazine

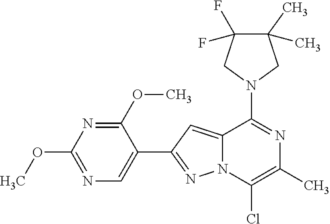

The title compound was synthesised from 4-(3,3-difluoro-4,4-dimethyl-pyrrolidin-1-yl)-2-(2,4-dimethoxypyrimidin-5-yl)-6-methyl-pyrazolo[1,5-a]pyrazine (step 5) and N-chlorosuccinimide analogously to Example 25.14 step 1.

LC-MS-2 (Method 2A): Rt 2.05 mins; MS m/z 439.0=[M+H]+

1H NMR (400 MHz, Chloroform-d) δ 9.05 (s, 1H), 7.17 (s, 1H), 4.23 (t, J=13.3 Hz, 2H), 4.15 (s, 3H), 4.07 (s, 3H), 3.82 (s, 2H), 2.47 (s, 3H), 1.27 (s, 6H).

### Step 7: 5-[7-chloro-4-(3,3-difluoro-4,4-dimethyl-pyrrolidin-1-yl)-6-methyl-pyrazolo[1,5-a]pyrazin-2-yl]-1H-pyrimidine-2,4-dione

The title compound was synthesised from 7-chloro-4-(3,3-difluoro-4,4-dimethyl-pyrrolidin-1-yl)-2-(2,4-dimethoxypyrimidin-5-yl)-6-methyl-pyrazolo[1,5-a]pyrazine (step 6) and 1M HCl analogously to Example 25 step 3.

LC-MS-1 (Method 8B): Rt 2.92 mins; MS m/z 411.2=[M+H]+

1H NMR (400 MHz, DMSO-d6) δ 11.43 (s, 1H), 11.32 (s, 1H), 8.03 (s, 1H), 7.51 (s, 1H), 4.25 (t, J=13.5 Hz, 2H), 3.79 (s, 2H), 2.38 (s, 3H), 1.20 (s, 6H).

### Example 25.16

### 5-[7-chloro-4-(3,3-dimethylpyrrolidin-1-yl)pyrazolo[1,5-a]pyrazin-2-yl]-1H-pyrimidine-2,4-dione

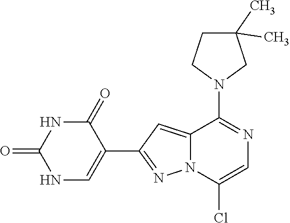 [SMILES: CC1(C)CCN(c2ncc(Cl)n3nc(-c4c[nH]c(=O)[nH]c4=O)cc23)C1]

### Step 1: 2-bromo-4-(3,3-dimethylpyrrolidin-1-yl)pyrazolo[1,5-a]pyrazine

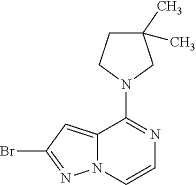 [SMILES: CC1(C)CCN(c2nccn3nc(Br)cc23)C1]

The title compound was synthesised from 2-bromo-4-chloro-pyrazolo[1,5-a]pyrazine (Intermediate L) and 3,3-dimethylpyrrolidine; hydrochloride analogously to Example 25 step 1.

LC-MS-1 (Method 2A): Rt 0.99 mins; MS m/z 294.9=[M+H]+

1H NMR (400 MHz, Chloroform-d) δ 7.65 (dd, J=4.7, 0.9 Hz, 1H), 7.30 (d, J=4.7 Hz, 1H), 6.79 (d, J=0.9 Hz, 1H), 3.89 (t, J=7.0 Hz, 2H), 3.55 (s, 2H), 1.84 (t, J=7.0 Hz, 2H), 1.17 (s, 6H).

### Step 2: 2-(2,4-dimethoxypyrimidin-5-yl)-4-(3,3-dimethylpyrrolidin-1-yl)pyrazolo[1,5-a]pyrazine

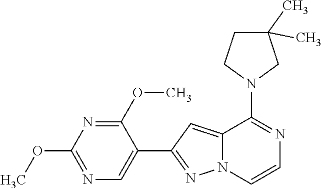

The title compound was synthesised from 2-bromo-4-(3,3-dimethylpyrrolidin-1-yl)pyrazolo[1,5-a]pyrazine (step 1) and (2,4-dimethoxypyrimidin-5-yl)boronic acid analogously to Example 25 step 2.

LC-MS-1 (Method 2A): Rt 0.99 mins; MS m/z 355.0=[M+H]+

1H NMR (400 MHz, Chloroform-d) δ 8.95 (s, 1H), 7.74 (dd, J=4.7, 0.9 Hz, 1H), 7.34 (d, J=4.7 Hz, 1H), 7.19 (s, 1H), 4.14 (s, 3H), 4.06 (s, 3H), 4.02-3.96 (m, 2H), 3.61 (s, 2H), 1.87 (t, J=7.0 Hz, 2H), 1.20 (s, 6H).

### Step 3: 7-chloro-2-(2,4-dimethoxypyrimidin-5-yl)-4-(3,3-dimethylpyrrolidin-1-yl)pyrazolo[1,5-a]pyrazine

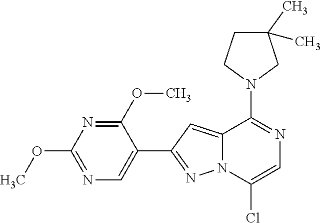

The title compound was synthesised from 2-(2,4-dimethoxypyrimidin-5-yl)-4-(3,3-dimethylpyrrolidin-1-yl)pyrazolo[1,5-a]pyrazine (step 2) and N-chlorosuccinimide analogously to Example 25.14 step 1.

LC-MS-1 (Method 2A): Rt 1.68 mins; MS m/z 388.9=[M+H]+

1H NMR (500 MHz, Chloroform-d) δ 9.05 (s, 1H), 7.39 (s, 1H), 7.31 (s, 1H), 4.14 (s, 3H), 4.06 (s, 3H), 3.99-3.93 (m, 2H), 3.58 (s, 2H), 1.88 (t, J=7.1 Hz, 2H), 1.20 (s, 6H).

### Step 4: 5-[7-chloro-4-(3,3-dimethylpyrrolidin-1-yl)pyrazolo[1,5-a]pyrazin-2-yl]-1H-pyrimidine-2,4-dione

The title compound was synthesised from 7-chloro-2-(2,4-dimethoxypyrimidin-5-yl)-4-(3,3-dimethylpyrrolidin-1-yl)pyrazolo[1,5-a]pyrazine (step 3) and 1M HCl analogously to Example 25 step 3.

LC-MS-1 (Method 8B): Rt 2.23 mins; MS m/z 361.2=[M+H]+

1H NMR (500 MHz, DMSO-d6) δ 11.49-11.40 (m, 1H), 11.37-11.26 (m, 1H), 8.06-8.01 (m, 1H), 7.59 (s, 1H), 7.47 (s, 1H), 3.89-3.79 (m, 2H), 3.52 (s, 2H), 1.81 (t, J=7.0 Hz, 2H), 1.13 (s, 6H).

### Example 25.17

### 5-[4-(3,3-dimethylpyrrolidin-1-yl)-7-fluoro-pyrazolo[1,5-a]pyrazin-2-yl]-1H-pyrimidine-2,4-dione

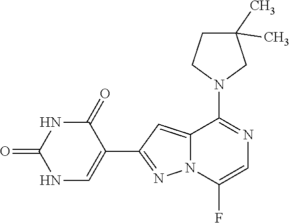 [SMILES: CC1(C)CCN(c2ncc(F)n3nc(-c4c[nH]c(=O)[nH]c4=O)cc23)C1]

### Step 1: 2-bromo-4-(3,3-dimethylpyrrolidin-1-yl)pyrazolo[1,5-a]pyrazine

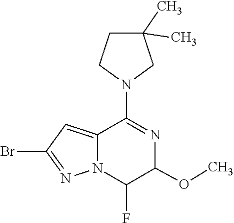 [SMILES: COC1N=C(N2CCC(C)(C)C2)c2cc(Br)nn2C1F]

The title compound was synthesised from 2-bromo-4-(3,3-dimethylpyrrolidin-1-yl)pyrazolo[1,5-a]pyrazine (Example 25.16 step 1), selectfluor and MeOH analogously to Example 25.10 step 1.

LC-MS-1 (Method 2A): Rt 0.90 mins; MS m/z 345.4=[M+H]+

### Step 2: 2-bromo-4-(3,3-dimethylpyrrolidin-1-yl)-7-fluoro-pyrazolo[1,5-a]pyrazine

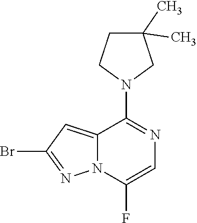 [SMILES: CC1(C)CCN(c2ncc(F)n3nc(Br)cc23)C1]

The title compound was synthesised from 2-bromo-4-(3,3-dimethylpyrrolidin-1-yl)-7-fluoro-6-methoxy-6,7-dihydropyrazolo[1,5-a]pyrazine (step 1) and 4M HCl in dioxane analogously to Example 25.10 step 2.

LC-MS-1 (Method 2A): Rt 1.62 mins; MS m/z 315.3=[M+H]+

1H NMR (500 MHz, DMSO-d6) δ 7.48 (d, J=3.6 Hz, 1H), 7.43 (s, 1H), 3.81 (t, J=7.1 Hz, 2H), 3.51 (s, 2H), 1.80 (t, J=7.0 Hz, 2H), 1.12 (s, 6H).

### Step 3: 2-(2,4-dimethoxypyrimidin-5-yl)-4-(3,3-dimethylpyrrolidin-1-yl)-7-fluoro-pyrazolo[1,5-a]pyrazine

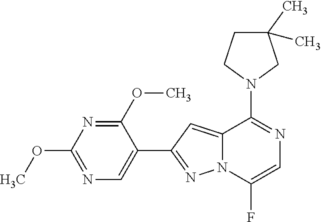

The title compound was synthesised from 2-bromo-4-(3,3-dimethylpyrrolidin-1-yl)-7-fluoro-pyrazolo[1,5-a]pyrazine (step 2) and (2,4-dimethoxypyrimidin-5-yl)boronic acid analogously to Example 25 step 2.

LC-MS-1 (Method 2A): Rt 1.48 mins; MS m/z 373.3=[M+H]+

1H NMR (500 MHz, Chloroform-d) δ 9.04 (s, 1H), 7.29 (s, 1H), 7.24-7.20 (m, 1H), 4.15 (s, 3H), 4.07 (s, 3H), 3.97-3.92 (m, 2H), 3.56 (s, 2H), 1.87 (t, J=7.0 Hz, 2H), 1.20 (s, 6H).

### Step 4: 5-[4-(3,3-dimethylpyrrolidin-1-yl)-7-fluoro-pyrazolo[1,5-a]pyrazin-2-yl]-1H-pyrimidine-2,4-dione

The title compound was synthesised from 2-(2,4-dimethoxypyrimidin-5-yl)-4-(3,3-dimethylpyrrolidin-1-yl)-7-fluoro-pyrazolo[1,5-a]pyrazine (step 3) and 1M HCl analogously to Example 25 step 3.

LC-MS-1 (Method 8B): Rt 2.03 mins; MS m/z 345.2=[M+H]+

1H NMR (500 MHz, DMSO-d6) δ 11.21 (s, 1H), 11.10 (s, 1H), 8.05 (d, J=5.9 Hz, 1H), 7.55 (d, J=4.0 Hz, 1H), 7.31 (d, J=1.6 Hz, 1H), 3.84 (t, J=7.0 Hz, 2H), 3.51 (s, 2H), 1.81 (t, J=7.0 Hz, 2H), 1.14 (s, 6H).

### Example 26

### 5-[4-(3,3-Difluoro-4,4-dimethyl-pyrrolidin-1-yl)-7-fluoro-pyrazolo[4,3-c]pyridin-2-yl]-1H-pyrimidine-2,4-dione

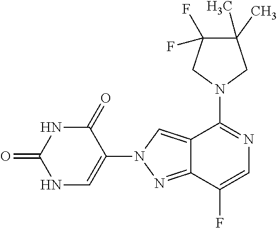 [SMILES: CC1(C)CN(c2ncc(F)c3nn(-c4c[nH]c(=O)[nH]c4=O)cc23)CC1(F)F]

### Step 1: 4-Chloro-5-fluoro-pyridine-3-carbaldehyde

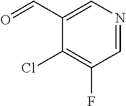

The title compound was synthesised according to the procedure of HOFFMANN LA ROCHE, BLENCH, Toby; GOODACRE, Simon; LAI, Yingjie; LIANG, Yun; MACLEOD, Calum; MAGNUSON, Steven; TSUI, Vickie; WILLIAMS, Karen; ZHANG, Birong. WO2012066061, 2012, A1, Page 130.

LC-MS-1 (Method 2B): Rt 0.87 mins; MS m/z 178.0=[M+H]+

1H NMR (400 MHz, Chloroform-d) δ 10.46 (s, 1H), 8.88 (s, 1H), 8.69 (s, 1H).

### Step 2: 4-Azido-5-fluoro-pyridine-3-carbaldehyde

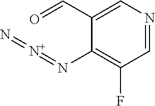 [SMILES: N=[N+]=Nc1c(F)cncc1C=O]

The title compound was synthesised according to the procedure of HOFFMANN LA ROCHE; BLENCH, Toby; GOODACRE, Simon; LAI, Yingjie; LIANG, Yun; MACLEOD, Calum; MAGNUSON, Steven; TSUI, Vickie; WILLIAMS, Karen; ZHANG, Birong. WO2012066061, 2012, A1, Page 130.

LC-MS-1 (Method 2B): Rt 0.88 mins; MS m/z 157.0=[M−2×N+H₂O+H]+

1H NMR (400 MHz, Chloroform-d) δ 10.32 (s, 1H), 8.79 (s, 1H), 8.58 (d, J=3.1 Hz, 1H).

### Step 3: 2-(2,4-Dimethoxypyrimidin-5-yl)-7-fluoro-5-oxido-pyrazolo[4,3-c]pyridin-5-ium

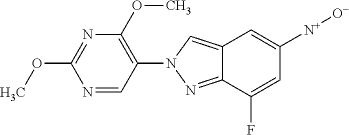 [SMILES: COc1ncc(-n2cc3cc([NH2+][O-])cc(F)c3n2)c(OC)n1]

4-azido-5-fluoro-pyridine-3-carbaldehyde (step 2) (2.28 g, 13.8 mmol) and 2,4-dimethoxypyrimidin-5-amine (2.14 g, 13.8 mmol) were dispersed in DCM (58 mL). triethylamine (5.76 mL, 41.3 mmol) was added and the solution was cooled to 0° C. Titanium tetrachloride (0.91 mL, 8.3 mmol) in DCM (8 mL) was added dropwise and after 20 mins at 0° C., the reaction was warmed to room temperature and stirred for 1.5 h before concentrating in vacuo. The obtained residue was dispersed in toluene (60 mL) and heated at reflux for 20 mins. The reaction was then cooled to room temperature and was concentrated in vacuo. The residue was suspended in DCM (100 mL) and mCPBA (3.17 g, 14.2 mmol) was added, forming a solution. After 3 h, additional mCPBA (3.17 g, 14.2 mmol) was added and the reaction was stirred at room temperature for 18 h. The reaction mixture was dissolved in EtOAc (250 mL), washed with sat. aq. sodium thiosulfate (200 mL) and sat. aq. NaHCO₃ (200 mL) before being dried over magnesium sulfate and concentrated in vacuo to afford the title compound as a beige solid.

LC-MS-1 (Method 2A): Rt 0.82 mins; MS m/z 292.1=[M+H]+

1H NMR (400 MHz, DMSO-d6) δ 8.93 (d, J=2.6 Hz, 1H), 8.83-8.77 (m, 2H), 8.31 (dd, J=6.4, 1.4 Hz, 1H), 4.04 (s, 3H), 4.02 (s, 3H)

### Step 4: 4-Chloro-2-(2,4-dimethoxypyrimidin-5-yl)-7-fluoro-pyrazolo[4,3-c]pyridine

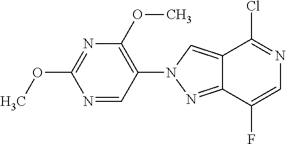 [SMILES: COc1ncc(-n2cc3c(Cl)ncc(F)c3n2)c(OC)n1]

To a mixture of 2-(2,4-dimethoxypyrimidin-5-yl)-7-fluoro-5-oxido-pyrazolo[4,3-c]pyridin-5-ium (step 3) (469 mg, 1.61 mmol) in DME (15 mL) was added phosphorus (V) oxychloride (0.35 mL, 4.83 mmol) and heating at 65° C. for 5 mins. The reaction was cooled to room temperature before being diluted with DCM (150 mL) and washed with water (100 mL). The aq. phase was extracted with DCM (100 mL) and the combined organic extracts were dried over magnesium sulfate and concentrated in vacuo. Purification by chromatography on silica eluting with a gradient of 0 to 20% EtOAc in DCM afforded the title compound as a colourless solid.

LC-MS-2 (Method 2A): Rt 1.47 mins; MS m/z 309.9=[M+H]+

1H NMR (400 MHz, DMSO-d6) δ 9.26 (d, J=2.5 Hz, 1H), 8.83 (s, 1H), 8.15 (d, J=3.4 Hz, 1H), 4.04 (s, 3H), 4.03 (s, 3H).

### Step 5: 5-[4-(3,3-Difluoro-4,4-dimethyl-pyrrolidin-1-yl)-7-fluoro-pyrazolo[4,3-c]pyridin-2-yl]-1H-pyrimidine-2,4-dione

A mixture of 4-chloro-2-(2,4-dimethoxypyrimidin-5-yl)-7-fluoro-pyrazolo[4,3-c]pyridine (step 4) (79 mg, 0.26 mmol) and 1 M HCl (3 mL, 3 mmol) in MeOH (1.5 mL) was heated at 70° C. for 120 mins. Additional 2M HCl (1.5 mL, 3 mmol) and MeOH (6 mL) were introduced and the reaction was stirred at 70° C. for a further 3.5 h before being cooled to room temperature. The reaction mixture was filtered, and the filtrate was concentrated in vacuo. The residue was triturated with MeCN (4 mL) and the solid was collected by filtration. The filtrate was concentrated in vacuo, the residue was triturated with MeCN (2 mL) and the solid was collected by filtration. The combined solids were dried in vacuo.

A mixture of the isolated solids, 3,3-difluoro-4,4-dimethyl-pyrrolidine hydrochloride (85 mg, 0.50 mmol) and DIPEA (0.26 mL, 1.49 mmol) in NMP (1 mL) was heated in a sealed vial for 18 h. Additional 3,3-difluoro-4,4-dimethyl-pyrrolidine hydrochloride (85 mg, 0.50 mmol) and DIPEA (0.26 mL, 1.49 mmol) were added and the reaction was stirred at 120° C. for a further 24 h before cooling to room temperature. The reaction mixture was purified by C18 reverse phase chromatography eluting with a gradient of 5 to 25% MeCN in water (+0.1% formic acid) to afford the title compound as a colourless solid.

LC-MS-2 (Method 8B): Rt 2.19 mins; MS m/z 381.0=[M+H]+

1H NMR (400 MHz, DMSO-d6) δ 11.85 (s, 1H), 11.62 (s, 1H), 9.15 (s, 1H), 8.23 (d, J=6.2 Hz, 1H), 7.70 (s, 1H), 4.30-4.16 (m, 2H), 3.77 (s, 2H), 1.21 (s, 6H).

### Example 26.1

### 5-[4-(8,8-difluoro-6-azaspiro[3.4]octan-6-yl)-7-fluoro-pyrazolo[4,3-c]pyridin-2-yl]-1H-pyrimidine-2,4-dione

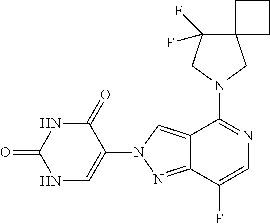 [SMILES: O=c1[nH]cc(-n2cc3c(N4CC(F)(F)C5(CCC5)C4)ncc(F)c3n2)c(=O)[nH]1]

### Step 1: 5-[4-(8,8-difluoro-6-azaspiro[3.4]octan-6-yl)-7-fluoro-pyrazolo[4,3-c]pyridin-2-yl]-1H-pyrimidine-2,4-dione

To a stirred solution of 4-chloro-2-(2,4-dimethoxypyrimidin-5-yl)-7-fluoro-pyrazolo[4,3-c]pyridine (Example 26 step 4) (550 mg, 1.78 mmol) in chloroform (15 mL) was added iodo(trimethyl)silane (1.26 mL, 8.88 mmol). The reaction was stirred at room temperature for 24 h. The reaction was cooled in an ice bath and water (2 mL) was added. The reaction was concentrated in vacuo and azeotroped with MeCN (5×25 mL) to afford the deprotected intermediate which was taken forward without purification.

The residue (125 mg, 0.24 mmol), 8,8-difluoro-6-azaspiro[3.4]octane hydrochloride (135 mg, 0.73 mmol) and DIPEA (0.21 mL, 1.22 mmol) in NMP (1 mL) was heated to 120° C. for 24 h.

The reaction was cooled to room temperature and purified by C18 reverse phase chromatography eluting with a gradient of 4 to 40% MeCN in water (+0.1% formic acid) affording the title compound as a beige solid.

LC-MS-2 (Method 8A): Rt 1.84 mins; MS m/z 393.0=[M+H]+

1H NMR (400 MHz, DMSO) δ 11.83 (s, 1H), 11.58 (s, 1H), 9.08 (d, J=2.8 Hz, 1H), 8.22 (s, 1H), 7.65 (d, J=3.7 Hz, 1H), 4.06 (t, J=13.1 Hz, 2H), 3.98 (s, 2H), 2.39-2.28 (m, 2H), 2.10-1.81 (m, 4H).

### Example 26.2

### 5-[4-(3,3-difluoro-4-methoxy-pyrrolidin-1-yl)-7-fluoro-pyrazolo[4,3-c]pyridin-2-yl]-1H-pyrimidine-2,4-dione

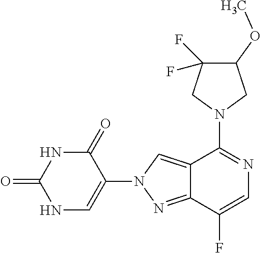 [SMILES: COC1CN(c2ncc(F)c3nn(-c4c[nH]c(=O)[nH]c4=O)cc23)CC1(F)F]

The title compound was synthesised from 4-chloro-2-(2,4-dimethoxypyrimidin-5-yl)-7-fluoro-pyrazolo[4,3-c]pyridine (Example 26 step 4) and 3,3-difluoro-4-methoxy-pyrrolidine analogously to Example 26.1 step 1.

LC-MS-2 (Method 8A): Rt 1.45 mins; MS m/z 383.0=[M+H]+

1H NMR (400 MHz, DMSO) δ 11.83 (s, 1H), 11.59 (s, 1H), 9.11 (d, J=2.8 Hz, 1H), 8.23 (d, J=3.7 Hz, 1H), 7.67 (d, J=3.7 Hz, 1H), 4.37-4.28 (m, 1H), 4.20-4.00 (m, 3H), 3.84-3.75 (m, 1H), 3.49 (s, 3H).

### Example 26.3

### 5-[4-(3,3-difluoro-4-hydroxy-pyrrolidin-1-yl)-7-fluoro-pyrazolo[4,3-c]pyridin-2-yl]-1H-pyrimidine-2,4-dione

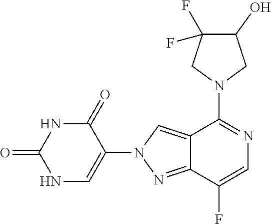 [SMILES: O=c1[nH]cc(-n2cc3c(N4CC(O)C(F)(F)C4)ncc(F)c3n2)c(=O)[nH]1]

The title compound was synthesised from 4-chloro-2-(2,4-dimethoxypyrimidin-5-yl)-7-fluoro-pyrazolo[4,3-c]pyridine (Example 26 step 4) and 4,4-difluoropyrrolidin-3-ol hydrochloride analogously to Example 26.1 step 1.

LC-MS-1 (Method 8B): Rt 1.23 mins; MS m/z 369.2=[M+H]+

1H NMR (400 MHz, DMSO) δ 11.76-11.43 (m, 2H), 9.09 (d, J=2.8 Hz, 1H), 8.23 (s, 1H), 7.65 (d, J=3.7 Hz, 1H), 6.21 (d, J=5.4 Hz, 1H), 4.41 (m, 1H), 4.11-3.98 (m, 3H), 3.77-3.63 (m, 1H).

### Example 26.4

### 5-[7-fluoro-4-(2-oxa-7-azaspiro[3.4]octan-7-yl)pyrazolo[4,3-c]pyridin-2-yl]-1H-pyrimidine-2,4-dione

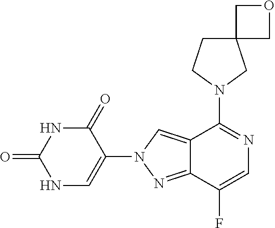 [SMILES: O=c1[nH]cc(-n2cc3c(N4CCC5(COC5)C4)ncc(F)c3n2)c(=O)[nH]1]

The title compound was synthesised from 4-chloro-2-(2,4-dimethoxypyrimidin-5-yl)-7-fluoro-pyrazolo[4,3-c]pyridine (Example 26 step 4) and 2-oxa-7-azaspiro[3.4]octane oxalic acid analogously to Example 26.1 step 1.

LC-MS-1 (Method 8B): Rt 1.25 mins; MS m/z 359.3=[M+H]+

1H NMR (400 MHz, DMSO) δ 11.76-11.31 (m, 2H), 9.05 (d, J=2.9 Hz, 1H), 8.21 (s, 1H), 7.60 (d, J=3.8 Hz, 1H), 4.62 (d, J=6.0 Hz, 2H), 4.54 (d, J=6.0 Hz, 2H), 3.92 (s, 2H), 3.67 (t, J=6.9 Hz, 2H), 2.29 (t, J=6.9 Hz, 2H).

### Example 26.5

### 5-[4-(7,7-difluoro-5-azaspiro[2.4]heptan-5-yl)-7-fluoro-pyrazolo[4,3-c]pyridin-2-yl]-1H-pyrimidine-2,4-dione

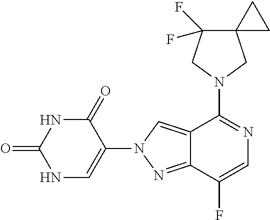 [SMILES: O=c1[nH]cc(-n2cc3c(N4CC(F)(F)C5(CC5)C4)ncc(F)c3n2)c(=O)[nH]1]

The title compound was synthesised from 4-chloro-2-(2,4-dimethoxypyrimidin-5-yl)-7-fluoro-pyrazolo[4,3-c]pyridine (Example 26 step 4) and 7,7-difluoro-5-azaspiro[2.4]heptane hydrochloride analogously to Example 26.1 step 1.

LC-MS-1 (Method 8A): Rt 1.61 mins; MS m/z 379.2=[M+H]+

1H NMR (400 MHz, DMSO-d6) δ 11.50 (m, 2H), 9.03 (d, J=2.8 Hz, 1H), 8.16 (s, 1H), 7.66 (d, J=3.7 Hz, 1H), 4.26 (t, J=12.7 Hz, 2H), 3.96 (s, 2H), 1.11-1.05 (m, 2H), 1.00-0.94 (m, 2H).

### Example 26.6

### 5-[4-[3,3-difluoro-4-[(4-methylmorpholin-2-yl)methoxy]pyrrolidin-1-yl]-7-fluoro-pyrazolo[4,3-c]pyridin-2-yl]-1H-pyrimidine-2,4-dione

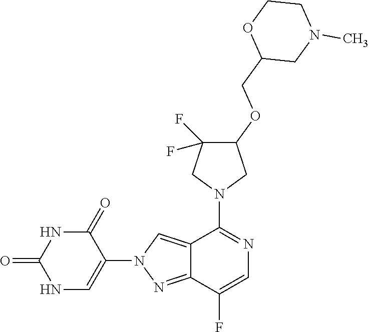 [SMILES: CN1CCOC(COC2CN(c3ncc(F)c4nn(-c5c[nH]c(=O)[nH]c5=O)cc34)CC2(F)F)C1]

The title compound was synthesised from 4-chloro-2-(2,4-dimethoxypyrimidin-5-yl)-7-fluoro-pyrazolo[4,3-c]pyridine (Example 26 step 4) and 2-[(4,4-difluoropyrrolidin-3-yl)oxymethyl]-4-methyl-morpholine (Intermediate N) analogously to Example 26.1 step 1.

LC-MS-1 (Method 8A): Rt 1.16 mins; MS m/z 482.3=[M+H]+

1H NMR (400 MHz, DMSO-d6) δ 11.49 (s, 2H), 9.09 (d, J=2.8 Hz, 1H), 8.16 (s, 1H), 7.64 (d, J=3.8 Hz, 1H), 4.46-4.38 (m, 1H), 4.19-3.99 (m, 3H), 3.85-3.70 (m, 3H), 3.70-3.58 (m, 3H), 3.55-3.47 (m, 1H), 2.72-2.67 (m, 1H), 2.17 (d, J=4.4 Hz, 3H), 2.03-1.94 (m, 1H), 1.85-1.75 (m, 1H).

### Example 27

### 5-[4-(3,3-Difluoro-4,4-dimethyl-pyrrolidin-1-yl)pyrazolo[4,3-c]pyridin-2-yl]-1H-pyrimidine-2,4-dione

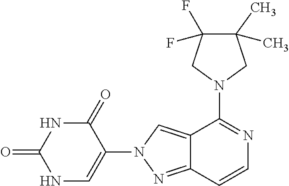 [SMILES: CC1(C)CN(c2nccc3nn(-c4c[nH]c(=O)[nH]c4=O)cc23)CC1(F)F]

### Step 1: 4-Azidopyridine-3-carbaldehyde

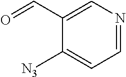 [SMILES: [N-]=[N+]=Nc1ccncc1C=O]

The title compound was synthesised according to the procedure of HOFFMANN LA ROCHE; BLENCH, Toby; GOODACRE, Simon; LAI, Yingjie; LIANG, Yun; MACLEOD, Calum; MAGNUSON, Steven; TSUI, Vickie; WILLIAMS, Karen; ZHANG, Birong. WO2012066061, 2012, A1, Page 79.

LC-MS-1 (Method 2B): Rt 0.76 mins; MS m/z 139.1=[M−N₂+H₂O+H]+

1H NMR (400 MHz, DMSO-d6) δ 10.23 (s, 1H), 8.85 (s, 1H), 8.73 (d, J=5.7 Hz, 1H), 7.56 (d, J=5.7 Hz, 1H).

### Step 2: 2-(2,4-Dimethoxypyrimidin-5-yl)pyrazolo[4,3-c]pyridine

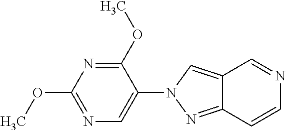 [SMILES: COc1ncc(-n2cc3cnccc3n2)c(OC)n1]

A solution of titanium tetrachloride (1.48 mL, 13.47 mmol) in DCM (10 mL) was added dropwise to a solution of 4-azidopyridine-3-carbaldehyde (step 1) (3.33 g, 22.45 mmol), 2,4-dimethoxypyrimidin-5-amine (Intermediate M) (3.48 g, 22.45 mmol) and triethylamine (9.39 mL, 67.34 mmol) in DCM (60 mL). After stirring for 30 mins, the reaction mixture was concentrated in vacuo. Toluene (60 mL) was then added to the reaction mixture which was stirred at 105° C. for 30 mins before being cooled to room temperature. The crude mixture was filtered through celite, eluting with DCM (300 mL), and the filtrate was concentrated in vacuo. Purification by chromatography on silica eluting with a gradient of 0 to 10% 1 M NH3 in MeOH in DCM afforded the title compound as a brown solid.

LC-MS-1 (Method 2B): Rt 0.94 mins; MS m/z 258.4=[M+H]+

1H NMR (400 MHz, DMSO-d6) δ 9.28 (d, J=1.4 Hz, 1H), 9.07 (s, 1H), 8.83 (s, 1H), 8.25 (d, J=6.3 Hz, 1H), 7.63-7.59 (m, 1H), 4.04 (s, 3H), 4.01 (s, 3H).

### Step 3: 2-(2,4-Dimethoxypyrimidin-5-yl)-5-oxido-pyrazolo[4,3-c]pyridin-5-ium

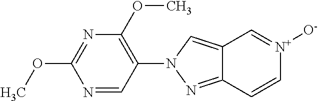 [SMILES: COc1ncc(-n2cc3c[n+]([O-])ccc3n2)c(OC)n1]

mCPBA (5.32 g, 21.57 mmol) was added to a solution of 2-(2,4-dimethoxypyrimidin-5-yl)pyrazolo[4,3-c]pyridine (step 2) (3.7 g, 14.38 mmol) in DCM (80 mL) at 0° C., before stirring at this temperature for 1.5 h. The reaction mixture was concentrated in vacuo and the residue purified by chromatography on silica eluting with a gradient of 0 to 20% 1 M NH3 in MeOH in DCM to afford the title compound as an orange solid.

LC-MS-2 (Method 2B): Rt 0.94 mins; MS m/z 273.9=[M+H]+

1H NMR (400 MHz, DMSO-d6) δ 8.86-8.83 (m, 1H), 8.82-8.78 (m, 2H), 7.92 (dd, J=7.5, 1.7 Hz, 1H), 7.80 (d, J=7.5 Hz, 1H), 4.04 (s, 3H), 4.01 (s, 3H).

### Step 4: 4-Chloro-2-(2,4-dimethoxypyrimidin-5-yl)pyrazolo[4,3-c]pyridine

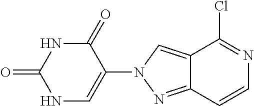 [SMILES: O=c1[nH]cc(-n2cc3c(Cl)nccc3n2)c(=O)[nH]1]

phosphorus (V) oxychloride (1.03 mL, 10.98 mmol) was added to a solution of 2-(2,4-dimethoxypyrimidin-5-yl)-5-oxido-pyrazolo[4,3-c]pyridin-5-ium (step 3) (1.0 g, 3.66 mmol) in DCE (30 mL) at 65° C. before stirring for 10 mins. The reaction mixture was then cooled to room temperature before dropwise addition of NaHCO₃ (6148.95 mg, 73.19 mmol) in water (20 mL). The phases were separated and the aqueous phase was extracted with 10% MeOH in DCM (30 mL). The combined organic extracts were washed with water (3×40 mL), dried over magnesium sulfate and concentrated in vacuo. The residue was purified by chromatography on silica eluting with a gradient of 0 to 80% EtOAc in DCM to afford the title compound as a white solid.

LC-MS-1 (Method 2B): Rt 1.19 mins; MS m/z 292.2=[M+H]+

1H NMR (400 MHz, DMSO-d6) δ 9.12 (s, 1H), 8.81 (s, 1H), 8.08 (d, J=6.3 Hz, 1H), 7.68 (d, J=6.3 Hz, 1H), 4.04 (s, 3H), 4.02 (s, 3H).

### Step 5: 5-[4-(3,3-Difluoro-4,4-dimethyl-pyrrolidin-1-yl)pyrazolo[4,3-c]pyridin-2-yl]-1H-pyrimidine-2,4-dione

Iodo(trimethyl)silane (0.38 mL, 2.67 mmol) was added to a solution of 4-chloro-2-(2,4-dimethoxypyrimidin-5-yl)pyrazolo[4,3-c]pyridine (step 4) (259 mg, 0.89 mmol) in chloroform (6 mL) and stirred at room temperature for 1 hour. Additional iodo(trimethyl)silane (0.63 mL, 4.45 mmol) was then added and the reaction was stirred at room temperature for a further 72 hours. Water (0.1 mL, 5.56 mmol) was then added before concentrating the mixture in vacuo.

DIPEA (0.66 mL, 3.79 mmol) was added to a solution of 3,3-difluoro-4,4-dimethyl-pyrrolidine hydrochloride (260 mg, 1.52 mmol) and the crude residue (200 mg, 0.76 mmol) in NMP (2 mL) before the mixture was heated at 120° C. for 90 mins. The reaction mixture was cooled to room temperature and the residue was purified by C18 reverse phase chromatography eluting with a gradient of 5 to 40% MeCN in H₂O (+0.1% NH4OH) to afford the title compound as an off-white solid.

LC-MS-1 (Method 8A): Rt 1.58 mins; MS m/z 363.2=[M+H]+

1H NMR (400 MHz, DMSO-d6) δ 11.79 (s, 1H), 11.59-11.42 (m, 1H), 8.99 (s, 1H), 8.16 (d, J=4.0 Hz, 1H), 7.67 (d, J=6.4 Hz, 1H), 6.77 (d, J=6.4 Hz, 1H), 4.20 (t, J=13.7 Hz, 2H), 3.77 (s, 2H), 1.30-1.13 (m, 6H).

### Example 28

### 5-[7-chloro-4-(3,3-difluoro-4,4-dimethyl-pyrrolidin-1-yl)pyrazolo[4,3-c]pyridin-2-yl]-1H-pyrimidine-2,4-dione

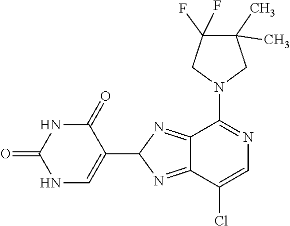 [SMILES: CC1(C)CN(c2ncc(Cl)c3c2=NC(c2c[nH]c(=O)[nH]c2=O)N=3)CC1(F)F]

### Step 1: 4-azido-5-chloro-pyridine-3-carbaldehyde

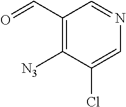 [SMILES: [N-]=[N+]=Nc1c(Cl)cncc1C=O]

4,5-Dichloropyridine-3-carbaldehyde (10 g, 56.82 mmol) and sodium azide (4.06 g, 62.45 mmol) were dispersed in DMF (31 mL) at room temperature. After 10 mins brine (200 ml) was added and extracted with EtOAc (2×100 ml). The combined organic extracts were dried over Na2SO4 and concentrated in vacuo. Purification by chromatography on silica eluting with a gradient of 1 to 50% EtOAc in petroleum ether afforded the title compound an off-white solid.

LC-MS-2 (Method 2A): Rt 1.15 min; MS m/z 182.9=[M+H]+

1H NMR (400 MHz, CDCl3) δ 10.36 (s, 1H), 8.84 (s, 1H), 8.65 (s, 1H).

### Step 2: 7-chloro-2-(2,4-dimethoxypyrimidin-5-yl)pyrazolo[4,3-c]pyridine

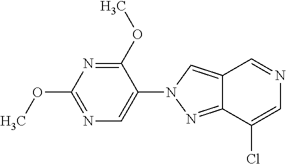 [SMILES: COc1ncc(-n2cc3cncc(Cl)c3n2)c(OC)n1]

4-azido-5-chloro-pyridine-3-carbaldehyde (step 1) (9.7 g, 53.13 mmol) and 2,4-dimethoxypyrimidin-5-amine (Intermediate M) (8.24 g, 53.13 mmol) and Et3N (22.22 mL, 159.39 mmol) were dispersed in DCM (200 mL). The solution was cooled in an ice bath before dropwise addition of titanium tetrachloride (3.5 mL, 31.88 mmol) in DCM (30 mL). The reaction was stirred in the ice bath for 30 min before warming to room temperature and stirring for 3 h. The reaction was then concentrated in vacuo and the residue was suspended in toluene (300 mL) and heated to 100° C. for 1 h. The reaction was cooled to room temperature and solvent removed in vacuo. The residue was taken into DCM (400 mL) and MeOH (200 mL) before filtering through celite, washing with 2:1 DCM:MeOH (250 mL). The filtrate was concentrated in vacuo. Purification by chromatography on silica eluting with a gradient of 0 to 10% MeOH in DCM afforded the title compound as an off-white solid.

LC-MS-1 (Method 2B): Rt 1.09 mins; MS m/z 292.1=[M+H]+

1H NMR (400 MHz, DMSO) δ 9.24 (s, 1H), 9.19 (s, 1H), 8.83 (s, 1H), 8.36 (s, 1H), 4.04 (s, 3H), 4.02 (s, 3H).

### Step 3: 7-chloro-2-(2,4-dimethoxypyrimidin-5-yl)-5-oxido-pyrazolo[4,3-c]pyridin-5-ium

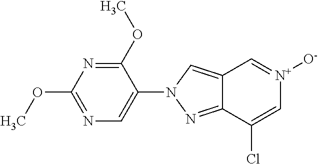 [SMILES: COc1ncc(-n2cc3c[n+]([O-])cc(Cl)c3n2)c(OC)n1]

mCPBA (16.6 g, 67.35 mmol) was added portion wise to a mixture of 7-chloro-2-(2,4-dimethoxypyrimidin-5-yl)pyrazolo[4,3-c]pyridine (step 2) (13 g, 44.57 mmol) in DCM (250 mL) at 0° C. The reaction was warmed to room temperature and stirred for 5 h. The reaction was concentrated in vacuo and purified by chromatography on silica eluting with a gradient of 0 to 10% MeOH in DCM affording the title compound as an off-white solid.

LC-MS-1 (Method 2A): Rt 0.86 mins; MS m/z 308.1=[M+H]+

1H NMR (400 MHz, DMSO) δ 8.91 (s, 1H), 8.87 (d, J=1.5 Hz, 1H), 8.79 (s, 1H), 8.32 (d, J=1.5 Hz, 1H), 4.03 (s, 3H), 4.01 (s, 3H).

### Step 4: 4,7-dichloro-2-(2,4-dimethoxypyrimidin-5-yl)pyrazolo[4,3-c]pyridine

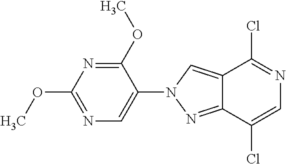 [SMILES: COc1ncc(-n2cc3c(Cl)ncc(Cl)c3n2)c(OC)n1]

To a mixture of 7-chloro-2-(2,4-dimethoxypyrimidin-5-yl)-5-oxido-pyrazolo[4,3-c]pyridin-5-ium (step 3) (7.91 g, 25.71 mmol) in DCE (300 mL) was added phosphorus (V) oxychloride (2.42 mL, 25.71 mmol). The reaction was warmed to 65° C. for 10 mins before further addition phosphorus (V) oxychloride (1.21 mL, 12.85 mmol) was added whilst heating. The reaction was stirred at 65° C. for 17 mins before cooling to room temperature. The reaction mixture was then poured over NaHCO3 (2.16 g, 25.71 mmol) in water (50 mL) cooled in an ice bath. The organic phase was then separated and the aqueous phase was extracted with DCM/MeOH (5%, 2×300 mL). The combined organic extracts were dried over magnesium sulfate and concentrated in vacuo. Purification by chromatography on silica eluting with 10% MeOH in DCM afforded the title compound as an off-white solid.

LC-MS-2 (Method 2A): Rt 1.47 mins; MS m/z 325.9=[M+H]+

1H NMR (400 MHz, DMSO) δ 9.26 (s, 1H), 8.81 (s, 1H), 8.23 (s, 1H), 4.04 (s, 3H), 4.03 (s, 3H).

### Step 5: 5-[7-chloro-4-(3,3-difluoro-4,4-dimethyl-pyrrolidin-1-yl)pyrazolo[4,3-c]pyridin-2-yl]-1H-pyrimidine-2,4-dione

To a stirred solution of 4,7-dichloro-2-(2,4-dimethoxypyrimidin-5-yl)pyrazolo[4,3-c]pyridine (step 4) (3.9 g, 11.96 mmol) in chloroform (80 mL) was added iodo(trimethyl)silane (8.51 mL, 59.79 mmol). Reaction was stirred at room temperature for 3 h, before cooling in an ice bath and addition of Water (5 mL). Reaction mixture was concentrated in vacuo, azeotroping with MeCN (3×10 mL). MeOH (50 mL) was added to the crude mixture giving a suspension. The solid was collected by filtration, washing with cold MeOH (20 mL) and ether (50 mL). The solid was dried in vacuo. A mixture of the residue, 3,3-difluoro-4,4-dimethyl-pyrrolidine hydrochloride (3.56 g, 20.76 mmol) and DIPEA (8.44 mL, 48.43 mmol) in NMP (12 mL) was heated to 120° C. for 80 mins. The reaction was cooled to room temperature and then added dropwise to ice cold water (25 mL). The mixture was partially concentrated in vacuo, forming a suspension. The mixture was cooled to 5° C. for 18 h, before the solid was collected by filtration, washing with water (25 mL), cold MeCN (2×25 mL) and diethyl ether (2×25 mL). This isolation was repeated 4 more times on the mother liquor. The combined solid was recrystallised from IPA to afford the title compound as a beige solid.

LC-MS-2 (Method 8A): Rt 1.98 mins; MS m/z 397.0=[M+H]+

1H NMR (400 MHz, DMSO) δ 11.79 (s, 1H), 11.59 (s, 1H), 9.05 (s, 1H), 8.19 (s, 1H), 7.73 (s, 1H), 4.18 (t, J=13.6 Hz, 2H), 3.75 (s, 2H), 1.20 (s, 6H).

### Example 28.1

### 5-[7-chloro-4-(7,7-difluoro-5-azaspiro[2.4]heptan-5-yl)pyrazolo[4,3-c]pyridin-2-yl]-1H-pyrimidine-2,4-dione

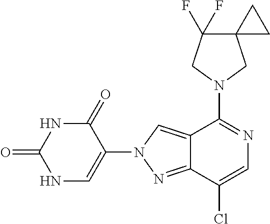 [SMILES: O=c1[nH]cc(-n2cc3c(N4CC(F)(F)C5(CC5)C4)ncc(Cl)c3n2)c(=O)[nH]1]

The title compound was synthesised from 4,7-dichloro-2-(2,4-dimethoxypyrimidin-5-yl)pyrazolo[4,3-c]pyridine (Example 28 step 4) and 7,7-difluoro-5-azaspiro[2.4]heptane hydrochloride analogously to Example 28 step 5.

LC-MS-2 (Method 8A): Rt 1.91 mins; MS m/z 395.0=[M+H]+

1H NMR (400 MHz, DMSO) δ 11.85-11.51 (m, 2H), 9.02 (s, 1H), 8.19 (s, 1H), 7.75 (s, 1H), 4.26 (t, J=12.5 Hz, 2H), 3.96 (s, 2H), 1.07-1.03 (m, 2H), 0.99-0.93 (m, 2H).

### Example 28.2

### 5-[7-chloro-4-(8,8-difluoro-6-azaspiro[3.4]octan-6-yl)pyrazolo[4,3-c]pyridin-2-yl]-1H-pyrimidine-2,4-dione

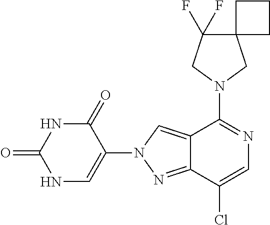 [SMILES: O=c1[nH]cc(-n2cc3c(N4CC(F)(F)C5(CCC5)C4)ncc(Cl)c3n2)c(=O)[nH]1]

The title compound was synthesised from 4,7-dichloro-2-(2,4-dimethoxypyrimidin-5-yl)pyrazolo[4,3-c]pyridine (Example 28 step 4) and 8,8-difluoro-6-azaspiro[3.4]octane hydrochloride analogously to Example 28 step 5.

LC-MS-2 (Method 8A): Rt 2.10 mins; MS m/z 409.0=[M+H]+

1H NMR (400 MHz, DMSO) δ 11.79 (s, 1H), 11.58 (s, 1H), 9.07 (s, 1H), 8.19 (s, 1H), 7.73 (s, 1H), 4.08 (t, J=13.0 Hz, 2H), 4.00 (s, 2H), 2.34-2.30 (m, 2H), 2.06-1.97 (m, 3H), 1.93-1.83 (m, 1H).

### Example 29

### 4-(3,3-difluoro-4,4-dimethyl-pyrrolidin-1-yl)-2-(2,4-dioxo-1H-pyrimidin-5-yl)pyrazolo[4,3-c]pyridine-7-carbonitrile

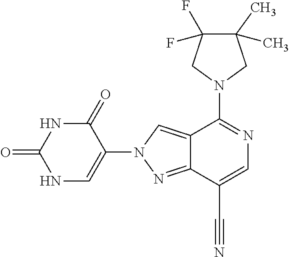 [SMILES: CC1(C)CN(c2ncc(C#N)c3nn(-c4c[nH]c(=O)[nH]c4=O)cc23)CC1(F)F]

### Step 1: 2-(2,4-dimethoxypyrimidin-5-yl)pyrazolo[4,3-c]pyridine-7-carbonitrile

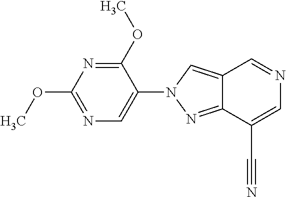 [SMILES: COc1ncc(-n2cc3cncc(C#N)c3n2)c(OC)n1]

A mixture of 7-chloro-2-(2,4-dimethoxypyrimidin-5-yl)pyrazolo[4,3-c]pyridine (Example 28 step 2) (1.65 g, 5.85 mmol), zinc cyanide (1.65 g, 14.03 mmol) and zinc dust (765 mg, 11.69 mmol) in DMA (40 mL) was degassed under a flow of N2. Pd(tBu3P)2 (299 mg, 0.58 mmol) was added before heating to 120° C. for 25 min.

The reaction was cooled to room temperature before dissolving in DCM (300 mL) and MeOH (200 mL) and passing through pad of celite, washing with 10% MeOH in DCM (500 mL). The filtrate was concentrated in vacuo to give a suspension. The residue was added dropwise to stirring water (250 mL) cooled in an ice bath, and the resulting solid was collected by filtration. The solid was triturated with ether (100 mL), and the solid was collected by filtration. This was dried in vacuo to afford the title compound as a white solid.

LC-MS-1 (Method 2A): Rt 1.01 mins; MS m/z 283.2=[M+H]+

1H NMR (400 MHz, DMSO) δ 9.57 (s, 1H), 9.34 (s, 1H), 8.91-8.82 (m, 2H), 4.05 (s, 3H), 4.03 (s, 3H).

### Step 2: 2-(2,4-dimethoxypyrimidin-5-yl)-5-oxido-pyrazolo[4,3-c]pyridin-5-ium-7-carbonitrile

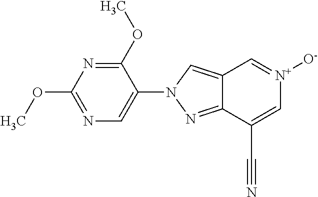 [SMILES: COc1ncc(-n2cc3c[n+]([O-])cc(C#N)c3n2)c(OC)n1]

To a solution of 2-(2,4-dimethoxypyrimidin-5-yl)pyrazolo[4,3-c]pyridine-7-carbonitrile (step 1) (1.5 g, 5.31 mmol) in DCM (150 mL) was added hydrogen peroxide (1.09 mL, 10.63 mmol) and then methyltrioxorhenium (132 mg, 0.53 mmol). The reaction was stirred at room temperature for 20 h. Hydrogen peroxide (1.09 mL, 10.63 mmol) and methyltrioxorhenium (10 mg, 0.04 mmol) were added and the reaction was stirred at rt for 2 h before further addition of hydrogen peroxide (1.09 mL, 10.63 mmol). After a further 18 h hydrogen peroxide (1.09 mL, 10.63 mmol) was added and reaction was stirred for a further 6 h. The reaction was cooled in an ice bath and MnO2 (462 mg, 5.31 mmol) was added then stirred for 30 mins. The mixture was filtered through celite, washing with DCM (100 mL) and then MeCN (100 mL). The filtrate was concentrated in vacuo to afford the title compound as an impure brown solid which was taken forward without further purification.

LC-MS-2 (Method 2A): Rt 0.95 mins; MS m/z 299.0=[M+H]+

### Step 3: 4-chloro-2-(2,4-dimethoxypyrimidin-5-yl)pyrazolo[4,3-c]pyridine-7-carbonitrile

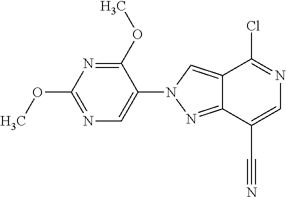 [SMILES: COc1ncc(-n2cc3c(Cl)ncc(C#N)c3n2)c(OC)n1]

A mixture of 2-(2,4-dimethoxypyrimidin-5-yl)-5-oxido-pyrazolo[4,3-c]pyridin-5-ium-7-carbonitrile (step 2) (1 g, 2.35 mmol) in DCE (14 mL) was added phosphorus (V) oxychloride (0.31 mL, 4.22 mmol). The reaction was then heated to 65° C. for 20 mins before cooling to room temperature. The reaction was then added dropwise to a solution of NaHCO3 (1.06 g, 12.62 mmol) in water (150 mL) cooled in an ice bath. The mixture was then extracted with DCM (150 mL), then re-extracted with DCM (100 mL). The combined organic extracts were washed with brine (150 mL), dried over magnesium sulfate and concentrated in vacuo. Purification by chromatography on silica eluting with a gradient of 0 to 100% EtOAc in petroleum ether afforded the title compound as a white solid.

LC-MS-2 (Method 2A): Rt 1.32 mins; MS m/z 316.9=[M+H]+

1H NMR (400 MHz, Chloroform-d) δ 9.06 (s, 1H), 8.87 (s, 1H), 8.48 (s, 1H), 4.22 (s, 3H), 4.12 (s, 3H).

### Step 4: 4-(3,3-difluoro-4,4-dimethyl-pyrrolidin-1-yl)-2-(2,4-dimethoxypyrimidin-5-yl)pyrazolo[4,3-c]pyridine-7-carbonitrile

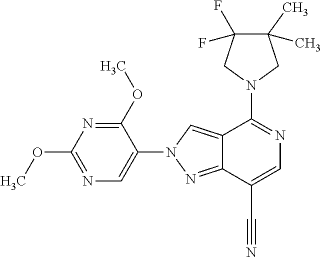

A suspension of 4-chloro-2-(2,4-dimethoxypyrimidin-5-yl)pyrazolo[4,3-c]pyridine-7-carbonitrile (step 3) (136 mg, 0.43 mmol), 3,3-difluoro-4,4-dimethyl-pyrrolidine hydrochloride (110 mg, 0.65 mmol) and DIPEA (0.37 mL, 2.15 mmol) in MeCN (2.5 mL) was heated to 70° C. for 4 h. The reaction was cooled to room temperature and concentrated in vacuo. The residue was taken into DCM (30 mL). The organics were washed with 50% aqueous brine (30 mL). The aqueous phase was extracted with DCM (30 mL) and the combined organic extracts were dried over magnesium sulfate and concentrated in vacuo. Purification by chromatography on silica eluting with a gradient of 0 to 100% EtOAc in DCM afforded the title compound as a white solid.

LC-MS-2 (Method 2A): Rt 1.40 mins; MS m/z 416.1=[M+H]+

1H NMR (400 MHz, DMSO-d6) δ 9.15 (s, 1H), 8.71 (s, 1H), 8.31 (s, 1H), 4.55-4.37 (m, 1H), 4.31-4.14 (m, 1H), 4.06-3.98 (m, 6H), 3.94 (br s, 1H), 3.77 (br s, 1H), 1.21 (s, 6H).

### Step 5: 4-(3,3-difluoro-4,4-dimethyl-pyrrolidin-1-yl)-2-(2,4-dioxo-1H-pyrimidin-5-yl)pyrazolo[4,3-c]pyridine-7-carbonitrile

2 M HCl (1.81 mL, 3.61 mmol) was added to a solution of 4-(3,3-difluoro-4,4-dimethyl-pyrrolidin-1-yl)-2-(2,4-dimethoxypyrimidin-5-yl)pyrazolo[4,3-c]pyridine-7-carbonitrile (step 4) (150 mg, 0.36 mmol) in Methanol (2 mL) and heated at 70° C. for 15 h before cooling to room temperature. 2 M HCl (1.81 mL, 3.61 mmol) was added and the reaction stirred at 70° C. for 24 h before cooling to room temperature. The resulting suspension was filtered and the solid was washed with H₂O (3×2 mL), MeCN (3×2 mL) and diethyl ether (3×2 mL) before drying in vacuo to afford the title compound as a white solid.

LC-MS-2 (Method 8A): Rt 2.60 mins; MS m/z 388.0=[M+H]+

1H NMR (400 MHz, DMSO-d6) δ 11.63 (s, 1H), 11.40 (s, 1H), 9.12 (s, 1H), 8.24 (s, 1H), 8.15 (s, 1H), 4.30 (t, J=13.4 Hz, 2H), 3.86 (s, 2H), 1.23 (s, 6H).

Broad signals were observed in the 1H NMR therefore date reported was recorded at 70° C.

### Example 30

### 5-[4-(3,3-difluoro-4,4-dimethyl-pyrrolidin-1-yl)pyrazolo[3,4-b]pyridine-2-yl]-1H-pyrimidine-2,4-dione

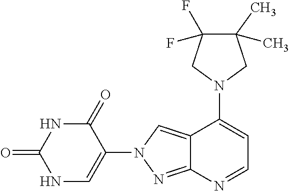 [SMILES: CC1(C)CN(c2ccnc3nn(-c4c[nH]c(=O)[nH]c4=O)cc23)CC1(F)F]

### Step 1: 2-chloro-4-(3,3-difluoro-4,4-dimethyl-pyrrolidin-1-yl)pyridine-3-carbaldehyde

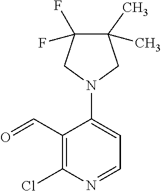 [SMILES: CC1(C)CN(c2ccnc(Cl)c2C=O)CC1(F)F]

A mixture of 2-chloro-4-fluoro-pyridine-3-carbaldehyde (2.75 g, 17.24 mmol), 2-chloro-4-fluoro-pyridine-3-carbaldehyde (2.75 g, 17.24 mmol) and triethylamine (7.21 mL, 51.71 mmol) in MeCN (50 mL) was stirred at room temperature for 45 mins. The reaction mixture was concentrated in vacuo. Purification by chromatography on silica eluting with a gradient of 0 to 25% EtOAc in DCM afforded the title compound as a as a yellow solid.

LC-MS-1 (Method 2A): Rt 1.36 mins; MS m/z 274.8=[M+H]+

1H NMR (400 MHz, DMSO) δ 10.32 (s, 1H), 8.09 (d, J=6.1 Hz, 1H), 6.89 (d, J=6.2 Hz, 1H), 3.74 (t, J=13.6 Hz, 2H), 3.32 (s, 2H), 1.13 (s, 6H).

### Step 2: 2-azido-4-(3,3-difluoro-4,4-dimethyl-pyrrolidin-1-yl)pyridine-3-carbaldehyde

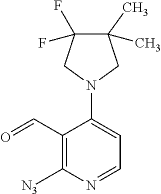 [SMILES: CC1(C)CN(c2ccnc(N=[N+]=[N-])c2C=O)CC1(F)F]

Sodium azide (1.33 g, 20.42 mmol) was added to a solution of 2-chloro-4-(3,3-difluoro-4,4-dimethyl-pyrrolidin-1-yl)pyridine-3-carbaldehyde (step 1) (5.61 g, 20.42 mmol) in DMF (60 mL) and stirred at 65° C. for 18 hrs. The reaction mixture was diluted with water (100 mL) and extracted into EtOAc (2×100 mL). The combined organic extracts were washed with water (100 mL), dried over magnesium sulfate and concentrated in vacuo. Purification by chromatography on silica eluting with a gradient of 0 to 20% EtOAc in DCM afforded the desired compound as a yellow solid.

LC-MS-1 (Method 2A): Rt 1.09 mins; MS m/z 282.1=[M+H]+

1H NMR (400 MHz, DMSO) δ 10.47 (s, 1H), 9.20 (d, J=7.9 Hz, 1H), 7.16 (d, J=7.8 Hz, 1H), 4.08 (t, J=13.6 Hz, 2H), 3.65 (s, 2H), 1.18 (s, 6H).

### Step 3: 5-[4-(3,3-difluoro-4,4-dimethyl-pyrrolidin-1-yl)pyrazolo[3,4-b]207yridine-2-yl]-1H-pyrimidine-2,4-dione

A solution of titanium tetrachloride (0.17 mL, 1.54 mmol) in DCM (5 mL) was added dropwise to a solution of 2-azido-4-(3,3-difluoro-4,4-dimethyl-pyrrolidin-1-yl)pyridine-3-carbaldehyde (step 2) (720 mg, 2.56 mmol), 2,4-dimethoxypyrimidin-5-amine (Intermediate M) (397 mg, 2.56 mmol) and triethylamine (1.07 mL, 7.68 mmol) in DCM (20 mL) and stirred at room temperature for 1 hr. The reaction mixture was then concentrated in vacuo, suspended in toluene (20 mL) and stirred at 115° C. for 18 hrs. The reaction mixture was then concentrated in vacuo. The residue was taken into methanol (25 mL) and 1M HCl (25.59 mL, 25.59 mmol) and stirred at 70° C. for 3 hrs, before concentrating in vacuo. The residue was taken into hot water (300 mL) and left to cool to rt overnight. The resulting solid was collected by filtration, and washed with water (50 mL) and MeCN (50 mL). This residue was purified by C18 reverse phase chromatography eluting with a gradient of 0 to 35% MeCN in H₂O (+0.1% NH4OH). Impure fraction were then repurified by C18 reverse phase chromatography eluting with gradients of 5 to 35% MeCN in H₂O (+0.1% NH4OH), and 5 to 35% MeCN in H₂O (+0.1% formic acid), affording impure material. The combined material was triturated with MeCN (3 mL), diethyl ether (5 mL), MeOH (1 mL) then diethyl ether (5 mL). The solid was dried in vacuo to afford the title compound as a beige solid.

LC-MS-1 (Method 8B): Rt 1.73 mins; MS m/z 363.3=[M+H]+

1H NMR (400 MHz, DMSO) δ 11.83-11.41 (m, 2H), 8.93 (s, 1H), 8.24-8.13 (m, 2H), 5.94 (d, J=5.4 Hz, 1H), 4.16 (t, J=14.1 Hz, 2H), 3.67 (s, 2H), 1.22 (s, 6H).

### Example 31

### 5-[4-(3,3-difluoro-4,4-dimethyl-pyrrolidin-1-yl)pyrazolo[3,4-c]pyridine-2-yl]-1H-pyrimidine-2,4-dione

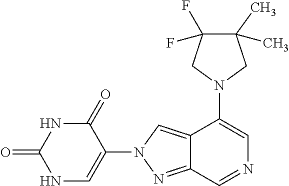 [SMILES: CC1(C)CN(c2cncc3nn(-c4c[nH]c(=O)[nH]c4=O)cc23)CC1(F)F]

### Step 1: 3-bromo-5-nitro-pyridine-4-carbaldehyde

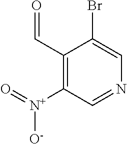 [SMILES: O=Cc1c(Br)cncc1[N+](=O)[O-]]

The title compound was synthesised according to the procedure of NOVARTIS AG, DU-CUNY, Lei; XIAO, Qitao; XUN, Guoliang; ZHENG, Qiangang, WO2018234978, 2018, page 42.

LC-MS-2 (Method 2B): Rt 0.98 mins; MS m/z 231.0=[M−H]−

1H NMR (400 MHz, Chloroform-d) δ 10.26 (s, 1H), 9.29 (s, 1H), 9.10 (s, 1H).

### Step 2: E-1-(3-bromo-5-nitro-4-pyridyl)-N-(2,4-dimethoxypyrimidin-5-yl)methanimine

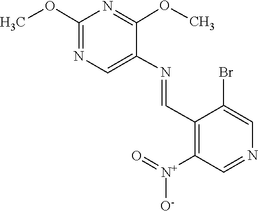 [SMILES: COc1ncc(/N=C/c2c(Br)cncc2[N+](=O)[O-])c(OC)n1]

A mixture of 3-bromo-5-nitro-pyridine-4-carbaldehyde (step 1) (3 g, 12.99 mmol), 2,4-dimethoxypyrimidin-5-amine (Intermediate M) (2.12 g, 13.64 mmol), pTSA (296 mg, 1.56 mmol) and molecular sieves in toluene (30 mL) was heated to 110° C. for 2.5 h. The reaction was cooled to room temperature and filtered, washing with toluene (2×30 mL). The filtrate was concentrated in vacuo to afford the title compounds as a brown solid.

LC-MS-1 (Method 2A): Rt 1.31 mins; MS m/z 368.2=[M+H]+

1H NMR (400 MHz, DMSO) δ 9.22-9.20 (m, 2H), 9.06 (s, 1H), 8.26 (s, 1H), 4.02 (s, 3H), 3.95 (s, 3H).

### Step 3: 4-bromo-2-(2,4-dimethoxypyrimidin-5-yl)pyrazolo[3,4-c]pyridine

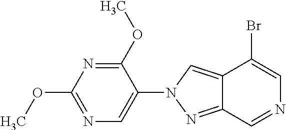 [SMILES: COc1ncc(-n2cc3c(Br)cncc3n2)c(OC)n1]

A mixture E-1-(3-bromo-5-nitro-4-pyridyl)-N-(2,4-dimethoxypyrimidin-5-yl)methanimine (step 2) (5 g, 13.58 mmol), triethyl phosphite (18.63 mL, 108.65 mmol) in toluene (80 mL) was heated to 80° C. for 3 days. The reaction was cooled to room temperature and diluted in DCM (200 mL) and washed with brine (40 mL). The organic phase was dried over magnesium sulfate and concentrated in vacuo. Purification by chromatography on silica eluting with a gradient of 0 to 100% EtOAc afforded impure material. The residue was triturated with diethyl ether (30 mL) and the solid was collected by filtration, washing with diethyl ether (2×5 mL). The solid was dried in vacuo to afford the title compound as a beige solid.

LC-MS-1 (Method 2A): Rt 1.22 mins; MS m/z 336.1=[M+H]+

1H NMR (400 MHz, DMSO) δ 9.29 (s, 1H), 8.94 (d, J=0.9 Hz, 1H), 8.81 (s, 1H), 8.32 (s, 1H), 4.04 (s, 3H), 4.02 (s, 3H).

### Step 4: 4-(3,3-difluoro-4,4-dimethyl-pyrrolidin-1-yl)-2-(2,4-dimethoxypyrimidin-5-yl)pyrazolo[3,4-c]pyridine

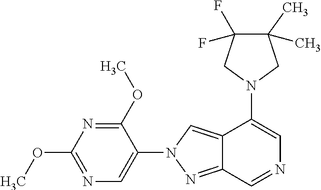

A mixture of 4-bromo-2-(2,4-dimethoxypyrimidin-5-yl)pyrazolo[3,4-c]pyridine (step 3) (500 mg, 1.49 mmol), 3,3-difluoro-4,4-dimethyl-pyrrolidine hydrochloride (306 mg, 1.78 mmol), cesium carbonate (969 mg, 2.97 mmol) and X-phos (142 mg, 0.30 mmol) in DMF (10 mL) was degassed under a flow of N2. Tris(dibenzylideneacetone)dipalladium (0) (136 mg, 0.15 mmol) was added and the reaction was heated to 100° C. for 5 h before cooling to room temperature. The reaction was added slowly to water (100 mL), and the precipitate was collected by filtration. The residue was taken into 5% MeOH in DCM (200 mL) and washed with water (40 mL). The aqueous phase was re-extracted with 5% MeOH in DCM (150 mL), and the combined organic extracts were dried over magnesium sulfate and concentrated in vacuo. Purification by chromatography on silica eluting with a gradient of 0 to 100% EtOAc in DCM afforded the title compound as an orange solid.

LC-MS-2 (Method 2A): Rt 1.09 mins; MS m/z 391.1=[M+H]+

1H NMR (400 MHz, DMSO) δ 9.00 (d, J=1.0 Hz, 1H), 8.74 (s, 1H), 8.53 (s, 1H), 7.20 (s, 1H), 4.12 (t, J=13.9 Hz, 2H), 4.03-4.00 (m, 6H), 3.62 (s, 2H), 1.21 (s, 6H).

### Step 5: 5-[4-(3,3-difluoro-4,4-dimethyl-pyrrolidin-1-yl)pyrazolo[3,4-c]210yridine-2-yl]-1H-pyrimidine-2,4-dione

A mixture of 4-(3,3-difluoro-4,4-dimethyl-pyrrolidin-1-yl)-2-(2,4-dimethoxypyrimidin-5-yl)pyrazolo[3,4-c]pyridine (step 4) (65 mg, 0.17 mmol) in 1 M HCl (2 mL, 2 mmol) and MeOH (1 mL) was heated to 70° C. for 90 min before cooling to room temperature. The reaction was concentrated in vacuo. Purification by C18 reverse phase chromatography eluting with a gradient of 5 to 35% MeCN in water (+0.1% formic acid) afforded the title compound as a yellow solid.

LC-MS-1 (Method 8A): Rt 1.78 mins; MS m/z 363.2=[M+H]+

1H NMR (400 MHz, DMSO) δ 11.84 (s, 1H), 11.63 (s, 1H), 9.08 (s, 1H), 8.61 (s, 1H), 8.24 (s, 1H), 7.18 (s, 1H), 4.13 (t, J=13.7 Hz, 2H), 3.65 (s, 2H), 1.22 (s, 6H).

### Preparation of Intermediates

### Intermediate A

### 4-Chloro-6-(2,4-dimethoxypyrimidin-5-yl)thieno[2,3-d]pyrimidine

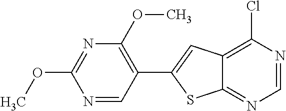 [SMILES: COc1ncc(-c2cc3c(Cl)ncnc3s2)c(OC)n1]

6-Bromo-4-chloro-thieno[2,3-d]pyrimidine (1.0 g, 4.01 mmol), (2,4-dimethoxypyrimidin-5-yl)boronic acid (0.77 g, 4.21 mmol) and cesium carbonate (3.91 g, 12.02 mmol) were dispersed in 1,4-dioxane (30 mL) and water (8 mL) and degassed under a flow of nitrogen. After 25 mins, Pd(dppf)Cl₂ (0.29 g, 0.40 mmol) was added and the reaction mixture was heated at 100° C. for 15 mins. The resulting mixture was cooled to room temperature before being concentrated in vacuo. The residue was dissolved into DCM (100 mL) and washed with water (75 mL). The aqueous phase was extracted with DCM (75 mL) and the combined organic extracts were dried over magnesium sulfate and concentrated in vacuo. Purification by chromatography on silica eluting with a gradient of 0 to 3% MeOH in DCM afforded the title compound as an off-white solid.

LC-MS-2 (Method 2B): Rt 1.58 mins; MS m/z 308.9=[M+H]+

1H NMR (400 MHz, DMSO-d6) δ 9.05 (s, 1H), 8.92 (s, 1H), 8.02 (s, 1H), 4.12 (s, 3H), 4.00 (s, 3H).

### Intermediate B

### 4,4-Difluoro-1-(2-pyrrolidin-3-yloxyethyl)piperidine dihydrochloride

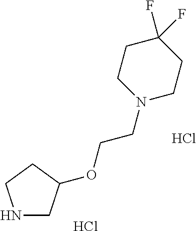 [SMILES: FC1(F)CCN(CCOC2CCNC2)CC1 ; Cl ; Cl]

### Step 1: Benzyl 3-[2-[tert-butyl(dimethyl)silyl]oxyethoxy]pyrrolidine-1-carboxylate

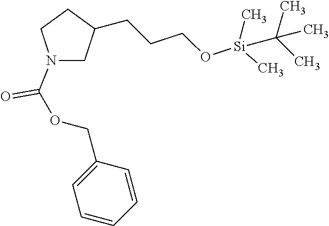 [SMILES: CC(C)(C)[Si](C)(C)OCCCC1CCN(C(=O)OCc2ccccc2)C1]

A solution of sodium hydride (60% dispersion in mineral oil) (8.34 g, 208.46 mmol) in DMF (300 mL) at 0° C. was treated portion wise with benzyl 3-hydroxypyrrolidine-1-carboxylate (14.41 g, 65.14 mmol) over 35 mins and stirred at this temperature for a further 15 mins. The mixture was allowed to warm to room temperature over 15 mins and then treated slowly with 2-bromoethoxy-tert-butyl-dimethyl-silane (41.74 mL, 195.43 mmol) added over 30 mins. The reaction mixture was allowed to warm to room temperature and stirred overnight. The resulting mixture was cooled to 0° C. and water (20 mL) was carefully added. After warming to room temperature, additional water (1000 mL) was added and the layers were separated. The aqueous portion was extracted with diethyl ether (3×500 mL) and the combined organic extracts were washed with brine (5×1000 mL), dried over magnesium sulfate and concentrated in vacuo. Purification by chromatography on silica eluting with a gradient of 0 to 50% EtOAc in petroleum ether afforded the title compound as a yellow liquid.

LC-MS-2 (Method 2A): Rt 2.01 mins; MS m/z 380.2=[M+H]+

1H NMR (500 MHz, CDCl3) δ 7.40-7.28 (m, 5H), 5.15-5.11 (m, 2H), 4.12-4.08 (m, 1H), 3.73 (t, J=5.2 Hz, 2H), 3.57-3.45 (m, 6H), 2.02 (br s, 1H), 1.97-1.87 (m, 1H), 0.88 (s, 9H), 0.05 (s, 6H).

### Step 2: Benzyl 3-(2-hydroxyethoxy)pyrrolidine-1-carboxylate

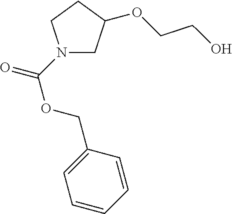 [SMILES: O=C(OCc1ccccc1)N1CCC(OCCO)C1]

Benzyl 3-[2-[tert-butyl(dimethyl)silyl]oxyethoxy]pyrrolidine-1-carboxylate (step 1) (6.5 g, 17.12 mmol) was dispersed in 2M HCl (128 mL, 256.87 mmol) and EtOH (130 mL) and stirred at 75° C. for 10 mins. The resulting mixture was cooled to room temperature before being diluted with water (300 mL) and extracted with EtOAc (2×300 mL). The organic extracts were combined, washed with brine (250 mL), dried over magnesium sulfate and concentrated in vacuo. Purification by chromatography on silica eluting with a gradient of 20 to 100% EtOAc in DCM afforded the title compound as a pale yellow liquid.

LC-MS-2 (Method 2A): Rt 1.20 mins; MS m/z 266.0=[M+H]+

1H NMR (500 MHz, CDCl3) δ 7.39-7.28 (m, 5H), 5.19-5.09 (m, 2H), 4.12-4.06 (m, 1H), 3.72 (t, J=4.5 Hz, 2H), 3.60-3.44 (m, 6H), 2.10-2.01 (m, 1H), 2.01-1.88 (m, 1H), 1.61 (br s, 1H).

### Step 3: Benzyl 3-[2-(p-tolylsulfonyloxy)ethoxy]pyrrolidine-1-carboxylate

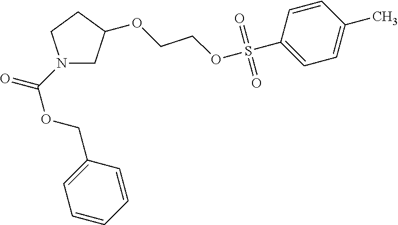 [SMILES: Cc1ccc(S(=O)(=O)OCCOC2CCN(C(=O)OCc3ccccc3)C2)cc1]

To a mixture of benzyl 3-(2-hydroxyethoxy)pyrrolidine-1-carboxylate (step 2) (3.7 g, 13.96 mmol) and triethylamine (15.6 mL, 111.69 mmol) in DCM (75 mL) was added tosyl chloride (5.3 g, 27.92 mmol) and the reaction mixture was stirred at room temperature for 4.5 h. The resulting mixture was diluted with DCM (100 mL) and washed with 50% sat. aq. NaHCO₃ (200 mL). The aqueous portion was further extracted with DCM (100 mL) and the combined organic extracts were dried over magnesium sulfate and concentrated in vacuo. Purification by chromatography on silica eluting with a gradient of 0 to 100% EtOAc in petroleum ether afforded the title compound as a pale yellow oil.

LC-MS-2 (Method 2B): Rt 1.64 mins; MS m/z 420.1=[M+H]+

1H NMR (500 MHz, CDCl3) δ 7.78 (d, J=7.9 Hz, 2H), 7.39-7.28 (m, 7H), 5.18-5.08 (m, 2H), 4.13 (t, J=4.7 Hz, 2H), 4.01 (br s, 1H), 3.66-3.57 (m, 2H), 3.54-3.33 (m, 4H), 2.44-2.40 (m, 3H), 2.00-1.81 (m, 2H).

### Step 4: Benzyl 3-[2-(4,4-difluoro-1-piperidyl)ethoxy]pyrrolidine-1-carboxylate

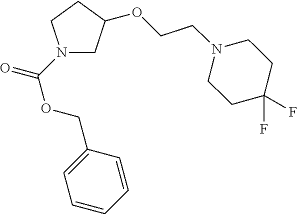 [SMILES: O=C(OCc1ccccc1)N1CCC(OCCN2CCC(F)(F)CC2)C1]

A mixture of benzyl 3-[2-(p-tolylsulfonyloxy)ethoxy]pyrrolidine-1-carboxylate (step 3) (0.5 g, 1.19 mmol), 4,4-difluoropiperidine hydrochloride (0.38 g, 2.39 mmol) and potassium carbonate (0.99 g, 7.15 mmol) in MeCN (10 mL) was heated at 75° C. overnight. Additional, 4,4-difluoropiperidine hydrochloride (376 mg, 2.39 mmol) was added and stirring continued for a further 4 h. A further portion of 4,4-difluoropiperidine hydrochloride (188 mg, 1.19 mmol) was added and stirring was continued for 1 h. The resulting mixture was allowed to cool to room temperature and concentrated in vacuo. The residue was diluted with DCM (100 mL) and washed with water (40 mL). The aqueous phase was re-extracted with DCM (60 mL) and the combined organic extracts were dried over magnesium sulfate and concentrated in vacuo. Purification by chromatography on silica eluting with EtOAc afforded the title compound as a colourless oil.

LC-MS-1 (Method 2B): Rt 1.46 mins; MS m/z 369.4=[M+H]+

1H NMR (500 MHz, CDCl3) δ 7.39-7.33 (m, 4H), 7.31 (td, J=6.4, 5.8, 2.5 Hz, 1H), 5.17-5.09 (m, 2H), 4.04 (qd, J=4.8, 3.0, 1.9 Hz, 1H), 3.55 (dd, J=12.1, 6.3 Hz, 3H), 3.49 (dd, J=12.2, 6.5 Hz, 3H), 2.62 (ddd, J=13.8, 6.9, 3.6 Hz, 6H), 2.04-1.91 (m, 6H).

### Step 5: 4,4-Difluoro-1-(2-pyrrolidin-3-yloxyethyl)piperidine dihydrochloride

A mixture of benzyl 3-[2-(4,4-difluoro-1-piperidyl)ethoxy]pyrrolidine-1-carboxylate (step 4) (365 mg, 0.99 mmol), 2M HCl (1.32 mL, 2.64 mmol) and 10% Pd/C (40 mg, 0.04 mmol) in EtOH (5 mL) was stirred under hydrogen for 1.5 h. The resulting mixture was filtered through Celite® (filter material), eluting with EtOAc and the filtrate was concentrated in vacuo to afford the title compound as a yellow oil.

1H NMR (500 MHz, Methanol-d4) δ 4.39 (t, J=4.1 Hz, 1H), 3.89 (t, J=4.8 Hz, 2H), 3.76 (s, 2H), 3.57 (d, J=12.5 Hz, 1H), 3.50-3.43 (m, 3H), 3.42-3.36 (m, 1H), 3.28-3.22 (m, 1H), 2.66 (s, 2H), 2.58 (t, J=14.6 Hz, 2H), 2.42-2.26 (m, 3H), 2.11 (dtd, J=14.0, 9.7, 4.2 Hz, 1H).

### Intermediate C

### 4-Chloro-6-(2,4-dimethoxypyrimidin-5-yl)furo[2,3-d]pyrimidine

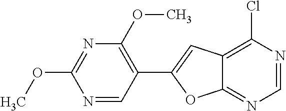 [SMILES: COc1ncc(-c2cc3c(Cl)ncnc3o2)c(OC)n1]

### Step 1: 4-Chloro-6-iodo-furo[2,3-d]pyrimidine

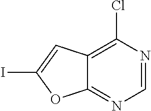 [SMILES: Clc1ncnc2oc(I)cc12]

To a solution of 4-chlorofuro[2,3-d]pyrimidine (1.0 g, 6.47 mmol) in THF (50 mL) at −78° C. was added LDA (2 M in THF/n-heptane/ethylbenzene, 3.56 mL, 7.12 mmol) dropwise and the reaction mixture was stirred at −78° C. for 1 h. To this mixture was added iodine (1.8 g, 7.12 mmol) in THF (10 mL) dropwise and after stirring at −78° C. for 15 mins, the mixture was allowed to warm to room temperature and stirred for 22 h. The resulting mixture was treated with 10% aq. sodium thiosulfate (60 mL) and stirred for 1 h. The THF was removed in vacuo and the aqueous phase was extracted with DCM (2×100 mL). The combined organic extracts were dried over magnesium sulfate and concentrated in vacuo. Trituration of the crude material with diethyl ether afforded a solid which was collected by filtration and dried to afford the title compound as a pale brown solid.

LC-MS-2 (Method 2A): Rt 1.42 mins; MS m/z 280.7=[M+H]+

1H NMR (400 MHz, DMSO-d6) δ 8.76 (s, 1H), 7.58 (s, 1H).

### Step 2: 4-Chloro-6-(2,4-dimethoxypyrimidin-5-yl)furo[2,3-d]pyrimidine

The title compound was prepared from 4-chloro-6-iodo-furo[2,3-d]pyrimidine (step 1) and (2,4-dimethoxypyrimidin-5-yl)boronic acid analogously to Intermediate A.

LC-MS-2 (Method 2A): Rt 1.51 mins; MS m/z 292.9=[M+H]+

1H NMR (400 MHz, DMSO-d6) δ 8.95 (s, 1H), 8.84 (s, 1H), 7.35 (s, 1H), 4.16 (s, 3H), 4.01 (s, 3H).

### Intermediate D

### 5-(4-Chlorothieno[2,3-d]pyrimidin-6-yl)-1H-pyrimidine-2,4-dione

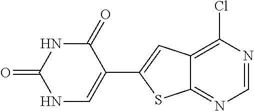 [SMILES: O=c1[nH]cc(-c2cc3c(Cl)ncnc3s2)c(=O)[nH]1]

### Step 1: 4-Chloro-6-(2,4-ditert-butoxypyrimidin-5-yl)thieno[2,3-d]pyrimidine

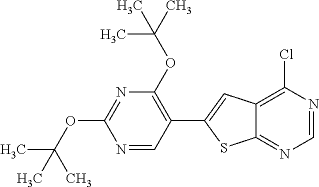 [SMILES: CC(C)(C)Oc1ncc(-c2cc3c(Cl)ncnc3s2)c(OC(C)(C)C)n1]

The title compound was prepared from 6-bromo-4-chloro-thieno[2,3-d]pyrimidine and (2,4-ditert-butoxypyrimidin-5-yl)boronic acid analogously to Intermediate A.

LC-MS-1 (Method 2A): Rt 2.00 mins; MS m/z 281.1=[M−C₈H₁₆+H]+

1H NMR (400 MHz, DMSO-d6) δ 9.04 (s, 1H), 8.90 (s, 1H), 8.04 (s, 1H), 1.72 (s, 9H), 1.61 (s, 9H).

### Step 2: 5-(4-Chlorothieno[2,3-d]pyrimidin-6-yl)-1H-pyrimidine-2,4-dione

A suspension of 4-chloro-6-(2,4-ditert-butoxypyrimidin-5-yl)thieno[2,3-d]pyrimidine (step 1) (2.57 g, 6.54 mmol) in DCM (50 mL) at 0° C. was treated dropwise with TFA (1.25 mL, 16.32 mmol) and the cooled mixture was stirred at this temperature for 30 mins and then allowed to warm to room temperature. After stirring at room temperature for 30 mins, the reaction mixture was re-cooled to 0° C. and treated with additional TFA (1.25 mL, 16.32 mmol). The mixture was stirred at this temperature for 10 mins and then at room temperature for 4 h. The resulting suspension was cooled and the solid was collected by filtration. The solid was washed with cold DCM (10 mL) followed by water (3×10 mL), MeCN (3×10 mL), diethyl ether (3×10 mL) and dried under vacuum to afford the title compound as a colourless solid.

LC-MS-1 (Method 2A): Rt 0.90 mins; MS m/z 281.1=[M+H]+

1H NMR (400 MHz, DMSO-d6) δ 11.83 (s, 1H), 11.74 (s, 1H), 8.84 (s, 1H), 8.62 (s, 1H), 7.99 (s, 1H).

### Intermediate E

### 4-Chloro-2-(2,4-dimethoxypyrimidin-5-yl)pyrazolo[3,4-d]pyrimidine

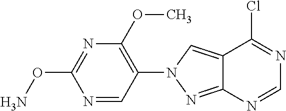 [SMILES: COc1nc(ON)ncc1-n1cc2c(Cl)ncnc2n1]

### Step 1: 4,6-Dichloropyrimidine-5-carbaldehyde (Purification of Commercial Reagent)

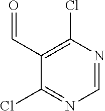

4,6-Dichloropyrimidine-5-carbaldehyde (10.55 g, 59.61 mmol) was suspended in DCM (30 mL) and stirred for 5 mins before filtering under vacuum and washing through with DCM (2×10 mL). The filtrate was concentrated in vacuo to afford the title compound as an off-white solid.

1H NMR (500 MHz, CDCl3) δ 10.46 (s, 1H), 8.89 (s, 1H).

### Step 1a: (2,4-Dimethoxypyrimidin-5-yl)hydrazine hydrochloride

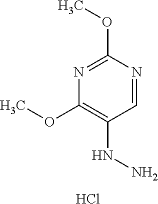

The title compound was prepared according to the procedure of NOVARTIS AG; BLANK, Jutta; BORDAS, Vincent; COTESTA, Simona; GUAGNANO, Vito; RUEEGER, Heinrich; VAUPEL, Andrea. US2014/349990, 2014, A1. Page 70, paragraph 0753; 0754.

LC-MS (Method 2B): Rt 0.33 mins; MS m/z 170.6=[M+H]+

1H NMR (400 MHz, DMSO-d6) δ 10.09 (s, 3H), 8.13 (s, 1H), 3.97 (s, 3H), 3.86 (s, 3H).

### Step 2: 4-Chloro-2-(2,4-dimethoxypyrimidin-5-yl)pyrazolo[3,4-d]pyrimidine

4,6-Dichloropyrimidine-5-carbaldehyde (step 1) (1.59 g, 9 mmol), (2,4-dimethoxypyrimidin-5-yl)hydrazine hydrochloride (step 1a) (2.38 g, 9.44 mmol) and TEA (4.94 mL, 35.42 mmol) were dissolved in anhydrous THF (55 mL) and stirred at 65° C. for 4 h. The resulting mixture was cooled to room temperature and filtered under vacuum. The filtrate was concentrated in vacuo to afford a black gum which was dissolved in DCM (50 mL). Water (10 mL) and more DCM (˜50 mL) were added and the mixture was filtered under vacuum. The layers were separated and the aqueous layer was further extracted with DCM (2×20 mL). The combined organic extracts were washed with water (2×10 mL), passed through a hydrophobic frit and concentrated in vacuo. Attempts to dissolve the crude material in DCM (˜5 mL) resulted in the formation of a precipitate. Additional DCM (10 mL) was added and the resulting suspension was filtered and the solids were dried in a vacuum oven to afford the title compound as a brown solid.

LC-MS (Method 2B): Rt 0.57 mins; MS m/z 293.1/295.1=[M+H]+

1H NMR (500 MHz, CDCl3) δ 9.08 (s, 1H), 8.89 (s, 1H), 8.78 (s, 1H), 4.22 (s, 3H), 4.11 (s, 3H).

### Intermediate F

### 5-(4-Chlorothieno[2,3-b]pyridin-2-yl)-1H-pyrimidine-2,4-dione hydrochloride

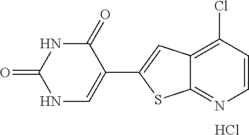 [SMILES: O=c1[nH]cc(-c2cc3c(Cl)ccnc3s2)c(=O)[nH]1 ; Cl]

### Step 1: 4-Chloro-2-iodo-thieno[2,3-b]pyridine

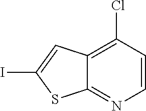 [SMILES: Clc1ccnc2sc(I)cc12]

The title compound was prepared from 4-chlorothieno[2,3-b]pyridine and iodine analogously to Intermediate C step 1.

LC-MS-2 (Method 2A): Rt 1.79 mins; MS m/z 295.7=[M+H]+

1H NMR (400 MHz, DMSO-d6) δ 8.48 (d, J=5.1 Hz, 1H), 7.85 (s, 1H), 7.55 (d, J=5.1 Hz, 1H).

### Step 2: 4-Chloro-2-(2,4-dimethoxypyrimidin-5-yl)thieno[2,3-b]pyridine

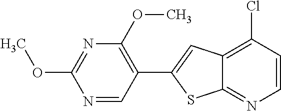 [SMILES: COc1ncc(-c2cc3c(Cl)ccnc3s2)c(OC)n1]

The title compound was prepared from 4-chloro-2-iodo-thieno[2,3-b]pyridine (step 1) and (2,4-dimethoxypyrimidin-5-yl)boronic acid analogously to Intermediate C step 2.

LC-MS-2 (Method 2A): Rt 1.68 mins; MS m/z 307.9=[M+H]+

1H NMR (400 MHz, DMSO-d6) δ 8.90 (s, 1H), 8.47 (d, J=5.1 Hz, 1H), 7.86 (s, 1H), 7.58 (d, J=5.1 Hz, 1H), 4.09 (s, 3H), 3.97 (s, 3H).

### Step 3: 5-(4-Chlorothieno[2,3-b]pyridin-2-yl)-1H-pyrimidine-2,4-dione hydrochloride

The title compound was prepared from 4-chloro-2-(2,4-dimethoxypyrimidin-5-yl)thieno[2,3-b]pyridine (step 2) and 1M HCl analogously to Example 7 step 2.

LC-MS-2 (Method 2A): Rt 1.12 mins; MS m/z 279.9=[M+H]+

1H NMR (400 MHz, DMSO-d6) δ 11.70 (s, 1H), 11.64 (s, 1H), 8.50-8.36 (m, 2H), 7.92 (s, 1H), 7.54 (d, J=5.2 Hz, 1H).

### Intermediate G

### 5-(4-Chlorofuro[2,3-d]pyrimidin-6-yl)-1H-pyrimidine-2,4-dione

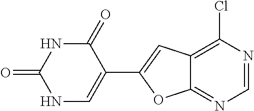 [SMILES: O=c1[nH]cc(-c2cc3c(Cl)ncnc3o2)c(=O)[nH]1]

### Step 1: 4-Chloro-6-(2,4-ditert-butoxypyrimidin-5-yl)furo[2,3-d]pyrimidine

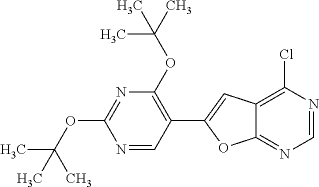 [SMILES: CC(C)(C)Oc1ncc(-c2cc3c(Cl)ncnc3o2)c(OC(C)(C)C)n1]

The title compound was prepared from 4-chloro-6-iodo-furo[2,3-d]pyrimidine (Intermediate C step 1) and (2,4-di-tert-butoxypyrimidin-5-yl)boronic acid analogously to Intermediate C step 2.

LC-MS-2 (Method 2A): Rt 2.07 mins; MS m/z 377.1=[M+H]+

1H NMR (400 MHz, Chloroform-d) δ 8.92 (s, 1H), 8.71 (s, 1H), 7.15 (s, 1H), 1.79 (s, 9H), 1.66 (s, 9H).

### Step 2: 5-(4-Chlorofuro[2,3-d]pyrimidin-6-yl)-1H-pyrimidine-2,4-dione

The title compound was prepared from 4-chloro-6-(2,4-ditert-butoxypyrimidin-5-yl)furo[2,3-d]pyrimidine (step 1) and TFA analogously to Intermediate D step 2.

LC-MS-2 (Method 2A): Rt 1.02 mins; MS m/z 264.9=[M+H]+

S1H NMR (400 MHz, DMSO-d6) δ 11.75 (br s, 1H), 11.71 (s, 1H), 8.77 (s, 1H), 8.19 (s, 1H), 7.30 (s, 1H).

### Intermediate H

### 2-[(4,4-Difluoropyrrolidin-3-yl)oxymethyl]-4-methyl-morpholine dihydrochloride

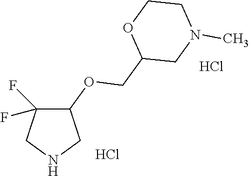 [SMILES: CN1CCOC(COC2CNCC2(F)F)C1 ; Cl ; Cl]

### Step 1: (4-Methylmorpholin-2-yl)methyl 4-methylbenzenesulfonate

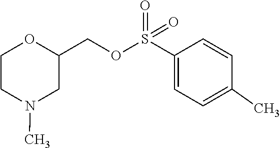 [SMILES: Cc1ccc(S(=O)(=O)OCC2CN(C)CCO2)cc1]

To a solution of (4-methylmorpholin-2-yl)methanol (1.90 g, 14.49 mmol) in THF (10 mL) at 0° C. was added triethylamine (2.02 mL, 14.49 mmol) and the mixture was stirred for 10 mins. 4-Methylbenzenesulfonyl chloride (2.76 g, 14.49 mmol) was added and stirring continued at 0° C. for 15 mins and then the mixture was allowed to warm to room temperature overnight.

The resulting mixture was filtered, the solid was washed with EtOAc (50 mL) and the filtrate was concentrated in vacuo. The residue was dissolved in EtOAc (50 mL) and washed with sat. aq. NaHCO₃ solution (20 mL), dried over magnesium sulfate and concentrated in vacuo to afford the title compound as a brown solid.

LC-MS-2 (Method 2B): Rt 1.07 mins; MS m/z 285.9=[M+H]+

1H NMR (500 MHz, CDCl3) δ 7.79 (dt, 2H), 7.34 (d, J=8.0 Hz, 2H), 4.05-3.95 (m, 2H), 3.84-3.79 (m, 1H), 3.77-3.71 (m, 1H), 3.61 (td, J=11.4, 2.5 Hz, 1H), 2.69 (d, J=11.2, 2.0 Hz, 1H), 2.60 (d, J=11.6, 2.1 Hz, 1H), 2.44 (s, 3H), 2.27 (s, 3H), 2.10 (td, J=11.4, 3.4 Hz, 1H), 1.89 (t, J=10.7 Hz, 1H).

### Step 2: tert-Butyl 3,3-difluoro-4-[(4-methylmorpholin-2-yl)methoxy]pyrrolidine-1-carboxylate

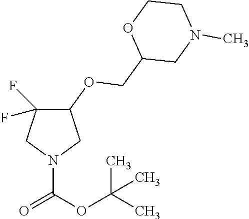 [SMILES: CN1CCOC(COC2CN(C(=O)OC(C)(C)C)CC2(F)F)C1]

A stirred solution of tert-butyl 3,3-difluoro-4-hydroxy-pyrrolidine-1-carboxylate (950 mg, 4.26 mmol) in toluene (15 mL) was treated with (4-methylmorpholin-2-yl)methyl 4-methylbenzenesulfonate (step 1) (1.33 g, 4.68 mmol) and tetrabutylammonium bromide (412 mg, 1.28 mmol) and the reaction mixture was heated at 80° C. for 22 h. The resulting mixture was cooled to room temperature and partitioned between EtOAc (50 mL) and water (20 mL). The layers were separated and the aqueous portion was further extracted with EtOAc (2×50 mL). The combined organic extracts were washed with brine, dried over magnesium sulfate and concentrated in vacuo. Purification by chromatography on silica eluting with a gradient of 0 to 10% MeOH in DCM afforded the title compound as an orange oil.

1H NMR (500 MHz, DMSO-d6) δ 4.24-4.16 (m, 1H), 3.77 (ddd, J=11.3, 3.4, 1.9 Hz, 1H), 3.72-3.47 (m, 7H), 3.36-3.27 (m, 1H), 2.70-2.63 (m, 1H), 2.57 (dd, J=11.3, 2.3 Hz, 1H), 2.19 (s, 3H), 1.99 (td, J=11.3, 3.3 Hz, 1H), 1.82-1.71 (m, 1H), 1.43 (s, 9H).

### Step 3: 2-[(4,4-Difluoropyrrolidin-3-yl)oxymethyl]-4-methyl-morpholine dihydrochloride

A solution of tert-butyl 3,3-difluoro-4-[(4-methylmorpholin-2-yl)methoxy]pyrrolidine-1-carboxylate (step 2) (964 mg, 2.87 mmol) in 4M HCl in 1,4-dioxane (7.16 mL, 28.66 mmol) and 1,4-dioxane (7 mL) was stirred at room temperature for 2 h. The resulting mixture was concentrated in vacuo and the crude material was triturated with diethyl ether to give a gummy colourless solid. The solid and liquors were recombined and concentrated in vacuo to afford the title compound as a colourless gummy foam.

1H NMR (500 MHz, Acetic Acid-d4) δ 4.33 (dd, J=12.7, 6.3 Hz, 1H), 4.25 (t, J=12.4 Hz, 1H), 4.17-4.09 (m, 2H), 4.03-3.82 (m, 5H), 3.81-3.73 (m, 1H), 3.70 (d, J=11.9 Hz, 1H), 3.58 (t, J=11.9 Hz, 1H), 3.45 (t, 1H), 3.26 (t, J=11.0 Hz, 1H), 3.18-3.08 (m, 1H), 2.97 (d, J=5.1 Hz, 3H).

### Intermediate I

### 4-Methyl-2-(pyrrolidin-3-yloxymethyl)morpholine

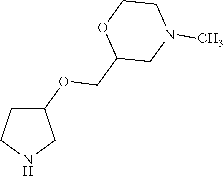

### Step 1: Benzyl 3-[(4-methylmorpholin-2-yl)methoxy]pyrrolidine-1-carboxylate

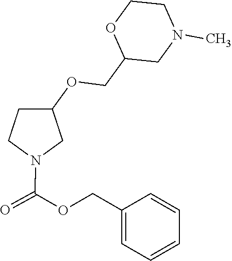 [SMILES: CN1CCOC(COC2CCN(C(=O)OCc3ccccc3)C2)C1]

The title compound was prepared from (4-methylmorpholin-2-yl)methyl 4-methylbenzenesulfonate (Intermediate H step 1) and benzyl 3-hydroxypyrrolidine-1-carboxylate analogously to Intermediate H step 2.

LC-MS-2 (Method 2A): Rt 1.01 mins; MS m/z 335.3=[M+H]+

1H NMR (500 MHz, CDCl3) δ 7.39-7.27 (m, 5H), 5.17-5.08 (m, 2H), 4.07 (h, J=3.3, 2.5 Hz, 1H), 3.91-3.85 (m, 1H), 3.76-3.66 (m, 2H), 3.57-3.37 (m, 6H), 2.71 (dd, J=40.6, 11.4 Hz, 2H), 2.31 (s, 3H), 2.20-2.09 (m, 1H), 2.09-1.99 (m, 1H), 1.98-1.85 (m, 2H).

### Step 2: 4-Methyl-2-(pyrrolidin-3-yloxymethyl)morpholine

A flask containing benzyl 3-[(4-methylmorpholin-2-yl)methoxy]pyrrolidine-1-carboxylate (step 1) (460 mg, 1.38 mmol) in EtOH (7 mL) was purged with nitrogen and treated with Pd/C (10%, 47 mg, 0.44 mmol). The flask was sealed and the contents evacuated and backfilled with nitrogen (3× cycles) followed by hydrogen (3 cycles). The reaction mixture was stirred at room temperature for 2 h. The resulting mixture was filtered through a pad of Celite®, eluting with EtOH (50 mL). The filtrate was concentrated in vacuo to afford the title compound as an orange oil.

¹H NMR (500 MHz, CDCl₃) δ 4.08-3.96 (m, 1H), 3.93-3.80 (m, 1H), 3.78-3.54 (m, 2H), 3.51-3.40 (m, 1H), 3.40-3.31 (m, 1H), 3.12-2.99 (m, 2H), 2.84-2.75 (m, 2H), 2.71 (dt, J=11.3, 2.0 Hz, 1H), 2.63 (dq, J=12.1, 2.3 Hz, 1H), 2.28 (s, 3H), 2.21-2.07 (m, 1H), 1.96-1.74 (m, 3H).

### Intermediate J

### 1-(2-Pyrrolidin-3-yloxyethyl)piperidine

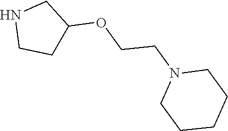 [SMILES: C1CCN(CCOC2CCNC2)CC1]

### Step 1: 1-(2-Chloroethyl)piperidine

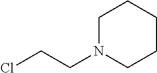

1-(2-Chloroethyl)piperidine hydrochloride (50.0 g, 271.58 mmol) was dissolved in water (100 mL) and basified with Na₂CO₃ (57.6 g, 543.15 mmol). The solution was extracted with diethyl ether (4×100 mL) and the combined organic extracts were dried over magnesium sulfate and concentrated in vacuo to afford the title compound as a colourless oil.

1H NMR (400 MHz, CDCl3) δ 3.58 (td, J=7.3, 3.7 Hz, 2H), 2.69 (td, J=7.3, 3.6 Hz, 2H), 2.51-2.38 (m, 4H), 1.64-1.54 (m, 4H), 1.48-1.38 (m, 2H).

### Step 2: Benzyl 3-[2-(1-piperidyl)ethoxy]pyrrolidine-1-carboxylate

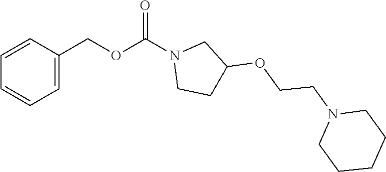 [SMILES: O=C(OCc1ccccc1)N1CCC(OCCN2CCCCC2)C1]

To a stirred slurry of sodium hydride (60% dispersion in mineral oil) (4.34 g, 108.47 mmol) in DMF (50 mL) at 0° C. under nitrogen was added dropwise a solution of benzyl 3-hydroxypyrrolidine-1-carboxylate (20.0 g, 90.4 mmol) in DMF (50 mL). The reaction mixture was stirred for 30 mins and then treated dropwise with 1-(2-chloroethyl)piperidine (step 1) (30.9 g, 198.87 mmol) in DMF (50 mL). The mixture was allowed to warm to room temperature and stirred under a flow of nitrogen for 20 h. The reaction was quenched slowly with water (100 mL) and extracted with EtOAc (4×120 mL). The combined organic extracts were washed with sat. aq. NaHCO₃ (100 mL), brine (100 mL), dried over sodium sulfate and concentrated in vacuo. Purification by chromatography on silica eluting with 0 to 10% MeOH in DCM afforded the title compound as an orange oil.

LC-MS (Method 2B): Rt 0.81 mins; MS m/z 333.4=[M+H]+

1H NMR (500 MHz, CDCl3) δ 7.30-7.18 (m, 5H), 5.07-4.98 (m, 2H), 3.98-3.91 (m, 1H), 3.53-3.44 (m, 2H), 3.42-3.34 (m, 4H), 2.49-2.42 (m, 2H), 2.39-2.27 (m, 4H), 1.97-1.87 (m, 1H), 1.86-1.77 (m, 1H), 1.55-1.44 (m, 4H), 1.38-1.28 (m, 2H).

### Step 3: 1-(2-Pyrrolidin-3-yloxyethyl)piperidine

A solution of benzyl 3-[2-(1-piperidyl)ethoxy]pyrrolidine-1-carboxylate (step 2) (2.3 g, 6.86 mmol) in EtOH (50 mL) was degassed with nitrogen (3 cycles) and treated with 10% Pd on carbon (109 mg, 0.10 mmol). The reaction mixture was degassed further with nitrogen (3 cycles) followed by 1 atmosphere of hydrogen (3 cycles) and stirred at room temperature for 20 h. Additional 10% Pd on carbon (10%, 109 mg, 0.10 mmol) was added and stirring continued under hydrogen at room temperature for 6 h. The resulting mixture was filtered through glass fibre filter paper and the filtrate was concentrated in vacuo to afford the title compound as a dark yellow oil.

LC-MS (Method 2B): Rt 0.50 mins; MS m/z 199.2=[M+H]+

1H NMR (400 MHz, DMSO-d6) δ 3.96 (tt, J=5.6, 2.8 Hz, 1H), 3.65-3.62 (m, 2H), 3.44-3.41 (m, 2H), 2.88-2.80 (m, 2H), 2.80-2.71 (m, 2H), 2.35-2.29 (m, 4H), 1.81-1.72 (m, 1H), 1.72-1.64 (m, 1H), 1.49-1.42 (m, 4H), 1.39-1.30 (m, 2H

### Intermediate K

### 1-[6-(2,4-Dimethoxypyrimidin-5-yl)furo[2,3-d]pyrimidin-4-yl]-4,4-difluoro-pyrrolidin-3-ol

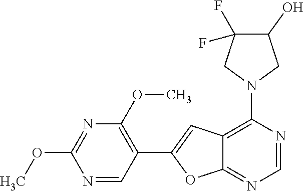 [SMILES: COc1ncc(-c2cc3c(N4CC(O)C(F)(F)C4)ncnc3o2)c(OC)n1]

A mixture of DIPEA (2.23 mL, 12.81 mmol), 4-chloro-6-(2,4-dimethoxypyrimidin-5-yl)furo[2,3-d]pyrimidine (750 mg, 2.56 mmol) (Intermediate C) and 4,4-difluoropyrrolidin-3-ol hydrochloride (613 mg, 3.84 mmol) in MeCN (25 mL) was stirred at 80° C. overnight. The mixture was cooled to 0° C., and the solid was collected by filtration, washing with MeCN (20 mL) and diethyl ether (20 mL) to afford the title compound as a grey solid.

LC-MS-2 (Method 2A): Rt 1.45 mins; MS m/z 380.0=[M+H]+

1H NMR (400 MHz, DMSO-d6) δ 8.79 (s, 1H), 8.33 (s, 1H), 7.35 (s, 1H), 6.32-6.11 (m, 1H), 4.43 (s, 1H), 4.24-4.02 (m, 5H), 3.98 (s, 3H), 3.85-3.74 (m, 1H).

### Intermediate L

### 2-Bromo-4-chloro-pyrazolo[1,5-a]pyrazine

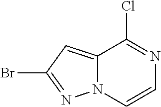 [SMILES: Clc1nccn2nc(Br)cc12]

### Step 1: 3-Bromo-N-(2,2-dimethoxyethyl)-1H-pyrazole-5-carboxamide

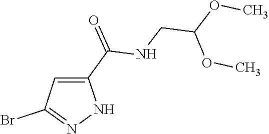

To a mixture of 3-bromo-1H-pyrazole-5-carboxylic acid (12.5 g, 65.45 mmol), 2,2-dimethoxyethanamine (8.56 mL, 78.54 mmol) and triethylamine (18.25 mL, 130.9 mmol) in MeCN (500 mL) at 0° C. was added HATU (37.31 g, 98.18 mmol) portion wise. The reaction was warmed to room temperature and stirred for 20 h. Additional 2,2-dimethoxyethanamine (3.57 mL, 32.73 mmol) was added and the reaction cooled to 0° C. before the addition of a further portion of HATU (12.44 g, 32.73 mmol). The reaction was stirred at room temperature for 72 h before concentrating in vacuo. The residue was dissolved in EtOAc (200 mL) and washed with 1M HCl (200 mL) and sat. aq. NaHCO₃ (200 mL). The organic phase was dried over magnesium sulfate and concentrated in vacuo. Purification by chromatography on silica eluting with a gradient of 0 to 100% EtOAc in petroleum ether afforded the title compound as a colourless solid.

LC-MS-2 (Method 2A): Rt 1.00 mins; MS m/z 275.9/278.0=[M−H]−

1H NMR (400 MHz, DMSO-d6) δ 13.94-13.83 (m, 1H), 8.58 (s, 1H), 6.95 (s, 1H), 4.46 (t, J=5.4 Hz, 1H), 3.34 (m, 2H), 3.30 (s, 6H).

### Step 2: 2-Bromo-7-hydroxy-6,7-dihydro-5H-pyrazolo[1,5-a]pyrazin-4-one

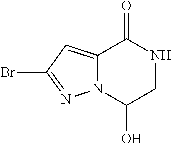 [SMILES: O=C1NCC(O)n2nc(Br)cc21]

A mixture of 3-bromo-N-(2,2-dimethoxyethyl)-1H-pyrazole-5-carboxamide (step 1) (500 mg, 1.8 mmol) and 35% HCl (0.48 mL, 5.39 mmol) in 1,4-dioxane (2 mL) was stirred at room temperature for 5 mins before concentrating in vacuo. Water (50 mL) was added, and the formed solid collected by filtration. The solid was suspended in MeCN and concentrated in vacuo to afford the title compound as a colourless solid.

LC-MS-2 (Method 2A): Rt 0.77 mins; MS m/z 232.0/233.8=[M+H]+

1H NMR (400 MHz, DMSO-d6) δ 8.30 (d, J=4.7 Hz, 1H), 7.39 (d, J=5.9 Hz, 1H), 6.86 (s, 1H), 5.76-5.71 (m, 1H), 3.79 (dd, J=13.8, 3.3 Hz, 1H), 3.41 (dd, J=13.8, 1.9 Hz, 1H).

### Step 3: 2-Bromo-5H-pyrazolo[1,5-a]pyrazin-4-one

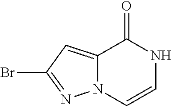 [SMILES: O=c1[nH]ccn2nc(Br)cc12]

To a mixture of 2-bromo-7-hydroxy-6,7-dihydro-5H-pyrazolo[1,5-a]pyrazin-4-one (step 2) (3.1 g, 13.36 mmol) in toluene (30 mL) was added thionyl chloride (0.97 mL, 13.36 mmol) followed by DMF (150 uL, 1.95 mmol). The reaction was heated at 105° C. for 90 mins before cooling to 0° C. The formed solid was collected by filtration before suspending in MeCN and concentrating in vacuo to afford the title compound as a colourless solid.

LC-MS-2 (Method 2A): Rt 0.97 mins; MS m/z 213.9/215.8=[M+H]+

1H NMR (400 MHz, DMSO-d6) δ 11.42 (s, 1H), 7.67 (d, J=5.8 Hz, 1H), 7.15 (s, 1H), 6.93-6.87 (m, 1H).

### Step 4: 2-Bromo-4-chloro-pyrazolo[1,5-a]pyrazine

A mixture of 2-bromo-5H-pyrazolo[1,5-a]pyrazin-4-one (step 3) (2.57 g, 12.01 mmol) in phosphorus (V) oxychloride (30 mL, 319.18 mmol) was heated at 100° C. for 15 mins before cooling to room temperature. The phosphorus (V) oxychloride was removed in vacuo before addition of water (50 mL) caused a solid to form. The suspension was stirred at room temperature for 5 mins and then 10 mins at 0° C. The solid was then collected by filtration, before dissolving in MeCN and concentrating in vacuo to afford the title compound as a beige solid.

LC-MS-2 (Method 2A): Rt 1.41 mins; MS m/z 231.8/233.8=[M+H]+

1H NMR (400 MHz, DMSO-d6) δ 8.84 (d, J=4.7 Hz, 1H), 7.81 (d, J=4.7 Hz, 1H), 7.29 (s, 1H).

### Intermediate M

### 2,4-Dimethoxypyrimidin-5-amine

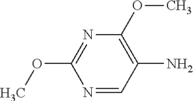

The title compound was synthesised according to the procedure of DU PONT PHARM CO, ALDRICH, Paul; ARVANITIS Argyrios; BAKTHAVATCHALAM Rajagopal; BECK James; CHEESEMAN Robert; CHORVAT Robert; GILLIGAN Paul; HODGE Carl; WASSERMAN Zelda, U.S. Pat. No. 6,107,301A, 2000, page 59.

LC-MS-1 (Method 2A): Rt 0.71 mins; MS m/z 156.0=[M+H]+

1H NMR (400 MHz, CDCl3) δ 7.75 (s, 1H), 4.05 (s, 3H), 3.94 (s, 3H).

### Intermediate N

### 2-[(4,4-Difluoropyrrolidin-3-yl)oxymethyl]-4-methyl-morpholine

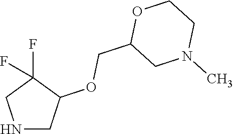 [SMILES: CN1CCOC(COC2CNCC2(F)F)C1]

### Step 1: benzyl 3,3-difluoro-4-[(4-methylmorpholin-2-yl)methoxy]pyrrolidine-1-carboxylate

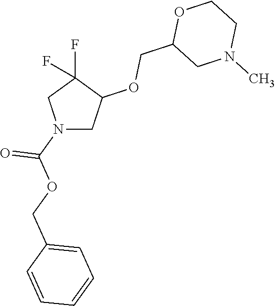 [SMILES: CN1CCOC(COC2CN(C(=O)OCc3ccccc3)CC2(F)F)C1]

A mixture of benzyl 3,3-difluoro-4-hydroxy-pyrrolidine-1-carboxylate (219 mg, 0.85 mmol), (4-methylmorpholin-2-yl)methyl 4-methylbenzenesulfonate (1 mL, 0.94 mmol) (Intermediate H step 1), NaOH (511 mg, 12.77 mmol) and TBAB (82 mg, 0.26 mmol) in toluene (8 mL) was heated to 80° C. for 3 h. (4-methylmorpholin-2-yl)methyl 4-methylbenzenesulfonate (1 mL, 0.94 mmol) (Intermediate H step 1), NaOH (511 mg, 12.77 mmol) and TBAB (82 mg, 0.26 mmol) were added and the reaction was stirred at 80° C. for 18 h. The reaction was cooled to room temperature and diluted in EtOAc (30 mL) and washed with water (15 mL). The aqueous phase was re-extracted with EtOAc (30 mL) and the combined organic extracts were dried over magnesium sulfate and concentrated in vacuo. Purification by chromatography twice on silica eluting with a gradient of 0 to 5% MeOH in DCM afforded the title compound as a colourless oil.

LC-MS-1 (Method 2A): Rt 0.90 mins; MS m/z 370.9=[M+H]+

1H NMR (400 MHz, DMSO) δ 7.41-7.30 (m, 5H), 5.10 (s, 2H), 4.31-4.21 (m, 1H), 3.88-3.36 (m, 8H), 2.69-2.62 (m, 1H), 2.60-2.53 (m, 2H), 2.16 (s, 3H), 2.01-1.86 (m, 1H), 1.79-1.67 (m, 1H).

### Step 2: 2-[(4,4-difluoropyrrolidin-3-yl)oxymethyl]-4-methyl-morpholine

A mixture of 10% Pd/C (49 mg, 0.05 mmol) and benzyl 3,3-difluoro-4-[(4-methylmorpholin-2-yl)methoxy]pyrrolidine-1-carboxylate (step 1) (170 mg, 0.46 mmol) in Ethanol (4 mL) was hydrogenated under 1 atmosphere of hydrogen for 1 h.

The reaction was filtered through celite, washing with IMS (200 mL) before concentrating the filtrate in vacuo to afford the title compound as a colourless oil.

1H NMR (400 MHz, DMSO-d6) δ 3.90-3.81 (m, 1H), 3.80-3.72 (m, 1H), 3.65-3.43 (m, 4H), 3.23 (dd, J=11.9, 6.8 Hz, 1H), 3.19-3.09 (m, 1H), 3.00-2.86 (m, 1H), 2.70-2.54 (m, 3H), 2.15 (s, 3H), 1.93 (td, J=11.4, 3.3 Hz, 1H), 1.80-1.66 (m, 1H).

### Biological Example A

### CD73 Inhibition Assay

Compound inhibition of human (h)CD73 (amino acids Trp27-Lys547, R&D Systems 5795-EN-500) enzymatic activity was tested via the Malachite Green Phosphate Detection Kit (R&D DY996). Experiments were performed in 20 μL Tris buffer (25 mM Tris pH 7.5, 5 mM MgCl₂, 0.002% Tween20), with 250 pM of hCD73. Test compounds of interest were prepared in a threefold serial dilution in DMSO and added to the assay to give the appropriate final concentration, with the assay DMSO concentration being 1%. Compounds were pre-incubated for 20 minutes with hCD73 in a 384 well clear plate (Greiner 781101), followed by the addition of adenosine monophosphate (AMP) to 12.5 μM. The enzymatic reaction was carried out for 20 min at room temperature. 5 μL of Malachite kit reagent A was added to stop the reaction (contains 3M H₂SO₄) and after 10 min, 5 μL of Malachite kit reagent B was added (Malachite Green). After 30 min, absorbance at 620 nm was read on a BMG Pherastar FSX.

Data were normalised to measurements in the absence of competing compound (0% inhibition) and in the presence of 10 μM control compound (100% inhibition). Competition curves were then fit to a 4-parameter logistic equation including a variable slope factor to determine the IC₅₀ (compound concentration that inhibits 50% of 0073 activity).

The results are shown in Table A below.

TABLE A

| Example No. | IC₅₀ (nM) |
|---|---|
| 1 | 715 |
| 1.1 | 677 |
| 1.2 | 428 |
| 1.3 | 489 |
| 2 | 11.1 |
| 2.1 | 401 |
| 3 | 59.6 |
| 4 | 87.3 |
| 4.1 | 96 |
| 4.2 | 197 |
| 4.3 | 269 |
| 4.4 | 74.3 |
| 5 | 32 |
| 6 | 57.3 |
| 6.1 | 94 |
| 7 | 22.8 |
| 7.1 | 25.4 |
| 8 | 20.1 |
| 9 | 283 |
| 10 | 20.7 |
| 10.1 | 85.4 |
| 10.2 | 130 |
| 11 | 103 |
| 12 | 33.7 |
| 13 | 44.8 |
| 13.1 | 37.1 |
| 13.2 | 60.7 |
| 13.3 | 124 |
| 13.4 | 95 |
| 13.5 | 84.7 |
| 13.6 | 31.1 |
| 13.7 | 73.9 |
| 13.8 | 47.6 |
| 13.9 | 303 |
| 13.10 | 898 |
| 13.11 | 49.2 |
| 14 | 62.8 |
| 14.1 | 256 |
| 15 | 139 |
| 16 | 297 |
| 17 | 59.4 |
| 18 | 62.5 |
| 19 | 58.2 |
| 19.1 | 96.2 |
| 19.2 | 9730 |
| 19.3 | 137 |
| 20 | 834 |
| 21 | 100 |
| 22 | 136 |
| 22.1 | 152 |
| 22.2 | 170 |
| 23 | 152 |
| 24 | 35.5 |
| 25 | 34.2 |
| 25.1 | 42.3 |
| 25.2 | 83.7 |
| 25.3 | 54.7 |
| 25.4 | 141.4 |
| 25.5 | 71.9 |
| 25.6 | 99.0 |
| 25.7 | 132.0 |
| 25.8 | 16 |
| 25.9 | 58 |
| 26 | 55.6 |
| 26.1 | 22.89 |
| 26.2 | 43.33 |
| 26.3 | 35.11 |
| 26.4 | 68.25 |
| 26.5 | 46.0 |
| 26.6 | 59.0 |
| 27 | 65 |
| 28 | 29.3 |
| 28.1 | 15 |
| 28.2 | 20 |
| 29 | 52.0 |
| 30 | 115.0 |
| 31 | 20.8 |

### Biological Example B

### U138-MG Inhibition Assay

U138-MG cells from ATCC are cultured in DMEM (Gibco 31966) with 10% FBS. Cells are seeded into 96-well culture plates (5,000 cell/well) in 100 μL of media for 24 hr. Cells are washed twice with 200 μL of assay buffer (20 mM HEPES, pH 7.4; 137 mM NaCl; 5.4 mM KCl; 1.3 mM CaCl₂; 4.2 mM NaHCO₃; 1 mg/mL glucose) to remove residual inorganic phosphate. CD73 inhibitor is added to each well in 100 μl volume and preincubated for 30 mins at 37° C. An equal volume of AMP substrate (Sigma A1572) is added to each well at a final concentration of 100 μM and incubated at 37° C. for 1 hour. Control wells containing cells but lacking added AMP, define the assay signal equivalent to 100% inhibition of CD73 activity. Supernatant is removed for measurement of inorganic phosphate using the PiColorLock Gold kit (Abcam, ab 270004).

The IC₅₀ values of test compounds are determined as the inflection point of the sigmoidal concentration response curve.

TABLE B

| Example No. | IC₅₀ (nM) |
|---|---|
| 1 | 2340 |
| 1.1 | 2312 |
| 1.2 | 1014 |
| 1.3 | 716.6 |
| 2 | 46.1 |
| 2.1 | 1121 |
| 3 | 588.3 |
| 4 | 733.6 |
| 4.1 | 427.7 |
| 4.2 | 3142 |
| 4.3 | 497.7 |
| 4.4 | 76.6 |
| 5 | 178.7 |
| 6 | 18.7 |
| 6.1 | 66 |
| 7 | 7.2 |
| 7.1 | 22.8 |
| 8 | 34.7 |
| 10 | 51.9 |
| 10.1 | 61.1 |
| 10.2 | 312.3 |
| 11 | 246.1 |
| 12 | 2.7 |
| 13 | 1.8 |
| 13.1 | 2.4 |
| 13.2 | 2.1 |
| 13.3 | 4.5 |
| 13.4 | 35.3 |
| 13.5 | 10.5 |
| 13.6 | 8.6 |
| 13.7 | 20.9 |
| 13.8 | 12.1 |
| 13.9 | 49.2 |
| 13.10 | 2495 |
| 13.11 | 3.2 |
| 14 | 176.9 |
| 14.1 | 1323 |
| 15 | 65.6 |
| 16 | 1345 |
| 17 | 107.9 |
| 18 | 91.1 |
| 19 | 68.7 |
| 19.1 | 104 |
| 19.3 | 28.3 |
| 20 | 743.6 |
| 21 | 70.8 |
| 22 | 84.5 |
| 22.1 | 61.9 |
| 22.2 | 54.9 |
| 23 | 10.5 |
| 24 | 11.8 |
| 25 | 38.6 |
| 25.1 | 38.3 |
| 25.2 | 58.8 |
| 25.3 | 91 |
| 25.4 | 115 |
| 25.5 | 170 |
| 25.6 | 46 |
| 25.7 | 65 |
| 25.8 | 2.8 |
| 25.9 | 22 |
| 25.10 | 5 |
| 25.11 | 23 |
| 25.12 | 38 |
| 25.13 | 12 |
| 25.14 | 7 |
| 25.15 | 18 |
| 25.16 | 11 |
| 25.17 | 39 |
| 26 | 2.9 |
| 26.1 | 5 |
| 26.2 | 12 |
| 26.3 | 16 |
| 26.4 | 17 |
| 26.5 | 14 |
| 26.6 | 11 |
| 27 | 20 |
| 28 | 4.0 |
| 28.1 | 3.6 |
| 28.2 | 1.9 |
| 29 | 8.0 |
| 30 | 12.0 |
| 31 | 12.3 |

### Biological Example C

### Whole Blood AMP Glo Assay

Blood is drawn from healthy volunteers by venepuncture and anti-coagulated with Lithium Heparin (10 IU/mi). 150 μl of blood is added to U-bottom polypropylene 96-well assay plates pre-filled with 20 μl of compound dilutions prepared in RPMI also containing the Tissue Non-specific Alkaline Phosphatase inhibitor Levamisole (final assay concentration 1.5 mM). Control wells to define uninhibited CD73 contain DMSO in place of compound. Control wells to define complete stabilization of AMP attributable to CD73 contain LY3475070 (CAS: 2375815-63-5) at 30 μM final assay concentration.

Compounds are pre-incubated with whole blood at 37° C. for 30 minutes. Following this, AMP is added in a 30 μl volume for a final assay concentration of 10 μM, mixed, and the plates are incubated for 15 minutes at 37° C. Final DMSO concentration/well is 0.6% and final volume/well is 200 μL. After incubating with AMP, the plates are placed on ice and then centrifuged at 2000 g for 10 minutes at 4° C. Plasma is removed and then diluted 1:10 with pre-chilled HBSS (Ca2+& Mg2+free) and kept on ice.

The stabilized AMP concentration in diluted plasma is measured using AMP-Glo kit (Promega), in parallel with a standard curve of known AMP concentrations, with a final volume of 40 μl in white flat bottom half-well 96-well plates (Corning). Signal is measured by Luminescence. The IC₅₀ values of test compounds are determined as the inflection point of the sigmoidal concentration response curve.

TABLE C

| Example No. | IC₅₀ (nM) |
|---|---|
| 1 | 8750 |
| 1.1 | 8760 |
| 1.3 | 3160 |
| 2 | 47 |
| 2.1 | 6990 |
| 3 | 2220 |
| 4 | 4010 |
| 4.1 | 782 |
| 4.3 | 516 |
| 4.4 | 254 |
| 5 | 184 |
| 6 | 27.5 |
| 7 | 12.5 |
| 7.1 | 444 |
| 8 | 50.7 |
| 10 | 29.6 |
| 10.1 | 50.1 |
| 10.2 | 1590 |
| 11 | 686 |
| 12 | 0.6 |
| 13 | 0.47 |
| 13.1 | 1.15 |
| 13.2 | 1.13 |
| 13.3 | 3.52 |
| 13.4 | 14.1 |
| 13.5 | 2.12 |
| 13.6 | 1.19 |
| 13.7 | 7.58 |
| 13.8 | 1.5 |
| 13.9 | 5.5 |
| 13.11 | 1.5 |
| 14 | 549 |
| 15 | 56.5 |
| 16 | 1550 |
| 17 | 119 |
| 18 | 118 |
| 19 | 1240 |
| 19.1 | 1990 |
| 19.3 | 652 |
| 21 | 40.2 |
| 22 | 65.5 |
| 22.1 | 139 |
| 23 | 1.5 |
| 24 | 1.4 |
| 25 | 155 |
| 25.1 | 645 |
| 25.8 | 38 |
| 25.9 | 18000 |
| 25.10 | 56 |
| 25.11 | 200 |
| 25.13 | 5 |
| 25.14 | 20 |
| 25.16 | 78 |
| 25.17 | 450 |
| 26 | 16 |
| 26.1 | 37 |
| 26.4 | 69 |
| 26.5 | 254 |
| 27 | 16 |
| 28 | 13 |
| 28.1 | 20 |
| 28.2 | 26 |
| 29 | 47 |
| 30 | 20 |
| 31 | 32 |

### Numbered Paragraphs

The following numbered paragraphs define particular aspects and embodiments of the invention:
Paragraph 1. A compound, or pharmaceutically acceptable salt thereof, having the structural formula I shown below:

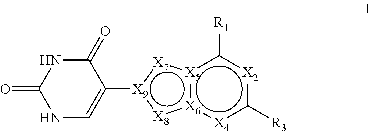

wherein:
R₁ is selected from:
(i) —NR_{1A}R_{1B};
   wherein R_{1A} and R_{1B} are each independently selected from hydrogen, (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl;
   or
   or R_{1A} and R_{1B} are linked such that, together with the nitrogen atom to which they are attached, they form a heterocyclic ring;
      and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
(ii) a carbon-linked heterocyclyl optionally substituted by one or more Rₓ;
(iii) —O—R_{1C};
   wherein R_{1C} is selected from (1-6C)alkyl, (3-8C)cycloalkyl, (3-8C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
(iv) (1-6C)alkyl optionally substituted by one or more Rₓ;
(v) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, wherein any alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl or heterocyclyl(1-2C)alkyl group is optionally substituted by one or more Rₓ;
(vi) a carbon or nitrogen-linked heteroaryl ring optionally substituted by one or two substituents independently selected from fluoro, (1-4C)alkyl, (1-4C)haloalkyl, (1-4C)alkoxy, (1-4C)haloalkoxy, hydroxy or (1-4C)hydroxyalkyl;
wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, (CH₂)_{q1}C(O)OR_{1D}, O(CH₂)_{q1}C(O)R_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
      wherein q1 is 0, 1, 2 or 3; and
      wherein R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl;
      and R_{1E} is selected from hydrogen, (1-4C)alkyl, (3-6C)cycloalkyl, (3-6C)cycloalkyl(1-4C)alkyl, aryl or aryl(1-4C)alkyl;
      or R_{1D} and R_{1E} are linked such that, together with the atom or atoms to which they are attached, they form a 4 to 10 membered heterocyclic ring;
   and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
      wherein q2 is 0, 1, 2 or 3; and
      wherein R_{1F} and R_{1G} are each independently selected from hydrogen or (1-4C)alkyl optionally substituted by halo; and
X₂ is selected from N or CR₂; wherein R₂ is selected from hydrogen, halo, (1-3C)alkyl, (1-3C)alkoxy, (1-3C)haloalkyl, (1-3C)haloalkoxy or cyano;
R₃ is selected from hydrogen, halo (1-3C)alkyl, (1-3C)alkoxy, (1-3C)haloalkyl, (1-3C)haloalkoxy or cyano,
X₄ is selected from N or CR₄; wherein R₄ is selected from hydrogen, halo, (1-3C)alkyl, (1-3C)alkoxy, (1-3C)haloalkyl, (1-3C)haloalkoxy or cyano;
X₅ is selected from N or C;
X₆ is selected from N or C;
X₇ is selected from N, NR_{7N}, CR₇, O or S; wherein:
   R₇ is selected from hydrogen, halo, methyl or methoxy;
   R_{7N} is selected from hydrogen or methyl;
X₈ is selected from N, NR_{8N}, CR₈, O or S; wherein:
   R₈ is selected from hydrogen, halo, methyl or methoxy;
   R_{8N} is selected from hydrogen or methyl;
X₉ is selected from N or C;
with the proviso that:
   (i) X₅ and X₆ cannot both be N;
   (ii) only one of X₇ and X₈ can be O or S;
   (iii) no more than four of the atoms in groups X₂, X₄, X₅, X₆, X₇, X₈ and X₉ which, together with CR₁ and CR₃, make up the bicyclic ring, may be a heteroatom.
Paragraph 2. A compound according to Paragraph 1, or a pharmaceutically acceptable salt thereof, wherein R₁ is selected from:
   (i) —NR_{1A}R_{1B};
      wherein R_{1A} and R_{1B} are each independently selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, or R_{1A} and R_{1B} are linked such that, together with the nitrogen atom to which they are attached, they form a heterocyclic ring;
      and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
   (ii) a carbon-linked heterocyclyl optionally substituted by one or more Rₓ;
   (iii) —O—R_{1C};
      wherein R_{1C} is selected from (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
   (iv) (1-6C)alkyl optionally substituted by one or more Rₓ;
   (v) (3-6C)cycloalkyl optionally substituted by one or two substituents independently selected from fluoro, (1-4C)alkyl, (1-3C)haloalkyl, (1-3C)alkoxy, (1-3C)haloalkoxy, hydroxy or (1-3C)hydroxyalkyl;
   (vi) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, aryl, heteroaryl, or heterocyclyl;
   (vii) a carbon or nitrogen-linked monocyclic heteroaryl ring optionally substituted by one or two substituents independently selected from fluoro, (1-3C)alkyl, (1-3C)haloalkyl, (1-3C)alkoxy, (1-3C)haloalkoxy, hydroxy or (1-3C)hydroxyalkyl;
   wherein each Rₓ is independently selected from:
      halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
      wherein
         q1 is 0, 1, or 2;
         R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl; and
         R_{1E} is selected from hydrogen, (1-4C)alkyl, (3-6C)cycloalkyl, (3-6C)cycloalkyl(1-4C)alkyl, aryl or aryl(1-4C)alkyl;
      and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
      wherein q2 is 0, 1, or 2; and
      wherein R_{1F} and R_{1G} are each independently selected from hydrogen or (1-4C)alkyl optionally substituted by halo.
Paragraph 3. A compound according to Paragraph 1 or Paragraph 2, or a pharmaceutically acceptable salt thereof, wherein R₁ is selected from:
   (i) —NR_{1A}R_{1B};
      wherein R_{1A} and R_{1B} are each independently selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl,
      or R_{1A} and R_{1B} are linked such that, together with the nitrogen atom to which they are attached, they form a heterocyclic ring;
      and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl or heterocyclyl group formed by R_{1A} and R_{1B} is optionally substituted by one or more Rₓ;
   (ii) a carbon-linked heterocyclyl optionally substituted by one or more Rₓ;
   (iii) —O—R_{1C};
      wherein R_{1C} is selected from (1-6C)alkyl, (3-6C)cycloalkyl, (3-6C)cycloalkyl(1-2C)alkyl, phenyl, or phenyl(1-2C)alkyl, each of which is optionally substituted by one or more Rₓ;
   (iv) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl or phenyl;
   wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 10-membered heterocyclyl, (4- to 10-membered)heterocyclyl(1-4C)alkyl, or a spiro-fused (3-6C)cycloalkyl or 4- to 6-membered heterocyclyl;
      wherein q1 is 0, 1, or 2; and
      wherein R_{1D} is selected from hydrogen, (1-4C)alkyl, (3-6C)cycloalkyl, (3-6C)cycloalkyl(1-4C)alkyl, phenyl, phenyl(1-4C)alkyl, heteroaryl, heteroaryl(1-4C)alkyl, heterocyclyl, or heterocyclyl(1-4C)alkyl;
      and R_{1E} is selected from hydrogen or (1-4C)alkyl;
   and wherein any (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 10-membered heterocyclyl, (4- to 10-membered)heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-2C)alkyl, (1-2C)haloalkyl, (1-2C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F},
   wherein q2 is 0 or 1; and
   wherein R_{1F} and R_{1G} are each independently selected from hydrogen or (1-2C)alkyl.
Paragraph 4. A compound according to any one of Paragraphs 1 to 3, or a pharmaceutically acceptable salt thereof, wherein R₁ is selected from:
   (i) —NR_{1A}R_{1B};
      wherein one of R_{1A} and R_{1B} hydrogen, and the other is selected from (1-4C)alkyl, aryl(1-2C)alkyl or heteroaryl(1-2C)alkyl, each of which being optionally substituted by one or more Rₓ; or
      R_{1A} and R_{1B} are linked such that, together with the nitrogen atom to which they are attached, they form a 4-, 5- or 6-membered monocyclic heterocyclic ring, a 5 to 10-membered fused heterocyclic ring, a 5 to 10-membered bridged heterocyclic ring or a 5 to 10-membered spirocyclic heterocyclic ring, each of which being optionally substituted by one or more Rₓ;
   (ii) a carbon-linked 4 to 10-membered heterocyclyl (including 4-, 5- or 6-membered monocyclic heterocyclic rings, fused heterocyclic rings, bridged heterocyclic rings and spirocyclic heterocyclic rings) optionally substituted by one or more Rₓ;
   (iii) (3C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl or phenyl;
   wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl(1-3C)alkyl, phenyl, phenyl(1-3C)alkyl, (5- or 6-membered)heteroaryl(1-3C)alkyl, (4- to 6-membered)heterocyclyl(1-3C)alkyl, or a spiro-fused (3-6C)cycloalkyl or 4- to 6-membered heterocyclyl;
   wherein q1 is 0 or 1;
      wherein R_{1D} is selected from hydrogen, (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
      and R_{1E} is selected from hydrogen or (1-2C)alkyl;
   and wherein any (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, NR_{1F}R_{1G}, OR_{1F}, C(O)R_{1F} and C(O)OR_{1F} (e.g. halo, cyano, hydroxy or (1-4C)alkyl);
   wherein R_{1F} and R_{1G} are each independently selected from hydrogen or (1-2C)alkyl.
Paragraph 5. A compound according to any one of the preceding Paragraphs, or a pharmaceutically acceptable salt thereof, wherein R₁ is selected from:
   (i) —NR_{1A}R_{1B};
      wherein one of R_{1A} and R_{1B} hydrogen, and the other is selected from (1-4C)alkyl, aryl(1-2C)alkyl or heteroaryl(1-2C)alkyl, each of which being optionally substituted by one or more Rₓ; or
      R_{1A} and R_{1B} are linked such that, together with the nitrogen atom to which they are attached, they form a 4-, 5- or 6-membered monocyclic heterocyclic ring, a 5 to 10-membered fused heterocyclic ring, a 5 to 10-membered bridged heterocyclic ring or a 5 to 10-membered spirocyclic heterocyclic ring, each of which being optionally substituted by one or more Rₓ;
   (ii) a carbon-linked 4 to 10-membered heterocyclyl (including 4-, 5- or 6-membered monocyclic heterocyclic rings, fused heterocyclic rings, bridged heterocyclic rings and spirocyclic heterocyclic rings) optionally substituted by one or more Rₓ;
   (iii) (3C)cycloalkyl optionally substituted by one or two substituents independently selected from fluoro, (1-2C)alkyl, (1-2C)haloalkyl, (1-2C)alkoxy, (1-2C)haloalkoxy, hydroxy, (1-2C)hydroxyalkyl or phenyl;
   wherein each Rₓ is independently selected from:
   halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl(1-3C)alkyl, phenyl, phenyl(1-3C)alkyl, (5- or 6-membered)heteroaryl(1-3C)alkyl, (4- to 6-membered)heterocyclyl(1-3C)alkyl, or a spiro-fused (3-6C)cycloalkyl or 4- to 6-membered heterocyclyl;
   wherein q1 is 0 or 1;
      wherein R_{1D} is selected from hydrogen, (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
      and R_{1E} is selected from hydrogen or (1-2C)alkyl;
   and wherein any (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, NR_{1F}R_{1G}, OR_{1F}, C(O)R_{1F} and C(O)OR_{1F} (e.g. halo, cyano, hydroxy or (1-4C)alkyl);
      wherein R_{1F} and R_{1G} are each independently selected from hydrogen or (1-2C)alkyl.
Paragraph 6. A compound according to any one of the preceding Paragraphs, or a pharmaceutically acceptable salt thereof, wherein R₁ is selected from:
   (i) —NR_{1A}R_{1B};
      wherein one of R_{1A} and R_{1B} hydrogen, and the other is selected from (1-4C)alkyl or phenyl(1-2C)alkyl, each of which being optionally substituted by one or more Rₓ; or
      R_{1A} and R_{1B} are linked such that, together with the nitrogen atom to which they are attached, they form a 4-, 5- or 6-membered monocyclic heterocyclic ring, a 6 to 10 membered fused heterocyclic ring, a 6 to 10 membered bridged heterocyclic ring or a 6 to 10 membered spirocyclic heterocyclic ring, each of which being optionally substituted by one or more Rₓ; or
   (ii) a carbon-linked a 4-, 5- or 6-membered monocyclic heterocyclic ring, a 6 to 10 membered fused heterocyclic ring, a 6 to 10 membered bridged heterocyclic ring or a 6 to 10 membered spirocyclic heterocyclic ring, each of which being optionally substituted by one or more Rₓ;
   (iii) (3C)cycloalkyl optionally substituted by one or two substituents independently selected from fluoro, (1-2C)alkyl, (1-2C)haloalkyl, (1-2C)alkoxy, (1-2C)haloalkoxy, hydroxy, (1-2C)hydroxyalkyl or phenyl;
   wherein each Rₓ is independently selected from halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D} or (CH₂)_{q1} N(R_{1E})C(O)R_{1D};
      wherein q1 is 0 or 1;
      wherein R_{1D} is selected from (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
      and R_{1E} is selected from hydrogen or (1-2C)alkyl;
      and wherein any (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-2C)alkyl, (1-2C)haloalkyl, (1-2C)hydroxyalkyl, NR_{1F}R_{1G}, OR_{1F}, C(O)R_{1F} and C(O)OR_{1F} (e.g. halo, cyano, hydroxy or (1-4C)alkyl);
      wherein R_{1F} and R_{1G} are each independently selected from hydrogen or (1-2C)alkyl.
Paragraph 7. A compound according to any one of the preceding Paragraphs, or a pharmaceutically acceptable salt thereof, wherein R₁ is selected from:
   (i) —NR_{1A}R_{1B};
      wherein one of R_{1A} and R_{1B} hydrogen, and the other is selected from (1-4C)alkyl or phenyl(1-2C)alkyl, each of which being optionally substituted by one or more Rₓ; or
      R_{1A} and R_{1B} are linked such that, together with the nitrogen atom to which they are attached, they form a 4-, 5- or 6-membered monocyclic heterocyclic ring, a 7 to 9 membered fused heterocyclic ring or a 7 to 9 membered spirocyclic heterocyclic ring, each of which being optionally substituted by one or more Rₓ; or
   (ii) a carbon-linked 4-, 5- or 6-membered monocyclic heterocyclic ring, a 7 to 9 membered fused heterocyclic ring or a 7 to 9 membered spirocyclic heterocyclic ring, each of which being optionally substituted by one or more Rₓ;
   (iii) (3C)cycloalkyl optionally substituted by one or two substituents independently selected from fluoro, (1-2C)alkyl, (1-2C)haloalkyl, (1-2C)alkoxy, (1-2C)haloalkoxy, hydroxy, (1-2C)hydroxyalkyl or phenyl;
   wherein each Rₓ is independently selected from halo, cyano, (1-4C)alkyl, (CH₂)_{q1}OR_{1D}, O(CH₂)_{q1}C(O)R_{1D} or O(CH₂)_{q1}C(O)N(R_{1E})R_{1D}, wherein q1 is 0 or 1;
      wherein R_{1D} is selected from (1-4C)alkyl, phenyl, phenyl(1-2C)alkyl, 5- or 6-membered heteroaryl, (5- or 6-membered)heteroaryl(1-2C)alkyl, 4- to 9-membered heterocyclyl, or (4- to 9-membered)heterocyclyl(1-2C)alkyl;
      and R_{1E} is selected from hydrogen or methyl;
      and wherein any (1-4C)alkyl, phenyl, phenyl(1-2C)alkyl, (5- or 6-membered)heteroaryl, (5- or 6-membered)heteroaryl(1-2C)alkyl, (4- to 9-membered)heterocyclyl or (4- to 9-membered)heterocyclyl(1-2C)alkyl group present in a Rₓ or R_{1D} substituent, or a heterocyclyl groups formed when R_{1D} and R_{1E} are linked, is optionally further substituted by halo, cyano or hydroxy (1-2C)alkyl or (1-2C)haloalkyl.
Paragraph 8. A compound according to any one of the preceding Paragraphs, or a pharmaceutically acceptable salt thereof, wherein R₁ is —NR_{1A}R_{B};
   wherein one of R_{1A} and R_{1B} hydrogen, and the other is other is selected from (1-4C)alkyl or phenyl(1-2C)alkyl, each of which being optionally substituted by one or more Rₓ; or
   wherein R_{1A} and R_{1B} are linked such that, together with the nitrogen atom to which they are attached, they form a 4- or 5-membered heterocyclic ring system, a 7 to 9 membered fused heterocyclic ring or a 7 to 9 membered spirocyclic heterocyclic ring each of which being optionally substituted by one or more Rₓ;
   wherein each Rₓ is independently selected from halo, cyano, (1-2C)alkyl, OR_{1D}, OC(O)R_{1D} or OC(O)N(R_{1E})R_{1D};
      wherein R_{1D} is selected from (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
      and R_{1E} is selected from hydrogen or methyl;
   and wherein any (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ or R_{1D} substituent, or a heterocyclyl groups formed when R_{1D} and R_{1E} are linked, is optionally further substituted by halo, cyano or hydroxy (1-2C)alkyl or (1-2C)haloalkyl.;
and optionally wherein R_{1A} and R_{1B} are linked such that, together with the nitrogen atom to which they are attached, they form a 4- or 5-membered nitrogen-linked heterocyclic ring, a 7 to 9 membered fused heterocyclic ring or a 7 to 9 membered spirocyclic heterocyclic ring, each of which being optionally substituted by one or more Rₓ;
   wherein each Rₓ is independently selected from halo, (1-2C)alkyl, OR_{1D}, OC(O)R_{1D} or OC(O)N(R_{1E})R_{1D};
      wherein R_{1D} is selected from (1-4C)alkyl, phenyl(1-2C)alkyl, 6-membered heteroaryl, (5- or 6-membered)heteroaryl(1-2C)alkyl, 6- to 9-membered heterocyclyl, or (6- to 9-membered)heterocyclyl(1-2C)alkyl;
      and R_{1E} is hydrogen,
   and wherein any (1-4C)alkyl, phenyl(1-2C)alkyl, 5- or 6-membered heteroaryl, (5- or 6-membered)heteroaryl(1-2C)alkyl, 4- to 9-membered heterocyclyl, or (4- to 9-membered)heterocyclyl(1-2C)alkyl group present in a Rₓ or R_{1D} substituent, or a heterocyclyl groups formed when R_{1D} and R_{1E} are linked, is optionally further substituted by halo, cyano or hydroxy (1-2C)alkyl or (1-2C)haloalkyl.
Paragraph 9. A compound according to any one of the preceding Paragraphs, or a pharmaceutically acceptable salt thereof, wherein R₁ is a group of the formula:

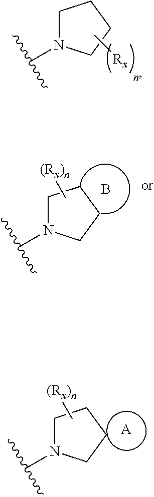

wherein n is 0, 1, 2, 3 or 4; and
each occurrence of Rₓ is independently as defined in any one of the preceding paragraphs;
Ring A, together with the carbon atom to which it is attached, is a spiro-fused 3 to 6 membered cycloalkyl or heterocyclyl ring; and
Ring B, together with the carbon atoms to which it is attached, is a fused 3 to 6 membered cycloalkyl or heterocyclyl ring;
optionally wherein R₁ is a group of the formula:

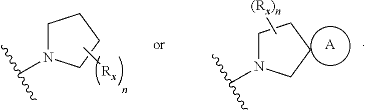

Paragraph 10. A compound according to any one of the preceding Paragraphs, or a pharmaceutically acceptable salt thereof, wherein R₁ is a group of the formula:

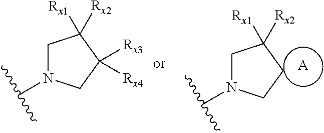

wherein:
Rₓ₁and Rₓ₂ are independently selected from hydrogen or halo;
Rₓ₃ and Rₓ₄ are independently selected from hydrogen, halo, methyl or methoxy;
or one of Rₓ₃ and Rₓ₄ is hydrogen, and the other is OR_{1D}, wherein R_{1D} is selected from phenyl(1-2C)alkyl, (5- or 6-membered)heteroaryl(1-2C)alkyl, or (4- to 6-membered)heterocyclyl(1-2C)alkyl; wherein the phenyl(1-2C)alkyl, (5- or 6-membered)heteroaryl(1-2C)alkyl, or (4- to 6-membered)heterocyclyl(1-2C)alkyl are each optionally substituted by one or more of halo, cyano, hydroxy or methyl; and
Ring A is a spiro-fused 3 to 6 membered cycloalkyl or heterocyclyl ring;
optionally wherein:
   Rₓ₁ and Rₓ₂ are independently selected from hydrogen or fluoro;
   Rₓ₃ and Rₓ₄ are independently selected from hydrogen, halo or methyl;
   or one of Rₓ₃ and Rₓ₄ is hydrogen, and the other is OR_{1D}, wherein R_{1D} is (6-membered)heterocyclyl(1-2C)alkyl; wherein the (6-membered)heterocyclyl(1-2C)alkyl is optionally substituted by one or more of halo, cyano or methyl; and
   Ring A is a spiro-fused 3 to 5 membered cycloalkyl or heterocyclyl ring.
Paragraph 11. A compound according to any one of Paragraphs 1 to 5, or a pharmaceutically acceptable salt thereof, wherein R₁ is selected from:

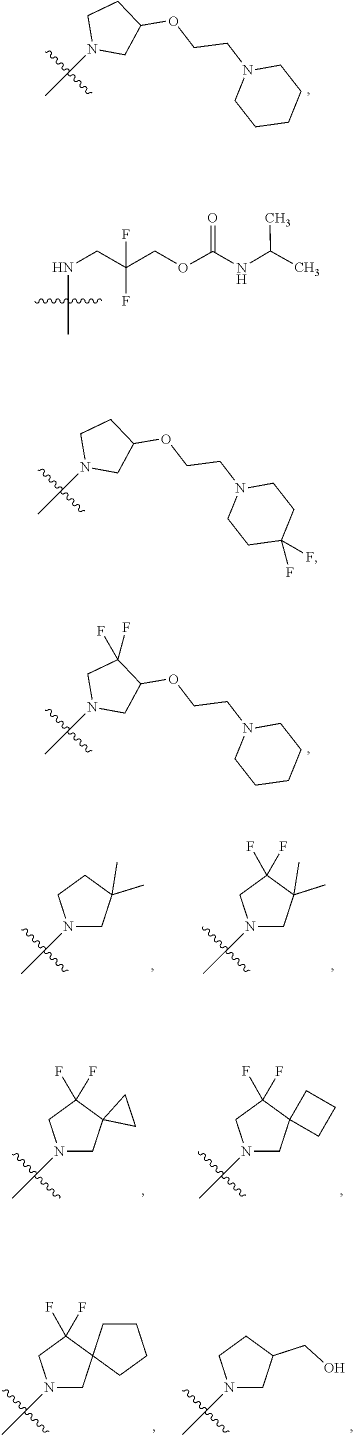

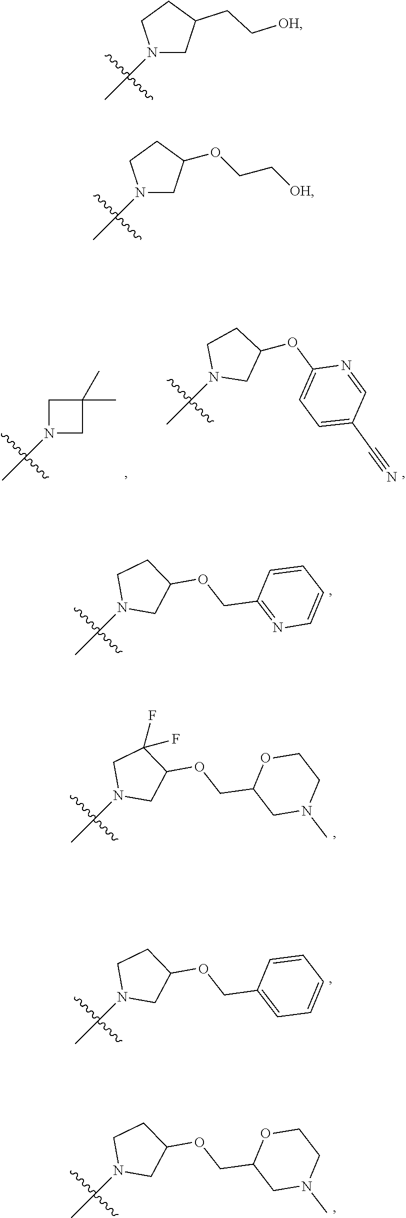

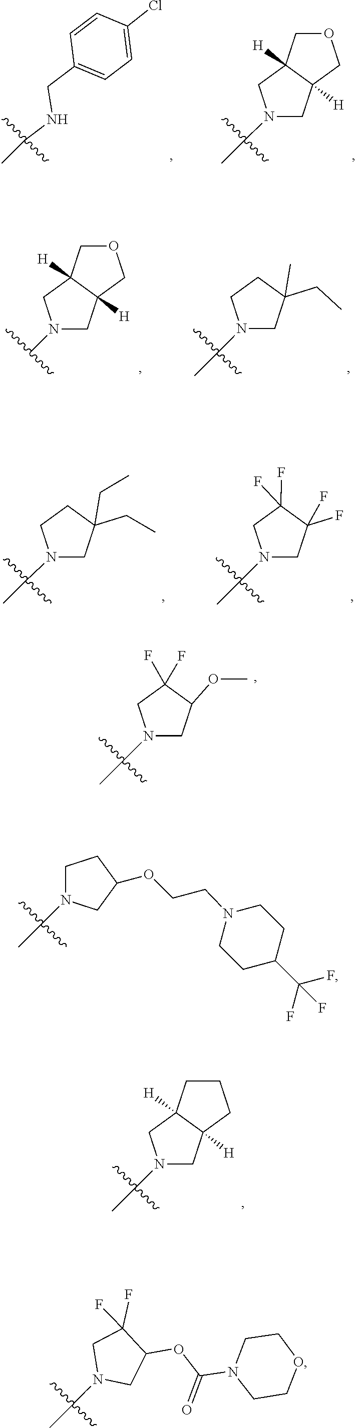

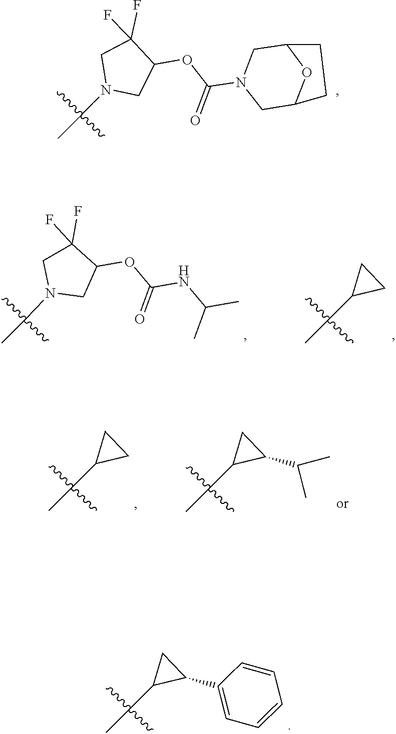

Paragraph 12. A compound according to any one of Paragraphs 1 to 5, or a pharmaceutically acceptable salt thereof, wherein R₁ is selected from:

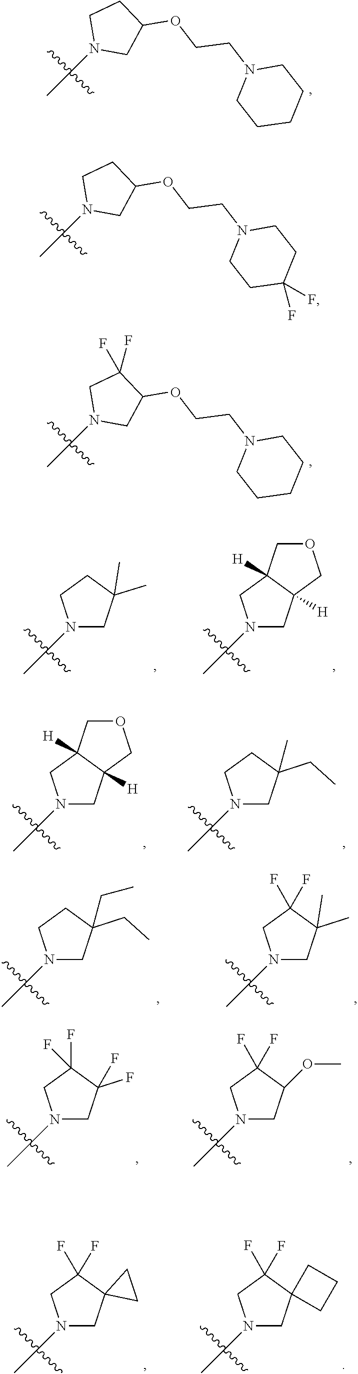

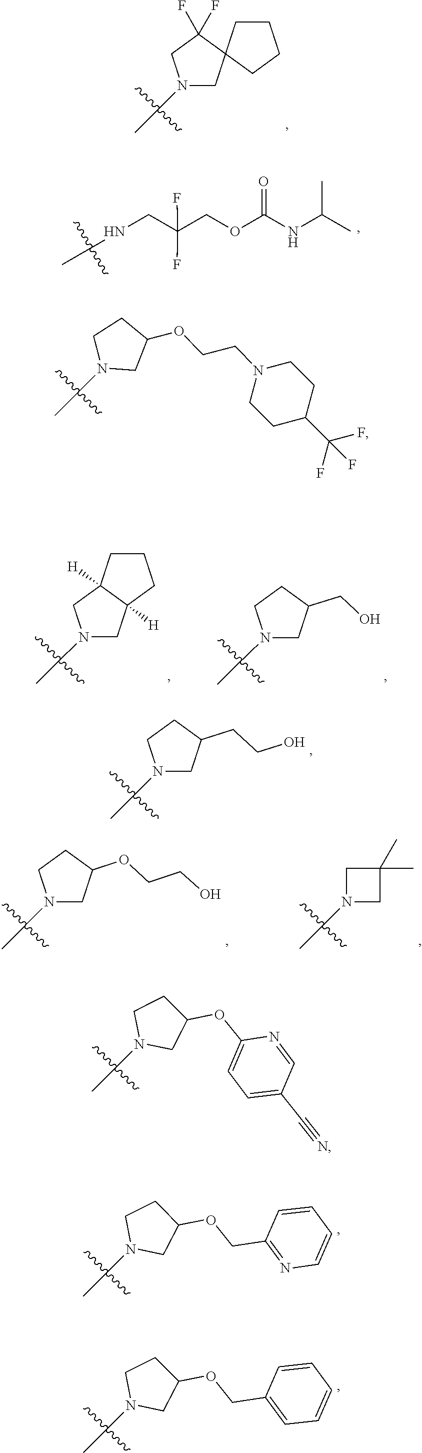

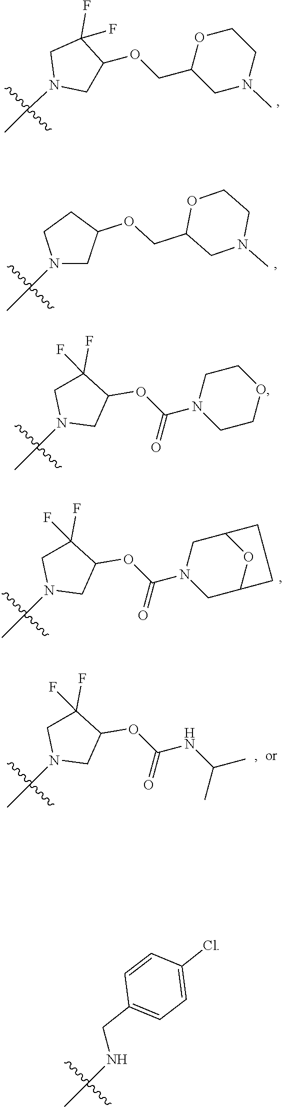 [SMILES: *CN1CC(OCC2CN(C)CCO2)C(F)(F)C1 ; *N1CCC(OCC2CN(C)CCO2)C1 ; *N1CC(OC(=O)N2CCOCC2)C(F)(F)C1 ; *N1CC(OC(=O)N2CC3CCC(C2)O3)C(F)(F)C1 ; *N1CC(OC(=O)NC(C)C)C(F)(F)C1 ; *NCc1ccc(Cl)cc1]

Paragraph 13. A compound according to any one of the preceding Paragraphs, or a pharmaceutically acceptable salt thereof, wherein:
X₂ is selected from N or CR₂; wherein R₂ is selected from hydrogen, halo, methyl, methoxy, halomethyl, halomethoxy or cyano;
R₃ is selected from hydrogen, halo methyl, methoxy, halomethyl, halomethoxy or cyano,
X₄ is selected from N or CR₄; wherein R₄ is selected from hydrogen, halo, (1-2C)alkyl, (1-2C)alkoxy, (1-2C)haloalkyl, (1-2C)haloalkoxy or cyano;
X₅ is selected from N or C;
X₆ is selected from N or C:
X₇ is selected from N, NR_{7N}, CR₇, O or S; wherein:
   R₇ is selected from hydrogen, halo, methyl or methoxy;
   R_{7N} is selected hydrogen;
X₈ is selected from N, NH, CH, O or S;
X₉ is selected from N or C;
with the proviso that:
   (i) X₅ and X₆ cannot both be N;
   (ii) only one of X₇ and X₈ can be O or S;
   (iii) no more than four of the groups X₂, X₄, X₅, X₆, X₇, X₈ and X₉ may comprise a heteroatom other than carbon
Paragraph 14. A compound according to any one of the preceding Paragraphs, or a pharmaceutically acceptable salt thereof, wherein:
X₂ is selected from N or CR₂; wherein R₂ is selected from hydrogen or cyano;
R₃ is selected from hydrogen, halo or cyano,
X₄ is selected from N or CR₄; wherein R₄ is selected from hydrogen, fluoro, chloro, methyl, methoxy, fluoromethyl (e.g. CH₂F, CHF₂ or CF₃) or cyano;
X₅ is C;
X₆ is selected from N or C;
X₇ is selected from N, CH, or S; wherein:
X₈ is selected from N, NH, CH, O or S;
X₉ is selected from N or C;
with the proviso that:
   (i) X₅ and X₆ cannot both be N;
   (ii) only one of X₇ and X₈ can be O or S;
   (iii) no more than four of the groups X₂, X₄, X₅, X₆, X₇, X₈ and X₉ which make up the bicyclic ring may be a heteroatom.
Paragraph 15. A compound of the formula Ia, Ib, Ic, Id, Ie, If, Ig, Ih or Ii:

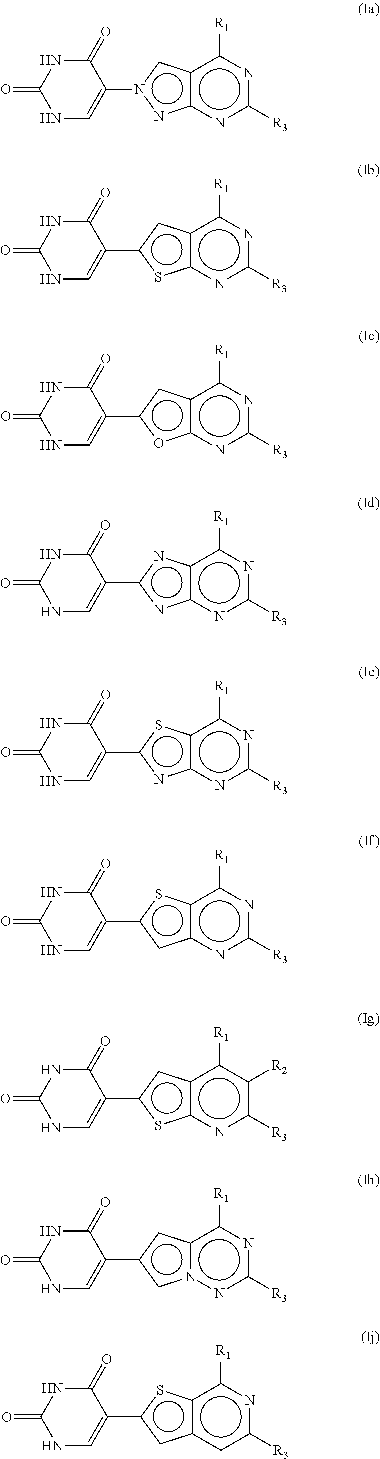

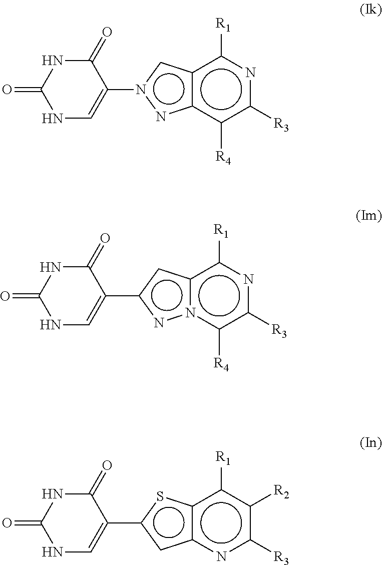

wherein R₁, R₂, R₃ and Ra are as defined in any one of of the preceding Paragraphs; or a pharmaceutically acceptable salt thereof.
Paragraph 16. A compound, or a pharmaceutically acceptable salt thereof, selected from any one of the following:
5-[4-[3-(Hydroxymethyl)pyrrolidin-1-yl]thieno[2,3-d]pyrimidin-6-yl]-1H-pyrimidine-2,4-dione;
5-[4-[3-(2-Hydroxyethyl)pyrrolidin-1-yl]thieno[2,3-d]pyrimidin-6-yl]-1H-pyrimidine-2,4-dione;
5-[4-(3,3-Dimethylazetidin-1-yl)thieno[2,3-d]pyrimidin-6-yl]-1H-pyrimidine-2,4-dione;
5-[4-[3-(2-Hydroxyethoxy)pyrrolidin-1-yl]thieno[2,3-d]pyrimidin-6-yl]-1H-pyrimidine-2,4-dione;
5-[4-[3-[2-(1-Piperidyl)ethoxy]pyrrolidin-1-yl]thieno[2,3-d]pyrimidin-6-yl]-1H-pyrimidine-2,4-dione;
5-[4-(3,3-Dimethylpyrrolidin-1-yl)thieno[2,3-d]pyrimidin-6-yl]-1H-pyrimidine-2,4-dione;
6-[1-[6-(2,4-Dioxo-1H-pyrimidin-5-yl)thieno[2,3-d]pyrimidin-4-yl]pyrrolidin-3-yl]oxypyridine-3-carbonitrile;
5-[4-(3,3-Difluoro-4,4-dimethyl-pyrrolidin-1-yl)thieno[2,3-d]pyrimidin-6-yl]-1H-pyrimidine-2,4-dione;
5-[4-[3-(2-pyridylmethoxy)pyrrolidine-1-yl]thieno[2,3-d]pyrimidin-6-yl]-1H-pyrimidine-2,4-dione;
5-[4-(3-Benzyloxypyrrolidin-1-yl)thieno[2,3-d]pyrimidin-6-yl]-1H-pyrimidine-2,4-dione;
5-[4-[3,3-Difluoro-4-[(4-methylmorpholin-2-yl)methoxy]pyrrolidine-1-yl]thieno[2,3-d]pyrimidin-6-yl]-1H-pyrimidine-2,4-dione;
5-[4-[3-[(4-Methylmorpholin-2-yl)methoxy]pyrrolidine-1-yl]thieno[2,3-d]pyrimidin-6-yl]-1H-pyrimidine-2,4-dione;
5-[4-[3,3-Difluoro-4-[2-(1-piperidyl)ethoxy]pyrrolidine-1-yl]thieno[2,3-d]pyrimidin-6-yl]-1H-pyrimidine-2,4-dione;
5-[7-[3-[2-(1-Piperidyl)ethoxy]pyrrolidin-1-yl]thiazolo[4,5-d]pyrimidin-2-yl]-1H-pyrimidine-2,4-dione;
5-[4-[3-[2-(1-Piperidyl)ethoxy]pyrrolidin-1-yl]furo[2,3-d]pyrimidin-6-yl]-1H-pyrimidine-2,4-dione;
5-[4-(3,3-Difluoro-4,4-dimethyl-pyrrolidin-1-yl)furo[2,3-d]pyrimidin-6-yl]-1H-pyrimidine-2,4-dione;
5-[4-[3-[2-(1-Piperidyl)ethoxy]pyrrolidin-1-yl]thieno[3,2-d]pyrimidin-6-yl]-1H-pyrimidine-2,4-dione;
5-[4-[3-[2-(1-Piperidyl)ethoxy]pyrrolidin-1-yl]thieno[3,2-c]pyridin-2-yl]-1H-pyrimidine-2,4-dione;
5-[4-[3-[2-(1-Piperidyl)ethoxy]pyrrolidin-1-yl]pyrrolo[2,1-f][1,2,4]triazin-6-yl]pyrimidine-2,4-diol; 5-[4-[3-[2-(4,4-Difluoro-1-piperidyl)ethoxy]pyrrolidin-1-yl]pyrrolo[2,1-f][1,2,4]triazin-6-yl]-1H-pyrimidine-2,4-dione;
5-[4-(3,3-Difluoro-4,4-dimethyl-pyrrolidin-1-yl)pyrrolo[2,1-f][1,2,4]triazin-6-yl]-1H-pyrimidine-2,4-dione;
2-(2,4-Dioxo-1H-pyrimidin-5-yl)-4-[3-[2-(1-piperidyl)ethoxy]pyrrolidin-1-yl]thieno[2,3-b]pyridine-5-carbonitrile;
5-[4-[3-[2-(1-Piperidyl)ethoxy]pyrrolidin-1-yl]pyrazolo[3,4-d]pyrimidin-2-yl]-1H-pyrimidine-2,4-dione hydrochloride;
5-[4-(3,3-Difluoro-4,4-dimethyl-pyrrolidin-1-yl)pyrazolo[3,4-d]pyrimidin-2-yl]-1H-pyrimidine-2,4-dione;
5-[4-(7,7-Difluoro-5-azaspiro[2.4]heptan-5-yl)pyrazolo[3,4-d]pyrimidin-2-yl]-1H-pyrimidine-2,4-dione;
5-[4-(8,8-Difluoro-6-azaspiro[3.4]octan-6-yl)pyrazolo[3,4-d]pyrimidin-2-yl]-1H-pyrimidine-2,4-dione;
5-[4-(4,4-Difluoro-2-azaspiro[4.4]nonan-2-yl)pyrazolo[3,4-d]pyrimidin-2-yl]-1H-pyrimidine-2,4-dione;
5-[4-[(3aR,6aS)-3,3a,4,5,6,6a-Hexahydro-1H-cyclopenta[c]pyrrol-2-yl]pyrazolo[3,4-d]pyrimidin-2-yl]-1H-pyrimidine-2,4-dione;
5-[4-(3-Ethyl-3-methyl-pyrrolidin-1-yl)pyrazolo[3,4-d]pyrimidin-2-yl]-1H-pyrimidine-2,4-dione;
5-[4-(3,3-Dimethylpyrrolidin-1-yl)pyrazolo[3,4-d]pyrimidin-2-yl]-1H-pyrimidine-2,4-dione;
5-[4-(3,3-Diethylpyrrolidin-1-yl)pyrazolo[3,4-d]pyrimidin-2-yl]-1H-pyrimidine-2,4-dione;
5-[4-(3,3,4,4-Tetrafluoropyrrolidin-1-yl)pyrazolo[3,4-d]pyrimidin-2-yl]-1H-pyrimidine-2,4-dione;
5-[4-[(3aR,6aS)-1,3,3a,4,6,6a-Hexahydrofuro[3,4-c]pyrrol-5-yl]pyrazolo[3,4-d]pyrimidin-2-yl]-1H-pyrimidine-2,4-dione;
5-[4-[(3aR,6aR)-1,3,3a,4,6,6a-Hexahydrofuro[3,4-c]pyrrol-5-yl]pyrazolo[3,4-d]pyrimidin-2-yl]-5H-pyrimidine-2,4-dione;
5-[4-(3,3-Difluoro-4-methoxy-pyrrolidin-1-yl)pyrazolo[3,4-d]pyrimidin-2-yl]-1H-pyrimidine-2,4-dione;
5-[4-[3-[2-(1-Piperidyl)ethoxy]pyrrolidin-1-yl]thieno[2,3-b]pyridin-2-yl]-1H-pyrimidine-2,4-dione;
5-[4-(3,3-Difluoro-4,4-dimethyl-pyrrolidin-1-yl)thieno[2,3-b]pyridin-2-yl]-1H-pyrimidine-2,4-dione;
5-[6-[3-[2-(1-Piperidyl)ethoxy]pyrrolidin-1-yl]-9H-purin-8-yl]-1H-pyrimidine-2,4-dione;
5-[7-[3-[2-(1-Piperidyl)ethoxy]pyrrolidin-1-yl]thieno[3,2-b]pyridin-2-yl]-1H-pyrimidine-2,4-dione;
5-[4-[3,3-Difluoro-4-[2-(1-piperidyl)ethoxy]pyrrolidin-1-yl]pyrrolo[2,1-f][1,2,4]triazin-6-yl]-1H-pyrimidine-2,4-dione;
5-[2-Chloro-4-[3-[2-(1-piperidyl)ethoxy]pyrrolidin-1-yl]pyrrolo[2,1-f][1,2,4]triazin-6-yl]-5H-pyrimidine-2,4-dione;
5-[4-(8,8-Difluoro-6-azaspiro[3.4]octan-6-yl)furo[2,3-d]pyrimidin-6-yl]-1H-pyrimidine-2,4-dione;
5-[4-(4,4-Difluoro-2-azaspiro[4.4]nonan-2-yl)furo[2,3-d]pyrimidin-6-yl]-1H-pyrimidine-2,4-dione;
5-[4-[(4-Chlorophenyl)methylamino]furo[2,3-d]pyrimidin-6-yl]-1H-pyrimidine-2,4-dione;
5-[4-(7,7-Difluoro-5-azaspiro[2.4]heptan-5-yl)furo[2,3-d]pyrimidin-6-yl]-1H-pyrimidine-2,4-dione;
[3-[[6-(2,4-Dioxo-1H-pyrimidin-5-yl)furo[2,3-d]pyrimidin-4-yl]amino]-2,2-difluoro-propyl]N-isopropylcarbamate;
5-[4-[3-[2-[4-(Trifluoromethyl)-1-piperidyl]ethoxy]pyrrolidin-1-yl]furo[2,3-d]pyrimidin-6-yl]-1H-pyrimidine-2,4-dione;
[1-[6-(2,4-Dioxo-1H-pyrimidin-5-yl)furo[2,3-d]pyrimidin-4-yl]-4,4-difluoro-pyrrolidin-3-yl]morpholine-4-carboxylate;
[1-[6-(2,4-Dioxo-1H-pyrimidin-5-yl)furo[2,3-d]pyrimidin-4-yl]-4,4-difluoro-pyrrolidin-3-yl]8-oxa-3-azabicyclo[3.2.1]octane-3-carboxylate;
[1-[6-(2,4-Dioxo-1H-pyrimidin-5-yl)furo[2,3-d]pyrimidin-4-yl]-4,4-difluoro-pyrrolidin-3-yl]N-isopropylcarbamate;
5-[4-(3,3-Difluoro-4,4-dimethyl-pyrrolidin-1-yl)-6-methyl-pyrazolo[3,4-d]pyrimidin-2-yl]-1H-pyrimidine-2,4-dione;
6-[1-[2-(2,4-Dioxo-1H-pyrimidin-5-yl)pyrazolo[3,4-d]pyrimidin-4-yl]pyrrolidin-3-yl]oxypyridine-3-carbonitrile;
5-[4-(3,3-Difluoro-4,4-dimethyl-pyrrolidin-1-yl)pyrazolo[1,5-a]pyrazin-2-yl]-1H-pyrimidine-2,4-dione;
5-[4-(8,8-Difluoro-6-azaspiro[3.4]octan-6-yl)pyrazolo[1,5-a]pyrazin-2-yl]-1H-pyrimidine-2,4-dione;
5-[4-(7,7-Difluoro-5-azaspiro[2.4]heptan-5-yl)pyrazolo[1,5-a]pyrazin-2-yl]-1H-pyrimidine-2,4-dione;
5-[4-(3,3-Difluoro-4,4-dimethyl-pyrrolidin-1-yl)-7-fluoro-pyrazolo[4,3-c]pyridin-2-yl]-1H-pyrimidine-2,4-dione; and
5-[4-(3,3-Difluoro-4,4-dimethyl-pyrrolidin-1-yl)pyrazolo[4,3-c]pyridin-2-yl]-1H-pyrimidine-2,4-dione.
   Paragraph 17. A pharmaceutical composition comprising a compound according to any one of Paragraphs 1 to 16, or a pharmaceutically acceptable salt thereof, in admixture with a pharmaceutically acceptable diluent or carrier.
   Paragraph 18. A compound according to any one of Paragraphs 1 to 16, or a pharmaceutically acceptable salt thereof, or a pharmaceutical composition according to Paragraph 17, for use in therapy.
   Paragraph 19. A compound according to any one of Paragraphs 1 to 16, or a pharmaceutically acceptable salt thereof, or a pharmaceutical composition according to Paragraph 17, for use:
   (i) in the treatment of a proliferative condition;
   (ii) in the treatment of cancer;
   (iii) in the treatment of cancer, wherein the compound or pharmaceutical composition is administered in combination with one or more additional anticancer agents;
   (iv) in the treatment of cancer, wherein the compound or pharmaceutical composition is administered in combination with one or more additional anticancer agents selected from the group consisting of:
      1) other forms of cancer immunotherapy and anti-cancer chemotherapeutic agents;
      2) A2b antagonists;
      3) anti-PD-1 and PDL-1 antibodies (e.g. pembrolizumab, nivolumab, durvalumab, avelumab and atezolizumab); and
      4) anti-CTLA4 antibodies (e.g ipilimumab).
   Paragraph 20. A method of treating a proliferative disorder in a patient in need of such treatment, the method comprising administering a therapeutically effective amount of a compound according to any one of Paragraphs 1 to 16, or a pharmaceutically acceptable salt thereof, or a pharmaceutical composition according to Paragraph 17.
   Paragraph 21. A method of treating cancer in a patient in need of such treatment, the method comprising administering a therapeutically effective amount of a compound according to any one of Paragraphs 1 to 16, or a pharmaceutically acceptable salt thereof, or a pharmaceutical composition according to Paragraph 17.
   Paragraph 22. A method of treating a proliferative disorder in a patient in need of such treatment, the method comprising administering a therapeutically effective amount of a compound according to any one of Paragraphs 1 to 16, or a pharmaceutically acceptable salt thereof, or a pharmaceutical composition according to Paragraph 17 in combination with one or more additional anticancer agents.
   Paragraph 23. A method according to Paragraph 22, wherein the one or more additional anticancer agents is selected from:
      1) other forms of cancer immunotherapy and anti-cancer chemotherapeutic agents; 2) A2b antagonists;
      3) anti-PD-1 and PDL-1 antibodies (e.g. pembrolizumab, nivolumab, durvalumab, avelumab and atezolizumab); and
      4) anti-CTLA4 antibodies (e.g ipilimumab).

1. A compound, or pharmaceutically acceptable salt thereof, having the structural formula I shown below: 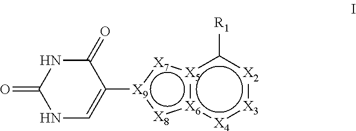
wherein:
R₁ is selected from:
(i) —NR_{1A}R_{1B};
wherein R_{1A} and R_{1B} are each independently selected from hydrogen, (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl;
or
or R_{1A} and R_{1B} are linked such that, together with the nitrogen atom to which they are attached, they form a heterocyclic ring;
and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
(ii) a carbon-linked heterocyclyl optionally substituted by one or more Rₓ;
(iii) —O—R_{1C};
wherein R_{1C} is selected from (1-6C)alkyl, (3-8C)cycloalkyl, (3-8C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
(iv) (1-6C)alkyl optionally substituted by one or more Rₓ;
(v) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, wherein any alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl or heterocyclyl(1-2C)alkyl group is optionally substituted by one or more Rₓ;
(vi) a carbon or nitrogen-linked heteroaryl ring optionally substituted by one or two substituents independently selected from fluoro, (1-4C)alkyl, (1-4C)haloalkyl, (1-4C)alkoxy, (1-4C)haloalkoxy, hydroxy or (1-4C)hydroxyalkyl;
wherein each Rₓ is independently selected from:
halo, cyano, (1-4C)alkyl, (CH₂)_{q1}NR_{1D}R_{1E}, (CH₂)_{q1}OR_{1D}, (CH₂)_{q1}C(O)R_{1D}, (CH₂)_{q1}C(O)OR_{1D}, O(CH₂)_{q1}C(O)R_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
wherein q1 is 0, 1, 2 or 3; and
wherein R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl;
and R_{1E} is selected from hydrogen, (1-4C)alkyl, (3-6C)cycloalkyl, (3-6C)cycloalkyl(1-4C)alkyl, aryl or aryl(1-4C)alkyl;
or R_{1D} and R_{1E} are linked such that, together with the atom or atoms to which they are attached, they form a 4 to 10 membered heterocyclic ring;
and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
wherein q2 is 0, 1, 2 or 3; and
wherein R_{1F} and R_{1G} are each independently selected from hydrogen or (1-4C)alkyl optionally substituted by halo; and
X₂ is selected from N or CR₂; wherein R₂ is selected from hydrogen, halo, (1-3C)alkyl, (1-3C)alkoxy, (1-3C)haloalkyl, (1-3C)haloalkoxy or cyano;
X₃ is selected from N or CR₃; wherein R₃ is selected from hydrogen, halo (1-3C)alkyl, (1-3C)alkoxy, (1-3C)haloalkyl, (1-3C)haloalkoxy or cyano,
X₄ is selected from N or CR₄; wherein R₄ is selected from hydrogen, halo (e.g. fluoro or chloro), (1-3C)alkyl, (1-3C)alkoxy, (1-3C)haloalkyl, (1-3C)haloalkoxy or cyano;
X₅ is selected from N or C;
X₆ is selected from N or C;
X₇ is selected from N, NR_{7N}, CR₇, O or S; wherein:
R₇ is selected from hydrogen, halo, methyl or methoxy;
R_{7N} is selected from hydrogen or methyl;
X₈ is selected from N, NR_{8N}, CR₈, O or S; wherein:
R₈ is selected from hydrogen, halo, methyl or methoxy;
R_{8N} is selected from hydrogen or methyl;
X₉ is selected from N or C;
with the proviso that:
(i) X₅ and X₆ cannot both be N;
(ii) only one of X₇ and X₈ can be O or S;
(iii) no more than four of the atoms in groups X₂, X₃, X₄, X₅, X₆, X₇, X₈ and X₉ which, together with the carbon atom attached to R₁, make up the bicyclic ring, may be a heteroatom.

2. A compound according to claim 1, or pharmaceutically acceptable salt thereof, having the structural formula I-1 shown below: 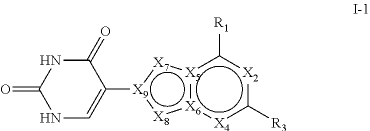
wherein:
R₁ is selected from:
(i) —NR_{1A}R_{1B};
wherein R_{1A} and R_{1B} are each independently selected from hydrogen, (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl;
or
or R_{1A} and R_{1B} are linked such that, together with the nitrogen atom to which they are attached, they form a heterocyclic ring;
and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
(ii) a carbon-linked heterocyclyl optionally substituted by one or more Rₓ;
(iii) —O—R_{1C};
wherein R_{1C} is selected from (1-6C)alkyl, (3-8C)cycloalkyl, (3-8C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
(iv) (1-6C)alkyl optionally substituted by one or more Rₓ;
(v) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, wherein any alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl or heterocyclyl(1-2C)alkyl group is optionally substituted by one or more Rₓ;
(vi) a carbon or nitrogen-linked heteroaryl ring optionally substituted by one or two substituents independently selected from fluoro, (1-4C)alkyl, (1-4C)haloalkyl, (1-4C)alkoxy, (1-4C)haloalkoxy, hydroxy or (1-4C)hydroxyalkyl;
wherein each Rₓ is independently selected from:
halo, cyano, (1-4C)alkyl, (CH₂)_{q1}NR_{1D}R_{1E}, (CH₂)_{q1}OR_{1D}, (CH₂)_{q1}C(O)R_{1D}, (CH₂)_{q1}C(O)OR_{1D}, O(CH₂)_{q1}C(O)R_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
wherein q1 is 0, 1, 2 or 3; and
wherein R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl;
and R_{1E} is selected from hydrogen, (1-4C)alkyl, (3-6C)cycloalkyl, (3-6C)cycloalkyl(1-4C)alkyl, aryl or aryl(1-4C)alkyl;
or R_{1D} and R_{1E} are linked such that, together with the atom or atoms to which they are attached, they form a 4 to 10 membered heterocyclic ring;
and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
wherein q2 is 0, 1, 2 or 3; and
wherein R_{1F} and R_{1G} are each independently selected from hydrogen or (1-4C)alkyl optionally substituted by halo; and
X₂ is selected from N or CR₂; wherein R₂ is selected from hydrogen, halo, (1-3C)alkyl, (1-3C)alkoxy, (1-3C)haloalkyl, (1-3C)haloalkoxy or cyano;
R₃ is selected from hydrogen, halo (1-3C)alkyl, (1-3C)alkoxy, (1-3C)haloalkyl, (1-3C)haloalkoxy or cyano,
X₄ is selected from N or CR₄; wherein R₄ is selected from hydrogen, halo, (1-3C)alkyl, (1-3C)alkoxy, (1-3C)haloalkyl, (1-3C)haloalkoxy or cyano;
X₅ is selected from N or C;
X₆ is selected from N or C;
X₇ is selected from N, NR_{7N}, CR₇, O or S; wherein:
R₇ is selected from hydrogen, halo, methyl or methoxy;
R_{7N} is selected from hydrogen or methyl;
X₈ is selected from N, NR_{8N}, CR₈, O or S; wherein:
R₈ is selected from hydrogen, halo, methyl or methoxy;
R_{8N} is selected from hydrogen or methyl;
X₉ is selected from N or C;
with the proviso that:
(i) X₅ and X₆ cannot both be N;
(ii) only one of X₇ and X₈ can be O or S;
(iii) no more than four of the atoms in groups X₂, X₄, X₅, X₆, X₇, X₈ and X₉ which, together with CR₁ and CR₃, make up the bicyclic ring, may be a heteroatom.

3. A compound according to claim 1 or claim 2, or a pharmaceutically acceptable salt thereof, wherein R₁ is selected from:
(i) —NR_{1A}R_{1B};
wherein R_{1A} and R_{1B} are each independently selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, or R_{1A} and R_{1B} are linked such that, together with the nitrogen atom to which they are attached, they form a heterocyclic ring;
and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl (including those formed by R_{1A} and R_{1B}) or heterocyclyl(1-2C)alkyl group is optionally substituted by one or more Rₓ;
(ii) a carbon-linked heterocyclyl optionally substituted by one or more Rₓ;
(iii) —O—R_{1C};
wherein R_{1C} is selected from (1-6C)alkyl, (3-6C)cycloalkyl, (3-6C)cycloalkyl(1-2C)alkyl, aryl, aryl(1-2C)alkyl, heteroaryl, heteroaryl(1-2C)alkyl, heterocyclyl, heterocyclyl(1-2C)alkyl, each of which is optionally substituted by one or more Rₓ;
(iv) (1-6C)alkyl optionally substituted by one or more Rₓ;
(v) (3-6C)cycloalkyl optionally substituted by one or two substituents independently selected from fluoro, (1-4C)alkyl, (1-3C)haloalkyl, (1-3C)alkoxy, (1-3C)haloalkoxy, hydroxy or (1-3C)hydroxyalkyl;
(vi) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl, a (3-6C)cycloalkyl, aryl, heteroaryl, or heterocyclyl;
(vii) a carbon or nitrogen-linked monocyclic heteroaryl ring optionally substituted by one or two substituents independently selected from fluoro, (1-3C)alkyl, (1-3C)haloalkyl, (1-3C)alkoxy, (1-3C)haloalkoxy, hydroxy or (1-3C)hydroxyalkyl;
wherein each Rₓ is independently selected from:
halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (CH₂)_{q1}S(O)ₚR_{1D} (where p is 0, 1 or 2), (CH₂)_{q1}SO₂N(R_{1E})R_{1D}, (CH₂)_{q1}N(R_{1E})SO₂R_{1D}, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl, or a spiro-fused (3-6C)cycloalkyl or heterocyclyl;
wherein
q1 is 0, 1, or 2;
R_{1D} is selected from hydrogen, (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, or heterocyclyl(1-4C)alkyl; and
R_{1E} is selected from hydrogen, (1-4C)alkyl, (3-6C)cycloalkyl, (3-6C)cycloalkyl(1-4C)alkyl, aryl or aryl(1-4C)alkyl;
and wherein any (1-4C)alkyl, (3-6C)cycloalkyl, (3-6C)cycloalkyl(1-4C)alkyl, aryl, aryl(1-4C)alkyl, heteroaryl, heteroaryl(1-4C)alkyl, heterocyclyl, heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F}
wherein q2 is 0, 1, or 2; and
wherein R_{1F} and R_{1G} are each independently selected from hydrogen or (1-4C)alkyl optionally substituted by halo.

4. A compound according to any one of the preceding claims, or a pharmaceutically acceptable salt thereof, wherein R₁ is selected from:
(i) —NR_{1A}R_{1B};
wherein R_{1A} and R_{1B} are each independently selected from hydrogen, (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl,
or R_{1A} and R_{1B} are linked such that, together with the nitrogen atom to which they are attached, they form a heterocyclic ring;
and wherein any (1-4C)alkyl, (3-6C)cycloalkyl, (3-6C)cycloalkyl(1-2C)alkyl, aryl, aryl(1-2C)alkyl, heteroaryl, heteroaryl(1-2C)alkyl or heterocyclyl group formed by R_{1A} and R_{1B} is optionally substituted by one or more Rₓ;
(ii) a carbon-linked heterocyclyl optionally substituted by one or more Rₓ;
(iii) —O—R_{1C};
wherein R_{1C} is selected from (1-6C)alkyl, (3-6C)cycloalkyl, (3-6C)cycloalkyl(1-2C)alkyl, phenyl, or phenyl(1-2C)alkyl, each of which is optionally substituted by one or more Rₓ;
(iv) (3-6C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl or phenyl;
wherein each Rₓ is independently selected from:
halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 10-membered heterocyclyl, (4- to 10-membered)heterocyclyl(1-4C)alkyl, or a spiro-fused (3-6C)cycloalkyl or 4- to 6-membered heterocyclyl;
wherein q1 is 0, 1, or 2; and
wherein R_{1D} is selected from hydrogen, (1-4C)alkyl, (3-6C)cycloalkyl, (3-6C)cycloalkyl(1-4C)alkyl, phenyl, phenyl(1-4C)alkyl, heteroaryl, heteroaryl(1-4C)alkyl, heterocyclyl, or heterocyclyl(1-4C)alkyl;
and R_{1E} is selected from hydrogen or (1-4C)alkyl;
and wherein any (3-6C)cycloalkyl, (3-6C)cycloalkyl(1-4C)alkyl, phenyl, phenyl(1-4C)alkyl, 5- or 6-membered heteroaryl, (5- or 6-membered)heteroaryl(1-4C)alkyl, 4- to 10-membered heterocyclyl, (4- to 10-membered)heterocyclyl(1-4C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-2C)alkyl, (1-2C)haloalkyl, (1-2C)hydroxyalkyl, (CH₂)_{q2}NR_{1F}R_{1G}, (CH₂)_{q2}OR_{1F}, (CH₂)_{q2}C(O)R_{1F}, (CH₂)_{q2}C(O)OR_{1F}, (CH₂)_{q2}OC(O)R_{1F}, (CH₂)_{q2}C(O)N(R_{1G})R_{1F}, (CH₂)_{q2}N(R_{1G})C(O)R_{1F}, (CH₂)_{q2}S(O)ₚR_{1F} (where p is 0, 1 or 2), (CH₂)_{q2}SO₂N(R_{1G})R_{1F}, (CH₂)_{q2}N(R_{1G})SO₂R_{1F},
wherein q2 is 0 or 1; and
wherein R_{1F} and R_{1G} are each independently selected from hydrogen or (1-2C)alkyl.

5. A compound according to any one of claims 1 to 3, or a pharmaceutically acceptable salt thereof, wherein R₁ is selected from:
(i) —NR_{1A}R_{1B};
wherein one of R_{1A} and R_{1B} hydrogen, and the other is selected from (1-4C)alkyl, aryl(1-2C)alkyl or heteroaryl(1-2C)alkyl, each of which being optionally substituted by one or more Rₓ; or
R_{1A} and R_{1B} are linked such that, together with the nitrogen atom to which they are attached, they form a 4-, 5- or 6-membered monocyclic heterocyclic ring, a 5 to 10-membered fused heterocyclic ring, a 5 to 10-membered bridged heterocyclic ring or a 5 to 10-membered spirocyclic heterocyclic ring, each of which being optionally substituted by one or more Rₓ;
(ii) a carbon-linked 4 to 10-membered heterocyclyl (including 4-, 5- or 6-membered monocyclic heterocyclic rings, fused heterocyclic rings, bridged heterocyclic rings and spirocyclic heterocyclic rings) optionally substituted by one or more Rₓ;
(iii) (3C)cycloalkyl optionally substituted by one or two substituents independently selected from fluoro, (1-4C)alkyl, (1-4C)haloalkyl, (1-4C)alkoxy, (1-4C)haloalkoxy, hydroxy, (1-4C)hydroxyalkyl or phenyl;
wherein each Rₓ is independently selected from:
halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl(1-3C)alkyl, phenyl, phenyl(1-3C)alkyl, (5- or 6-membered)heteroaryl(1-3C)alkyl, (4- to 6-membered)heterocyclyl(1-3C)alkyl, or a spiro-fused (3-6C)cycloalkyl or 4- to 6-membered heterocyclyl;
wherein q1 is 0 or 1;
wherein R_{1D} is selected from hydrogen, (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
and R_{1E} is selected from hydrogen or (1-2C)alkyl;
and wherein any (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, NR_{1F}R_{1G}, OR_{1F}, C(O)R_{1F} and C(O)OR_{1F} (e.g. halo, cyano, hydroxy or (1-4C)alkyl);
wherein R_{1F} and R_{1G} are each independently selected from hydrogen or (1-2C)alkyl.

6. A compound according to any one of the preceding claims, or a pharmaceutically acceptable salt thereof, wherein R₁ is selected from:
(i) —NR_{1A}R_{1B};
wherein one of R_{1A} and R_{1B} hydrogen, and the other is selected from (1-4C)alkyl, aryl(1-2C)alkyl or heteroaryl(1-2C)alkyl, each of which being optionally substituted by one or more Rₓ; or
R_{1A} and R_{1B} are linked such that, together with the nitrogen atom to which they are attached, they form a 4-, 5- or 6-membered monocyclic heterocyclic ring, a 5 to 10-membered fused heterocyclic ring, a 5 to 10-membered bridged heterocyclic ring or a 5 to 10-membered spirocyclic heterocyclic ring, each of which being optionally substituted by one or more Rₓ;
(ii) a carbon-linked 4 to 10-membered heterocyclyl (including 4-, 5- or 6-membered monocyclic heterocyclic rings, fused heterocyclic rings, bridged heterocyclic rings and spirocyclic heterocyclic rings) optionally substituted by one or more Rₓ;
(iii) (3C)cycloalkyl optionally substituted by one or two substituents independently selected from fluoro, (1-2C)alkyl, (1-2C)haloalkyl, (1-2C)alkoxy, (1-2C)haloalkoxy, hydroxy, (1-2C)hydroxyalkyl or phenyl;
wherein each Rₓ is independently selected from:
halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D}, (CH₂)_{q1}N(R_{1E})C(O)R_{1D}, (3-6C)cycloalkyl(1-3C)alkyl, phenyl, phenyl(1-3C)alkyl, (5- or 6-membered)heteroaryl(1-3C)alkyl, (4- to 6-membered)heterocyclyl(1-3C)alkyl, or a spiro-fused (3-6C)cycloalkyl or 4- to 6-membered heterocyclyl;
wherein q1 is 0 or 1;
wherein R_{1D} is selected from hydrogen, (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
and R_{1E} is selected from hydrogen or (1-2C)alkyl;
and wherein any (1-4C)alkyl, (3-6C)cycloalkyl, (3-6C)cycloalkyl(1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-4C)alkyl, (1-4C)haloalkyl, (1-4C)hydroxyalkyl, NR_{1F}R_{1G}, OR_{1F}, C(O)R_{1F} and C(O)OR_{1F} (e.g. halo, cyano, hydroxy or (1-4C)alkyl);
wherein R_{1F} and R_{1G} are each independently selected from hydrogen or (1-2C)alkyl.

7. A compound according to any one of the preceding claims, or a pharmaceutically acceptable salt thereof, wherein R₁ is selected from:
(i) —NR_{1A}R_{1B};
wherein one of R_{1A} and R_{1B} hydrogen, and the other is selected from (1-4C)alkyl or phenyl(1-2C)alkyl, each of which being optionally substituted by one or more Rₓ; or
R_{1A} and R_{1B} are linked such that, together with the nitrogen atom to which they are attached, they form a 4-, 5- or 6-membered monocyclic heterocyclic ring, a 6 to 10 membered fused heterocyclic ring, a 6 to 10 membered bridged heterocyclic ring or a 6 to 10 membered spirocyclic heterocyclic ring, each of which being optionally substituted by one or more Rₓ; or
(ii) a carbon-linked a 4-, 5- or 6-membered monocyclic heterocyclic ring, a 6 to 10 membered fused heterocyclic ring, a 6 to 10 membered bridged heterocyclic ring or a 6 to 10 membered spirocyclic heterocyclic ring, each of which being optionally substituted by one or more Rₓ;
(iii) (3C)cycloalkyl optionally substituted by one or two substituents independently selected from fluoro, (1-2C)alkyl, (1-2C)haloalkyl, (1-2C)alkoxy, (1-2C)haloalkoxy, hydroxy, (1-2C)hydroxyalkyl or phenyl;
wherein each Rₓ is independently selected from halo, cyano, (1-4C)alkyl, (CH₂)_{q1}NR_{1D}R_{1E}, (CH₂)_{q1}OR_{1D}, (CH₂)_{q1}C(O)R_{1D}, O(CH₂)_{q1}C(O)R_{1D}, (CH₂)_{q1}C(O)OR_{1D}, (CH₂)_{q1}OC(O)R_{1D}, (CH₂)_{q1}C(O)N(R_{1E})R_{1D}, O(CH₂)_{q1}C(O)N(R_{1E})R_{1D} or (CH₂)_{q1}N(R_{1E})C(O)R_{1D};
wherein q1 is 0 or 1;
wherein R_{1D} is selected from (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
and R_{1E} is selected from hydrogen or (1-2C)alkyl;
and wherein any (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ, R_{1D} and R_{1E} substituent, or a heterocyclyl groups formed when R_{1D} and R_{1E} are linked, is optionally further substituted by halo, cyano, hydroxy, oxo, (1-2C)alkyl, (1-2C)haloalkyl, (1-2C)hydroxyalkyl, NR_{1F}R_{1G}, OR_{1F}, C(O)R_{1F} and C(O)OR_{1F} (e.g. halo, cyano, hydroxy or (1-4C)alkyl);
wherein R_{1F} and R_{1G} are each independently selected from hydrogen or (1-2C)alkyl.

8. A compound according to any one of the preceding claims, or a pharmaceutically acceptable salt thereof, wherein R₁ is selected from:
(i) —NR_{1A}R_{1B};
wherein one of R_{1A} and R_{1B} hydrogen, and the other is selected from (1-4C)alkyl or phenyl(1-2C)alkyl, each of which being optionally substituted by one or more Rₓ; or
R_{1A} and R_{1B} are linked such that, together with the nitrogen atom to which they are attached, they form a 4-, 5- or 6-membered monocyclic heterocyclic ring, a 7 to 9 membered fused heterocyclic ring or a 7 to 9 membered spirocyclic heterocyclic ring, each of which being optionally substituted by one or more Rₓ; or
(ii) a carbon-linked 4-, 5- or 6-membered monocyclic heterocyclic ring, a 7 to 9 membered fused heterocyclic ring or a 7 to 9 membered spirocyclic heterocyclic ring, each of which being optionally substituted by one or more Rₓ;
(iii) (3C)cycloalkyl optionally substituted by one or two substituents independently selected from fluoro, (1-2C)alkyl, (1-2C)haloalkyl, (1-2C)alkoxy, (1-2C)haloalkoxy, hydroxy, (1-2C)hydroxyalkyl or phenyl;
wherein each Rₓ is independently selected from halo, cyano, (1-4C)alkyl, (CH₂)_{q1}OR_{1D}, O(CH₂)_{q1}C(O)R_{1D} or O(CH₂)_{q1}C(O)N(R_{1E})R_{1D}, wherein q1 is 0 or 1;
wherein R_{1D} is selected from (1-4C)alkyl, phenyl, phenyl(1-2C)alkyl, 5- or 6-membered heteroaryl, (5- or 6-membered)heteroaryl(1-2C)alkyl, 4- to 9-membered heterocyclyl, or (4- to 9-membered)heterocyclyl(1-2C)alkyl;
and R_{1E} is selected from hydrogen or methyl;
and wherein any (1-4C)alkyl, phenyl, phenyl(1-2C)alkyl, (5- or 6-membered)heteroaryl, (5- or 6-membered)heteroaryl(1-2C)alkyl, (4- to 9-membered)heterocyclyl or (4- to 9-membered)heterocyclyl(1-2C)alkyl group present in a Rₓ or R_{1D} substituent, or a heterocyclyl groups formed when R_{1D} and R_{1E} are linked, is optionally further substituted by halo, cyano or hydroxy (1-2C)alkyl or (1-2C)haloalkyl.

9. A compound according to any one of the preceding claims, or a pharmaceutically acceptable salt thereof, wherein R₁ is —NR₁AR_{1B};
wherein one of R_{1A} and R_{1B} hydrogen, and the other is other is selected from (1-4C)alkyl or phenyl(1-2C)alkyl, each of which being optionally substituted by one or more Rₓ; or
wherein R_{1A} and R_{1B} are linked such that, together with the nitrogen atom to which they are attached, they form a 4- or 5-membered heterocyclic ring system, a 7 to 9 membered fused heterocyclic ring or a 7 to 9 membered spirocyclic heterocyclic ring each of which being optionally substituted by one or more Rₓ;
wherein each Rₓ is independently selected from halo, cyano, (1-2C)alkyl, OR_{1D}, OC(O)R_{1D} or OC(O)N(R_{1E})R_{1D};
wherein R_{1D} is selected from (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl;
and R_{1E} is selected from hydrogen or methyl;
and wherein any (1-4C)alkyl, phenyl, phenyl(1-3C)alkyl, 5- or 6-membered heteroaryl, (5- or 6-membered)heteroaryl(1-3C)alkyl, 4- to 9-membered heterocyclyl, or (4- to 9-membered)heterocyclyl(1-3C)alkyl group present in a Rₓ or R_{1D} substituent, or a heterocyclyl groups formed when R_{1D} and R_{1E} are linked, is optionally further substituted by halo, cyano or hydroxy (1-2C)alkyl or (1-2C)haloalkyl;
and optionally wherein R_{1A} and R_{1B} are linked such that, together with the nitrogen atom to which they are attached, they form a 4- or 5-membered nitrogen-linked heterocyclic ring, a 7 to 9 membered fused heterocyclic ring or a 7 to 9 membered spirocyclic heterocyclic ring, each of which being optionally substituted by one or more Rₓ;
wherein each Rₓ is independently selected from halo, (1-2C)alkyl, OR_{1D}, OC(O)R_{1D} or OC(O)N(R_{1E})R_{1D};
wherein R_{1D} is selected from (1-4C)alkyl, phenyl(1-2C)alkyl, 6-membered heteroaryl, (5- or 6-membered)heteroaryl(1-2C)alkyl, 6- to 9-membered heterocyclyl, or (6- to 9-membered)heterocyclyl(1-2C)alkyl;
and R_{1E} is hydrogen,
and wherein any (1-4C)alkyl, phenyl(1-2C)alkyl, 5- or 6-membered heteroaryl, (5- or 6-membered)heteroaryl(1-2C)alkyl, 4- to 9-membered heterocyclyl, or (4- to 9-membered)heterocyclyl(1-2C)alkyl group present in a Rₓ or R_{1D} substituent, or a heterocyclyl groups formed when R_{1D} and R_{1E} are linked, is optionally further substituted by halo, cyano or hydroxy (1-2C)alkyl or (1-2C)haloalkyl.

10. A compound according to any one of the preceding claims, or a pharmaceutically acceptable salt thereof, wherein R₁ is a group of the formula: 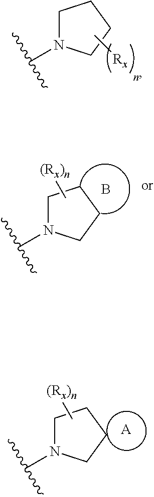
wherein n is 0, 1, 2, 3 or 4; and
each occurrence of Rₓ is independently as defined in any one of the preceding paragraphs;
Ring A, together with the carbon atom to which it is attached, is a spiro-fused 3 to 6 membered cycloalkyl or heterocyclyl ring, optionally substituted by one or more Rₓ; and
Ring B, together with the carbon atoms to which it is attached, is a fused 3 to 6 membered cycloalkyl or heterocyclyl ring, optionally substituted by one or more Rₓ;
optionally wherein R₁ is a group of the formula:

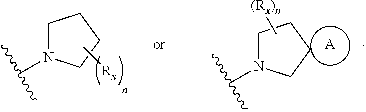

11. A compound according to any one of the preceding claims, or a pharmaceutically acceptable salt thereof, wherein R₁ is a group of the formula: 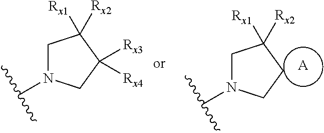
wherein:
Rₓ₁ and Rₓ₂ are independently selected from hydrogen or halo;
Rₓ₃ and Rₓ₄ are independently selected from hydrogen, halo, methyl or methoxy;
or one of Rₓ₃ and Rₓ₄ is hydrogen, and the other is OR_{1D}, wherein R_{1D} is selected from phenyl(1-2C)alkyl, (5- or 6-membered)heteroaryl(1-2C)alkyl, or (4- to 6-membered)heterocyclyl(1-2C)alkyl; wherein the phenyl(1-2C)alkyl, (5- or 6-membered)heteroaryl(1-2C)alkyl, or (4- to 6-membered)heterocyclyl(1-2C)alkyl are each optionally substituted by one or more of halo, cyano, hydroxy or methyl; and
Ring A is a spiro-fused 3 to 6 membered cycloalkyl or heterocyclyl ring, each of which being optionally substituted with one or more substituents selected from halo, methyl or methoxy;
optionally wherein:
Rₓ₁ and Rₓ₂ are independently selected from hydrogen or fluoro;
Rₓ₃ and Rₓ₄ are independently selected from hydrogen, halo or methyl;
or one of Rₓ₃ and Rₓ₄ is hydrogen, and the other is OR_{1D}, wherein R_{1D} is (6-membered)heterocyclyl(1-2C)alkyl; wherein the (6-membered)heterocyclyl(1-2C)alkyl is optionally substituted by one or more of halo, cyano or methyl; and
Ring A is a spiro-fused 3 to 5 membered cycloalkyl or heterocyclyl ring; each of which being optionally substituted with one or more substituents selected from halo, methyl or methoxy.

12. A compound according to any one of claims 1 to 6, or a pharmaceutically acceptable salt thereof, wherein R₁ is selected from: 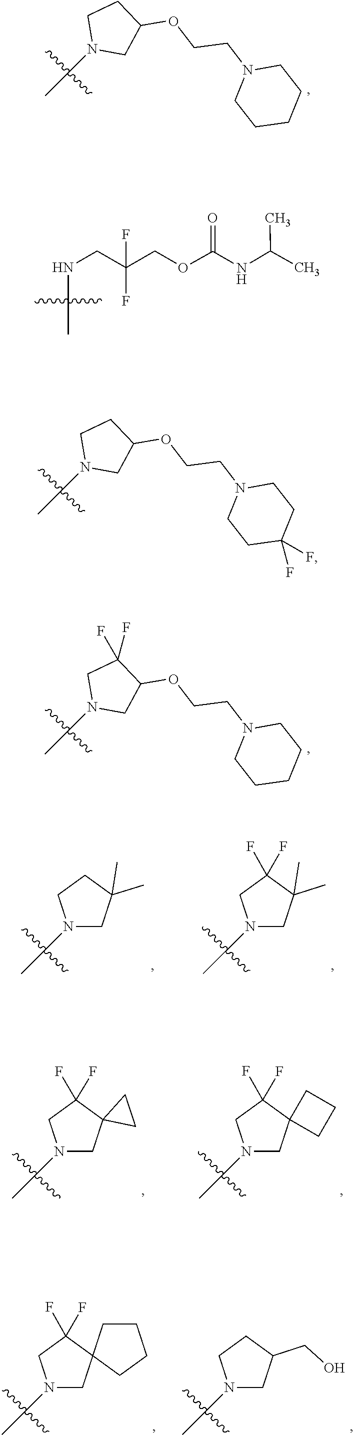 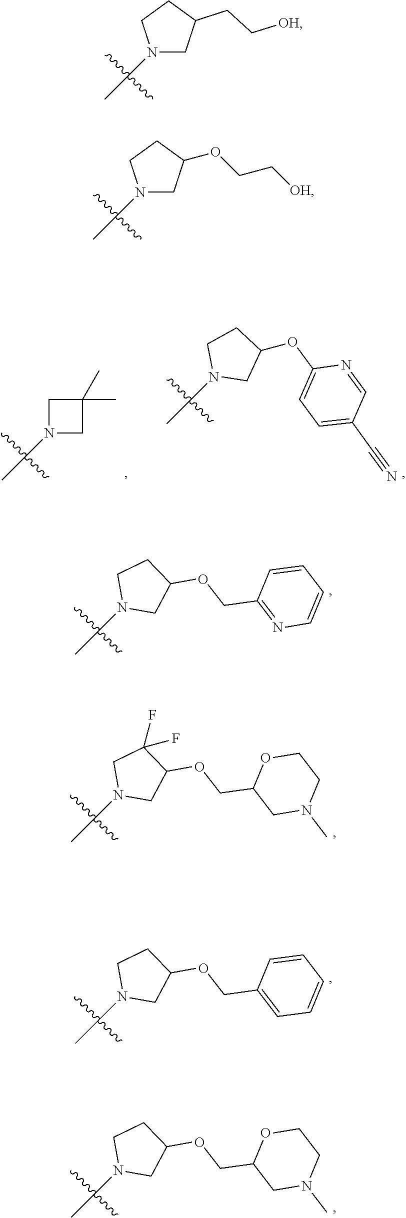 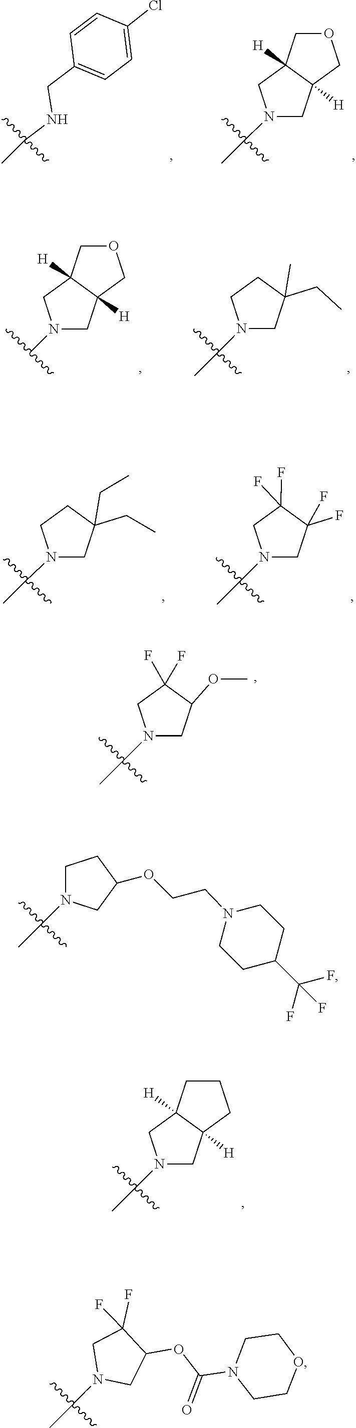 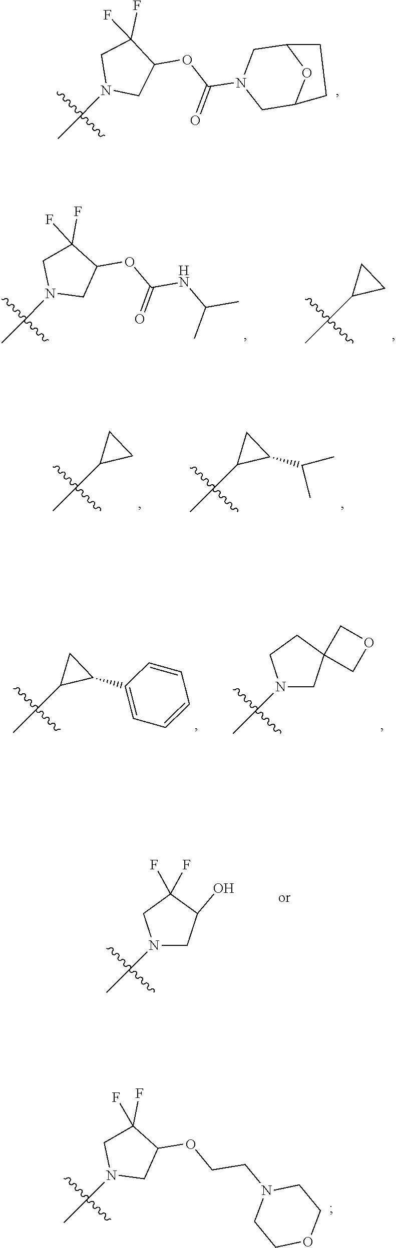
optionally wherein R₁ is selected from:

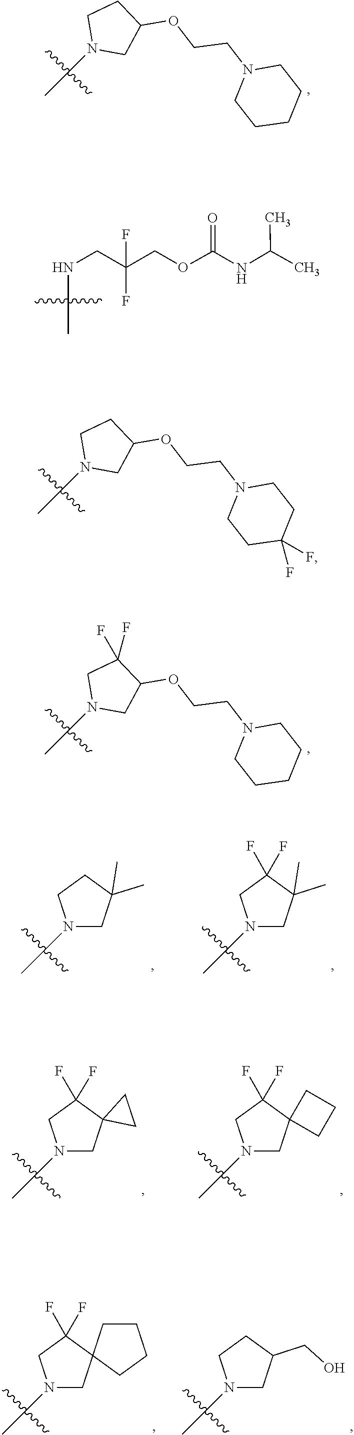

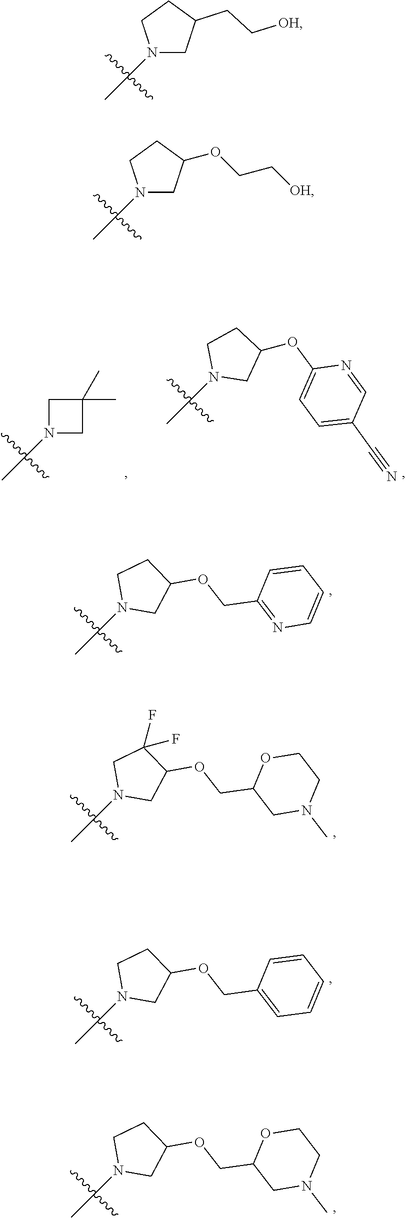

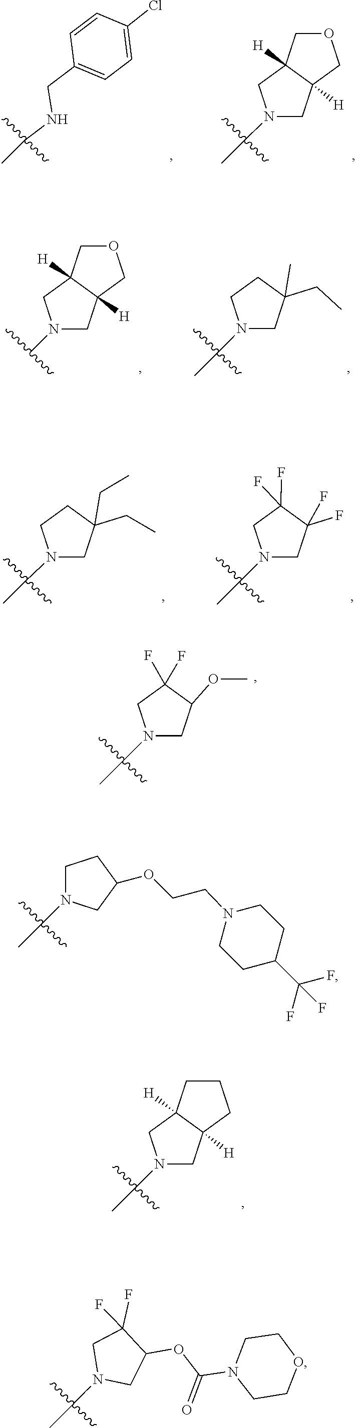

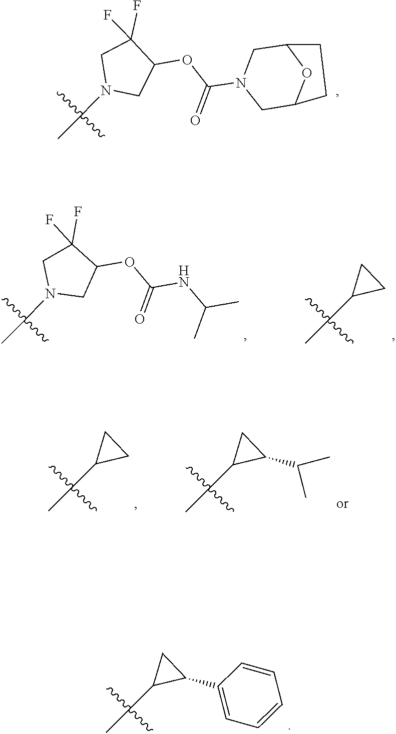

13. A compound according to any one of claims 1 to 6, or a pharmaceutically acceptable salt thereof, wherein R₁ is selected from:

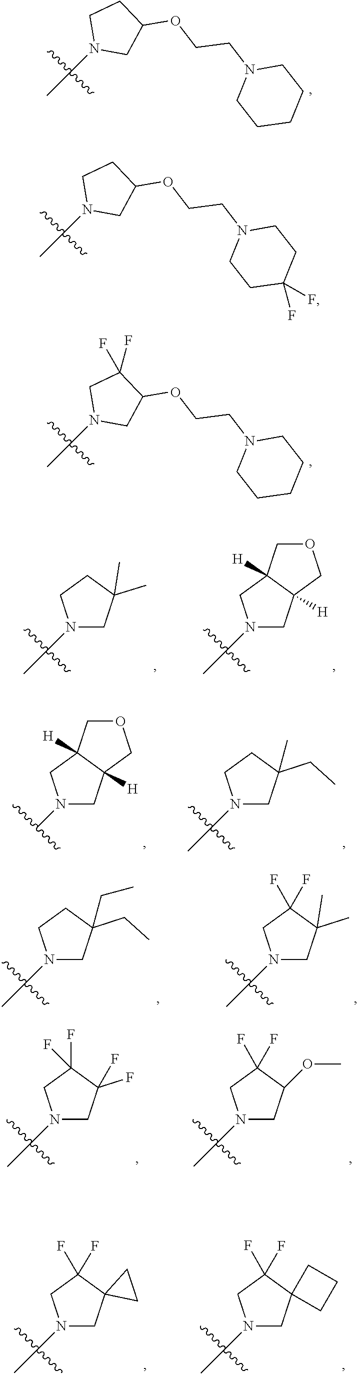

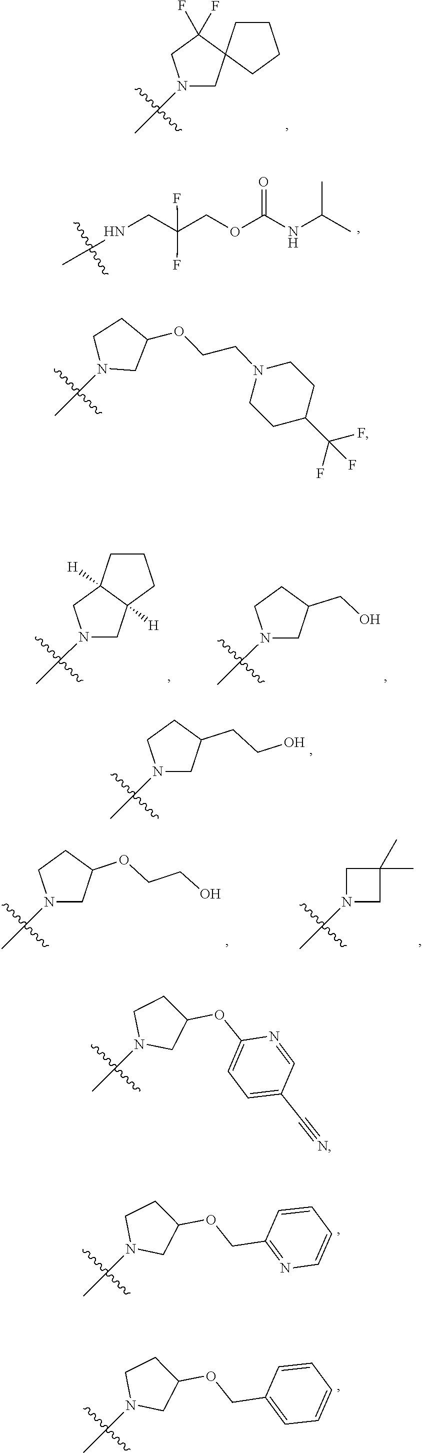

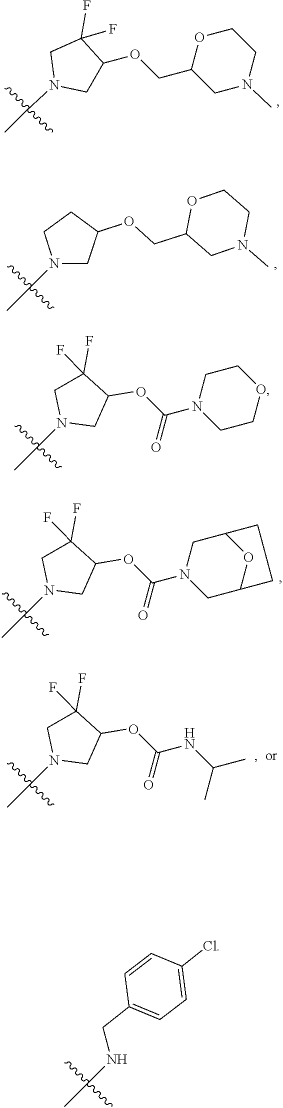 [SMILES: *CN1CC(OCC2CN(C)CCO2)C(F)(F)C1 ; *N1CCC(OCC2CN(C)CCO2)C1 ; *N1CC(OC(=O)N2CCOCC2)C(F)(F)C1 ; *N1CC(OC(=O)N2CC3CCC(C2)O3)C(F)(F)C1 ; *N1CC(OC(=O)NC(C)C)C(F)(F)C1 ; *NCc1ccc(Cl)cc1]

14. A compound according to any one of the preceding claims, or a pharmaceutically acceptable salt thereof, wherein:
X₂ is selected from N or CR₂; wherein R₂ is selected from hydrogen, halo, methyl, methoxy, halomethyl, halomethoxy or cyano;
X₃ is selected from N or CR₃; wherein R₃ is selected from hydrogen, halo methyl, methoxy, halomethyl, halomethoxy or cyano,
X₄ is selected from N or CR₄; wherein R₄ is selected from hydrogen, halo, (1-2C)alkyl, (1-2C)alkoxy, (1-2C)haloalkyl, (1-2C)haloalkoxy or cyano;
X₅ is selected from N or C;
X₆ is selected from N or C;
X₇ is selected from N, NR_{7N}, CR₇, O or S; wherein:
R₇ is selected from hydrogen, halo, methyl or methoxy;
R_{7N} is selected from hydrogen or methyl;
X₈ is selected from N, NR_{8N}, CR₈, O or S, wherein:
R₈ is selected from hydrogen or halo;
R_{8N} is selected from hydrogen or methyl;
X₉ is selected from N or C;
with the proviso that:
(i) X₅ and X₆ cannot both be N;
(ii) only one of X₇ and X₈ can be O or S; and
(iii) no more than four of the atoms in groups X₂, X₃, X₄, X₅, X₆, X₇, X₈ and X₉ which together with the carbon atom attached to R₁, make up the bicyclic ring, may be a heteroatom;
optionally wherein:
X₂ is selected from N or CR₂; wherein R₂ is selected from hydrogen or cyano;
X₃ is selected from N or CR₃; wherein R₃ is selected from hydrogen, halo or cyano, X₄ is selected from N or CR₄; wherein R₄ is selected from hydrogen, fluoro, chloro, methyl, methoxy, fluoromethyl (e.g. CH₂F, CHF₂ or CF₃) or cyano;
X₅ is C;
X₆ is selected from N or C;
X₇ is selected from N, CR₇, or S, wherein R₇ is selected from hydrogen, halo or methyl;
X₈ is selected from N, NR_{8N}, CR₈, O or S; wherein:
R₈ is selected from hydrogen or halo;
R_{8N} is hydrogen or methyl; and
X₉ is selected from N or C.

15. A compound according to any one of the preceding claims, or a pharmaceutically acceptable salt thereof, wherein:
X₂ is N;
X₃ is CR₃, wherein R₃ is selected from hydrogen, halo or cyano,
X₄ is selected from N or CR₄; wherein R₄ is selected from hydrogen, chloro or fluoro (e.g. hydrogen or fluoro);
X₅ is C;
X₆ is C or N;
X₇ is CR₇; wherein R₇ is hydrogen:
X₈ is selected from N, CR₈ or S, wherein R₈ is hydrogen
X₉ is selected from N or C;
optionally wherein:
X₂ is N;
X₃ is CR₃, wherein R₃ is selected from hydrogen, halo or cyano,
X₄ is N or CR₄; wherein R₄ is selected from hydrogen, chloro or fluoro (e.g. hydrogen or fluoro);
X₅ is C;
X₆ is C or N;
X₇ is CR₇; wherein R₇ is hydrogen:
X₈ is N;
X₉ is selected from N or C;
further optionally wherein:
X₂ is N;
X₃ is CR₃, wherein R₃ is hydrogen,
X₄ is CR₄; wherein R₄ is selected from hydrogen, chloro or fluoro;
X₅ is C;
X₆ is N;
X₇ is CR₇; wherein R₇ is hydrogen:
X₈ is N;
X₉ is C.

16. A compound of the formula Ia, Ib, Ic, Id, Ie, If, Ig, Ih, Ii, Ij, Ik, Im, In, Io or Ip: 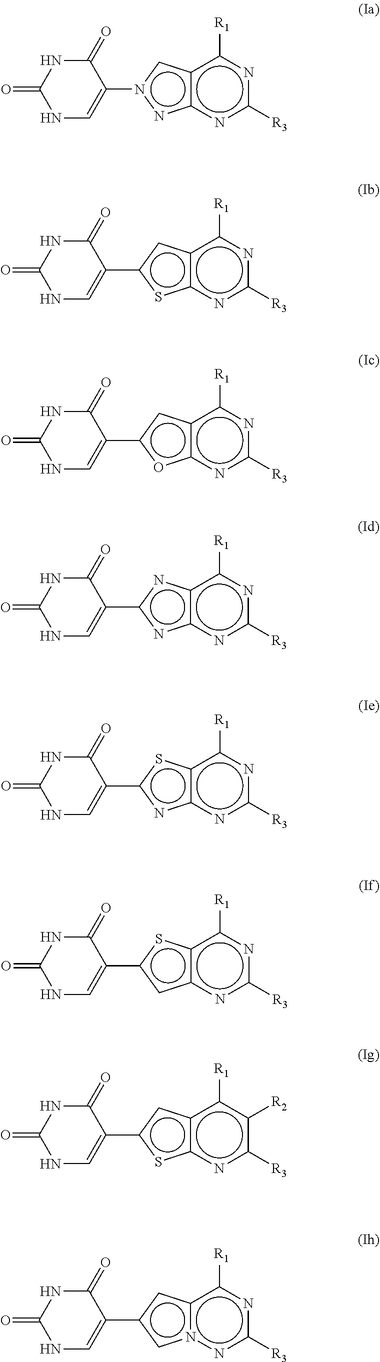 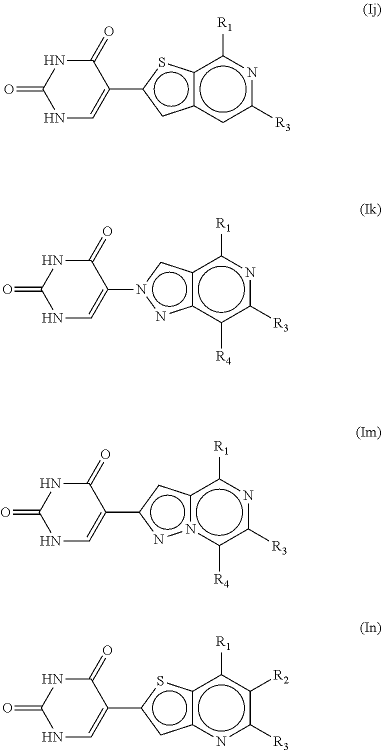
wherein R₁, R₂, R₃ and R₄ are as defined in any one of of the preceding claims; or a pharmaceutically acceptable salt thereof.

17. A compound, or a pharmaceutically acceptable salt thereof, selected from any one of the following:
5-[4-[3-(Hydroxymethyl)pyrrolidin-1-yl]thieno[2,3-d]pyrimidin-6-yl]-1H-pyrimidine-2,4-dione;
5-[4-[3-(2-Hydroxyethyl)pyrrolidin-1-yl]thieno[2,3-d]pyrimidin-6-yl]-1H-pyrimidine-2,4-dione;
5-[4-(3,3-Dimethylazetidin-1-yl)thieno[2,3-d]pyrimidin-6-yl]-1H-pyrimidine-2,4-dione 5-[4-[3-(2-Hydroxyethoxy)pyrrolidin-1-yl]thieno[2,3-d]pyrimidin-6-yl]-1H-pyrimidine-2,4-dione;
5-[4-[3-[2-(1-Piperidyl)ethoxy]pyrrolidin-1-yl]thieno[2,3-d]pyrimidin-6-yl]-1H-pyrimidine-2,4-dione;
5-[4-(3,3-Dimethylpyrrolidin-1-yl)thieno[2,3-d]pyrimidin-6-yl]-1H-pyrimidine-2,4-dione;
6-[1-[6-(2,4-Dioxo-1H-pyrimidin-5-yl)thieno[2,3-d]pyrimidin-4-yl]pyrrolidin-3-yl]oxypyridine-3-carbonitrile;
5-[4-(3,3-Difluoro-4,4-dimethyl-pyrrolidin-1-yl)thieno[2,3-d]pyrimidin-6-yl]-1H-pyrimidine-2,4-dione;
5-[4-[3-(2-pyridylmethoxy)pyrrolidine-1-yl]thieno[2,3-d]pyrimidin-6-yl]-1H-pyrimidine-2,4-dione;
5-[4-(3-Benzyloxypyrrolidin-1-yl)thieno[2,3-d]pyrimidin-6-yl]-1H-pyrimidine-2,4-dione;
5-[4-[3,3-Difluoro-4-[(4-methylmorpholin-2-yl)methoxy]pyrrolidine-1-yl]thieno[2,3-d]pyrimidin-6-yl]-1H-pyrimidine-2,4-dione;
5-[4-[3-[(4-Methylmorpholin-2-yl)methoxy]pyrrolidine-1-yl]thieno[2,3-d]pyrimidin-6-yl]-1H-pyrimidine-2,4-dione;
5-[4-[3,3-Difluoro-4-[2-(1-piperidyl)ethoxy]pyrrolidine-1-yl]thieno[2,3-d]pyrimidin-6-yl]-1H-pyrimidine-2,4-dione;
5-[7-[3-[2-(1-Piperidyl)ethoxy]pyrrolidin-1-yl]thiazolo[4,5-d]pyrimidin-2-yl]-1H-pyrimidine-2,4-dione;
5-[7-(3,3-difluoro-4,4-dimethyl-pyrrolidin-1-yl)thiazolo[4,5-d]pyrimidin-2-yl]-1H-pyrimidine-2,4-dione;
5-[4-[3-[2-(1-Piperidyl)ethoxy]pyrrolidin-1-yl]furo[2,3-d]pyrimidin-6-yl]-1H-pyrimidine-2,4-dione;
5-[4-(3,3-Difluoro-4,4-dimethyl-pyrrolidin-1-yl)furo[2,3-d]pyrimidin-6-yl]-1H-pyrimidine-2,4-dione;
5-[4-[3-[2-(1-Piperidyl)ethoxy]pyrrolidin-1-yl]thieno[3,2-d]pyrimidin-6-yl]-1H-pyrimidine-2,4-dione;
5-[4-[3-[2-(1-Piperidyl)ethoxy]pyrrolidin-1-yl]thieno[3,2-c]pyridin-2-yl]-1H-pyrimidine-2,4-dione;
5-[4-[3-[2-(1-Piperidyl)ethoxy]pyrrolidin-1-yl]pyrrolo[2,1-f][1,2,4]triazin-6-yl]pyrimidine-2,4-diol;
5-[4-[3-[2-(4,4-Difluoro-1-piperidyl)ethoxy]pyrrolidin-1-yl]pyrrolo[2,1-f][1,2,4]triazin-6-yl]-1H-pyrimidine-2,4-dione;
5-[4-(3,3-Difluoro-4,4-dimethyl-pyrrolidin-1-yl)pyrrolo[2,1-f][1,2,4]triazin-6-yl]-1H-pyrimidine-2,4-dione;
2-(2,4-Dioxo-1H-pyrimidin-5-yl)-4-[3-[2-(1-piperidyl)ethoxy]pyrrolidin-1-yl]thieno[2,3-b]pyridine-5-carbonitrile;
5-[4-[3-[2-(1-Piperidyl)ethoxy]pyrrolidin-1-yl]pyrazolo[3,4-d]pyrimidin-2-yl]-1H-pyrimidine-2,4-dione;
5-[4-(3,3-Difluoro-4,4-dimethyl-pyrrolidin-1-yl)pyrazolo[3,4-d]pyrimidin-2-yl]-1H-pyrimidine-2,4-dione;
5-[4-(7,7-Difluoro-5-azaspiro[2.4]heptan-5-yl)pyrazolo[3,4-d]pyrimidin-2-yl]-1H-pyrimidine-2,4-dione;
5-[4-(8,8-Difluoro-6-azaspiro[3.4]octan-6-yl)pyrazolo[3,4-d]pyrimidin-2-yl]-1H-pyrimidine-2,4-dione;
5-[4-(4,4-Difluoro-2-azaspiro[4.4]nonan-2-yl)pyrazolo[3,4-d]pyrimidin-2-yl]-1H-pyrimidine-2,4-dione;
5-[4-[(3aR,6aS)-3,3a,4,5,6,6a-Hexahydro-1H-cyclopenta[c]pyrrol-2-yl]pyrazolo[3,4-d]pyrimidin-2-yl]-1H-pyrimidine-2,4-dione;
5-[4-(3-Ethyl-3-methyl-pyrrolidin-1-yl)pyrazolo[3,4-d]pyrimidin-2-yl]-1H-pyrimidine-2,4-dione;
5-[4-(3,3-Dimethylpyrrolidin-1-yl)pyrazolo[3,4-d]pyrimidin-2-yl]-1H-pyrimidine-2,4-dione;
5-[4-(3,3-Diethylpyrrolidin-1-yl)pyrazolo[3,4-d]pyrimidin-2-yl]-1H-pyrimidine-2,4-dione;
5-[4-(3,3,4,4-Tetrafluoropyrrolidin-1-yl)pyrazolo[3,4-d]pyrimidin-2-yl]-1H-pyrimidine-2,4-dione;
5-[4-[(3aR,6aS)-1,3,3a,4,6,6a-Hexahydrofuro[3,4-c]pyrrol-5-yl]pyrazolo[3,4-d]pyrimidin-2-yl]-1H-pyrimidine-2,4-dione;
5-[4-[(3aR,6aR)-1,3,3a,4,6,6a-Hexahydrofuro[3,4-c]pyrrol-5-yl]pyrazolo[3,4-d]pyrimidin-2-yl]-5H-pyrimidine-2,4-dione;
5-[4-(3,3-Difluoro-4-methoxy-pyrrolidin-1-yl)pyrazolo[3,4-d]pyrimidin-2-yl]-1H-pyrimidine-2,4-dione;
5-[4-[3-[2-(1-Piperidyl)ethoxy]pyrrolidin-1-yl]thieno[2,3-b]pyridin-2-yl]-1H-pyrimidine-2,4-dione;
5-[4-(3,3-Difluoro-4,4-dimethyl-pyrrolidin-1-yl)thieno[2,3-b]pyridin-2-yl]-1H-pyrimidine-2,4-dione;
5-[6-[3-[2-(1-Piperidyl)ethoxy]pyrrolidin-1-yl]-9H-purin-8-yl]-1H-pyrimidine-2,4-dione;
5-[7-[3-[2-(1-Piperidyl)ethoxy]pyrrolidin-1-yl]thieno[3,2-b]pyridin-2-yl]-1H-pyrimidine-2,4-dione;
5-[4-[3,3-Difluoro-4-[2-(1-piperidyl)ethoxy]pyrrolidin-1-yl]pyrrolo[2,1-f][1,2,4]triazin-6-yl]-1H-pyrimidine-2,4-dione;
5-[2-Chloro-4-[3-[2-(1-piperidyl)ethoxy]pyrrolidin-1-yl]pyrrolo[2,1-f][1,2,4]triazin-6-yl]-5H-pyrimidine-2,4-dione;
5-[4-(8,8-Difluoro-6-azaspiro[3.4]octan-6-yl)furo[2,3-d]pyrimidin-6-yl]-1H-pyrimidine-2,4-dione;
5-[4-(4,4-Difluoro-2-azaspiro[4.4]nonan-2-yl)furo[2,3-d]pyrimidin-6-yl]-1H-pyrimidine-2,4-dione;
5-[4-[(4-Chlorophenyl)methylamino]furo[2,3-d]pyrimidin-6-yl]-1H-pyrimidine-2,4-dione;
5-[4-(7,7-Difluoro-5-azaspiro[2.4]heptan-5-yl)furo[2,3-d]pyrimidin-6-yl]-1H-pyrimidine-2,4-dione;
[3-[[6-(2,4-Dioxo-1H-pyrimidin-5-yl)furo[2,3-d]pyrimidin-4-yl]amino]-2,2-difluoro-propyl]N-isopropylcarbamate;
5-[4-[3-[2-[4-(Trifluoromethyl)-1-piperidyl]ethoxy]290yrrolidine-1-yl]furo[2,3-d]pyrimidin-6-yl]-1H-pyrimidine-2,4-dione;
[1-[6-(2,4-Dioxo-1H-pyrimidin-5-yl)furo[2,3-d]pyrimidin-4-yl]-4,4-difluoro-pyrrolidin-3-yl]morpholine-4-carboxylate;
[1-[6-(2,4-Dioxo-1H-pyrimidin-5-yl)furo[2,3-d]pyrimidin-4-yl]-4,4-difluoro-pyrrolidin-3-yl]8-oxa-3-azabicyclo[3.2.1]octane-3-carboxylate;
[1-[6-(2,4-Dioxo-1H-pyrimidin-5-yl)furo[2,3-d]pyrimidin-4-yl]-4,4-difluoro-pyrrolidin-3-yl]N-isopropylcarbamate;
5-[4-(3,3-Difluoro-4,4-dimethyl-pyrrolidin-1-yl)-6-methyl-pyrazolo[3,4-d]pyrimidin-2-yl]-1H-pyrimidine-2,4-dione;
6-[1-[2-(2,4-Dioxo-1H-pyrimidin-5-yl)pyrazolo[3,4-d]pyrimidin-4-yl]290yrrolidine-3-yl]oxypyridine-3-carbonitrile;
5-[4-(3,3-Difluoro-4,4-dimethyl-pyrrolidin-1-yl)pyrazolo[1,5-a]pyrazin-2-yl]-1H-pyrimidine-2,4-dione;
5-[4-(8,8-Difluoro-6-azaspiro[3.4]octan-6-yl)pyrazolo[1,5-a]pyrazin-2-yl]-1H-pyrimidine-2,4-dione;
5-[4-(7,7-Difluoro-5-azaspiro[2.4]heptan-5-yl)pyrazolo[1,5-a]pyrazin-2-yl]-1H-pyrimidine-2,4-dione;
5-[4-(3,3-difluoro-4-methoxy-pyrrolidin-1-yl)pyrazolo[1,5-a]pyrazin-2-yl]-1H-pyrimidine-2,4-dione;
5-[4-(2-oxa-7-azaspiro[3.4]octan-7-yl)pyrazolo[1,5-a]pyrazin-2-yl]-1H-pyrimidine-2,4-dione;
5-[4-(3,3-difluoro-4-hydroxy-pyrrolidin-1-yl)pyrazolo[1,5-a]pyrazin-2-yl]-1H-pyrimidine-2,4-dione;
5-[4-[3,3-difluoro-4-[(4-methylmorpholin-2-yl)methoxy]pyrrolidin-1-yl]pyrazolo[1,5-a]pyrazin-2-yl]-1H-pyrimidine-2,4-dione;
5-[4-[3,3-difluoro-4-(2-morpholinoethoxy)pyrrolidin-1-yl]pyrazolo[1,5-a]pyrazin-2-yl]-1H-pyrimidine-2,4-dione;
5-[7-chloro-4-(3,3-difluoro-4,4-dimethyl-pyrrolidin-1-yl)pyrazolo[1,5-a]pyrazin-2-yl]-1H-pyrimidine-2,4-dione;
5-[4-(3,3-difluoro-4,4-dimethyl-pyrrolidin-1-yl)-7-(trifluoromethyl)pyrazolo[1,5-a]pyrazin-2-yl]-1H-pyrimidine-2,4-dione;
5-[4-(3,3-difluoro-4,4-dimethyl-pyrrolidin-1-yl)-7-fluoro-pyrazolo[1,5-a]pyrazin-2-yl]-1H-pyrimidine-2,4-dione;
5-[4-(3,3-difluoro-4,4-dimethyl-pyrrolidin-1-yl)-7-methyl-pyrazolo[1,5-a]pyrazin-2-yl]-1H-pyrimidine-2,4-dione;
5-[4-(3,3-Difluoro-4,4-dimethyl-pyrrolidin-1-yl)-7-fluoro-pyrazolo[4,3-c]pyridin-2-yl]-1H-pyrimidine-2,4-dione;
5-[4-(8,8-difluoro-6-azaspiro[3.4]octan-6-yl)-7-fluoro-pyrazolo[4,3-c]pyridin-2-yl]-1H-pyrimidine-2,4-dione;
5-[4-(3,3-difluoro-4-methoxy-pyrrolidin-1-yl)-7-fluoro-pyrazolo[4,3-c]pyridin-2-yl]-1H-pyrimidine-2,4-dione;
5-[4-(3,3-difluoro-4-hydroxy-pyrrolidin-1-yl)-7-fluoro-pyrazolo[4,3-c]pyridin-2-yl]-1H-pyrimidine-2,4-dione;
5-[7-fluoro-4-(2-oxa-7-azaspiro[3.4]octan-7-yl)pyrazolo[4,3-c]pyridin-2-yl]-1H-pyrimidine-2,4-dione;
5-[4-(7,7-difluoro-5-azaspiro[2.4]heptan-5-yl)-7-fluoro-pyrazolo[4,3-c]pyridin-2-yl]-1H-pyrimidine-2,4-dione;
5-[4-[3,3-difluoro-4-[(4-methylmorpholin-2-yl)methoxy]pyrrolidin-1-yl]-7-fluoro-pyrazolo[4,3-c]pyridin-2-yl]-1H-pyrimidine-2,4-dione;
5-[4-(3,3-Difluoro-4,4-dimethyl-pyrrolidin-1-yl)pyrazolo[4,3-c]pyridin-2-yl]-1H-pyrimidine-2,4-dione;
5-[7-chloro-4-(3,3-difluoro-4,4-dimethyl-pyrrolidin-1-yl)pyrazolo[4,3-c]pyridin-2-yl]-1H-pyrimidine-2,4-dione;
5-[7-chloro-4-(7,7-difluoro-5-azaspiro[2.4]heptan-5-yl)pyrazolo[4,3-c]pyridin-2-yl]-1H-pyrimidine-2,4-dione;
5-[7-chloro-4-(8,8-difluoro-6-azaspiro[3.4]octan-6-yl)pyrazolo[4,3-c]pyridin-2-yl]-1H-pyrimidine-2,4-dione;
4-(3,3-difluoro-4,4-dimethyl-pyrrolidin-1-yl)-2-(2,4-dioxo-1H-pyrimidin-5-yl)pyrazolo[4,3-c]pyridine-7-carbonitrile;
5-[4-(3,3-difluoro-4,4-dimethyl-pyrrolidin-1-yl)pyrazolo[3,4-b]pyridine-2-yl]-1H-pyrimidine-2,4-dione;
5-[4-(3,3-difluoro-4,4-dimethyl-pyrrolidin-1-yl)pyrazolo[3,4-c]pyridine-2-yl]-1H-pyrimidine-2,4-dione;
5-4-(3,3-difluoro-4,4-dimethyl-pyrrolidin-1-yl)-7-iodo-pyrazolo[1,5-a]pyrazin-2-yl]-1H-pyrimidine-2,4-dione;
5-[7-Chloro-4-(3,3-difluoro-4-hydroxy-pyrrolidin-1-yl)pyrazolo[1,5-a]pyrazin-2-yl]-1H-pyrimidine-2,4-dione;
5-[7-Chloro-4-(3,3-difluoro-4-methoxy-pyrrolidin-1-yl)pyrazolo[1,5-a]pyrazin-2-yl]-1H-pyrimidine-2,4-dione;
5-[7-Chloro-4-(3,3-difluoro-4,4-dimethyl-pyrrolidin-1-yl)-6-methyl-pyrazolo[1,5-a]pyrazin-2-yl]-1H-pyrimidine-2,4-dione;
5-[7-chloro-4-(3,3-dimethylpyrrolidin-1-yl)pyrazolo[1,5-a]pyrazin-2-yl]-1H-pyrimidine-2,4-dione; and
5-[4-(3,3-dimethylpyrrolidin-1-yl)-7-fluoro-pyrazolo[1,5-a]pyrazin-2-yl]-1H-pyrimidine-2,4-dione.

18. A pharmaceutical composition comprising a compound according to any one of claims 1 to 17, or a pharmaceutically acceptable salt thereof, in admixture with a pharmaceutically acceptable diluent or carrier.

19. A compound according to any one of claims 1 to 17, or a pharmaceutically acceptable salt thereof, or a pharmaceutical composition according to claim 18, for use in therapy.

20. A compound according to any one of claims 1 to 17, or a pharmaceutically acceptable salt thereof, or a pharmaceutical composition according to claim 18, for use:
(i) in the treatment of a proliferative condition;
(ii) in the treatment of cancer;
(iii) in the treatment of cancer, wherein the compound or pharmaceutical composition is administered in combination with one or more additional anticancer agents;
(iv) in the treatment of cancer, wherein the compound or pharmaceutical composition is administered in combination with one or more additional anticancer agents selected from the group consisting of:
1) other forms of cancer immunotherapy and anti-cancer chemotherapeutic agents;
2) A2b antagonists;
3) anti-PD-1 and PDL-1 antibodies (e.g. pembrolizumab, nivolumab, durvalumab, avelumab and atezolizumab); and
4) anti-CTLA4 antibodies (e.g ipilimumab).

21. A method of treating a proliferative disorder in a patient in need of such treatment, the method comprising administering a therapeutically effective amount of a compound according to any one of claims 1 to 17, or a pharmaceutically acceptable salt thereof, or a pharmaceutical composition according to claim 18.

22. A method of treating cancer in a patient in need of such treatment, the method comprising administering a therapeutically effective amount of a compound according to any one of claims 1 to 17, or a pharmaceutically acceptable salt thereof, or a pharmaceutical composition according to claim 18.

23. A method of treating a proliferative disorder in a patient in need of such treatment, the method comprising administering a therapeutically effective amount of a compound according to any one of claims 1 to 17, or a pharmaceutically acceptable salt thereof, or a pharmaceutical composition according to claim 18 in combination with one or more additional anticancer agents.

24. A method according to claim 23, wherein the one or more additional anticancer agents is selected from:
1) other forms of cancer immunotherapy and anti-cancer chemotherapeutic agents;
2) A2b antagonists;
3) anti-PD-1 and PDL-1 antibodies (e.g. pembrolizumab, nivolumab, durvalumab, avelumab and atezolizumab); and
4) anti-CTLA4 antibodies (e.g ipilimumab).
